(19) United States
(12) Patent Application Publication
(10) Pub. No.: US 2025/0179143 A1
(43) Pub. Date: Jun. 5, 2025

(54) SYSTEMS AND METHODS FOR CHAPERONE-MEDIATED LIGAND EXCHANGE ON MHC-I AND MHC-RELATED MOLECULES USING CHICKEN TAPBPR

(71) Applicants: The Children's Hospital of Philadelphia, Philadelphia, PA (US); The Board of Trustees of the University of Illinois, Urbana, IL (US); The Trustees of the University of Pennsylvania, Philadelphia, PA (US)

(72) Inventors: Nikolaos SGOURAKIS, Philadelphia, PA (US); George BURSLEM, Philadelpha, PA (US); Erik PROCKO, Urbana, IL (US); Christine A. DEVLIN, Urbana, IL (US)

(21) Appl. No.: 18/840,805

(22) PCT Filed: Feb. 22, 2023
(86) PCT No.: PCT/US2023/013605

§ 371 Date: Aug. 22, 2024

Related U.S. Application Data
(60) Provisional application No. 63/312,599, filed on Feb. 22, 2022.
(60) Provisional application No. 63/368,069, filed on Jul. 11, 2022.
(60) Provisional application No. 63/373,932, filed on Aug. 30, 2022.

(51) Int. Cl. C07K 14/705; A61K 39/00; A61K 39/385

(52) U.S. Cl. CPC C07K 14/705; A61K 39/385; A61K 2039/5154; A61K 2039/605; C07K 2319/03; C07K 2319/33

(57) ABSTRACT

This invention relates to ligand exchange proteins comprising the luminal domain of TAP-binding protein-related (TAPBPR), which functions as a MHC class I ligand-exchange catalyst when presented to mammalian cells either as a soluble extracellular protein or as a membrane bound cell surface protein. This may be useful in modulating immune responses, including for example loading immunogenic ligand onto tumors or other disease cells to induce their recognition by T cells. Ligand-exchange proteins and methods for their use are provided. The invention further relates to an approach for generating conditional peptide ligands for a range of disease-related MHC-I allotypes. The present invention relates to chicken and human TAPBPRs and tapasins as well as orthologs thereof, derivatives thereof, and any mutants thereof as well as any combinations thereof. The present invention also relates to placeholder conditional ligands for ligand-exchange reactions across multiple HLA allotypes.

### RELATED APPLICATIONS AND INCORPORATION BY REFERENCE

This application is a National Stage of International Application No. PCT/US2023/013605, filed Feb. 22, 2023, which claims benefit of and priority to Ser. Nos. 63/373,932 filed 30 Aug. 2022, 63/368,069 filed 11 Jul. 2022 and 63/312,599 filed 22 Feb. 2022. Reference is made to commonly-owned application Ser. No. 63/312,584, filed 22 Feb. 2022, entitled “Systems and Methods for Generating Chimeric Major Histocompatibility Complex (MHC) Molecules with Desired Peptide-Binding Specificities.” Reference is also made to Ser. No. 63/269,962 filed 25 Mar. 2022. Reference is made to US Patent Publication Nos. US 2021/0052695, US 2021/0155670, US 2021/0269503 and US 2021/0371498 A1; U.S. Pat. No. 10,816,543 and International Patent Publication Nos. WO 2019/145509, WO 2020/010261 and WO 2021/138688.

The foregoing patent publications and applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### FEDERAL FUNDING LEGEND

This invention was made with government support under Grant Nos. AI143997, GM125034, and GM142505 awarded by the National Institutes of Health. The government has certain rights in the invention.

### FIELD OF THE INVENTION

The present invention relates to ligand exchange catalysts and their use in modulating the peptide repertoire displayed by MHC class I and MHC-related molecules on the surface of mammalian cells.

### BACKGROUND OF THE INVENTION

Class I MHC (MHC-I) proteins are expressed in all nucleated cells, and they are implicated in aspects of most, if not all, adaptive immune responses. They function by detecting aberrantly expressed proteins and alerting the immune system to the presence of intracellular threats by interacting with specialized receptors on T cells and Natural Killer (NK) cells (Rossjohn et al. 2015). There are thousands of different allotypes of class I HLA proteins (Human Leucocyte Antigen, the human MHC), encoded by the HLA locus found at the short arm of chromosome 6, and clear disease associations with specific HLA alleles. Classical HLA genes are further classified in 3 sub-classes HLA-A, HLA-B and HLA-C (Vita et al. 2019). An individual's genotype therefore comprises 6 class-I HLA genes, which, given the highly polymorphic nature of the MHC-I peptide binding groove, can create an unlimited number of combinations at the population level, ensuring species adaptability to emerging pathogenic strains. HLA allotypes can be further grouped into supertypes, according to their peptide binding specificities (Sidney et al. 2008). The repertoire of peptides which can bind to a given HLA allotype is often represented in compact form as a sequence “logo”, where for each position P1 . . . P9 the relative frequency of different amino acid types is shown (Schneider and Stephens, 1990). An in silico functional clustering and classification of 121 common HLA-A, -B and -C allotypes based on their corresponding peptide sequence logo was performed by Rassmusen et al. Feb. 10, 2022 6:15:00 PM. This analysis has shown a wide distribution of peptides which can be displayed by different HLA molecules including charged, polar or hydrophobic amino acids and their combinations at defined anchor positions, indicating that the MHC-I structure can accommodate a diverse set of peptide sequences.

In addition, cells express non-classical MHC-I and MHC-I like molecules which play critical roles in immune surveillance against infectious diseases and cancer. For instance, in an analogous process to MHC-I, the non-classical MHC-I-related protein 1 (MR1) presents small molecule ligands derived from the endogenous and exogenous metabolome to innate-like mucosal-associated invariant T (MAIT) or MR1-restricted T (MR1T) lymphocytes (Corbett et al., 2020). While their function in the immune system remains to be fully characterized, MR1 is known to be important for recognition of microbial infections, distinguishing cancerous from healthy cells and regulation of autoimmune disease (Crowther et al., 2020, Chen et al., 2017, Rouxel et al., 2017). Known MR1 ligands include vitamin B9 metabolites, such as 6-formylpterin/acetyl-6-formylpterin (6-FP/Ac-6-FP), vitamin B2 metabolites, such as 5-(2-oxopropylideneamino)-6-D-ribitylaminouracil (5-OP-RU), and drug-like compounds (Kjer-Nielsen et al., 2012, Keller et al., 2017, Salio et al., 2020). An important feature of the pyrimidine- (e.g. 5-OP-RU) and pterin-based potent antigens (e.g. 6-FP) is their covalent association with the MR1 groove via Schiff base formation to the sidechain of K43 (Kjer-Nielsen et al., 2012, Corbett et al., 2020). Weaker antigens, such as ribityl lumazine derivatives and the drug diclofenac (DCF), are known to associate non-covalently with the A′ pocket leading to reduced fold stability and may downregulate MR1 surface expression through competition with other ligands (Keller et al., 2017, Salio et al., 2020). Recent studies suggest that MR1 can display a much broader range of ligands, including gut microbial and cancer-specific metabolites which could serve as internal sensors for disease (Lepore et al., 2017). Other non-classical MHC molecules include HLA-E, HLA-G which bind peptides, CD1 which binds lipids, endothelial protein C receptor (EPCR) which binds Phospholipids and Zn-a glycoprotein (ZAG) which binds fatty acids. Since non-classical MHC-I molecules are relatively monomorphic in the population, they provide targets for therapeutic development that would maximize patient coverage.

Ligand exchange technologies are essential for the generation of peptide:HLA tetramers which are commonly used as a screening tool to probe diverse, polyclonal T cell repertoires in various disease settings, as well as for the development of pHLA-targeted therapeutics such as single-chain antibody fragments (scFVs) with minimum off-target cross-reactivity (Altman et al. 1996). Alternatively, exchange of peptide ligands on the cell surface can be used to bypass the endogenous MHC-I antigen processing pathway, and methods to promote exchange have important ramifications for developing systems of antigen-presenting cells (APCs) for T cell expansion in vitro, an essential component of autologous T cell therapy. Previous approaches employ a range of methods to perform peptide exchange on recombinantly produced HLA molecules in vitro. Recently, a peptide exchange methodology using the human molecular chaperone TAPBPR (Boyle et al. 2013; Teng et al. 2002) has provided a valuable tool for generating p:HLA libraries (Overall et al. 2020; O'Rourke et al. 2019). However, its application has been limited to HLA molecules belonging to the A02, A68 and A24 supertypes. Similarly, the use of human TAPBPR to promote peptide loading on the surface of antigen presenting cells, although previously described, is subject to the same limitations (Ilca et al. 2018). With respect to MR1 and CD1 molecules there exists a large, unmet need for high-throughput ligand exchange technologies, both for generating screening reagents (e.g. tetramers) and for performing exchange on the cell surface, independently of the endogenous processing pathway.

Citation or identification of any document in this application is not an admission that such document is available as prior art to the present invention.

### SUMMARY OF THE INVENTION

Applicants provide an ortholog of the molecular chaperone TAPBPR from Gallus gallus (chicken) to enable the exchange of peptides on HLA molecules of diverse peptide-binding specificities (supertypes). Chicken TAPBPR interacts with multiple recombinantly expressed HLA proteins from the classical-A, -B, and -C types and likely also with the nonclassical MR1 type to promote ligand exchange in vitro, for MIC-I tetramer library generation. Alternatively, ligand exchange on the cell surface, promoted by chicken TAPBPR, can be used to enhance the display of properly conformed peptide:HLA or ligand:MR1 molecules on antigen-presenting cells for T cell stimulation in various settings.

Using an ortholog of the molecular chaperone TAPBPR from Gallus gallus (FIG. 1), Applicants identify interactions with multiple HLA molecules from the A01/A24, A02, B8, B58 supertypes, and several C allotypes (FIGS. 2, 3). The results using an in vitro fluorescent bead-based assay demonstrate a clear interaction between chicken TAPBPR with disease-relevant human allotypes, HLA-B*37:01, HLA-A*29:01, HLA-B*08:01, HLA-A*29:02, HLA-B*57:03, HLA-B*59:01, HLA-C*03:04, HLA-C*01:02, HLA-B*38:03, HLA-A*32:01 and HLA-C*03:03, with several other allotypes potentially amenable to lower-affinity interactions (FIGS. 2, 3). Applicants validate results using recombinantly expressed HLA-A*24:02 and HLA-B*08:01 proteins by binding to a TAPBPR-conjugated surface by Surface Plasmon Resonance spectroscopy, which confirms a direct interaction with chicken TAPBPR in the 100 nanomolar-range, in contrast to human TAPBPR which only recognizes HLA-A*24:02 with moderate affinity (FIG. 4). Alternatively, catalytic amounts of TAPBPR can be used to exchange placeholder ligands with high affinity ligands of interest, as shown in exemplar applications for the common allotype HLA-A*01:01 (FIG. 5), and monomorphic MR1 molecule.

Based on these findings, Applicants outline a protocol for the generation of p:HLA libraries encompassing multiple HLA supertypes, which can be readily tetramerized and loaded with peptides of choice in a high-throughput manner (FIG. 6). Using the chicken TAPBPR system, Applicants provide a high throughput DSF-based assay to validate binding of multiple candidate ligands on empty HLA/chTAPBPR complexes. Combined with tetramer-barcoding, this approach allows a combined analysis of TCR repertoires and other T cell transcription profiles together with their cognate antigen specificities in a single experiment across a broad range of disease-relevant MHC-I allotypes.

The present invention also relates to a method for generating conditional peptide ligands for a range of disease-related MHC-I allotypes, which form stable complexes with MHC-I molecules but can be exchanged for desired peptides through a chaperone-assisted process. Rather than using truncated high affinity peptides, the new approach involves introducing a modified unnatural amino acid near the peptide C-terminus, which results in a MHC-I protein complex with higher thermal stability and improved exchanged properties, relative to the conventional goldilocks peptides.

The invention relates to an isolated ligand exchange protein comprising a fragment of TAP-binding protein-related (TAPBPR), said fragment consisting of the TAPBPR luminal domain. In one embodiment, the TAPBPR fragment consists of an amino acid sequence having at least 50% or at least 60% or at least 70% or at least 80% or at least 85% or at least 86% or at least 87% or at least 88% or at least 89% or at least 90% or at least 91% or at least 92% or at least 93% or at least 94% or at least 95% or at least 96% or at least 97% at least or 98% or at least 99% sequence identity to the chicken or human TAPBR sequence of FIG. 1 or FIG. 21. Parts of sequence most amenable to substitution are located outside of the interface with MHC-I molecules on the modeled chicken TAPBPR/MHC-I structure (highlighted with stars * in the sequence alignment of FIG. 1 or FIG. 21 and shown as sticks in the structure diagram of FIG. 2a).

In another embodiment, the isolated ligand exchange protein further comprises a targeting domain. In an advantageous embodiment, the targeting domain is linked to the TAPBPR fragment via a linker. In another advantageous embodiment, the targeting domain specifically binds to target cells. In another advantageous embodiment, the target cells are cancer cells. In another advantageous embodiment, the targeting domain specifically binds to a target molecule on the surface of the cancer cells. In another advantageous embodiment, the target molecule is ErbB2, PDL1 or CD20. In another advantageous embodiment, the target cells are pathogen infected cells. In another advantageous embodiment, the pathogen infected cells are cells infected with HIV, CMV, EBV, HPV, Influenza or hepatitis. In another advantageous embodiment, the targeting domain specifically binds to a pathogen antigen or a molecule upregulated by pathogen infection on the surface of the infected cells. In another advantageous embodiment, the pathogen antigen is an HIV antigen selected from gp120 and gp41; a CMV antigen selected from UL 11, UL142, UL9, UL1, UL5, UL16, UL55, UL74, UL75 and UL155 (gL) or an influenza antigen selected from hemagglutinin and neuraminidase. In another advantageous embodiment, the target cells are antigen presenting cells. In another advantageous embodiment, the antigen presenting cells are dendritic cells. In another advantageous embodiment, the targeting domain is an antibody molecule, optionally an scFv or nanobody. In another advantageous embodiment, the targeting domain is a ligand for a surface receptor on the target cells. In another advantageous embodiment, the targeting domain is CD4, CD20, PD1 or an antibody Fc domain.

In an embodiment, the isolated ligand exchange protein comprises an amino acid sequence having least 50% or at least 60% or at least 70% or at least 80% or at least 85% or at least 86% or at least 87% or at least 88% or at least 89% or at least 90% or at least 91% or at least 92% or at least 93% or at least 94% or at least 95% or at least 96% or at least 97% at least or 98% or at least 99% sequence identity to the chicken or human TAPBR sequence of FIG. 1 or FIG. 21. Parts of sequence most amenable to substitution are located outside of the interface with MHC-I molecules on the modeled chicken TAPBPR/MHC-I structure (highlighted with stars * in the sequence alignment of FIG. 1 or FIG. 21 and shown as sticks in the structure diagram of FIG. 2a).

The invention relates to a ligand exchange protein comprising: (i) a fragment of TAP-binding protein-related (TAPBPR), said fragment comprising a TAPBPR luminal domain and a TAPBPR transmembrane domain; or (ii) a fragment of TAP-binding protein-related (TAPBPR) comprising a TAPBPR luminal domain and a heterologous transmembrane domain.

In one embodiment, said fragment lacks the TAPBPR cytoplasmic domain. In another embodiment, the ligand exchange protein further comprises a heterologous cell surface targeting sequence. In another embodiment, the heterologous cell surface targeting sequence comprises the cytoplasmic domain of CD8. In an advantageous embodiment, the isolated ligand exchange protein according to paragraph 22 comprises an amino acid sequence having at least 50% or at least 60% or at least 70% or at least 80% or at least 85% or at least 86% or at least 87% or at least 88% or at least 89% or at least 90% or at least 91% or at least 92% or at least 93% or at least 94% or at least 95% or at least 96% or at least 97% at least or 98% or at least 99% sequence identity to the chicken or human TAPBR sequence of FIG. 1 or FIG. 21. Parts of sequence most amenable to substitution are located outside of the interface with MHC-I molecules on the modeled chicken or human TAPBPR/MHC-I structure (highlighted with stars * in the sequence alignment of FIG. 1 or FIG. 21 and shown as sticks in the structure diagram of FIG. 2a).

The invention encompasses a nucleic acid encoding any one of the herein disclosed isolated ligand exchange protein. The invention also encompasses a vector comprising any one of these nucleic acids. The invention also encompasses a mammalian cell comprising any of these nucleic acids or vectors. In some embodiments, the mammalian cell comprises a ligand exchange protein at its surface.

The invention further relates to a method of increasing the immunogenicity of mammalian cells comprising contacting a population of mammalian cells having surface MHC class I molecules with an immunogenic ligand and any of the herein disclosed ligand exchange proteins or a herein disclosed mammalian cell, such that the ligand exchange protein loads the immunogenic ligand onto the surface MHC class I molecules of the cells in the population thereby increasing the immunogenicity of the mammalian cells.

The invention also relates to use of an immunogenic ligand and any of the herein disclosed ligand exchange proteins or a herein disclosed mammalian cell, such that the ligand exchange protein loads the immunogenic ligand onto the surface MHC class I molecules of the cells in the population, in or for a method of increasing the immunogenicity of mammalian cells comprising; contacting a population of mammalian cells having surface MHC class I molecules with the immunogenic ligand and the ligand exchange protein, such that the ligand exchange protein loads the immunogenic ligand onto the surface MHC class I molecules of the cells in the population, thereby increasing the immunogenicity of the mammalian cells.

The invention also relates to a method of producing antigen presenting cells to stimulate an immune response in an individual comprising contacting antigen presenting cells in vitro with any of the herein disclosed ligand exchange proteins or a herein disclosed mammalian cell and an immunogenic ligand, such that the ligand exchange protein loads the immunogenic ligand onto surface MHC class I molecules of the antigen presenting cells, wherein the population of antigen presenting cells are previously obtained from the individual.

The present invention also relates to a use of any of the herein disclosed ligand exchange proteins or a herein disclosed mammalian cell and an immunogenic ligand in or for a method of producing antigen presenting cells to stimulate an immune response in an individual comprising; contacting antigen presenting cells in vitro with the ligand exchange protein such that the ligand exchange protein loads the immunogenic ligand onto surface MHC class I molecules of the antigen presenting cells, wherein the population of antigen presenting cells are previously obtained from the individual.

In one embodiment, the antigen presenting cells are dendritic cells. In another embodiment, the use or method comprises activating a population of T cells with the antigen presenting cells. In another embodiment, the use or method is performed in vitro or ex vivo. In another embodiment, the use or method further comprises administering the mammalian cells, antigen presenting cells or activated T cells to an individual.

The invention further relates to a method of increasing the immunogenicity of target cells in an individual comprising administering any one of there herein disclosed ligand exchange proteins according to the individual, wherein the ligand exchange protein comprises a targeting domain that binds to target cells in the individual, and administering an immunogenic ligand to the individual, such that the ligand exchange protein loads the immunogenic ligand onto surface MHC class I molecules of the target cells, thereby increasing the immunogenicity of said target cells.

The invention also relates to a use of any one of there herein disclosed ligand exchange proteins to the individual in or for a method of increasing the immunogenicity of target cells in an individual comprising administering the ligand exchange protein, wherein the ligand exchange protein comprises a targeting domain that binds to target cells in the individual, and administering an immunogenic ligand to the individual, such that the ligand exchange protein loads the immunogenic ligand onto surface MHC class I molecules of the target cells, thereby increasing the immunogenicity of said target cells.

In one embodiment, any one of there herein disclosed ligand exchange proteins are utilized in a method of increasing the immunogenicity of target cells in an individual. The invention also relates to a method or use or protein wherein the target cells are disease cells. The invention also relates to a method or use of a protein wherein the disease cells are cancer cells or pathogen infected cells.

The invention also encompasses a method of stimulating an immune response in an individual comprising administering any one of the herein disclosed ligand exchange proteins, wherein the targeting domain of the ligand exchange protein binds to antigen presenting cells in the individual, and administering an immunogenic peptide to the individual, such that the ligand exchange protein loads the immunogenic peptide onto surface MHC class I molecules of the antigen presenting cells such that said antigen presenting cells stimulate an immune response in the individual.

The invention relates to a use of any one of the herein disclosed ligand exchange proteins in or for a method of stimulating an immune response in an individual comprising; administering the ligand exchange protein to the individual, wherein the targeting domain of the ligand exchange protein binds to antigen presenting cells in the individual, and administering to the individual an immunogenic peptide, such that the ligand exchange protein loads the immunogenic peptide onto surface MHC class I molecules of the antigen presenting cells, such that said antigen presenting cells stimulate an immune response in the individual.

In one embodiment, in the above method or use, the antigen presenting cells are dendritic cells. In another embodiment, the immunogenic peptide is a vaccine.

The present invention relates to a method of producing a MHC class I molecule displaying a target peptide comprising contacting an MHC class I molecule with any one of the herein disclosed ligand exchange proteins and a target peptide, such that the ligand exchange protein loads the target peptide onto the MHC class I molecule thereby producing an MHC class I molecule displaying the target peptide.

The present invention relates to use of any one of the herein disclosed ligand exchange proteins and a target peptide in or for a method of producing a MHC class I molecule displaying a target peptide comprising; contacting an MHC class I molecule with the ligand exchange protein, such that the ligand exchange protein loads the target peptide onto the MHC class I molecule, thereby producing an MHC class I molecule displaying the target peptide.

In one embodiment, in the method or use above, the MHC class I molecule displays an initial peptide that is replaced by the target peptide following contact with the ligand exchange protein. In another embodiment, the MHC class I molecule is immobilized on a solid support. In another embodiment, the MHC class I molecule is a sub-unit of a multimer comprising multiple MHC class I molecules. In another embodiment, the multimer is a tetramer comprising biotinylated MHC class I molecules linked by streptavidin. In another embodiment, the method or use further comprises contacting the MHC class I molecule displaying the target peptide with a population of T cells and determining the binding of the MHC class I molecule to T cells in the population.

The invention relates to a method for generating a conditional peptide ligand for a HLA allotype which forms a stable complex with a MHC-I molecule comprising: choosing a high affinity peptide for an HLA allele of interest, wherein the high affinity peptide has a melting temperature greater than 60 degrees centigrade, introducing a modified unnatural amino acid near the C-terminus of the high affinity peptide, thereby generating a placeholder peptide, performing a preliminary refolding reaction with the placeholder peptide, wherein the placeholder peptide has a melting temperature of about 50 degrees centigrade, dislodging the conditional peptide ligand when interacting with a chaperone with a high affinity peptide of interest such that the high affinity peptide of interest binds to an empty MHC groove, and selecting a top placeholder peptide to perform a chaperone-mediated exchange with the high affinity peptide of interest, wherein the top placeholder peptide is the conditional peptide ligand.

In one embodiment, the HLA allele of interest is selected from the Immune Epitope Database. In another embodiment, the modified unnatural amino acid is introduced at positions 8, 7 or 6 of the high affinity peptide. In another embodiment, the modified unnatural amino acid is a 3-amino acid such as 0-phenylalanine. In another embodiment, the chaperone is tapasin or TAPBPR. In another embodiment, peptide binding is analyzed by differential scanning fluorimetry (DSF) or fluorescence polarization (FP). In another embodiment, a method of generating a tetramer library further comprises repeating above method, wherein the library comprises a plurality of high affinity peptides of interest. The invention also encompasses any high affinity peptide of interest generated by any one of there herein disclosed methods.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1—Human and Chicken TAPBPR sequence alignment. Alignment of luminal domains of human (UniProtKB/Swiss-Prot: Q9BX59) and chicken (NP_001382952.1) TAPBPR, where conserved residues are marked in boxes. The secondary structure of human TAPBPR (PDB ID 5WER) is provided as reference and the interacting residues with H2-Dd and β₂m are indicated by the stars. Parts of sequence most amenable to substitution fall outside of these residues. Clustal Omega was used for the alignment and the output was further processed with ESPript 3.

FIGS. 2A-2D—TAPBPR orthologs have differences in stability and recognition of HLA allotypes. a. Structure diagram highlighting the differences in the interface of chicken TAPBPR (Robetta, Baker Lab) and the B*37:01/hβ₂m complex (PDB ID 6MT6), which is the strongest binder as shown from Applicants' LABScreen experiments in d. Conserved and polymorphic residues relative to human TAPBPR are marked. b. Native gel shift analysis of human (hu), mouse (mo) and chicken (chi) TAPBPR/MHC-I complex formation. Each well is loaded with free pMHC-I (A02: HLA-A*02:01 refolded with KILGFVFJV (SEQ ID NO: 25), A24: HLA-A*24:02 refolded with VYGJVRACL (SEQ ID NO: 3) and A01: HLA-A*01:01 refolded with STAPGJLEY (SEQ ID NO: 9)), free TAPBPR or 1:1 molar ratio mixture. Samples were UV-irradiated for 1 hour at 365 nm as indicated. The formed pMHC-I/TAPBPR complexes are indicated with arrows. J=3-amino-3-(2-nitrophenyl)-propionic acid. c. DSF results of human (hu), chicken (chi) and mouse (mo) TAPBPR proteins. Data shown are representative of replicate assays (n=3), and standard deviation is depicted with error bars. d. Bar graph showing the level of human and chicken TAPBPR binding to the single antigen HLA-I beads. Blocking with the W6/32 antibody was used as a negative control, and the MFI ratios of TAPBPR/control were determined. The plotted data including standard deviation were generated based on three independent experiments (n=3). e. Correlation plot of the human and chicken TAPBPR log (MFI ratio) plotted in d. The dotted line represents a conceptual 1:1 correlation (no effect).

FIGS. 3A-3C. a. Detection of MHC class I levels on single antigen beads using the Anti-HLA Class I Antigens Antibody W6/32. Similar levels of peptide-loaded MHC-I molecules are observed across the beads. b. Bar graphs of human (huTAPBPR) and c. chicken (chiTAPBPR) TAPBPR binding to HLA molecules upon treatment with tetramers at a 7 μM final concentration of the monomer. As a negative control, blocking with W6/32 was performed prior to tetramer addition. The plotted data were generated based on three independent experiments and standard deviation is depicted with error bars.

FIG. 4. Demonstration of direct interactions between chicken TAPBPR with recombinant HLA-A and -B molecules using Surface Plasmon Resonance spectroscopy. Representative surface plasmon resonance (SPR) sensorgrams for varying concentrations (0.1 to 10 μM) of HLA-A*24:02/VYGJVRACL (SEQ ID NO: 3) and HLA-B*08:01/FLRGRAJGL (SEQ ID NO: 4) flow over a streptavidin chip coupled with human or Chicken TAPBPR-biotin, as indicated. A flat sensogram is indicative of no interaction in vitro. The concentrations of analyte for the top and the bottom sensorgrams are noted. Data is shown in black. Fits from the surface-bound and the kinetic analysis are shown with solid and dotted lines, with errors in KD values indicates (n=3). Data is representative of triplicate experiments.

FIGS. 5A-5E. Demonstration of ligand exchange on HLA-A*01:01 by chicken TAPBPR using a Differential Scanning Fluorimetry assay. A: Thermal stabilities of HLA-A*01:01/STAPGJLEY (SEQ ID NO: 9) were determined with 1×PBS buffer (black) and a 10-fold molar excess of HLA-A*01:01 peptide CTDDNALAYY (SEQ ID NO: 5) under one-hour RT incubation (magenta) or UV irradiation, which is known to promote ligand exchange (Bakker et al. 2008). B-E: Thermal stabilities of HLA-A*01:01/STAPGJLEY (SEQ ID NO: 9) were determined in the presence of 10-fold molar excess CTDDNALAYY (SEQ ID NO: 5) (D), ESDPIVAQY (SEQ ID NO: 6) (E), PTDNYITTY (SEQ ID NO: 7) (F), and a non-specific peptide YPNVNIHNF (SEQ ID NO: 8) (G) for an hour incubation at RT with human or chicken TAPBPR.

FIG. 6. Process for rapid deployment of peptide/MHC-I (pMHC-I) multimers. The workflow translates peptide epitope predictions into pMHC-I multimers, which can be used to monitor antigen-specific CD8 T cell responses in patients, and to identify immunodominant epitopes. Epitopic peptide candidates compete for binding on MHC-I molecules refolded with a placeholder peptide in vitro, catalyzed by the molecular chaperone TAPBPR from Gallus. Each exchange reaction is incubated for two hours at room temperature, and the thermal stabilities of the resulting pMHC-I molecules are accessed using a Differential Scanning Fluorimetry (DSF) assay, to confirm that net ligand exchange has occurred. Selected pMHC-I are then loaded onto fluorophore-labelled streptavidin, with optional DNA oligo barcoding.

FIGS. 7A-7B. Demonstration of ligand exchange on HLA-B*08:01 by chicken TAPBPR using a Differential Scanning Fluorimetry assay. A. Thermal stabilities of HLA-A*08:01/RPHERNGFTVL (SEQ ID NO: 10) were determined with 1×PBS buffer (black) and a 10-fold molar excess of HLA-A*08:01 peptide epitope FMKQMNDAL (SEQ ID NO: 11) under one-hour RT incubation (magenta). B. Thermal stabilities of HLA-B*08:01/RPHERNGFTVL (SEQ ID NO: 10) were determined in the presence of 10-fold molar excess FMKQMNDAL (SEQ ID NO: 11) for an hour incubation at RT with human or chicken TAPBPR.

FIG. 8. Structure-guided design and synthesis of conditional peptide ligands for different human HLA allotypes based on the principle of destabilizing interactions with F-pocket residues. The conditional peptide ligand KILGFVFβFV (SEQ ID NO: 12) is based on the HLA-A*02:01-restricted influenza Matrix 1 (58-66) epitope GILGFVFTL (SEQ ID NO: 13), where Gly1 is replaced with lysine for solubility and where Thr8 is replaced with a DL-β-Phenylalanine residue termed βF and where anchor residue Leu9 is replaced with valine for increased HLA affinity. The conditional peptide ligands STAPGFLEY (SEQ ID NO: 14), VYGPFVRACL (SEQ ID NO: 15), FLRGRAPFGL (SEQ ID NO: 16), FEDLRVPFSF (SEQ ID NO: 17), and LNPSVPFATL (SEQ ID NO: 18) are based on the HLA-A*01:01 motif peptide STAPGVLEY (SEQ ID NO: 19), HLA-A*24:02 hTRT-derived peptide VYGFVRACL (SEQ ID NO: 20), HLA-B*08:01 EBV EBNA-3A peptide FLRGRAYGL (SEQ ID NO: 21), HLA-B*37:01 Influenza NP₃₃₈₋₃₄₆ peptide FEDLRVLSF (SEQ ID NO: 22), and HLA-C*01:02 Hepatitis C virus peptide LNPSVAATL (SEQ ID NO: 23).

FIGS. 9A-9C. Demonstration of conditional ligand exchange on HLA-A*02:01 using a Differential Scanning Fluorimetry (DSF) assay. a. Melting temperature (Tm, ° C.) obtained from DSF of refolded HLA-A*02:01 bound to LFGYPVYV (TAX8) (SEQ ID NO: 24), KILGFVFJV (SEQ ID NO: 25), KILGFVFβFV (SEQ ID NO: 12), and LLFGYPVYV (TAX9) (SEQ ID NO: 26). b. Thermal stabilities of HLA-A*02:01/KILGFVFJV (SEQ ID NO: 25) were determined in the presence of 10-fold molar excess of a high-affinity peptide TAX9 LLFGYPVYV (SEQ ID NO: 26), a non-specific peptide YPNVNIHNF (SEQ ID NO: 8), or buffer (black) for one hour UV irradiation. c. Thermal stabilities of HLA-A*02:01/KILGFVFβFV (SEQ ID NO: 12) were determined in the presence of 10-fold molar excess of a high-affinity peptide TAX9 LLFGYPVYV (SEQ ID NO: 26), a non-specific peptide YPNVNIHNF (SEQ ID NO: 8), or buffer for two-hour RT incubation with the catalytical amount of human TAPBPR. J=3-amino-3-(2-nitrophenyl)-propionic acid. βF=DL-β-Phenylalanine.

FIG. 10. Demonstration of conditional ligand exchange kinetics on HLA-A*02:01/KILGFVFβFV using a Fluorescence Polarization (FP) assay. 200 nM HLA-A*02:01/KILGFVFβFV (SEQ ID NO: 12) were incubated in the presence of 40 nM TAMRA-conjugated peptide _{TAMRA}KLFGYPVYV (SEQ ID NO: 27) with or without the molecular chaperone TAPBPR (20 nM). Fluorescently labeled peptide binding increases significantly within several hours of the incubation with TAPBPR while there is no fluorescence polarization (mP) change with the addition of fluorescently labeled peptide _{TAMRA}KLFGYPVYV (SEQ ID NO: 27) only. Measurements were recorded every 60 seconds across a 2-hour interval. Averages of at least three independent experiments±SEM are shown.

FIG. 11. Process for rapid deployment of peptide/MHC-I (pMHC-I) multimers. The workflow translates peptide epitope predictions into pMHC-I multimers, which can monitor antigen-specific CD8 T cell responses in patients and identify immunodominant epitopes. Epitopic peptide candidates compete for binding on MHC-I molecules refolded with the conditional peptide in vitro, catalyzed by the molecular chaperone TAPBPR. Each exchange reaction is incubated for two hours at room temperature. The net peptide exchange is directly confirmed through a Fluorescence Polarization (FP) assay via competitively binding to MHC-1 against a high-affinity TAMRA fluorescently labeled peptide. Selected pMHC-I are then loaded onto fluorophore-labeled streptavidin, with optional DNA oligo barcoding.

FIG. 12A-12B. Detection of antigen-specific T cells by peptide exchanged HLA-A*02:01/KILGFVFßFV tetramer using flow cytometry. a. Represent flow cytometric analysis using human T cells transduced with 1G4 T cell receptors (TCRs) that can recognize HLA-A*02:01/SLLMWITQV (NY-ESO-1) (SEQ ID NO: 28). Columns depict staining with a fixed excess of HLA-A*02:01/SLLMWITQV (SEQ ID NO: 28) tetramers prepared by conventional refolding (top left) and conditional peptide exchange (top middle). Tetramer prepared from HLA-A*02:01 refolded with a non-specific Rubella peptide ALLDRLRGVYA (SEQ ID NO: 29) (top right) was used as a negative control. HLA-A*02:01 tetramers loaded NY-ESO-1 peptides with alanine mutation at position 1 (ALLMWITQV (SEQ ID NO: 30), lower left), 4 (SLLAWITQV (SEQ ID NO: 31), lower middle), 5 (SLLMAITQV (SEQ ID NO: 32), lower right) were prepared through peptide exchange for the conditional peptide KILGFVFβFV (SEQ ID NO: 12) using a catalytic amount of TAPBPR. b. Bar graph summarizes the tetramer staining of T cells transduced with a NY-ESO-1 specific TCR 1G4. Error bars show mean fluorescence intensity (MFI)±SD from independent duplicates.

FIG. 13. Graphic abstract of Example 10.

FIGS. 14A-14E. Divergent TAPBPR sequences show differences in stability and recognition of HLA allotypes. a. Phylogenetic analysis of TAPBPR ectodomain sequences from selected species. Evolutionary analysis was conducted in MEGA7. The bootstrap values (500 bootstrap trials), depicted as spheres, indicate the percentage of trees in which the associated taxa clustered together. b. Surface representation of hTAPBPR in contact with mouse H2-D^{d}/human β₂m complex (PDB ID: 5WER; left). The predicted contact residues of hTAPBPR are highlighted for the conserved or polymorphic residues between human and chicken orthologs, respectively (right). For the polymorphic sites, the respective chicken residues are shown in red. c. Comparison of the electrostatic potential surfaces for human and chicken TAPBPR in the indicated ranges (down to −70 k_{B}T/e and up to +70 k_{B}T/e, where k_{B} is the Boltzmann constant, T the temperature, and e the unit charge) as calculated using the APBS solver in PyMOL. The structure model of chTAPBPR was generated using the BAKER-ROBETTA server. d. Tₘ (° C.) obtained from DSF for recombinantly expressed human, chicken, and mouse TAPBPR. The data shown represent replicate assays (n=3). e. Native gel shift analysis of TAPBPR orthologs and MHC-I complex formation. Each well is loaded with free pMHC-I (A02: HLA-A*02:01 refolded with KILGFVFJV (SEQ ID NO: 25), A24: HLA-A*24:02 refolded with VYGJVRACL (SEQ ID NO: 3), and A01: HLA-A*01:01 refolded with STAPGJLEY (SEQ ID NO: 9), where J=3-amino-3-(2-nitrophenyl)-propionic acid), free TAPBPR or 1:1 molar ratio mixture. Samples were UV-irradiated for 1 hour at 365 nm, when indicated. The formed MHC-I/TAPBPR complexes are indicated with arrows.

FIGS. 15A-15D. TAPBPR orthologs demonstrate distinct HLA binding profiles using single antigen beads (SABs). a. Schematic representation of the SABs assay used to measure levels of binding to individual HLA-I allotypes by streptavidin-PE conjugated TAPBPR orthologs. SABs are color-coded by different ratios of fluorescent dyes and are decorated with different HLA-I allotypes. The SABs were incubated with 7 μM of tetramerized TAPBPR for 1 hour at room temperature (RT). b. Box plot showing the distribution of the TAPBPR MFI ratios for the 96 HLA-I allotypes. hTAPBPR^{TN6} was used to determine significant interactions between TAPBPR orthologs and MHC-I molecules. The boundary of the box closest to zero indicates the 25^{th} percentile, the line within the box marks the median and the boundary of the box farthest from zero indicates the 75^{th} percentile. Whiskers above and below the box indicate the 10^{th} and 90^{th} percentiles. The points above the whiskers indicate outliers outside the 90^{th} percentile. c. Bar graph showing the levels of hTAPBPR and chTAPBPR binding to the SABs. Incubation with the W6/32 antibody prior to tetramer staining was used to control for the non-specific background binding, and the MFI ratio of TAPBPR/control was determined. A cut-off of log₁₀ 5.97 (0.77) was set to determine an interaction as significant, calculated from the mean value of hTAPBPR^{TN6}+3SD. Plotted data are mean±SD from 2 or 3 independent experiments. d. Correlation of hTAPBPR and chTAPBPR (c) log₁₀(MFI ratio) plotted in (d). The dotted line represents a conceptual 1:1 correlation (no difference between orthologs).

FIGS. 16A-16H. TAPBPR orthologs recognize peptide-deficient HLA molecules with a broad allelic specificity in vitro. (A) Schematic diagram showing TAPBPR orthologs immobilized on the SPR sensor chip. Different photolabile peptide-loaded HLA molecules at various concentrations with or without UV irradiation flow over the surface. (B) Representative sensorgram of HLA-B*08:01/FLRGRAJGL (SEQ ID NO: 4) flowed over a streptavidin chip coupled with chTAPBPR-biotin. (C) Log-scale comparison of K_{D} values for chTAPBPR or hTAPBPR interacting with HLA allotypes as indicated. (D) Schematic summary of peptide-loaded HLA molecules interacting with hTAPBPR or chTAPBPR in light blue or red, respectively. Allotypes that bind to hTAPBPR, chTAPBPR, or both are colored in blue, red, and black. (E)-(F) Representative sensorgrams of UV irradiated HLA-A*01:01 (E) and HLA-B*08:01 (F). (G) Log-scale comparison of K_{D} values for chTAPBPR or hTAPBPR interacting with peptide-deficient HLA allotypes as indicated. (H) Schematic summary of peptide-deficient HLA molecules interacting with hTAPBPR or chTAPBPR in light blue or red, respectively. Allotypes that bind to hTAPBPR, chTAPBPR, or both are colored in blue, red, and black. Correlation of peptide-loaded and -deficient HLA binding to hTAPBPR and chTAPBPR log₁₀ K_{D} plotted in (C) and (D). The dotted red line represents a conceptual 1:1 correlation (no difference between peptide-loaded and -deficient molecules). K_{D}, equilibrium constant; kₐ, association rate constant; k_{d}, dissociation rate constant; RU, resonance units. The concentrations of analyte for the top and the bottom sensorgrams are noted. Fits from the kinetic analysis are shown with red lines. Results of at least two technical replicates (mean±σ) are plotted.

FIGS. 17A-17H. Conditional peptide ligands reveal the effects of HLA micropolymorphisms and supertype dependence of peptide exchange by TAPBPR orthologs. (A) Conditional peptide ligand KILGFVFβFV (SEQ ID NO: 12) based on the HLA-A*02:01-restricted influenza Matrix 1 (58-66) epitope GILGFVFTL (SEQ ID NO: 13). Thr8 is replaced with L-β-Phenylalanine residue termed βF. (B) HLA-A*02:01 βF scanning peptide panel at each position as indicated. (C) Tₘ (° C.) values obtained from DSF of HLA-A*02:01 bound to KILGFVFTV (SEQ ID NO: 33) with βF substitution at indicated positions. (D) Comparison of kₐₚₚ for fluorescent peptide binding to HLA-A*02:01/KILGFVFTV (SEQ ID NO: 33) with βF substitution at indicated positions in the presence of 10 nM hTAPBPR. (E) Crystal structure of HLA-A*02:01 (PDB ID: 3MRE) indicates the polymorphic residues for HLA-A*02:06 (light pink), HLA-A*02:11 (light yellow), HLA-A*02:71 (light blue), and HLA-A*02:77 (light orange). (F) Log-scale comparison of k₂ₒᵥₑᵣₐₗₗ for HLA-A*02:01, A*02:06, A*02:11, A*02:71, and A*02:77 in the presence of hTAPBPR. The two-sample unequal variance Student's t test was performed, p>0.12 (ns), p<0.033(*), p<0.002(**), p<0.001(***). (G) Linear correlations between the apparent rate constants kₐₚₚ and the concentrations of TAPBPR orthologs for HLA-B*37:01. (H) Log-scale comparison of k₂ₒᵥₑᵣₐₗₗ for HLA-A*01:01, A*30:01, A*29:02, A*02:01, A*68:02, A*03:01, A*11:01, A*24:02, B*08:01, and B*37:01 in the presence of human or chicken TAPBPR. The apparent rate constant kₐₚₚ was determined by fitting the raw trace to a monoexponential association model. The extrapolation of the slope between kₐₚₚ and the concentrations of TAPBPR orthologs determines the overall rate k₂ₒᵥₑᵣₐₗₗ. NA indicates no k₂ₒᵥₑᵣₐₗₗ (no peptide exchange) determined. Results of three technical replicates (mean±σ) are plotted.

FIGS. 18A-18C. chTAPBPR stabilizes HLA-B*08:01 by recognizing conserved hTAPBPR interaction surfaces on MHC-I. a. Structural model of the HLA-B*08:01/chTAPBPR complex generated by RosettaCM (BAKER-ROBETTA server). b. The percent deuterium uptake resolved to individual peptide segments showing α₁ 68-79, α₂₋₁ 131-138, and β₂m 74-92 is plotted for each exposure time (0, 20, 60, 180, 600 s) and protein states. The plots reveal the local HDX profiles of HLA-B*08:01 for the states of peptide-deficient (black, UV irradiation for 40 minutes at 4° C.), peptide-loaded (grey, refolded with FLRGRAJGL (SEQ ID NO: 4)), and chTAPBPR-bound complex (UV irradiated for 40 minutes at room temperature in the presence of excess chTAPBPR followed by SEC purification of the complex peak). c. The percent deuterium uptake resolved to individual residues upon 60-second deuterium labeling for peptide-loaded (left), peptide-deficient (middle), and chTAPBPR-bound complex (right) states are mapped onto the HLA-B*08:01 crystal structure (PDB ID: 4QRU) for visualization. Shaded regions indicate segments containing peptides with 100% ΔHDX or 0% ΔHDX, respectively; black indicates regions where peptides were not obtained for both the empty and chTAPBPR-bound conditions.

FIGS. 19A-19F. Deep mutational scanning of TAPBPR surfaces leads to enhanced peptide exchange function for A*02 and A*01 a. hTAPBPR-TM was mutationally scanned based on promoting HLA-A*02:01 surface expression in tapasin-KO Expi293F cells. Sequence conservation from deep mutagenesis is mapped to the surface of hTAPBPR (PDB ID: 5WER, mouse H2-D^{d}/human β₂m shown as light and dark ribbons at left). Conserved residues and residues that are mutationally tolerant are shaded. Residues that were not mutated are grey. b. Based on the difference between conservation scores from the deep mutational scan of hTAPBPR-TM described here versus hTAPBPR previously reported⁵¹, residues preferentially conserved for intracellular HLA-A*02:01 processing and those preferentially conserved for surface HLA-A*02:01 interactions are shaded. Four amino acid positions (S104, R105, K211, and R270) are identified that are generally more conserved for intracellular HLA-A*02:01 processing and are sites of gain-of-function mutations for promoting surface HLA-A*02:01 trafficking. c. HLA-A*02:01 surface expression in tapasin-KO cells is enhanced by TAPBPR-TM mutants. Immunoblots comparing total expression levels for TAPBPR mutants (α-FLAG) versus PP1B as a loading control (Left). Surface expression of HLA-A*02:01 in the presence of the different TAPBPR-TM mutants (middle). Surface expression for the different TAPBPR-TM mutants (right). d. The log-scale comparison of k₂ₒᵥₑᵣₐₗₗ for HLA-A*02:01 and A*02:06 in the presence of hTAPBPR or hTAPBPR mutants. Results of three replicates (mean±SD) are plotted. The two-sample unequal variance Student's t test was performed, p>0.12 (ns), p<0.033(*), p<0.002(**), p<0.001(***). e. HLA-A*01:01 surface expression in tapasin-KO/HLA-A*01:01 knock in cells is enhanced by TAPBPR-TM mutants. Immunoblots comparing total expression levels for TAPBPR mutants (α-FLAG) versus PP1B as a loading control (Left). Surface expression of HLA-A*01:01 in the presence of the different TAPBPR-TM mutants (middle). Surface expression for the different TAPBPR-TM mutants (right). f. The comparison of kₐₚₚ for fluorescent peptide binding to HLA-A*01:01 in the presence of 5 μM chTAPBPR, hTAPBPR mutants, and hTAPBPR. Results of three replicates (mean±SD) are plotted.

FIGS. 20A-20F. Minimal mutations of TAPBPR interaction surfaces enhance binding affinities for HLA-I allotypes. a. Seq2logo⁶⁹ visualization of MHC-I residues that interact with TAPBPR calculated from the sequences of 75 different HLA allotypes. Sequence weighting used clustering, pseudo count with a weight of 0, and Kullback-Leibler logotype. The percentage frequency of amino acids on a specific position higher than 10% is shown on the positivey-axis and less than 10% amino acids on the negativey-axis. b. Structure models of HLA-A*01:01 or HLA-A*02:01 in complex with hTAPBPR generated by RosettaCM (BAKER-ROBETTA server). c. Sequence alignment of hTAPBPR, chTAPBPR, and hTapasin near positions 104, 105, 211, and 270. d. Sequence conservation patterns within the same region of β₂m from the amino acid 58 to 60 across 4 species. e. Log-scale comparison of equilibrium dissociation constants for peptide-loaded and empty HLA-A*02:01 with hTAPBPR and TAPBPR mutants as measured by SPR. Results of two replicates (mean±SD) are plotted. f. The log-scale comparison of SPR determined K_{D} values for MR1 C262S with hTAPBPR, TAPBPR mutants, and chTAPBPR. Results of at least two replicates (mean±SD) are plotted.

FIG. 21. TAPBPR orthologs sequence alignment. Alignment of the luminal domains of human (UniProtKB/Swiss-Prot: Q9BX59), mouse (XP_030111162.1), and chicken (NP_001382952.1) TAPBPR was performed using ClustalOmega and processed using ESPript. Conserved residues are marked in boxes and the residues that are in direct contact with the heavy chain are noted with asterisks (*). The secondary structure of human TAPBPR (PDB: 5WER) is provided as a reference.

FIGS. 22A-22B. TAPBPR orthologs expression and purification. a-b. SEC traces of (a.) hTAPBPR, chTAPBPR, moTAPBPR and (b.) hTAPBPR^{TN6} proteins. The protein peaks are indicated by the arrow and further confirmed by SDS/PAGE analysis.

FIGS. 23A-23E. Bar graph showing the levels of anti-HLA Class I antibody W6/32, hTAPBPR^{TN6}, hTAPBPR, chTAPBPR, and moTAPBPR binding to the SABs. a. MHC class I levels of the single antigen beads were detected using the primary anti-HLA Class I antibody W6/32 and a secondary PE-conjugated antibody. Similar levels of peptide-loaded MHC-I molecules are observed across the beads. b-e. Bar graphs of (b.) hTAPBPR^{TN6}; (c.) hTAPBPR; (d.) chTAPBPR; and (e.) moTAPBPR binding to HLA molecules upon treatment with tetramers at a 7 μM final concentration of the monomer. Incubation with the W6/32 antibody prior to tetramer addition was used to measure the background staining levels. The plotted data were generated based on two or three independent experiments and the standard deviation is depicted with error bars.

FIGS. 24A-24G. Allele and peptide dependence of the interactions between HLA-A allotypes and TAPBPR orthologs. a-g. SPR sensorgrams of various concentrations of peptide-loaded or -deficient (a.) HLA-A*01:01; (b.) HLA-A*30:01; (c.) HLA-A*29:02; (d.) HLA-A*02:01; (e.) HLA-A*68:02; (f.) HLA-A*11:01; (g.) HLA-A*24:02 flowed over a streptavidin chip coupled with hTAPBPR or chTAPBPR-biotin. The concentrations of analyte for the top and the bottom sensorgrams are noted. Data are mean±SD for n=2 or 3 technical replicates. K_{D}, equilibrium constant; kₐ, association rate constant; k_{d}, dissociation rate constant; RU, resonance units.

FIGS. 25A-25E. Ortholog dependence of the interactions between HLA-B allotypes, MHC-Ib, and MR1 and TAPBPR orthologs. a-d. SPR sensorgrams of vary concentrations of peptide-loaded or -deficient (a.) HLA-B*08:01; (b.) HLA-B*37:01; (c.) HLA-E*01:03; (d.) HLA-G*01:01 flowed over a streptavidin chip coupled with hTAPBPR or chTAPBPR-biotin. e. SPR sensorgrams of vary concentrations of Ac-6-FP loaded MRI C262S flowed over a streptavidin chip coupled with hTAPBPR or chTAPBPR-biotin. The concentrations of analyte for the top and the bottom sensorgrams are noted. Data are mean±SD for n=2 or 3 technical replicates. K_{D}, equilibrium constant; kₐ, association rate constant; k_{d}, dissociation rate constant; RU, resonance units.

FIGS. 26A-26C. Structure-guided design of conditional peptide ligands influences TAPBPR-mediated peptide exchange kinetics for different HLA allotypes. The conditional peptide ligand KILGFVFβFV (SEQ ID NO: 12) is based on the HLA-A*02:01-restricted influenza Matrix 1 (58-66) epitope GILGFVFTL (SEQ ID NO: 13), where Gly1 is replaced with lysine for solubility and where Thr8 is replaced with a L-β-Phenylalanine residue termed βF and where anchor residue Leu9 is replaced with valine for increased HLA affinity. βF=L-β-Phenylalanine. (A.) Assessing the effect of the L-β-Phenylalanine residue (βF) at different peptide position by fluorescence polarization (FP). The association profiles of the fluorophore-conjugated peptide _{TAMRA}KLFGYPVYV (SEQ ID NO: 27) to HLA-A*02:01/βFILGFVFTV (SEQ ID NO: 66) (upper left), KIβFGFVFTV (SEQ ID NO: 67) (upper middle), KILβFFVFTV (SEQ ID NO: 68) (upper right), KILGβFVFTV (SEQ ID NO: 69) (middle left), KILGFβFFTV (SEQ ID NO: 70) (middle middle), KILGFVβFTV (SEQ ID NO: 71) (middle right), KILGFVFβFV (SEQ ID NO: 12) (lower left) without hTAPBPR with 10 nM or 5000 nM hTAPBPR, as indicated. The data were fitted to a monoexponential association model to determine apparent rate constants kₐₚₚ. (B.) The comparison of kₐₚₚ for fluorescent peptide binding to HLA-A*02:01/KILGFVFTV (SEQ ID NO: 33) with βF substitution at indicated positions in the presence of 5000 nM hTAPBPR. The apparent rate constant kₐₚₚ was determined by fitting the raw trace to a monoexponential association model. Results of three replicates (mean±σ) are plotted. (C.) Structure-guided design of conditional peptide ligands for different HLA allotypes. The βF peptide ligands KLIETYFβFK (SEQ ID NO: 37), KTFPPTEβFK (SEQ ID NO: 38), ETAGIGILβFV (SEQ ID NO: 39), and FEDLRVPFSF (SEQ ID NO: 17) are based on the HLA-A*03:01 immunodominant proteolipid protein (PLP) epitope KLIETYFSK (SEQ ID NO: 40), HLA-A*30:01 SARS-CoV-2 nucleoprotein epitope KTFPPTEPK (SEQ ID NO: 41), HLA-A*68:02 peptide ETAGIGILTV (SEQ ID NO: 42), and HLA-B*37:01 Influenza NP338-346 peptide FEDLRVLSF (SEQ ID NO: 22).

FIG. 27A-27J. TAPBPR orthologs catalyze peptide exchange kinetics differently for HLA-A*02 subtypes. a-e. The association profiles of the fluorophore-conjugated peptide _{TAMRA}KLFGYPVYV (SEQ ID NO: 27) to (a.) HLA-A*02:01/KILGFVFβFV (SEQ ID NO: 12); (b.) A*02:06; (c.) A*02:11; (d.) A*02:71; and (e.) A*02:77 in the presence of hTAPBPR at various concentrations, as indicated. The data were fitted to a monoexponential association model to determine apparent rate constants kₐₚₚ. f-j. The apparent rate constant kₐₚₚ of fluorophore-conjugated peptide binding to (f.) HLA-A*02:01/KILGFVFβFV (SEQ ID NO: 12); (g.) A*02:06; (h.) A*02:11; (i.) A*02:71; and (j.) A*02:77 is correlated with the hTAPBPR concentrations. Extrapolation of the slope allowed calculating the overall rate k₂ₒᵥₑᵣₐₗₗ, describing the formation of fluorescent peptide-loaded MHC-I from diverse pathways under the effect of TAPBPR orthologs. Averages of at least three independent experiments±SEM are shown. βF=DL-β-Phenylalanine.

FIGS. 28A-28F. TAPBPR orthologs catalyze HLA peptide exchange kinetics differently. a-f. The association profiles of fluorophore-conjugated peptide _{FITC}KSDPIVAQY (SEQ ID NO: 34), _{TAMRA}KLFGYPVYV (SEQ ID NO: 27), _{FITC}KLIETYFSK (SEQ ID NO: 35). and _{TAMRA}KYNPIRTTF (SEQ ID NO: 36) to (a.) HLA-A*01:01/STAPGJLEY (SEQ ID NO: 9); (b.) A*02:01/KILGFVFβFV (SEQ ID NO: 12); (c.) A*03:01/KLIETYFβFK (SEQ ID NO: 37); (d.) A*11:01/KLIETYFβFK (SEQ ID NO: 72) (e.), A*24:02/VYGJVRACL (SEQ ID NO: 3); and (f.) A*68:02/ETAGIGILβFV (SEQ ID NO: 39) in the presence of human or chicken TAPBPR at various concentrations, as indicated. The data were fitted to a monoexponential association model to determine apparent rate constants kₐₚₚ. The apparent rate constant kₐₚₚ of fluorophore-conjugated peptide binding to each allotype is correlated with the human or chicken TAPBPR concentrations. Extrapolation of the slope allowed calculating the overall rate k₂ₒᵥₑᵣₐₗₗ, describing the formation of fluorescent peptide-loaded MHC-I from diverse pathways under the effect of TAPBPR orthologs. Averages of at least three independent experiments±SEM are shown. βF=L-β-Phenylalanine.

FIGS. 29A-29B. TAPBPR orthologs catalyze HLA-B peptide exchange kinetics differently. a-b. The association profiles of fluorophore-conjugated peptide _{FITC}KLRGRAJGL (SEQ ID NO: 85) and _{FITC}KEDLRVSSF (SEQ ID NO: 84) to (a.) B*08:01/FLRGRAJGL (SEQ ID NO: 4) and (b.) B*37:01/FEDLRVJSF (SEQ ID NO: 63) in the presence of human or chicken TAPBPR at various concentrations, as indicated. The data were fitted to a monoexponential association model to determine apparent rate constants kₐₚₚ. The apparent rate constant kₐₚₚ of fluorophore-conjugated peptide binding to each allotype is correlated with the human or chicken TAPBPR concentrations. Extrapolation of the slope allowed calculating the overall rate k₂ₒᵥₑᵣₐₗₗ, describing the formation of fluorescent peptide-loaded MHC-I from diverse pathways under the effect of TAPBPR orthologs. Averages of at least three independent experiments±SEM are shown. βF=L-β-Phenylalanine.

FIGS. 30A-30H. TAPBPR orthologs promote exchange of high-affinity peptides on HLA-A*01:01, HLA-B*08:01, HLA-A*02:01, and HLA-B*37:01 by DSF. a. Melting temperature (Tₘ, ° C.) obtained from DSF of HLA-A*01:01 bound to ILDTAGQEEY (SEQ ID NO: 83), ESDPIVAQY (SEQ ID NO: 6), CTDDNALAYY (SEQ ID NO: 5), or PTDNYITTY (SEQ ID NO: 7). Data are mean±SD for n=3 technical replicates. b. Thermal stabilities of HLA-A*01:01/ILDTAGQEEY (SEQ ID NO: 83) were determined in the presence of 10-fold molar excess ESDPIVAQY (SEQ ID NO: 6), CTDDNALAYY (SEQ ID NO: 5), PTDNYITTY (SEQ ID NO: 7), or a non-specific peptide YPNVNIHNF (SEQ ID NO: 8) for two-hour incubation at RT with catalytic amount of human or chicken TAPBPR. c. Melting temperature (Tₘ, ° C.) obtained from DSF of HLA-B*08:01 bound to peptide RPHERNGFTVL (SEQ ID NO: 10), FLRGRAYGL (SEQ ID NO: 21), ALWMRLLPL (SEQ ID NO: 79), or MLYQHLLPL (SEQ ID NO: 80). Data are mean±SD for n=3 technical replicates. d. Thermal stabilities of HLA-B*08:01/RPHERNGFTVL (SEQ ID NO: 10) were determined in the presence of 10-fold molar excess FLRGRAYGL (SEQ ID NO: 21), ALWMRLLPL (SEQ ID NO: 79), MLYQHLLPL (SEQ ID NO: 80), or a non-specific peptide AIFQSSMTK (SEQ ID NO: 81) for two-hour incubation at RT with a catalytic amount of human or chicken TAPBPR. e. Melting temperature (Tₘ, ° C.) obtained from DSF of HLA-A*02:01 bound to KILGFVFJV (SEQ ID NO: 25) and LLFGYPVYV (SEQ ID NO: 26). Data are mean±SD for n=3 technical replicates. f. Thermal stabilities of HLA-A*02:01/KILGFVFJV (SEQ ID NO: 25) in the presence of 10-fold molar excess LLFGYPVYV (SEQ ID NO: 26) and a non-specific peptide YPNVNIHNF (SEQ ID NO: 8) for two-hour incubation at RT with a catalytic amount of human or chicken TAPBPR. g. Melting temperature (Tₘ, ° C.) obtained from DSF of HLA-B*37:01 bound to FEDLRVPFSF (SEQ ID NO: 17) and FEDLRVLSF (SEQ ID NO: 22). Data are mean±SD for n=3 technical replicates. h. Thermal stabilities of HLA-B*37:01/FEDLRVPFSF (SEQ ID NO: 17) in the presence of 10-fold molar excess FEDLRVLSF (SEQ ID NO: 22) and a non-specific peptide NLVPMVATV (SEQ ID NO: 82) for two-hour incubation at RT with a catalytic amount of human or chicken TAPBPR.

FIGS. 31A-31C. ChTAPBPR interacts with both peptide-loaded and -receptive HLA-B*08:01. a. Representative ITC data titrating 150 μM HLA-B*08:01/FLRGRAJGL (SEQ ID NO: 4) with β-fold excess peptide FLRGRAJGL (SEQ ID NO: 4) into a sample containing 5 μM chTAPBPR with 0.45 mM FLRGRAJGL (SEQ ID NO: 4). Black lines are the fits of the isotherm. Fitted values for K_{D}, ΔH, −TΔS, and ΔG were determined using a 1-site binding model. b. ChTAPBPR and the mixture of chTAPBPR and HLA-B*08:01/FLRGRAJGL (SEQ ID NO: 4) after 40-minute UV irradiation were analyzed by size exclusion chromatography (SEC). The peaks of chTAPBPR, HLA-B*08:01/FLRGRAJGL (pB*08:01) (SEQ ID NO: 4), and chTAPBPR/HLA-B*08:01 complex were collected, concentrated, and analyzed by SDS/PAGE, which confirmed the identity of the complex peak. c. Native gel electrophoretic mobility shift assays of HLA-B*08:01/FLRGRAJGL (SEQ ID NO: 4) (lane 1) and in the presence of 10-fold molar excess of a non-specific peptide AIFQSSMTK (SEQ ID NO: 81) (HIV, lane 2) or a high affinity peptide FLRGRAYGL (SEQ ID NO: 21) (EBV, lane 3) incubated with UV irradiated HLA-B*08:01 for one hour. Peptide-receptive chTAPBPR/HLA-B*08:01 complexes were loaded alone (lane 4) and with 10-fold molar excess peptide AIFQSSMTK (SEQ ID NO: 81) (HIV, lane 5), FLRGRAYGL (SEQ ID NO: 21) (EBV, lane 6), ALWMRLLPL (SEQ ID NO: 79) (T1D30, lane 7), and MLYQHLLPL (SEQ ID NO: 80) (T1D95, lane 8). ChTAPBPR alone is also loaded as a control (lane 10). The data shown are representative of triplicate gel assays.

FIGS. 32A-32D. TAPBPR-TM promotes surface expression of HLA-A*02:01. a. Tapasin-KO Expi293F cells were transiently transfected with FLAG-tagged TAPBPR. (Top) Western blots show total expression levels of TAPBPR proteins. The housekeeping enzyme PPIB was detected as a loading control. (Center) Surface levels of endogenous HLA-A*02:01 were measured by flow cytometry. Fluorescence levels of vector-only transfected cells were subtracted. hTAPBPR mediates a small increase in surface HLA-A*02:01 that is not disrupted by the TN6 mutations, whereas hTAPBPR-TM mediates a large increase in surface HLA-A*02:01 that is decreased by the TN6 mutations. The data are consistent with intracellular TAPBPR proteins interacting with nascent HLA-A*02:01 (and sequestering a pool of HLA-A*02:01 intracellularly) in a manner independent of the TN6 mutations, whereas surface interactions are diminished by the TN6 mutations. (Bottom) Surface levels of FLAG-tagged TAPBPR measured by flow cytometry. TAPBPR with a native C-terminus is primarily intracellular, whereas TAPBPR-TM proteins traffic to the plasma membrane. Data are mean±SD, n=3 independent replicates. b. Tapasin-KO Expi293F cells were transfected with WT, S104F, and K211L hTAPBPR-TM. (Top) Total expression of hTAPBPR-TM proteins by western blot, (Center) surface expression of HLA-A*02:01 by flow cytometry, and (Bottom) surface expression of hTAPBPR-TM proteins by flow cytometry. Data are mean±SD, n=5 independent replicates. c. To generate an HLA-A*01:01 positive/tapasin-KO line, Expi293F cells (parental, black) were stably transfected with untagged HLA-A*01:01. The tapasin gene was then targeted by Cas9 as previously described to create the final line. Cells were stained with anti-HLA-A*01:01 for flow cytometry analysis. d. HLA-A*01:01 positive/tapasin-KO Expi293F cells were transfected with TAPBPR constructs. (Top) Total expression of TAPBPR proteins by western blot, (Center) surface expression of HLA-A*01:01 by flow cytometry, and (Bottom) surface expression of TAPBPR proteins by flow cytometry. The TAPBPR proteins have no effect on surface HLA-A*01:01 levels. Data are mean±SD, n=3 independent replicates.

FIGS. 33A-33F. The mutational landscape of human TAPBPR-TM based on promoting surface expression of HLA-A*02:01. a. Log (2) enrichment ratios from the deep mutational scan are plotted from ≤−3 (deleterious/depleted mutations) to ≥+3 (enriched mutations). Amino acid substitutions are on the horizontal axis, hTAPBPR-TM residue position is on the vertical axis. *, stop codon. Wild type amino acids are in black. b-d. Heat maps plot the log (2) enrichment ratios from ≤−3 to ≥+3 for mutations in hTAPBPR-TM in the (a.) core, (b.) on the surface away from the MHC-I interface, and (c.) in the 24-35 loop. Amino acid substitutions are on the vertical axes, hTAPBPR-TM residue positions are on the horizontal axes. e. Log (2) enrichment ratios for each mutation in hTAPBPR-TM are plotted from two independent sorting experiments. R² is indicated for the agreement between replicates for missense mutations in black. Nonsense mutations are shaded. f. Conservation scores, calculated by averaging the log (2) enrichment ratios for all amino acid substitutions at a given hTAPBPR-TM residue, are plotted showing close agreement between two independent sorting experiments.

FIGS. 34A-34D. Sorting strategy for the deep mutational scan of TAPBPR-TM. Tapasin-KO Expi293F cells were transfected with a plasmid library encoding TAPBPR-TM mutants, under conditions previously shown to result in cells typically expressing no more than a single coding sequence. Flow cytometry histograms show how the transfected cell cultures were gated for FACS to enrich for cells expressing TAPBPR-TM mutants that promote surface HLA-A*02:01 localization. Cells were gated for viability (a. DAPI negative), by forward scattering (FSC-A/area and FSC-W/width) to exclude doublets (b.), by forward (FSC) and side (SSC) scattering for the main population (c.), and by fluorescence for high levels of staining with anti-HLA-A*02:01 PE conjugate (d.). Gates are shown in magenta and the percentages of events that are gated are in parentheses.

FIGS. 35A-35B. Expression and purification of hTAPBPR mutants. a. SEC traces of hTAPBPR S104F K211L R270Q (hTAPBPR^{FLQ}), R105K K211R R270Q (hTAPBPR^{KRQ}), and K211R R270Q (hTAPBPR^{RQ}) mutants. The protein peak is indicated by the arrow and is further confirmed by SDS/PAGE analysis. b. DSF of hTAPBPR (Tₘ=53.0° C.) relative to hTAPBPR^{FLQ} (Tₘ=51.8° C.), hTAPBPR^{KRQ} (Tₘ=52.3° C.), and hTAPBPR^{RQ} (Tₘ=51.0° C.).

FIGS. 36A-36F. Human TAPBPR mutants show enhanced peptide exchange kinetics on HLA-A*02 and enable exchange on HLA-A*01:01 molecules. A-B. The association profiles of fluorophore-conjugated peptide _{TAMRA}KLFGYPVYV (SEQ ID NO: 27) to (A.) HLA-A*02:01/KILGFVFβFV (SEQ ID NO: 12) and (B.) HLA-A*02:06/KILGFVFβFV (SEQ ID NO: 12) in the presence of hTAPBPR, hTAPBPR^{RQ}, hTAPBPR^{KRQ}, and hTAPBPR^{FLQ} at various concentrations, as indicated. The data were fitted to a monoexponential association model to determine apparent rate constants kₐₚₚ. The apparent rate constant kₐₚₚ of fluorophore-conjugated peptide binding to each allotype is correlated with the human or chicken TAPBPR concentrations. Extrapolation of the slope allowed calculating the overall rate k₂ₒᵥₑᵣₐₗₗ, describing the formation of fluorescent peptide-loaded MHC-I from diverse pathways under the catalytic effect of TAPBPR orthologs. Averages of at least three independent experiments±SEM are shown. βF=DL-β-Phenylalanine. (C.) The association profiles of _{TAMRA}KLFGYPVYV (SEQ ID NO: 27) to HLA-A*02:01/βFILGFVFTV (p1) (SEQ ID NO: 66), KIβFGFVFTV (p3) (SEQ ID NO: 67), KILβFFVFTV (p4) (SEQ ID NO: 68), KILGβFVFTV (p5) (SEQ ID NO: 69), KILGFβFFTV (p6) (SEQ ID NO: 70), KILGFVβFTV (p7) (SEQ ID NO: 71), KILGFVFβFV (p8) (SEQ ID NO: 12) without hTAPBPR^{FLQ} (buffer), with 10 nM or 100 nM hTAPBPR, as indicated. The data were fitted to a monoexponential association model to determine the apparent rate constant kₐₚₚ. Results of three replicates are plotted. (D.), The comparison of kₐₚₚ for fluorescent peptide binding to HLA-A*02:01/KILGFVFTV (SEQ ID NO: 33) with βF substitution at indicated positions in the presence of 10 nM or 100 nM hTAPBPR. The apparent rate constant kₐₚₚ was determined by fitting the raw trace to a monoexponential association model. Results of three replicates (mean±6) are plotted. (E.) The association profiles of fluorophore-conjugated peptide _{FITC}KLRGRAYGL (SEQ ID NO: 78) to HLA-B*08:01/FLRGRAJGL (SEQ ID NO: 4) in the presence of hTAPBPR, hTAPBPR^{KRQ}, and hTAPBPR^{FLQ} at various concentrations, as indicated. J=3-amino-3-(2-nitrophenyl)-propionic acid. (F.) The association profiles of fluorophore-conjugated peptide _{FITC}KSDPIVAQY (SEQ ID NO: 34) to HLA-A*01:01/STAPGJLEY (SEQ ID NO: 9) in the presence of hTAPBPR, hTAPBPR^{RQ}, hTAPBPR^{KRQ}, and hTAPBPR^{FLQ} at various concentrations, as indicated. The data were fitted to a monoexponential association model to determine apparent rate constants kₐₚₚ. Averages of at least three independent experiments±SEM are shown.

FIGS. 37A-37C. Mutant dependence of the interactions between peptide-loaded or -deficient HLA-A*02:01 or MR1 and TAPBPR orthologs. a. SPR sensorgrams of vary concentrations of peptide-loaded HLA-A*02:01 flowed over a streptavidin chip coupled with hTAPBPR, hTAPBPR^{RQ}, hTAPBPR^{KRQ}, or hTAPBPR^{FLQ}-biotin. b. SPR sensorgrams of vary concentrations of peptide-deficient (empty) HLA-A*02:01 flowed over a streptavidin chip coupled with hTAPBPR, hTAPBPR^{RQ}, hTAPBPR^{KRQ}, or hTAPBPR^{FLQ}-biotin. c. SPR sensorgrams of vary concentrations of Ac-6-FP loaded MR1 C262S flowed over a streptavidin chip coupled with hTAPBPR, hTAPBPR^{RQ}, hTAPBPR^{KRQ}, or hTAPBPR^{FLQ}-biotin. The concentrations of analyte for the top and the bottom sensorgrams are noted. Data are mean±SD for n=2 or 3 technical replicates. K_{D}, equilibrium constant; kₐ, association rate constant; k_{d}, dissociation rate constant; RU, resonance units.

FIG. 38. Human vs. chicken Tapasin DSF. Differential Scanning Fluorimetry assay of recombinant proteins encompassing the luminal domain of human or chicken Tapasin. Human (hTapasin) has a melting temperature, Tₘ of 41.0° C. and chicken (chTapasin) a Tₘ of 58.0° C., demonstrating a significant increase in thermal stability.

FIGS. 39A-39B. chTapasin MFI ratios plot. Adapted Single Antigen Bead assay used to measure levels of binding to individual HLA-I allotypes by streptavidin-PE conjugated Tapasin orthologs. SABs are color-coded by different ratios of fluorescent dyes and are decorated with different HLA-I allotypes. The SABs were incubated with 7 μM of tetramerized Tapasin for 1 hour at room temperature (RT). Bar graph showing the levels of a. human Tapasin (hTapasin-a) and b. chicken Tapasin (chTapasin-b) binding to the SABs. Incubation with the W6/32 antibody prior to tetramer staining was used to control for the non-specific background binding, and the MFI ratio of Tapasin/control was determined. Plotted data are mean±SD from 2 independent experiments.

FIG. 40. chTapasin MFI ratios plot. Molecular chaperones and their engineered variants can interact with different sets of folded HLA-I allotypes. Box plot showing distributions of log₁₀ Mean Fluorescence Intensity (MFI) levels for binding of fluorescent tetramers conjugated with either human TAPBPR (hTAPBPR), three engineered mutants of human TAPBPR (FLQ, KRQ, RQ corresponding to the hTAPBPR^{FLQ} hTAPBPR^{KRQ} and hTAPBPR^{RQ} variants, respectively), chicken TAPBPR (chTAPBPR), human Tapasin (hTapasin) or chicken Tapasin (chTapasin) to single-antigen beads (SABs) conjugated with 97 common HLA-I allotypes. The left boundary of the box indicates the 25^{th} percentile, the line within the box marks the median and the right boundary of the box indicates the 75^{th} percentile. Whiskers above and below the box indicate the 10^{th} and 90^{th} percentiles. The points above the whiskers indicate outliers outside the 90^{th} percentile. A mutant form of the human chaperone TAPBPR, hTAPBPR^{TN6} (TN6) shown previously to abrogate binding to MIC-I molecules (Dong et al., Immunity; Hermann et al., J. Immunol) was used as a negative control for background staining levels, to determine significant interactions between TAPBPR orthologs and MHC-I molecules, and a cutoff value of 1.8 log₁₀(MFI) was used to determine significance levels. HLA allotypes detected to bind above this threshold and are therefore indicated to interact with each chaperone in either the peptide-loaded or peptide-deficient states are outlined in Table 4.

FIGS. 41A-41C. chTapasin MFI ratios plot. Molecular chaperones and their engineered variants can interact with different sets of folded HLA-I allotypes. Chicken Tapasin shows enhanced association with human HLA-I allotypes and forms a strong interaction with HLA-B*37:01. A. Differential Scanning Fluorimetry of recombinantly expressed chicken Tapasin (chTapasin) showing an increased thermal stability (Tₘ 58° C.), relative to its human ortholog (hTapasin, Tₘ 41.0° C.). B. Box plot showing distributions of Mean Fluorescence Intensity (MFI) levels for binding of fluorescent tetramers conjugated with either human or chicken Tapasin to single-antigen beads (SABs) conjugated with 97 common HLA-I allotypes. A mutant form of the human chaperone TAPBPR, hTAPBPR^{TN6}, shown previously to aburaage binding to MIC-I molecules (Dong et al., Immunity. 2009; 30:21-32; Hermann et al., J. Immunol 2013 Dec. 1; 191(11):5743-50) was used as a negative control for background staining levels, to determine significant interactions between TAPBPR orthologs and MIC-I molecules. The left boundary of the box indicates the 25^{th} percentile, the line within the box marks the median and the right boundary of the box indicates the 75^{th} percentile. Whiskers above and below the box indicate the 10^{th} and 90^{th} percentiles. The points above the whiskers indicate outliers outside the 90^{th} percentile. Overall, chicken Tapasin shows enhanced association with human MHC-I allotypes. HLA-B*37:01, which shows tight binding to chTapasin, is indicated. C. Bar graph showing log₁₀ Mean Fluorescence Intensity (MFI) levels for binding of fluorescent tetramers conjugated with either human or chicken Tapasin to single-antigen beads (SABs) conjugated with 97 common HLA-I allotypes. Overall, chicken Tapasin shows enhanced association with human MHC-I allotypes. HLA-B*37:01, which exhibits strong association with chicken Tapasin, is indicated. Plotted data are mean±SD from 2 independent experiments.

FIG. 42. The macaque MHC-I Mafa-A1*063 forms a tight complex with the human chaperone TAPBPR. Native polyacrylamide gel electrophoresis analysis of interactions between different TAPBPR or Tapasin orthologs and Mafa-A1*063. Each well in lanes 1-9 was loaded with Mafa-A1*063/hβ₂m/GPRKPIKCW (SEQ ID NO: 86) complex, hTAPBPR, chTAPBPR, hTapasin, and chTapasin, or 1:1 molar ratio mixture, as indicated. As a positive control (lanes 10 and 11), we incubated HLA-A*02:01/hβ₂m/KILGFVFJV (SEQ ID NO: 25), where J=3-amino-3-(2-nitrophenyl)-propionic acid with human TAPBPR for 1 hour under 365 nm UV light, leading to peptide dissociation and formation of an empty TAPBPR/HLA-A*02:01/hβ₂m protein complex. New protein bands corresponding to the formed Mafa-A1*063/hTAPBPR and HLA-A*02:01/hTAPBPR complexes are indicated with arrows.

FIGS. 43A-43D. A graphic depiction of the free energy landscape for TAPBPR-mediated peptide exchange. A. MHC-I loaded with a moderate affinity placeholder peptide can spontaneously but slowly generate empty molecules. B. These empty molecules overcome a high activation energy barrier (ΔG^{‡}_{uncat}) to be in the transient peptide-receptive state for peptide loading. C. A catalytic amount of TAPBPR functions to lower the activation energy barrier (ΔG^{‡}_{cat}) by recognizing the empty molecules with high affinity (binding free energy ΔG_{B2}) and stabilizing an “open” conformation for peptide loading. D. Loading of high affinity peptides induces a “closed” MHC-I groove conformation leading to TAPBPR dissociation, due to the substantially reduced affinity of TAPBPR towards peptide-loaded molecules (indicated by binding free energies ΔG_{B1} and ΔG_{B3}). The reaction coordinate diagram from left-to-right shows the exchange of a moderate affinity peptide to a high affinity peptide, which can happen in the opposite direction with a much higher activation energy barrier and thus slower kinetics.

FIGS. 44A-44B. The chicken chaperone TAPBPR forms a tight complex with the HLA-F*01:01 allotype. A. Native polyacrylamide gel electrophoresis analysis of interactions between human TAPBPR wild-type (hTAPBPR), hTAPBPR carrying the mutations S104F, K211L and K270Q (FLQ), and chicken TAPBPR with HLA-F*01:01. Each well in lanes 1-7 was loaded with HLA-F*01:01/hβ₂m/LILRWE(βF)D (SEQ ID NO: 112) complex, hTAPBPR, hTAPBPRFLQ and chTAPBPR, or 1:1 molar ratio mixture, as indicated. A new protein band corresponding to the formed HLA-F*01:01/chTAPBPR complex is indicated by the arrow (lane 7). B. Representative sensorgram of HLA-F*01:01/hβ₂m/LILRWE(βF)D (SEQ ID NO: 112) flowed over a streptavidin chip coupled with chTAPBPR-biotin. K_{D}, equilibrium constant; kₐ, association rate constant; k_{d}, dissociation rate constant; RU, resonance units. The concentrations of analyte for the top and the bottom sensorgrams are noted. Fits from the kinetic analysis are shown with red lines.

### DETAILED DESCRIPTION OF THE INVENTION

This invention relates to a recombinant ligand exchange protein that comprises a fragment of TAP-binding protein-related (TAPBPR). The luminal domain of TAPBPR is shown herein to function as a peptide editor and a ligand exchange protein comprising this domain acts as an extracellular or cell surface MHC class I ligand exchange catalyst that is capable of loading exogenous peptide onto MHC class I molecules on the surface of a cell.

The ligand exchange protein may comprise a fragment of TAP-binding protein-related (TAPBPR). A fragment is a truncated TAPBPR protein that lacks one or more amino acids of the full-length protein but retains ligand exchange activity. For example, a fragment may lack a contiguous sequence of 10 or more, 20 or more, 50 or more of 100 or more amino acids, relative to the full-length TABPR protein. In some embodiments, a TAPBPR fragment may lack the ectodomain and/or transmembrane domain of the full-length TAPBPR protein. A suitable TAPBPR fragment may comprise or consist of the luminal domain of the full-length TAPBPR protein.

A ligand exchange protein as described herein may be soluble and not bound to a membrane either at the surface or within a mammalian cell. In particular, the ligand exchange protein may lack transmembrane domains, membrane anchors or other features that might covalently attach it to an intracellular membrane or the cell membrane during or after expression.

In a soluble ligand exchange protein as described herein, the TAPBPR fragment may consist of the luminal domain of TAPBPR. A soluble ligand exchange protein may lack sequence from TAPBPR outside the luminal domain i.e. the TAPBPR fragment may be the only TAPBPR sequence in the ligand exchange protein. For example, the ligand exchange protein may lack the TAPBPR transmembrane domain, ectodomain or other non-luminal domains.

In other embodiments, a ligand exchange protein as described herein may be bound to the plasma membrane at the surface of a mammalian cell. For example, the ligand exchange protein may comprise a transmembrane domain (TMD) that attaches the protein to the plasma membrane. The TMD may be a TAPBPR TMD or a heterologous TMD. In some embodiments, the TMD may be sufficient to localize the ligand exchange protein to the plasma membrane after expression. In other embodiments, the surface bound ligand exchange protein may further comprise a cell surface targeting sequence that localizes the ligand exchange protein to the plasma membrane after expression.

In a surface bound ligand exchange protein comprising a heterologous transmembrane domain, the TAPBPR fragment may comprise the luminal domain of TAPBPR. In other surface bound ligand exchange proteins, the TAPBPR fragment may comprise both the luminal domain and TMD of TAPBPR. The TAPBPR fragment of the ligand exchange protein displays ligand exchange activity and is capable of loading cell-surface MHC class I molecules with an exogenous peptide.

A cell displaying MHC class I molecules may be exposed to (i) a soluble extracellular ligand exchange protein as described herein (ii) a cell having a surface bound ligand exchange protein as described herein or (iii) a chimeric ligand exchange protein as described herein that binds to the surface of the cells displaying the MHC class I molecules.

The loading of cell-surface MHC class I molecules as described herein may increase the number of MHC class I molecules on the surface of a cell which present the exogenous peptide relative to cells not treated with the ligand exchange protein. For example, the number of MHC class I molecules on the surface of a cell which present the exogenous peptide may be increased by 30 fold or more, 40 fold or more, 50 fold or more, 60 fold or more, 70 fold or more, 80 fold or more, 90 fold or more, 100 fold or more, 150 fold or more or 200 fold or more exogenous peptide in the presence relative to the absence of TAPBPR. Cells may present none or substantially none of the exogenous peptide in the absence of treatment with the ligand exchange protein.

In some embodiments, the endogenous peptides presented by the cell displaying MHC class I molecules may not have the same amino acid sequence as the exogenous peptide. In other embodiments, the cell may present low levels of the endogenous peptide with the same amino acid sequence as the exogenous peptide. The ligand exchange protein may increase the amount of peptide having the amino acid sequence that is presented by loading MHC class I molecules on the cell surface with exogenous peptide.

Sufficient exogenous peptide may be loaded onto cell-surface MHC class I molecules to stimulate a T cell response to the peptide in an individual.

In some embodiments, the ligand exchange protein may consist of the TAPBPR fragment. This may be useful for example in altering the immunogenicity of mammalian cells in vitro or ex vivo. In a soluble ligand exchange protein, the TAPBPR fragment may comprise the TAPBPR luminal domain. For example, the TAPBPR fragment may lack the TAPBPR TMD and the TAPBPR cytoplasmic tail and may for example consist of the luminal domain. In a surface bound ligand exchange protein, the TAPBPR fragment may comprise the TAPBPR luminal domain and TMD. The TAPBPR fragment may lack the TAPBPR cytoplasmic tail and may for example consist of the TAPBPR luminal domain and TMD.

In other embodiments, the ligand exchange protein may further comprise one or more domains in addition to the TAPBPR fragment. The one or more additional domains may be heterologous domains (i.e. amino acid sequences not derived from TAPBPR). For example, a surface-bound ligand exchange protein may comprise a heterologous TMD and/or cell surface targeting sequence. In some embodiments, the ligand exchange protein may be a fusion protein comprising the TAPBPR fragment and one or more heterologous domains.

The absence of the TAPBPR cytoplasmic tail may be sufficient to localize a ligand exchange protein comprising a TAPBPR or heterologous TMD to the cell membrane. Suitable heterologous TMDs may include the platelet derived growth factor receptor (PDGFR) TMD, the influenza hemagglutinin TMD and the influenza neuraminidase TMD. In other embodiments, the ligand exchange protein may further comprise a heterologous cell surface targeting sequence. A cell surface targeting sequence is an amino acid sequence that directs a protein expressed in a cell to the plasma membrane. Suitable cell surface targeting sequences may include the cytoplasmic domains of CD8, MHC class I molecules, Transferrin receptor, CD147, VSVG, NCAM, CD44 or E-cadherin.

“Ligand” is a small (<2,000 Da) molecule which can bind to a classical MHC or non-classical MHC-I molecule. Different HLAs have different classes of ligands, as outlined herein; for example, classical HLA-A, -B, -C all bind peptides; non classical can bind either peptides, metabolites, lipids, etc., depending on the molecule; and herein is provided several ligands, including for MR1. A subgenus of “ligand” is peptide, and in addition to “ligand” being read as being both as to the genus but also as to each subgenus and species thereof, e.g., as to peptide and each peptide species, including as provided for in this text In this regard, mention is made of Ly et al., “The CD1 size problem: lipid antigens, ligands and scaffolds,” Cell Mol Life Sci 71(16):3069-3070 (2014), doi: 10.1007/s00018-014-1603-6, see also https://www.ncbi.nlm.nih.gov/pmc/articles/PMC4160407/(author manuscript), both incorporated herein by reference and providing a review of lipid ligands that bind to CD1 molecules and without wishing to be bound by any one theory, TAPBPR, including or comprising of the instant invention, are envisioned as working with CD1 (e.g., CD1 c mycoketide, CD1d Short chain alpha-galactosylceramide, CD1b diacylsulfoglycolipid, CD1b glucose monomycolate).

The term “heterologous” refers to a polypeptide or nucleic acid that is foreign to a particular biological system, such as a host cell, and is not naturally occurring in that system. A heterologous polypeptide or nucleic acid may be introduced to a biological system by artificial means, for example using recombinant techniques. For example, heterologous nucleic acid encoding a polypeptide may be inserted into a suitable expression construct which is in turn used to transform a host cell to produce the polypeptide. A heterologous polypeptide or nucleic acid may be synthetic or artificial or may exist in a different biological system, such as a different species or cell type. A recombinant polypeptide may be expressed from heterologous nucleic acid that has been introduced into a cell by artificial means, for example using recombinant techniques. A recombinant polypeptide may be identical to a polypeptide that is naturally present in the cell or may be different from the polypeptides that are naturally present in that cell.

The term “endogenous” refers to a peptide, polypeptide or nucleic acid or other factor that is generated by natural processes in a biological system, such as a host cell. The term “exogenous” refers to a peptide, polypeptide or nucleic acid that is not generated by natural processes in a biological system and is produced and/or introduced to the system by artificial means, for example by administration or recombinant expression. An exogenous factor may be synthesised using conventional techniques, such as solid-phase synthesis. An exogenous factor may be identical to a factor that is naturally present in a biological system (i.e. an endogenous factor) or may be different from the factors that are naturally present in that biological system.

Preferably, the ligand exchange protein further comprises a targeting domain. The ligand exchange protein described herein may be a chimeric protein or fusion protein comprising a targeting domain and a TAPBPR fragment comprising or consisting of the TAPBPR luminal domain. Chimeric ligand exchange proteins as described herein are preferably soluble and may be useful for example in altering the immunogenicity of mammalian cells in vivo, as well as for in vitro and ex vivo applications.

In some embodiments, the TAPBPR fragment may be at the N terminal of the ligand exchange protein and the targeting domain may be at the C terminal of the ligand exchange protein. In other embodiments, the TAPBPR fragment may be at the C terminal of the ligand exchange protein and the targeting domain may be at the N terminal of the ligand exchange protein.

The targeting domain may be directly connected to the TAPBPR fragment or may be connected via a linker.

Suitable linkers are well-known in the art and include chemical and peptidyl linkers. For example, a peptidyl linker may comprise a sequence of amino acid residues, for example, 5 to 30 or 5 to 22 amino acid residues, preferably 10 to 20 amino acid residues, more preferably about 12 amino acid residues.

Any linker sequence may be employed. Preferably, the linker sequence is a heterologous sequence. Suitable linker amino acid sequences are well known in the art and may include the amino acid sequences GGGGS, (GGGGS)₃ or GSTVAAPSTVAAPSTVAAPSGS, HVGGGGSGGGGSGGGGSTS or variants thereof.

The targeting domain allows the chimeric ligand exchange protein to selectively target a specific population of target cells in an individual. The targeting domain of the chimeric ligand exchange protein binds specifically to the target cells. Preferably, the targeting domain of the chimeric ligand exchange protein binds selectively to target cells relative to non-target cells i.e. it shows increased binding to target cells relative to non-target cells. Binding of the targeting domain to the target cells allows the TAPBPR fragment of the chimeric protein to act selectively at the surface of the target cells relative to non-target cells (i.e. cells to which the targeting domain does not bind), for example to load MHC class I molecules on the surface of the target cell with exogenous peptide.

Target cells may comprise MHC class I molecules on the cell surface. MHC class I molecules are heterodimers comprising an α chain and δ2-microglobulin. MHC class I molecules are expressed on all nucleated human cells. An individual inherits a set of HLA-A, -B and -C genes from each parent. These genes are co-dominantly expressed and nucleated cells in mammals express up to 6 different classical MHC class I molecules. MHC class I molecules are highly polymorphic within the α chain and there is huge variation within the population. MHC class I molecules may include HLA-A molecules, HLA-B molecules, such as HLA-B51, HLA-B15, HLA-B38, and HLA-B57 and HLA-C molecules, such as HLA-Cw1. Preferred MHC class I molecules include HLA-A.

In some embodiments, the target cells may be disease cells, such as cancer cells, cells infected with a pathogen, or other cells that cause disease. Increasing the immunogenicity of disease cells in an individual using a chimeric ligand exchange protein may generate or increase the strength of immune responses against the disease cells in the individual. This may lead to a reduction or eradication of disease cells in the individual and may exert a therapeutic effect.

In other embodiments, the target cells may be antigen presenting cells. Loading the surface MHC I molecules of antigen presenting cells with an exogenous immunogenic peptide useful in increasing or eliciting immune responses, for example T cell immune responses, against disease cells in vivo, thereby exerting a therapeutic effect.

In other embodiments, the target cells may be host cells that elicit an immune reaction, such as an autoimmune or auto inflammatory response. Loading the surface MHC I molecules of the host cells with an exogenous non-immunogenic peptide may be useful in reducing or preventing autoimmune or immune mediated inflammatory responses against the cells in vivo, thereby exerting a therapeutic effect.

The targeting domain may specifically bind to a marker, such a receptor or antigen that is present on the surface of a target cell of the individual. The binding affinity of the targeting domain for its target cell marker may be higher than the binding affinity of TAPBPR for MHC class I molecules.

Suitable targeting domains include any molecule that are capable of specific binding to a cell marker. For example, the targeting domain may be a ligand for a receptor on the surface of the target cell or an antibody molecule that specifically binds to an antigen on the surface of the target cell.

In some preferred embodiments, a chimeric ligand exchange protein comprising a targeting domain may show no binding or substantially no binding to MHC class I molecules on the surface of a cell if the target cell marker that is bound by the targeting domain is not present on the surface of the cell.

An antibody molecule is a polypeptide or protein comprising an antibody antigen-binding site. The term encompasses any immunoglobulin whether natural or partly or wholly synthetically produced. Antibody molecules may have been isolated or obtained by purification from natural sources, or else obtained by genetic recombination, or by chemical synthesis, and that they may contain unnatural amino acids.

Suitable antibody molecules may include whole antibodies and fragments thereof. Fragments of a whole antibody can perform the function of binding antigens. Examples of binding fragments are (i) the Fab fragment consisting of VL, VH, CL and CHi domains; (ii) the Fd fragment consisting of the VH and CHi domains; (iii) the Fv fragment consisting of the VL and VH domains of a single antibody; (iv) single-domain antibodies (sdAb) (also called nanobodies (Nb)) (Ward et al. (1989) Nature 341, 544-546; McCafferty et al., (1990) Nature, 348, 552-554; Holt et al. (2003) Trends in Biotechnology 21, 484-490), which consist of either a monomeric VH domain or a monomeric VL domain; (v) isolated CDR regions; (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al. (1988) Science, 242, 423-426; Huston et al. (1988) PNAS USA, 85, 5879-5883); (viii) bispecific single chain Fv dimers (PCT/US92/09965) and (ix) “diabodies”, multivalent or multispecific fragments constructed by gene fusion (WO94/13804; Holliger et al. (1993a), Proc. Natl. Acad. Sci. USA 90 6444-6448).

Fv, scFv, diabody, sdAb and other antibody molecules may be stabilized by the incorporation of disulphide bridges, for example linking the VH and VL domains (Reiter et al. (1996), Nature Biotech, 14, 1239-1245). Minibodies comprising a scFv joined to a CH3 domain may also be made (Hu et al. (1996), Cancer Res., 56(13):3055-61). Other examples of binding fragments are Fab′, which differs from Fab fragments by the addition of a few residues at the carboxyl terminus of the heavy chain CHi domain, including one or more cysteines from the antibody hinge region, and Fab′-SH, which is a Fab′ fragment in which the cysteine residue(s) of the constant domains bear a free thiol group.

In some preferred embodiments, the targeting domain may specifically bind to a target molecule, such as a tumor antigen, on a cancer cell. For example, the targeting domain may be an antibody molecule that binds to a tumor antigen.

The expression of one or more antigens (i.e. tumor antigens) may distinguish cancer cells from normal somatic cells in an individual. Normal somatic cells in an individual may not express the one or more antigens or may express them in a different manner, for example at lower levels, in different tissue and/or at a different developmental stage. Tumor antigens may therefore be used to target chimeric ligand exchange proteins specifically to cancer cells.

Tumor antigens expressed by cancer cells may include, for example, cancer-testis (CT) antigens encoded by cancer-germ line genes, such as MAGE-A1, MAGE-A2, MAGE-A3, MAGE-A4, MAGE-A5, MAGE-A6, MAGE-A7, MAGE-A8, MAGE-A9, MAGE-A10, MAGE-All, MAGE-A12, GAGE-I, GAGE-2, GAGE-3, GAGE-4, GAGE-5, GAGE-6, GAGE-7, GAGE-8, BAGE-I, RAGE-1, LB33/MUM-1, PRAME, NAG, MAGE-Xp2 (MAGE-B2), MAGE-Xp3 (MAGE-B3), MAGE-Xp4 (MAGE-B4), MAGE-C1/CT7, MAGE-C2, NY-ESO-I, LAGE-I, SSX-I, SSX-2 (HOM-MEL-40), SSX-3, SSX-4, SSX-5, SCP-I and XAGE and immunogenic fragments thereof (Simpson et al. Nature Rev (2005) 5, 615-625, Gure et al., Clin Cancer Res (2005) 11, 8055-8062; Velazquez et al., Cancer Immun (2007) 7, 1 1; Andrade et al., Cancer Immun (2008) 8, 2; Tinguely et al., Cancer Science (2008); Napoletano et al., Am J of Obstet Gyn (2008) 198, 99 e91-97).

Other tumor antigens include, for example, overexpressed, upregulated or mutated proteins and differentiation antigens particularly melanocyte differentiation antigens such as p53, ras, CEA, MUC1, PMSA, PSA, tyrosinase, Melan-A, MART-1, gp100, gp75, alpha-actinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-All, hsp70-2, KIAAO205, Mart2, Mum-2, and 3, neo-PAP, myosin class I, OS-9, pml-RAR.alpha. fusion protein, PTPRK, K-ras, N-ras, Triosephosphate isomerase, GnTV, Herv-K-mel, NA-88, SP17, and TRP2-Int2, (MART-I), E2A-PRL, H4-RET, IGH-IGK, MYL-RAR, ErbB2/her2, Epstein Barr virus antigens, EBNA, human papillomavirus (HPV) antigens E6 and E7, TSP-180, MAGE-4, MAGE-5, MAGE-6, pi85erbB2, pi80erbB-3, c-met, nm-23H1, PSA, PDL1, CD20, TAG-72-4, CA 19-9, CA 72-4, CAM 17.1, NuMa, K-ras, alpha.-fetoprotein, 13HCG, BCA225, BTAA, CA 125, CA 15-3 (CA 27.29BCAA), CA 195, CA 242, CA-50, CAM43, CD68KP1, CO-029, FGF-5, G250, Ga733 (EpCAM), HTgp-175, M344, MA-50, MG7-Ag, MOV18, NB170K, NY-CO-1, RCAS1, SDCCAG16, TA-90 (Mac-2 binding protein\cyclophilin C-associated protein), TAAL6, TAG72, TLP, TPS, tyrosinase related proteins such as TRP-1, TRP-2 and ABC transporters expressed on the surface of tumors that are the mediators of drug resistance, such as. P-gp, BCRP and MRP1,

Other tumor antigens include out-of-frame peptide-MHC complexes generated by the non-AUG translation initiation mechanisms employed by “stressed” cancer cells (Malarkannan et al. Immunity (1999) 10(6):681-90).

Other tumor antigens are well-known in the art (see for example WO00/20581; Cancer Vaccines and Immunotherapy (2000) Eds Stern, Beverley and Carroll, Cambridge University Press, Cambridge) The sequences of these tumor antigens are readily available from public databases but are also found in WO1992/020356 A1, WO1994/005304 A1, WO1994/023031 A1, WO1995/020974 A1, WO1995/023874 A1 and WO1996/026214 A1.

Suitable targeting domains, such as antibody molecules that specifically bind to tumor antigens, are well known in the art and may be generated using conventional techniques. In other embodiments, the targeting domain may specifically bind to a marker, such as a receptor, on an antigen presenting cell, such as a dendritic cell. For example, the targeting domain may be an Fc region that binds to an Fc receptor on the antigen presenting cell. Suitable Fc regions are well known in the art.

The targeting domain may be an antibody molecule that binds to a surface marker on the antigen presenting cell or a ligand or binding protein of the surface marker. Antigen presenting cells may include dendritic cells of any sub-type. XCR1+ dendritic cells mediate the cross-presentation of antigen for the activation of effector CD8+ T cells. Surface markers on XCR1+ dendritic cells may include XCR1, DNGR1 (CLEC9A) and BDCA3 (also known as CD141). CD172α+ dendritic cells induce T helper 2 (TH2) or TH17 cells, and promote of humoral immune responses. Surface markers on CD172α+ dendritic cells include CD172a and BDCA1 (also known as CD1c). Plasmacytoid DCs produce of type I interferon (IFN) during viral infections. Surface markers on plasmacytoid DCs include BDCA2 and BDCA4. Monocyte-derived DCs promote local T cell responses and enhance inflammation and chemokine production. Surface markers on monocyte-derived DCs include FcεRI and FcγRI expression is upregulated on activation. Macrophages eliminate pathogens and promote tissue homeostasis. Surface markers on macrophages include CD68. Expression of FcγRI is also upregulated on activation. Other suitable markers for dendritic cells include CD19, CD20, CD38, CD14 and/or Langerin/CD207.

In other preferred embodiments, the targeting domain may specifically bind to an antigen on a pathogen-infected cell. For example, the targeting domain may bind to a pathogen protein or a host cell protein whose surface expression is up-regulated by pathogen infection. For an HIV infected cell, the targeting domain may specifically bind to a marker on the cell surface, such as gp120 or gp41. Suitable targeting domains include antibody molecules or CD4, which specifically binds to surface gp120. For a CMV infected cell, the targeting domain may specifically bind to a viral protein such as UL11, UL142, UL9, UL1, UL5, UL16, UL55 (gB), UL74 (gO), UL75 (gH), UL155 (gL), which are all found on the surface of infected cells (Weekes et al (2014) Cell 157:1460-1472). Host proteins whose expression is upregulated or induced on the surface of infected cells include inhibitory NK receptor KLRG-1, which may be specifically bound using an E-cadherin (CDH1) targeting domain (Weekes et al (2014) Cell 157:1460-1472).

A protein described herein that is a variant of a reference sequence, such as a ligand exchange protein sequence described above, may have 1 or more amino acid residues altered relative to the reference sequence. For example, 50 or fewer amino acid residues may be altered relative to the reference sequence, preferably 45 or fewer, 40 or fewer, 30 or fewer, 20 or fewer, 15 or fewer, 10 or fewer, 5 or fewer or 3 or fewer, 2 or 1. For example, a variant described herein may comprise the sequence of a reference sequence with 50 or fewer, 45 or fewer, 40 or fewer, 30 or fewer, 20 or fewer, 15 or fewer, 10 or fewer, 5 or fewer, 3 or fewer, 2 or 1 amino acid residues mutated. For example, a chimeric protein described herein may comprise an amino acid sequence with 50 or fewer, 45 or fewer, 40 or fewer, 30 or fewer, 20 or fewer, 15 or fewer, 10 or fewer, 5 or fewer, 3 or fewer, 2 or 1 amino acid residue altered relative to any one of the herein disclosed TAPBR sequences.

An amino acid residue in the reference sequence may be altered or mutated by insertion, deletion or substitution, preferably substitution for a different amino acid residue. Such alterations may be caused by one or more of addition, insertion, deletion or substitution of one or more nucleotides in the encoding nucleic acid.

A protein as described herein that is a variant of a reference sequence, such as a ligand exchange protein sequence described above, may share at least 50% sequence identity with the reference amino acid sequence, at least 55%, at least 60%, at least 65%, at least 70%, at least about 80%, or at least 80% or at least 85% or at least 86% or at least 87% or at least 88% or at least 89% or at least 90% or at least 91% or at least 92% or at least 93% or at least 94% or at least 95% or at least 96% or at least 97% at least or 98% or at least 99% sequence identity. For example, a variant of a protein described herein may comprise an amino acid sequence that has at least 50% sequence identity with the reference amino acid sequence, at least 55%, at least 60%, at least 65%, at least 70%, at least about 80%, or at least 85% or at least 86% or at least 87% or at least 88% or at least 89% or at least 90% or at least 91% or at least 92% or at least 93% or at least 94% or at least 95% or at least 96% or at least 97% at least or 98% or at least 99% sequence identity with the reference amino acid sequence, for example one or more of the herein disclosed sequences.

Sequence identity is commonly defined with reference to the algorithm GAP (Wisconsin GCG package, Accelerys Inc, San Diego USA). GAP uses the Needleman and Wunsch algorithm to align two complete sequences that maximizes the number of matches and minimizes the number of gaps. Generally, default parameters are used, with a gap creation penalty=12 and gap extension penalty=4. Use of GAP may be preferred but other algorithms may be used, e.g. BLAST (which uses the method of Altschul et al. (1990) J. Mol. Biol. 215: 405-410), FASTA (which uses the method of Pearson and Lipman (1988) PNAS USA 85: 2444-2448), or the Smith-Waterman algorithm (Smith and Waterman (1981) J. Mol Biol. 147: 195-197), or the TBLASTN program, of Altschul et al. (1990) supra, generally employing default parameters. In particular, the psi-Blast algorithm may be used (Nucl. Acids Res. (1997) 25 3389-3402). Sequence identity and similarity may also be determined using Genomequest™ software (Gene-IT, Worcester Mass. USA).

Sequence comparisons are preferably made over the full-length of the relevant sequence described herein.

A ligand exchange protein described herein may further comprise one or more heterologous amino acid sequences additional to the TAPBPR fragment and optional targeting domain and/or linker. For example, the ligand exchange protein may further comprise one or more additional domains which improve stability, pharmacokinetics, targeting, affinity, purification and/or production properties.

In some embodiments, the ligand exchange protein described herein may further comprise a protease recognition site located between the targeting domain and the TAPBPR fragment. This may be useful for example, in clearing TAPBPR from the target cell, if required. Suitable proteases may include trypsin, chymotrypsin, factor Xa, tobacco etch virus (TEV) protease, thrombin and papain. Other suitable site specific proteases are well-known in the art and any site-specific endoprotease may be used.

In some embodiments, the ligand exchange protein may further comprise a reactive moiety to permit the use of “click chemistry” for conjugation with the targeting domain or other domain. Click-chemistry may for example involve the Cu(I)-catalysed coupling between two components, one containing an azido group and the other a terminal acetylene group, to form a triazole ring. Since azido and alkyne groups are inert to the conditions of other coupling procedures and other functional groups found in proteins are inert to click chemistry conditions, click-chemistry allows the controlled attachment of almost any linker or chemical group to the ligand exchange protein under mild conditions and in particular allows the chemical conjugation of a targeting domain to a TAPBPR fragment. For example, cysteine residues of the ligand exchange protein may be reacted with a bifunctional reagent containing a thiol-specific reactive group at one end (e.g. iodoacetamide, maleimide or phenylthiosulfonate) and an azide or acetylene at the other end. Label groups may be attached to the terminal azide or acetylene using click-chemistry. For example, a second linker with either an acetylene or azide group on one end of a linker and a chelate (for metal isotopes) or leaving group (for halogen labelling) on the other end (Baskin, J. (2007) PNAS 104(43)16793-97) may be employed.

Ligand exchange proteins as described herein may be provided using synthetic or recombinant techniques which are standard in the art.

In some embodiments, the ligand exchange protein described herein may be produced with an affinity tag, which may, for example, be useful for purification. An affinity tag is a heterologous peptide sequence which forms one member of a specific binding pair. Polypeptides containing the tag may be purified by the binding of the other member of the specific binding pair to the polypeptide, for example in an affinity column. For example, the tag sequence may form an epitope which is bound by an antibody molecule. Suitable affinity tags include for example, glutathione-S-transferase, (GST), maltose binding domain (MBD), MRGS(H)₆, DYKDDDDK (FLAG™), T7-, S-(KETAAAKFERQHMDS), poly-Arg (R₅₋₆), poly-His (H₂₋₁₀), poly-Cys (C₄) poly-Phe(F₁₁) poly-Asp(D₅₋₁₆), SUMO tag (Invitrogen Champion pET SUMO expression system), Strept-tag II (WSHPQFEK), c-myc (EQKLISEEDL), Influenza-HA tag (Murray, P. J. et al (1995) Anal Biochem 229, 170-9), Glu-Glu-Phe tag (Stammers, D. K. et al (1991) FEBS Lett 283, 298-302), Tag. 100 (Qiagen; 12 aa tag derived from mammalian MAP kinase 2), Cruz tag 09™ (MKAEFRRQESDR, Santa Cruz Biotechnology Inc.) and Cruz tag 22™ (MRDALDRLDRLA, Santa Cruz Biotechnology Inc.). Known tag sequences are reviewed in Terpe (2003) Appl. Microbiol. Biotechnol. 60 523-533. In preferred embodiments, a poly-His tag such as (H)₆, His-SUMO tag (Invitrogen Champion pET SUMO expression system), or MRGS(H)₆ may be used.

The affinity tag sequence may be separated from the ligand exchange protein described herein after purification, for example, using a site-specific protease.

In some embodiments, the ligand exchange protein described herein may be coupled to a leader peptide to direct secretion of the ligand exchange protein from cell into the culture medium as a precursor protein.

A range of suitable leader peptides are known in the art. The leader peptide may be heterologous to the TAPBPR fragment described herein i.e. it may be a non-TAPBPR leader sequence. For example, an α-factor secretion signal or BiP leader sequence may be employed. The leader peptide is located at the N terminus of the precursor protein. After expression of the precursor, the leader peptide is then removed by post-translational processing after expression of the precursor to generate the mature ligand exchange protein.

Ligand exchange proteins as described herein may be isolated, in the sense of being free from contaminants, such as other polypeptides and/or cellular components.

Ligand exchange proteins load MHC class I molecules on the surface of the cells with exogenous peptide. An exogenous peptide is a peptide that is not generated naturally by the cells with the MHC class I molecules. For example, it may have been administered to the individual. Exogenous peptide may have the same amino acid sequence as an endogenous peptide that is generated naturally by the cells or a different amino acid sequence.

In some embodiments, the immunogenicity of the exogenous peptide may be different to the immunogenicity of endogenous peptides displayed in the MHC class I molecules (i.e. it may be higher or lower). For example, an exogenous peptide as described herein may be immunogenic or non-immunogenic, depending on the application. In other embodiments, the immunogenicity of the exogenous peptide may be the same as the immunogenicity of one or more endogenous peptides displayed in the MHC class I molecules. For example, the exogenous peptide may have the same amino acid sequence as one or more endogenous peptides. Loading of MHC class I molecules with the exogenous peptide as described here may increase the total amount of peptide with the amino acid sequence that is displayed on the cells and may thereby increase or reduce the immunogenicity of the cells.

Peptides that are displayed by MHC class I molecules are well-known in the art (see for example the on-line Immune Epitope Database and Analysis Resource (IEDB); Vita et al Nucl Acid Res 2014 Oct. 9 piii:gku938) and further peptides may be identified using immunopeptidomic techniques. Direct binding of peptides to MHC class I molecules may be confirmed by testing the binding of labelled peptides in cellular assays or using MHC beads. Binding of non-labelled peptide to MHC class I molecules may be determined by staining treated cells with TCR-tetramers specific for the peptide.

In additional to tetramers, all the other applications that can be enabled by exchanged pHLA molecules include, but are not limited to, HLA Sandwich ELISAs, HLA Direct ELISAs, HLA-Sera Antibody Assays, HLA Controls in ELISA Assays, HLA Assay Standards, HLA Blocking Assays, HLA Neutralizing Assays, HLA Competition Assays, HLA Bead Assays, HLA Immunization Procedures.

An immunogenic peptide is an exogenous peptide that is capable of generating an immune response in an individual when loaded onto an MHC class I molecule. For example, the immunogenic peptide/MHC class I complex may be recognized by T cells. The presence of MHC class I molecules loaded with immunogenic peptide on the surface of target cells may induce or increase immune responses against the target cells.

Suitable immunogenic peptides are known in the art and may for example be candidates in vaccines for cancer or infection. In some embodiments, immunogenic peptides for loading onto MHC class I may be antigens naturally expressed on a patient's own tumor; neoantigens or other peptides derived from tumors; or peptides derived from pathogens, such as viruses.

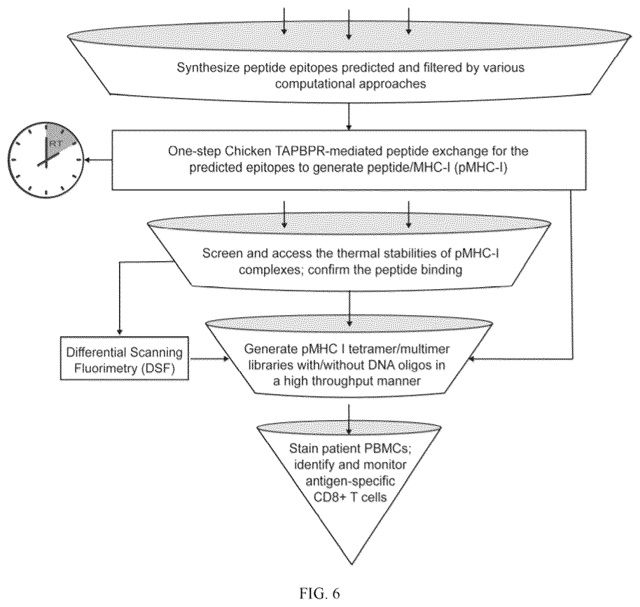

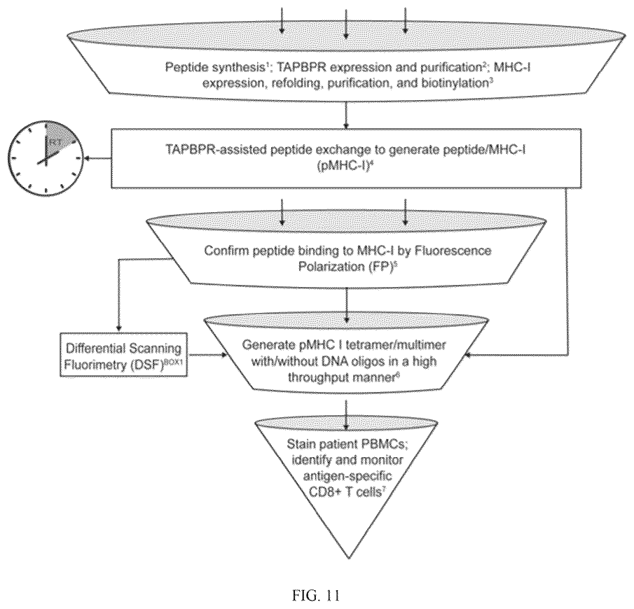

Reference is made to commonly-owned application Ser. No. 63/269,962, filed 25 Mar. 2022 and Ser. No. 63/312,584, filed 22 Feb. 2022, both entitled “Systems and Methods for Generating Chimeric Major Histocompatibility Complex (MHC) Molecules with Desired Peptide-Binding Specificities,” both of which are incorporated herein by reference in their entireties. In the present disclosure, where a MHC molecule is used, that MHC molecule can be a MHC molecule generated or obtained from a method of the commonly-owned, concurrently filed application, and likewise, embodiments of the present disclosure may be employed in practicing that which is disclosed in the concurrently-filed application. In particular, the chimera disclosed in the above applications, in particular, FIGS. 6, 11 and 16-19 and Appendices 1-3 of the '962 and '584 applications disclosed above are useful in the design of peptide-centric therapeutics disclosed herein. The chimera of the invention can be used to recognize peptide-MHC moieties, irrespective of the peptide, e.g., that the chimera can be used to recognize three dimensional structures, and hence can be used to screen for reagents that can touch, contact or interact with residues and form a complex. The tetramerized form is especially useful. Thus, products from any means of producing mass quantities of expression products that may be different, e.g., yeast display systems, phage display systems or directed evolution, can be screened using the chimera of the invention to determine alignment of three-dimensional structure with pMHC structure. Accordingly, the invention comprehends screening expression products, such as those from yeast display systems, phage display systems or directed evolution, by contacting expression products with the pMHC structure, or by aligning data as to the three dimensional structure of the expression products with the crystal structure(s) provided herewith, for instance using computer implementation to compare said three dimensional structure of the expression products with the crystal structure(s) provided herewith. Thus, the disclosure herein of the Figures and Appendices, e.g., FIGS. 11, 16-19, and Appendices 1-3, can be applied in the techniques discussed as to FIG. 6 of the '962 and '584 applications disclosed above as well as to the herein disclosed invention.

In some embodiments, the immunogenic peptide may comprise an antigen or an epitope that is characteristic of a disease cell. For example, the immunogenic peptide may comprise an antigen or an epitope that is characteristic of a cancer cell or a pathogen-infected cell.

Epitopes that are characteristic of cancer cells are well known in the art and include epitopes from tumor antigens. Suitable antigens and epitopes are described elsewhere herein. Preferred tumor antigens from which immunogenic peptides may be derived include neoantigens, tumor-specific, differentiation and overexpressed proteins, such as ErbB2/Her2 (e.g. RLLQETELV), gp100 (e.g. IMDQVPFSV and YLEPGPVTA), NY-Eso-1 (e.g. SLLMWITQC), p53 (e.g. LLGRNSFEV), MART1 (e.g. ELAGIGILTV), MAGE-10 (e.g. GLYDGMEHL), human AFP (e.g. FMNKFIYEI), Mesothelin (e.g. SLLFLLFSL), MAGE-A4 (e.g. GVYDGREHTV), MART-1 (e.g EAAGIGILTV, ELAGIGILTV) and 5T4 (e.g. FLTGNQLAV, RLARLALVL).

Other tumor antigens and epitopes are well known in the art (see for example the Cancer Research Institute NY on-line peptide database; Tumor T cell antigen database, Olsen et al (2017) Cancer Immunol Immunother. doi: 10.1007/s00262-017-1978-y; Immune Epitope and Analysis Resource, Vita et al Nucleic Acids Res. 2014 Oct. 9. pii: gku938).

Epitopes that are characteristic of pathogen-infected cells are well known in the art and include epitopes from viral proteins. Suitable epitopes are described elsewhere herein and may include influenza epitopes (e.g. GILGFVFTL (SEQ ID NO: 87), AIMDKNIIL (SEQ ID NO: 88)), HIV epitopes (e.g. ILKEPVHGV (SEQ ID NO: 89), SLYNTVATL (SEQ ID NO: 90), KLTPLCVTL (SEQ ID NO: 91)), hepatitis B epitopes (e.g. FLPSDFFPSV (SEQ ID NO: 92), WLSLLVPFV (SEQ ID NO: 93)), Human cytomegalovirus (CMV) epitopes (e.g. NLVPMVATV (SEQ ID NO: 82), VLEETSVML (SEQ ID NO: 94)), Epstein Barr virus (EBV) epitopes (e.g. YLLEMLWRL (SEQ ID NO: 95), CLGGLLTMV (SEQ ID NO: 96)), Varicella-zoster virus epitopes (e.g. ILIEGIFFV (SEQ ID NO: 97)), Measles epitopes (e.g. ILPGQDLQYV (SEQ ID NO: 98)), ZIKA (e.g. FLVEDHGFGV (SEQ ID NO: 99), KSYFVRAAK (SEQ ID NO: 100)), and Ebola virus epitopes. Other viral epitopes are well known in the art (see for example Immune Epitope and Analysis Resource, Vita et al Nucleic Acids Res. 2014 Oct. 9. pii: gku938).

In some embodiments, MHC class I molecules on cancer cells may be loaded with immunogenic peptides comprising one or more viral epitopes. This may be useful in eliciting anti-viral immune responses against the cancer cells.

In other embodiments, the immunogenic peptide may comprise an antigen or an epitope that is not characteristic of a disease cell but is still capable of eliciting an immune response against cells displaying it at the cell surface. For example, the immunogenic peptide may comprise a synthetic epitope. Suitable synthetic epitopes are well known in the art. A synthetic epitope may be generated for example by replacing an amino acid exposed to the TCR in a peptide displayed on MHC class I molecules with an artificial amino acid, such as 3-cyclohexylalanine (CHA).

A non-immunogenic peptide is an exogenous peptide that does not generate an immune response in an individual when loaded onto an MHC class I molecule. The presence of MHC class I molecules loaded with non-immunogenic peptide on the surface of target cells may prevent or reduce immune responses against the target cells.

Suitable exogenous peptides may be 8-15mers, for example 8-11mers, preferably 9mers. The exogenous peptide may be compatible with some or all of the MHC class I molecules present on the surface of the target cells. For example, the sequence of the exogenous peptide may include suitable anchor residues required for association with some or all of the MHC class I molecules on the surface of the target cells.

The MHC class I molecules on the surface of the target cells may include HLA-A molecules, such as HLA-A68 and HLA-A2. MHC class I molecules may be identified using conventional techniques, such as tissue typing or flow cytometry.

Suitable sequences for display by the MHC class I molecules on the surface of the target cells may be determined using standard techniques. For example, when HLA-A2 molecules are present on the surface of the target cells, the exogenous peptide may have the sequence xLxxxxxxV/L, where X is independently any amino acid; when HLA-A*03:01 molecules are present on the surface of the target cells, the exogenous peptide may have the sequence xL/INxxxxxxK/R, where X is independently any amino acid; when HLA-A*68:02 molecules are present on the surface of the target cells, the exogenous peptide may have the sequence xTNxxxxxxLN, where X is independently any amino acid; when HLA-B*27:05 molecules are present on the surface of the target cells, the exogenous peptide may have the sequence xRxxxxxxx, where X is independently any amino acid; when HLA-B*51:01 molecules are present on the surface of the target cells, the exogenous peptide may have the sequence xPxxxxxxlN, where X is independently any amino acid; and when HLA-B*15:03 molecules are present on the surface of the target cells, the exogenous peptide may have the sequence xQ/KxxxxxXFNL, where X is independently any amino acid.

The present invention also relates to an alternative approach for generating conditional peptide ligands for a range of disease-related MHC-I allotypes, which form stable complexes with MHC-I molecules but can be exchanged for desired peptides through a chaperone-assisted process. Rather than using truncated high affinity peptides, the new approach involves introducing a modified unnatural amino acid near the peptide C-terminus. This results in a MHC-I protein complex with higher thermal stability and improved exchanged properties, relative to the conventional goldilocks peptides. Applicants outline an exemplar application for the HLA-A*02:01 allotype and detailed protocols for: i) peptide exchange on conditional peptide ligand-loaded MHC-I molecules using human TAPBPR, ii) the analysis of peptide binding by complementary differential scanning fluorimetry (DSF) and fluorescence polarization (FP) assays, and iii) the parallel production of pMHC-I tetramers for T cell detection.

A brief outline of the process to generate conditional ligands for different HLA allotypes that maximizes the destabilization of the F pocket residue may comprise:

Start with a high affinity (Tm>60 degrees centigrade) peptide for an HLA allele of interest described in the Immune Epitope Database (IEDB, iedb.org, Nucleic Acids Res. 2019 Jan. 8; 47(Database issue): D339-D343, Published online 2018 Oct. 24. doi: 10.1093/nar/gky1006.).

Introduce a modified amino acid at either of the positions 8, 7, or 6 of the peptide sequence. An exemplar modification is an unnatural β-amino acid such as β-phenylalanine, but other modifications can be also used such as but not limited to gamma or delta amino acids, selenocysteine, pyrrolysine, photoreactive amino acid analogues such as photoleucine and photomethionine, N-formylmethionine or a non-proteinogenic amino acid such as hydroxyproline. The design principle is to detabilize interactions with the F-pocket of the MHC groove, which is also the area which is widened and further destabilized upon interactions with molecular chaperones, Tapasin and TAPBPR.

Do a preliminary refolding reaction with each candidate peptide. The ideal placeholder peptide should have a Tm of approximately 50 degrees centigrade, which is enough to form a stable peptide:MHC complex in the absence of chaperones.

Select the top modified peptide as a placeholder to perform chaperone-mediated exchange with high-affinity peptides of interest. Here, interactions with chaperones dislodge the placeholder peptide, such that an incoming high-affinity peptide can bind to the empty MHC groove.

Thus, the combination of a modified, placeholder peptide used as a conditional ligand, and a molecular chaperone enables high-throughput peptide exchange for applications in various settings (such as tetramer library generation).

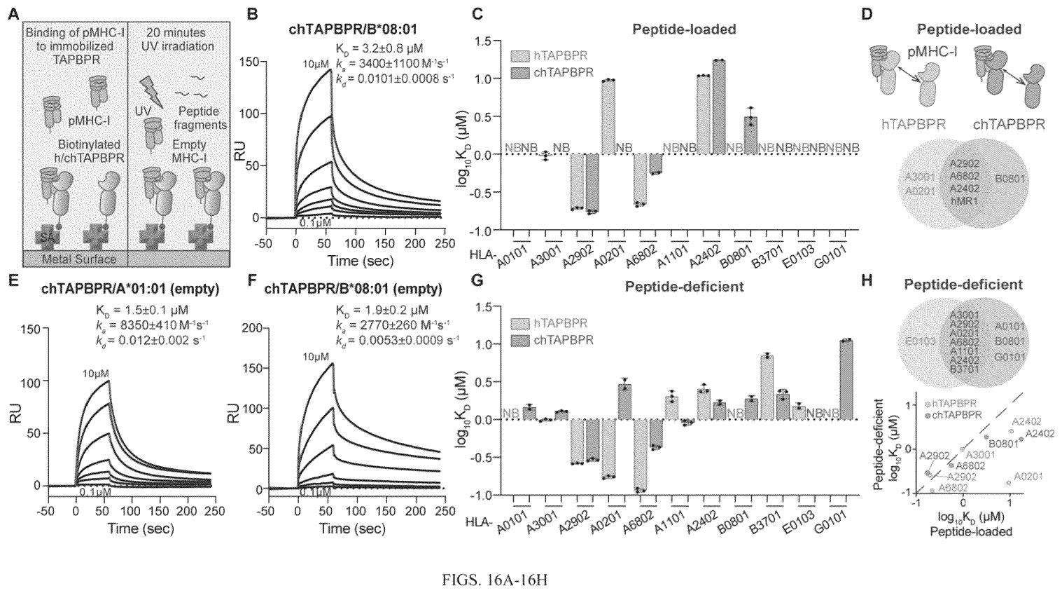

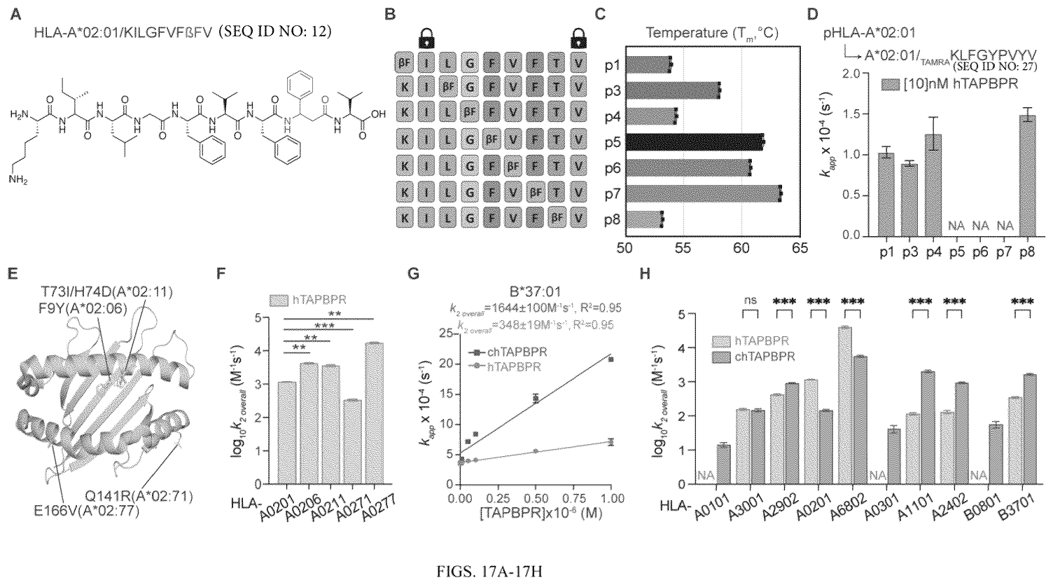

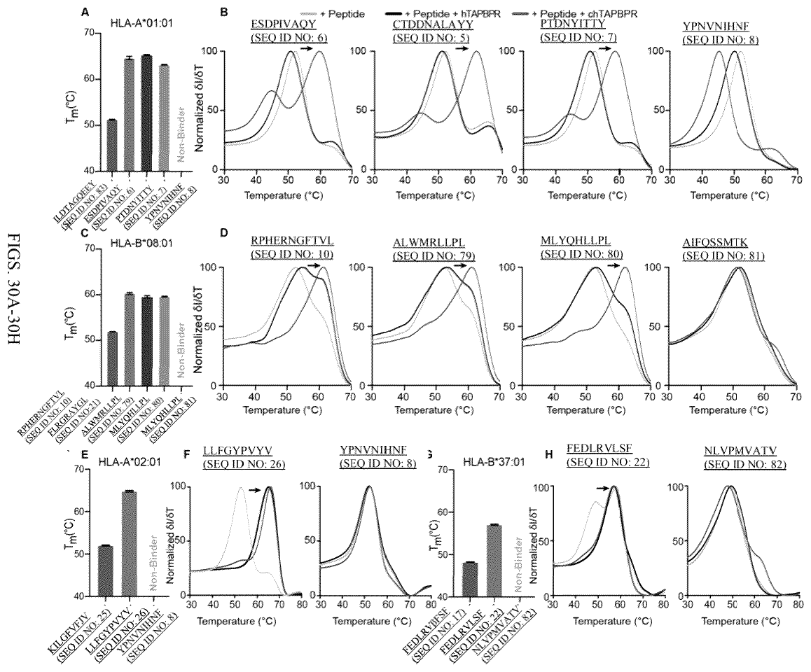

TAPBPR interactions with empty MHC-I promote exchange of placeholder for high-affinity peptides are shown in FIGS. 16, 17 and 30. Applicants established high-affinity peptides with increased thermal stabilities by at least 5 degrees relative to complexes refolded with suitable placeholder peptides for both systems (FIG. 17H, FIG. 30A, C). HLA-A*01:01 refolded with a moderate affinity 10mer RAS self-epitope (Tₘ=52° C.) cannot be exchanged in the presence of 10-fold molar excess of high-affinity 9mer or 10mer peptides, even upon incubation with hTAPBPR (FIGS. 30A-30D), consistent with the observed lack of binding between hTAPBPR and HLA-A*01:01 in either peptide-loaded or -deficient states by SPR (FIG. 16c, g). Notably, incubation with sub-stoichiometric amounts of chTAPBPR in addition to the incoming peptide led to significant (>10 degrees) thermal shifts for all three peptides, indicating net exchange of the placeholder for high affinity peptide epitopes (FIGS. 30A-30D). The minor destabilization effect observed for chTAPBPR upon addition of a non-specific peptide (FIG. 30B) likely results from the formation of transient pMHC-I/chTAPBPR complexes with destabilized peptide-binding grooves. HLA-B*08:01 showed a partial thermal shift upon incubation with excess high affinity peptide, revealing a spontaneous, partial peptide loading mediated by empty molecules in the sample which is not affect by the addition of hTAPBPR (FIGS. 30A-30D). However, addition of catalytic amounts of chTAPBPR promotes the complete exchange of placeholder for different high-affinity peptides, as indicated by a single, up-shifted Tₘ peak (FIGS. 30A-30D). Nonspecific peptides fail to bind in the presence of either human or chicken TAPBPR (FIG. 30D). Placed within the context of Applicants' SPR data, the DSF peptide exchange results demonstrate a trend where interactions with empty HLA-A*01:01 or HLA-B*08:01 molecules, as seen for chicken but not for human TAPBPR, promote the complete exchange of placeholder for higher affinity peptides. These findings suggest that both peptide unloading and chaperoning functions of TAPBPR are required for efficient catalysis of peptide exchange in vitro.

An exemplary TAPBPR-assisted peptide exchange is presented in FIG. 11 that illustrates peptide exchange and MHC-I tetramer formation. Biotinylated HLA-A*02:01/KILGFVFβFV (SEQ ID NO: 12) were mixed with TAPBPR (1:7 TAPBPR/pMHC-I molar ratio) and the desired peptide (1:10 pMHC-I/peptide molar ratio). Samples were incubated for 2 hours at room temperature, and the exchanged pMHC-I molecules were used for differential scanning fluorimetry (DSF), fluorescence polarization (FP), and tetramerization.

Frozen biotinylated HLA-A*02:01/KILGFVFβFV (SEQ ID NO: 12) samples were thawed on ice before setting up the peptide exchange reaction.

The biotinylated monomer concentration was confirmed and adjusted to 100 μM, the peptide solution concentration to 1 mM, and the TAPBPR concentration to 10 μM.

5 μL (500 picomol) HLA-A*02:01/KILGFVFβFV (SEQ ID NO: 12) and 5 μL (5000 picomol) peptide solution were mixed with 5 μL (50 picomol) of human TAPBPR in a 50 μL peptide exchange reaction (the final concentration of pMHC-I, peptide, and TAPBPR is 10 μM, 1 μM, and 100 μM). Such reactions may be scaled up for multiple uses of pMHC-I monomers.

The reaction was incubated in a drawer at room temperature for two hours.

The completed peptide exchange reaction may be used for a quality control by differential scanning fluorimetry or preferably by performing a fluorescence polarization assay. pMHC-I monomer with confirmed peptide exchange may be directly used for tetramerization.

Applicants envision this procedure for most HLA allotypes.

The present invention also encompasses the use of human TAPBR mutants, such as hTAPBPR mutants carrying different combinations such as hTAPBPR^{RQ}: K211R, R270Q, hTAPBPR^{KRQ}: R105K, K211R, R270Q, and hTAPBPR^{FLQ}: S104F, K211L, R270Q). Advantageously, the mutants are hTAPBPR^{RQ}, hTAPBPR^{KRQ} and hTAPBPR^{FLQ}.

Another embodiment of the present invention encompasses the use of a chicken Tapasin, advantageously a recombinant chicken Tapasin (a homolog of TAPBPR) in addition to chicken TAPBPR, human TAPBPR and mutants thereof. Applicants have found that chTapasin is a much more stable protein scaffold to use for peptide exchange and protein engineering applications according to its thermal stability measurements, which can interact with a range of disease-relevant HLAs.

Other aspects of the invention provide a nucleic acid encoding a ligand exchange protein or a high affinity peptide described herein as described above and a vector comprising such a nucleic acid.

Suitable vectors can be chosen or constructed, containing appropriate regulatory sequences, including promoter sequences, terminator fragments, polyadenylation sequences, enhancer sequences, marker genes and other sequences as appropriate. Preferably, the vector contains appropriate regulatory sequences to drive the expression of the nucleic acid in mammalian cells. A vector may also comprise sequences, such as origins of replication, promoter regions and selectable markers, which allow for its selection, expression and replication in bacterial hosts such as E. coli. Vectors may be plasmids, viral e.g. phage, or phagemid, as appropriate. For further details, see for example, Molecular Cloning: a Laboratory Manual: 3rd edition, Russell et al., 2001, Cold Spring Harbor Laboratory Press. Many known techniques and protocols for manipulation of nucleic acid, for example in preparation of nucleic acid constructs, mutagenesis, sequencing, introduction of DNA into cells and gene expression, are described in detail in Current Protocols in Molecular Biology, Ausubel et al. eds. John Wiley & Sons, 1992.

A nucleic acid or vector as described herein may be introduced into a host cell. Another aspect of the invention provides a recombinant cell comprising a nucleic acid or vector that expresses a ligand exchange protein or a high affinity peptide as described above. A range of host cells suitable for the production of recombinant ligand exchange protein or a high affinity peptide are known in the art. Suitable host cells may include prokaryotic cells, in particular bacteria such as Escherichia coli and Lactococcus lactis and eukaryotic cells, including mammalian cells such as CHO and CHO-derived cell lines (Lec cells), HeLa, COS, HEK293 and HEK-EBNA cells, amphibian cells such as Xenopus oocytes, insect cells such as Trichoplusia ni, Sf9 and Sf21 and yeast cells, such as Pichia pastoris.

Techniques for the introduction of nucleic acid into cells are well established in the art and any suitable technique may be employed, in accordance with the particular circumstances. For eukaryotic cells, suitable techniques may include calcium phosphate transfection, DEAE-Dextran, electroporation, liposome-mediated transfection and transduction using retrovirus or other virus, e.g. adenovirus, AAV, lentivirus or vaccinia. For bacterial cells, suitable techniques may include calcium chloride transformation, electroporation and transfection using bacteriophage. Marker genes such as antibiotic resistance or sensitivity genes may be used in identifying clones containing nucleic acid of interest, as is well-known in the art.

The introduction may be followed by expression of the nucleic acid to produce the encoded ligand exchange protein or a high affinity peptide protein. In some embodiments, host cells (which may include cells actually transformed although more likely the cells will be descendants of the transformed cells) may be cultured in vitro under conditions for expression of the nucleic acid, so that the encoded serpin polypeptide is produced. When an inducible promoter is used, expression may require the activation of the inducible promoter.

The expressed polypeptide comprising or consisting of the ligand exchange protein or a high affinity peptide protein may be isolated and/or purified, after production. This may be achieved using any convenient method known in the art. Techniques for the purification of recombinant polypeptides are well known in the art and include, for example HPLC, FPLC or affinity chromatography. In some embodiments, purification may be performed using an affinity tag on the polypeptide as described above.

Another aspect of the invention provides a method of producing a ligand exchange protein or a high affinity peptide described herein comprising expressing a heterologous nucleic acid encoding the ligand exchange protein or a high affinity peptide in a host cell and optionally isolating and/or purifying the ligand exchange protein or a high affinity peptide thus produced. After production, the ligand exchange protein or a high affinity peptide may be investigated further, for example the pharmacological properties and/or activity may be determined. Methods and means of protein analysis are well-known in the art.

A ligand exchange protein or a high affinity peptide described herein as described herein may be useful in therapy. For example, the ligand exchange protein or a high affinity peptide may be administered to an individual to modulate the immunogenicity of target cells or the ligand exchange protein or a high affinity peptide may be used to modulate the immunogenicity of cells in vitro or ex vivo, which are then administered to an individual. A ligand exchange protein or a high affinity peptide for administration to an individual is preferably a chimeric ligand exchange protein or a high affinity peptide comprising a targeting domain. This allows the immunogenicity of target cells in the individual to be modulated.

Whilst the ligand exchange protein or a high affinity peptide may be administered alone, it will usually be administered in the form of a pharmaceutical composition, which may comprise at least one component in addition to the chimeric ligand exchange protein or a high affinity peptide. Thus pharmaceutical compositions may comprise, in addition to the ligand exchange protein or a high affinity peptide itself, a pharmaceutically acceptable excipient, carrier, buffer, stabilizer or other materials well known to those skilled in the art. The term “pharmaceutically acceptable” as used herein pertains to compounds, materials, compositions, and/or dosage forms which are, within the scope of sound medical judgement, suitable for use in contact with the tissues of a subject (e.g., human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation. The precise nature of the carrier or other material will depend on the route of administration, which may be by bolus, infusion, injection or any other suitable route, as discussed below.

The ligand exchange protein or a high affinity peptide may be administered in combination with an exogenous peptide, preferably an immunogenic peptide. In some embodiments, the ligand exchange protein or a high affinity peptide and the exogenous peptide may be formulated in the same pharmaceutical composition. In other embodiments, the ligand exchange protein or a high affinity peptide and the exogenous peptide may be formulated in separate pharmaceutical compositions.

In some embodiments, the ligand exchange protein, high affinity peptide and/or exogenous peptide may be provided in a lyophilised form for reconstitution prior to administration. For example, a lyophilised ligand exchange protein, high affinity peptide and/or exogenous peptide may be re-constituted in sterile water and mixed with saline prior to administration to an individual.

For parenteral, for example sub-cutaneous, intra-tumoral, intra-muscular or intra-venous administration, e.g. by injection, the pharmaceutical composition comprising the ligand exchange protein, high affinity peptide and/or exogenous peptide described herein, nucleic acid or cell may be in the form of a parenterally acceptable aqueous solution which is pyrogen-free and has suitable pH, isotonicity and stability. Those of relevant skill in the art are well able to prepare suitable solutions using, for example, isotonic vehicles, such as Sodium Chloride Injection, Ringers Injection, and Lactated Ringers Injection. Preservatives, stabilizers, buffers, antioxidants and/or other additives may be employed as required including buffers such as phosphate, citrate and other organic acids; antioxidants, such as ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens, such as methyl or propyl paraben; catechol; resorcinol; cyclohexanol; 3′-pentanol; and m-cresol); low molecular weight polypeptides; proteins, such as serum albumin, gelatin or immunoglobulins; hydrophilic polymers, such as polyvinylpyrrolidone; amino acids, such as glycine, glutamine, asparagines, histidine, arginine, or lysine; monosaccharides, disaccharides and other carbohydrates including glucose, mannose or dextrins; chelating agents, such as EDTA; sugars, such as sucrose, mannitol, trehalose or sorbitol; salt-forming counter-ions, such as sodium; metal complexes (e.g. Zn-protein complexes); and/or non-ionic surfactants, such as TWEEN™, PLURONICS™ or polyethylene glycol (PEG). Suitable carriers, excipients, etc. can be found in standard pharmaceutical texts, for example, Remington's Pharmaceutical Sciences, 18th edition, Mack Publishing Company, Easton, Pa., 1990.

Pharmaceutical compositions and formulations may conveniently be presented in unit dosage form and may be prepared by any methods well known in the art of pharmacy. Such methods include the step of bringing into association the chimeric protein described herein with the carrier which constitutes one or more accessory ingredients. In general, the compositions are prepared by uniformly and intimately bringing into association the active compound with liquid carriers or finely divided solid carriers or both, and then if necessary shaping the product.

A pharmaceutical composition comprising a ligand exchange protein, high affinity peptide and/or exogenous peptide as described herein may be administered alone or in combination with other treatments, either simultaneously or sequentially dependent upon the condition to be treated.

Ligand exchange proteins described herein may be useful in modulating the immunogenicity of mammalian cells in vivo, in vitro or ex vivo. For example, a method may comprise providing a population of mammalian cells having surface MHC class I molecules, contacting the population of mammalian cells with an exogenous peptide and a ligand exchange protein comprising a TAPBPR fragment consisting of the luminal domain of TAPBPR, such that the ligand exchange protein loads the exogenous peptide onto the surface MHC class I molecules of the cells in the population, thereby modulating the immunogenicity of the mammalian cells.

Surface MHC class I molecules that have been loaded with immunogenic exogenous peptides are accessible to T cell receptors. The loading of the surface MHC class I molecules with immunogenic peptides may induce or increase T cell recognition of the cells of the mammalian cells and may increase the immunogenicity of the mammalian cells.

In some embodiments, the mammalian cells may be antigen presenting cells (APCs), such as dendritic cells. The loading of the surface MHC class I molecules with immunogenic exogenous peptides may increase the ability of the APCs to induce immune responses, for example immune responses against the antigenic epitopes contained in the immunogenic peptide. APCs loaded with immunogenic peptide as described above may be used to stimulate T cells in vitro or ex vivo or administered to an individual to stimulate T cells in vivo.

A method of producing antigen presenting cells for generating or increasing an immune response in an individual may comprise providing a population of antigen presenting cells previously obtained from the individual, and contacting the antigen presenting cells with a ligand exchange protein and an immunogenic peptide, such that the ligand exchange protein loads the immunogenic peptide onto surface MHC class I molecules of the antigen presenting cells, the antigen presenting cells being capable of stimulating T cells to generate an immune response

The method may an in vitro or an ex vivo method.

The immunogenic peptide may comprise one or more antigenic epitopes. The antigen presenting cells may activate T cells against the antigenic epitopes of the immunogenic peptide. For example, the antigenic epitopes of the immunogenic peptide may be present on disease cells in the individual. The antigen presenting cells may activate T cells capable of generating an immune response against the disease cells in the individual.

In some embodiments, following production, the antigen presenting cells may be administered to an individual to activate T cells and generate or increase a T cell immune response in the individual.

In other embodiments, following production, the antigen presenting cells may be contacted with a population of T cells to activate the T cells against the one or more antigenic epitopes of the immunogenic peptide in vivo or ex vivo. The activated T cells may be administered to an individual to generate a T cell immune response in the individual. The individual may be the individual from which the population of T cells was obtained (autologous) or a different individual (allogeneic).

Suitable antigen presenting cells include any cell that expresses the MHC class I molecules against which the immune response is to be directed. In some embodiments, dendritic cells may be preferred.

Ligand exchange proteins and/or high affinity peptides described herein may be useful in modulating the immunogenicity of mammalian cells in vivo. This may be useful in immunotherapeutic applications, for example in which the generation or enhancement of an immune response might have a therapeutic effect. Suitable applications might include the treatment of conditions associated with the presence of populations of disease cells in an individual. These conditions might include cancer and infection with an intracellular pathogen. Other suitable applications might include the treatment of autoimmune or auto inflammatory conditions in which the reduction in immunogenicity of a cell or tissue might have a therapeutic effect. The targeting domain of a ligand exchange protein as described above may preferentially or selectively direct the protein to target cells within the individual relative to non-target cells.

A ligand exchange protein or high affinity peptide described herein may be used in a method of treatment of the human or animal body, including therapeutic and prophylactic or preventative treatment (e.g. treatment before the onset of a condition in an individual to reduce the risk of the condition occurring in the individual; delay its onset; or reduce its severity after onset). Prophylactic or preventative treatment may include vaccination. The method of treatment may comprise administering a ligand exchange protein or high affinity peptide described herein and an immunogenic peptide to an individual in need thereof.

A method of increasing the immunogenicity of target cells in an individual may comprise administering a ligand exchange protein as described above to the individual, wherein ligand exchange protein comprises a targeting domain which binds to target cells in the individual, and administering an immunogenic peptide to the individual, such that the ligand exchange protein loads the immunogenic peptide onto surface MHC class I molecules of the target cells, thereby increasing the immunogenicity of the target cells.

An individual suitable for treatment as described above may be a mammal, such as a rodent (e.g. a guinea pig, a hamster, a rat, a mouse), murine (e.g. a mouse), canine (e.g. a dog), feline (e.g. a cat), equine (e.g. a horse), a primate, simian (e.g. a monkey or ape), a monkey (e.g. marmoset, baboon), an ape (e.g. gorilla, chimpanzee, orangutan, gibbon), or a human.

In some preferred embodiments, the individual is a human. In other preferred embodiments, non-human mammals, especially mammals that are conventionally used as models for demonstrating therapeutic efficacy in humans (e.g. murine, primate, porcine, canine, or rabbit animals) may be employed.

Suitable target cells may include disease cells i.e. cells that are associated with a disease condition in the individual, such as cells infected with virus or other intracellular pathogen, or cancer or tumor cells.

In some preferred embodiments, the target cells are cancer cells. A method of treatment of cancer in an individual may comprise administering a ligand exchange protein described above to the individual, wherein ligand exchange protein comprises a targeting domain which binds to cancer cells in the individual, and administering an immunogenic peptide to the individual, such that the ligand exchange protein loads the immunogenic peptide onto surface MHC class I molecules of the cancer cells of the individual, thereby eliciting or increasing an immune response in the individual against the cancer cells.

Cancer may be characterised by the abnormal proliferation of malignant cancer cells and may include leukaemias, such as AML, CML, ALL and CLL, lymphomas, such as Hodgkin lymphoma, non-Hodgkin lymphoma and multiple myeloma, and solid cancers such as sarcomas, skin cancer, melanoma, bladder cancer, brain cancer, breast cancer, uterus cancer, ovary cancer, prostate cancer, lung cancer, colorectal cancer, cervical cancer, liver cancer, head and neck cancer, oesophageal cancer, pancreas cancer, renal cancer, adrenal cancer, stomach cancer, testicular cancer, cancer of the gall bladder and biliary tracts, thyroid cancer, thymus cancer, cancer of bone, and cerebral cancer, as well as cancer of unknown primary (CUP).

In some embodiments, cancer cells within an individual may be immunologically distinct from normal somatic cells in the individual (i.e. the cancerous tumor may be immunogenic). For example, the cancer cells may be capable of eliciting a systemic immune response in the individual against one or more antigens expressed by the cancer cells. The tumor antigens that elicit the immune response may be specific to cancer cells or may be shared by one or more normal cells in the individual. In other embodiments, cancer cells within an individual may not be immunologically distinct from normal somatic cells in the individual until MHC class I molecules on the surface of the cancer cells are loaded with exogenous immunogenic peptide using a ligand exchange protein as described herein.

In some embodiments, the individual may have minimal residual disease (MRD) after an initial cancer treatment.

An individual with cancer may display at least one identifiable sign, symptom, or laboratory finding that is sufficient to make a diagnosis of cancer in accordance with clinical standards known in the art. Examples of such clinical standards can be found in textbooks of medicine such as Harrison's Principles of Internal Medicine, 15th Ed., Fauci A S et al., eds., McGraw-Hill, New York, 2001. In some instances, a diagnosis of a cancer in an individual may include identification of a particular cell type (e.g. a cancer cell) in a sample of a body fluid or tissue obtained from the individual.

In particular, treatment may include inhibiting cancer growth, including complete cancer remission, and/or inhibiting cancer metastasis. Cancer growth generally refers to any one of a number of indices that indicate change within the cancer to a more developed form. Thus, indices for measuring an inhibition of cancer growth include a decrease in cancer cell survival, a decrease in tumor volume or morphology (for example, as determined using computed tomographic (CT), sonography, or other imaging method), a delayed tumor growth, a destruction of tumor vasculature, improved performance in delayed hypersensitivity skin test, an increase in the activity of T cells, and a decrease in levels of tumor-specific antigens. Administration of T cells modified as described herein may improve the capacity of the individual to resist cancer growth, in particular growth of a cancer already present the subject and/or decrease the propensity for cancer growth in the individual.

In other preferred embodiments, the target cells are pathogen-infected cells.

A method of treatment of pathogen infection in an individual may comprise administering a ligand exchange protein described above to the individual, wherein ligand exchange protein comprises a targeting domain which binds to pathogen-infected cells in the individual, and administering an immunogenic peptide to the individual, such that the ligand exchange protein loads the immunogenic peptide onto surface MHC class I molecules of the pathogen-infected cells of the individual, thereby eliciting or increasing an immune response in the individual against the pathogen-infected cells.

Pathogen infection may include viral infection, for example HIV, EBV, CMV or hepatitis infection.

In other preferred embodiments, the target cells are antigen presenting cells, such as dendritic cells. Antigen presenting cells present antigenic epitopes to T cells to activate a T cell response against the antigen. A method of treatment of a condition associated with disease cells in an individual may comprise administering a ligand exchange protein described herein to the individual, wherein the ligand exchange protein comprises a targeting domain that binds to antigen presenting cells in the individual, and administering an immunogenic peptide to the individual, such that the ligand exchange protein loads the immunogenic peptide onto surface MHC class I molecules of the antigen presenting cells, such that said antigen presenting cells generate or increase an immune response in the individual against the disease cells.

Disease cells may include cancer cells or pathogen-infected cells. For example, this may be useful in treating pathogen infections in which a peptide vaccine is currently used to induce CD8+ T cells responses, such as infections of HIV, EBV, CMV, hepatitis viruses, influenza, polio, human papilloma virus, measles, mumps, rubella, chicken pox, ebola, or zika; or cancer, for example by boosting the number of T cells capable of recognising a particular antigen.

In other embodiments, methods described herein may be useful in reducing immunogenicity. A method of reducing an immune response in an individual may comprise administering a chimeric ligand exchange protein to the individual, wherein the targeting domain of the chimeric ligand exchange protein binds to target cells in the individual, administering a non-immunogenic peptide to the individual, such that the ligand exchange protein replaces immunogenic peptides in surface MHC class I molecules with non-immunogenic peptides and the immunogenicity of the target cells is reduced in the individual.

The chimeric ligand exchange protein may for example, reduce the immunogenicity of the donor organ and/or antigen presentation cells removing recognition of self/donor-peptides (e.g. alloantigens/minor histocompatibility antigens) which are the target of the immune recognition.

In some preferred embodiments, the individual may have an autoimmune disease or immune-mediated inflammatory disease. A method of treatment of autoimmune or immune-mediated inflammatory disease in an individual may comprise administering a ligand exchange protein described above to the individual, wherein ligand exchange protein comprises a targeting domain which binds to cells in the individual having surface MHC class I molecules displaying an immunogenic peptide, and administering a non-immunogenic peptide to the individual, such that the ligand exchange protein replaces the immunogenic peptide in the surface MHC class I molecules with the non-immunogenic peptide, thereby preventing or reducing an immune response in the individual against the cells.

In other preferred embodiments, methods described herein may be useful in organ or tissue transplantation. A method of treating diseases associated with MHC class I molecules in an individual may comprise administering a chimeric ligand exchange protein described herein to the individual, wherein the targeting domain of the chimeric ligand exchange protein binds to target cells in the individual which have disease associated MHC class I molecules on their surface, administering an exogenous peptide to the individual, such that the ligand exchange protein loads the surface MHC class I molecules with the exogenous peptide, such that the MHC class I molecules are stabilized by the exogenous peptide.

In other embodiments, methods described herein may be useful in treating diseases associated with MHC class I molecules. A method of treating diseases associated with MHC class I molecules in an individual may comprise administering a chimeric ligand exchange protein described herein to the individual, wherein the targeting domain of the chimeric ligand exchange protein binds to target cells in the individual which have disease associated MHC class I molecules on their surface, and administering an exogenous peptide to the individual, such that the ligand exchange protein loads the surface MHC class I molecules with the exogenous peptide, such that the MHC class I molecules are stabilized by the exogenous peptide.

MHC class I associated diseases may include the spondyloarthropathies (associated with HLA-B27), Behcet's disease (associated with HLA-B51), Birdshot Chorioretinopathy (associated with HLA-A29) psoriasis and psoriatic arthritis (associated with HLA-Cw6).

Administration is normally in a “therapeutically effective amount” or “prophylactically effective amount”, this being sufficient to show benefit to a patient. Such benefit may be at least amelioration of at least one symptom. The actual amount administered, and rate and time-course of administration, will depend on the nature and severity of what is being treated, the particular mammal being treated, the clinical condition of the individual patient, the cause of the disorder, the site of delivery of the composition, the method of administration, the scheduling of administration and other factors known to medical practitioners.

A composition may be administered alone or in combination with other treatments, either simultaneously or sequentially dependent upon the circumstances of the individual to be treated. For example, a composition may be administered in combination with vaccination, immune checkpoint inhibition, other immunotherapies and potentially chemotherapy and radiotherapy.

Prescription of treatment, e.g. decisions on dosage etc, is within the responsibility of general practitioners and other medical doctors and may depend on the severity of the symptoms and/or progression of a disease being treated. Appropriate doses of therapeutic polypeptides are well known in the art (Ledermann J. A. et al. (1991) Int. J. Cancer 47: 659-664; Bagshawe K. D. et al. (1991) Antibody, Immunoconjugates and Radiopharmaceuticals 4: 915-922). Specific dosages may be indicated herein or in the Physician's Desk Reference (2003) as appropriate for the type of medicament being administered may be used. A therapeutically effective amount or suitable dose of a chimeric protein described herein may be determined by comparing its in vitro activity and in vivo activity in an animal model. Methods for extrapolation of effective dosages in mice and other test animals to humans are known. The precise dose will depend upon a number of factors, including whether the chimeric protein described herein is for prevention or for treatment, the size and location of the area to be treated, the precise nature of the chimeric protein described herein and the nature of any detectable label or other molecule attached to the chimeric protein described herein.

A typical dose of a ligand exchange protein or high affinity peptide will be in the range of 0.1 mg/kg to 100 mg/kg. For example, a dose in the range 100 μg to 1 g may be used for systemic applications. An initial higher loading dose, followed by one or more lower doses, may be administered. This is a dose for a single treatment of an adult patient, which may be proportionally adjusted for children and infants. Treatments may be repeated at daily, twice-weekly, weekly or monthly intervals, at the discretion of the physician.

In some embodiments, pre-vaccination and/or re-vaccination with tumor or viral antigens may be required before administration of the TAPBPR/peptide combination intended to be delivered to the tumor/infection site. The vaccination strategy may employ TAPBPR to load peptides or may involve standard vaccination regimens.

The treatment schedule for an individual may be dependent on the pharmocokinetic and pharmacodynamic properties of the ligand exchange protein or high affinity peptide described herein composition, the route of administration and the nature of the condition being treated.

Treatment may be periodic, and the period between administrations may be about 12 hours or more, 24 hours or more, 36 hours or more, 48 hours or more, 96 hours or more, or one week or more. Suitable formulations and routes of administration are described above.

Treatment may be any treatment and therapy, whether of a human or an animal (e.g. in veterinary applications), in which some desired therapeutic effect is achieved, for example, the inhibition or delay of the progress of the condition, and includes a reduction in the rate of progress, a halt in the rate of progress, amelioration of the condition, cure or remission (whether partial or total) of the condition, preventing, delaying, abating or arresting one or more symptoms and/or signs of the condition or prolonging survival of a subject or patient beyond that expected in the absence of treatment.

Treatment may also be prophylactic (i.e. prophylaxis). For example, an individual susceptible to or at risk of the occurrence or re-occurrence of disease may be treated as described herein. Such treatment may prevent or delay the occurrence or re-occurrence of the disease in the individual.

Other aspects of the invention relate to kits for use in increasing immunogenicity or stimulating immune responses as described herein. A kit may comprise a ligand exchange protein or high affinity peptide and an immunogenic peptide as described above.

A kit may further comprise an additional therapeutic agent, such as a vaccine or immune checkpoint inhibitor.

Other aspects of the invention relate to methods and reagents for identifying, characterising or isolating T cells in vitro or ex vivo using MHC class I molecules that display a target peptide.

A method of producing a MHC class I molecule displaying a target peptide may comprise contacting an MHC class I molecule with a ligand exchange protein described above and a target peptide, such that the ligand exchange protein loads the target peptide onto the MHC class I molecule, thereby producing an MHC class I molecule displaying the target peptide.

Preferably, the MHC class I molecule is contacted with a soluble ligand exchange protein. Soluble ligand exchange proteins are described in detail above.

The target peptide may be a peptide which is capable of specific binding to a T cell when displayed by a MHC class I molecule. For example, the target peptide may comprise a viral, bacterial, cancer or autoimmune antigenic epitope. MHC class I molecules displaying the target peptide may be useful in identifying, quantifying, characterising or isolating T cells within a population of T cells that specifically bind to the MHC class I molecule/target peptide complex.

The MHC class I molecule may display an initial peptide that is replaced by the target peptide following contact with the ligand exchange protein. The sequence of the initial peptide is independent of the target peptide being employed, and any convenient peptide that can be displayed by MHC class I molecules may be employed.

In some embodiments, the MHC class I molecule may be immobilised on a solid support, such as a bead. The MHC class I molecule may be a member of a population of MHC class I molecules immobilised on the solid support. For example, the ligand exchange protein may be contacted with a population of MHC class I molecules immobilised on the solid support may load target peptide onto the MHC class I molecules in the immobilised population.

MHC class I molecules may be immobilised on the solid support by any convenient technique. For example, the MHC class I molecules may be biotinylated and may be bound to the support through a biotin/streptavidin interaction.

In other embodiments, the MHC class I molecule may be in solution, for example as a sub-unit of a multimer. The ligand exchange protein may be contacted in solution with a multimer that comprises multiple MHC class I molecules. Preferred multimers include tetramers of biotinylated MHC class I molecules linked by streptavidin. The streptavidin may be labelled, for example with a fluorophore such as phycoerythrin. Tetramers of biotinylated MHC class I molecules are well known in the art (Altman et al Science 1996, 274: 94-96).

In some embodiments, the MHC class I molecules displaying the target peptide may be contacted with a population of T cells, for example a population of T cells previously obtained from an individual. The binding of the MHC class I molecules to T cells in the population may be determined. Binding may be determined by any convenient technique, such as flow cytometry.

The frequency or number of T cells within the population that bind to the MHC class I molecules displaying the target molecule may be determined. This may be useful in research or for diagnostic or prognostic applications.

T cells that bind to the MHC class I molecules displaying the target molecule may be isolated and/or expanded in vitro, for example for use in therapeutic applications.

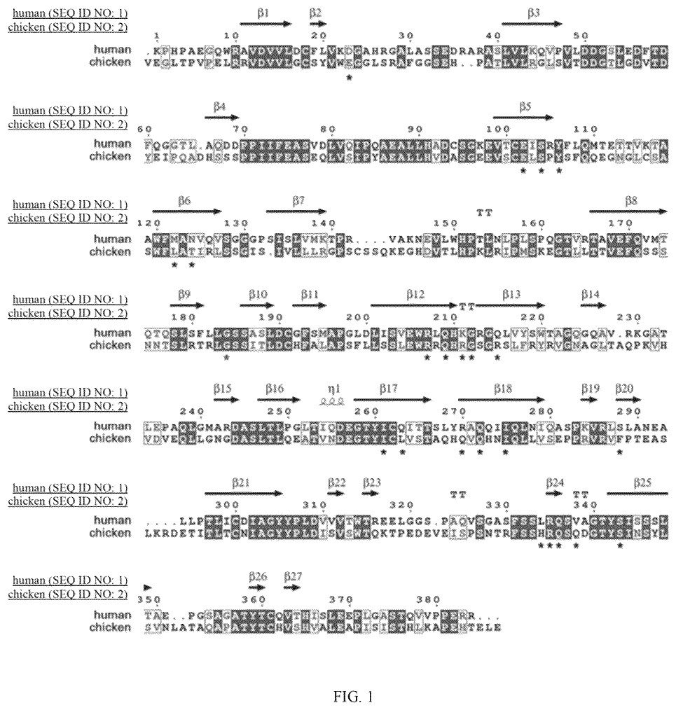

That the present invention, and especially the TAPBPR from chicken is useful and indeed can be useful beyond human TAPBPR previously known is surprising and unexpected. For example, genetically humans are more related to mice than to chickens, and yet, the chicken TAPBPR is more useful than that of the mouse. The invention comprehends the chicken TAPBPR sequence provided herein (e.g., in the figure(s) herewith), as well as nucleic acid molecules encoding such additional chicken TAPBPR sequences that are considered part of the inventions of this disclosure. The invention further comprehends amino acid sequences having a sequence identity or homology with the chicken TAPBPR sequence provided herein (e.g., in the figure(s) herewith, e.g., FIG. 1), advantageously at least 50% or at least 60% or at least 70% or at least 80% or at least 85% or at least 86% or at least 87% or at least 88% or at least 89% or at least 90% or at least 91% or at least 92% or at least 93% or at least 94% or at least 95% or at least 96% or at least 97% at least or 98% or at least 99%, sequence identity, or homology to the chicken TAPBPR sequence provided herein (e.g., in the figure(s) herewith) with activity of the chicken TAPBPR of the chicken TAPBPR sequence provided herein (e.g., in the figure(s) herewith, e.g., FIG. 1), as well as nucleic acid molecules encoding such additional chicken TAPBPR sequences that are considered part of the inventions of this disclosure. With respect to substitution within the chicken TAPBPR sequence provided herein (e.g., in the figure(s) herewith, e.g., FIG. 1), it is advantageous that substitution be a conservative replacement or conservative substitution, e.g., a given amino acid be changed or replaced or substituted with one having similar biochemical properties, e.g., as to charge and/or hydrophobicity and/or size. In this regard, amino acids and making conservative changes or replacements or substitutions, amino acids are generally grouped into six main classes based on their structure and general chemical characteristics of their side chains (R groups). (Aliphatic: Glycine, Alanine, Valine, Leucine, Isoleucine or G, A, V, L, I; Hydroxyl or sulfur/selenium-containing: Serine, Cysteine, Selenocysteine, Threonine, Methionine or S, C, U, T, M; Cyclic: Proline (P); Aromatic: Phenylalanine, Tyrosine, Tryptophan or F, Y, W; Basic: Histidine, Lysine, Arginine or H, K, R; and Acidic and their amides: Aspartate, Glutamate, Asparagine, Glutamine or D, E, N, Q, such that substitution of one amino acid with another with both being within any one group or both being among the cyclic and aromatic groups, may be considered a conservative change, replacement or substitution).

The below table provides a list of interaction MHC-I molecules. Underlined are the completely “new” allotypes that interact with the chicken and in black the ones that have been previously observed for the human, but there is better binding to chicken. So, in total there are 10 HLAs spanning 9 supertypes where there is better ligand exchange properties using chicken TAPBPR.

| Human | Supertype | Chicken | Supertype |
|---|---|---|---|
| A*02:01 | A02 | | |
| A*02:03 | A02 | A*01:01 | A01 |
| A*02:06 | A02 | A*29:01 | A01 A24 |
| A*23:01 | A24 | A*29:02 | A01 A24 |
| A*24:02 | A24 | B*08:01 | B08 |
| A*24:03 | A24 | B*37:01 | B44 |
| A*03:01 | A03 | B*57:03 | B58 |
| A*68:02 | A02 | B*59:01 | Unclassified |
| A*69:01 | A02 | C*01:02 | — |
| A*74:01 | A03 | C*03:04 | — |
| A*29:02 | A01 A24 | C*03:03 | — |
| A*03:01 | A03 | | |
| B*15:10 | B27 | | |
| B*27:09 | B27 | | |
| B*37:01 | B44 | | |
| B*38:01 | B27 | | |
| B*73:01 | B27 | | |
| B*35:03 | B07 | | |
| C*01:02 | — | | |

Underlined = new allotypes

The below table provides a list of disease associations of the HLA allotypes that are amenable to ligand exchange with chicken TAPBPR.

| Allotype | Supertype | Disease | References |
|---|---|---|---|
| A*01:01 | A01 | Psoriatic Arthritis; COVID-19 | (Chen et al. 2019; Shkurnikov et al. |
| | | | 2021) |
| A*29:01 | A01 A24 | Birdshot Retinochoroidopathy | (Nussenblatt et al. 1982) |
| B*08:01 | B08 | Myasthenia gravis; | (Varade et al. 2018; Regueiro et al. |
| | | Rheumatoid Arthriti; | 2021; Wadelius et al. 2018) |
| | | Sulfasalazine-Induced | |
| | | Agranulocytosis | |
| B*57:03 | B58 | HIV; Drug-induced liver | (Payne et al. 2014; Petros et al. 2017) |
| | | injury | |
| B*59:01 | unclassified | Stevens-Johnson Syndrome | (Kim et al. 2010) |

### REFERENCES FROM THE ABOVE TABLE

Chen, Jingjing, Fan Yang, Yan Zhang, Xing Fan, Hui Xiao, Wenjun Qian, Yuling Chang, et al. 2019. “HLA-A*01:01 in MHC Is Associated with Psoriatic Arthritis in Chinese Han Population.” Archives of Dermatological Research 311 (4): 277-85. https://doi.org/10.1007/s00403-019-01902-3.
Kim, Sae-Hoon, Myunghwa Kim, Kyung Wha Lee, Sang-Heon Kim, Hye-Ryun Kang, Heung-Woo Park, and Young-Koo Jee. 2010. “HLA-B*5901 Is Strongly Associated with Methazolamide-Induced Stevens-Johnson Syndrome/Toxic Epidermal Necrolysis.” Pharmacogenomics 11 (6): 879-84. https://doi.org/10.2217/pgs.10.54.
Nussenblatt, Robert B., Kamal K. Mittal, Stephen Ryan, W. Richard Green, and A. Edward Maumenee. 1982. “Birdshot Retinochoroidopathy Associated with Hla-A29 Antigen and Immune Responsiveness to Retinal S-Antigen.” American Journal of Ophthalmology 94 (2): 147-58. https://doi.org/10.1016/0002-9394(82)90069-1.
Payne, R. P., S. Branch, H. Kloverpris, P. C. Matthews, C. K. Koofhethile, T. Strong, E. Adland, et al. 2014. “Differential Escape Patterns within the Dominant HLA-B*57:03-Restricted HIV Gag Epitope Reflect Distinct Clade-Specific Functional Constraints.” Journal of Virology 88 (9): 4668-78. https://doi.org/10.1128/JVI.03303-13.
Petros, Zelalem, Junko Kishikawa, Eyasu Makonnen, Getnet Yimer, Abiy Habtewold, and Eleni Aklillu. 2017. “HLA-B*57 Allele Is Associated with Concomitant Anti-Tuberculosis and Antiretroviral Drugs Induced Liver Toxicity in Ethiopians.” Frontiers in Pharmacology 8 (February). https://doi.org/10.3389/fphar.2017.00090.
Regueiro, Cristina, Desire Casares-Marfil, Karin Lundberg, Rachel Knevel, Marialbert Acosta-Herrera, Luis Rodriguez-Rodriguez, Raquel Lopez-Mejias, et al. 2021. “HLA-B*08 Identified as the Most Prominently Associated Major Histocompatibility Complex Locus for Anti-Carbamylated Protein Antibody-Positive/Anti-Cyclic Citrullinated Peptide-Negative Rheumatoid Arthritis.” Arthritis & Rheumatology 73 (6): 963-69. https://doi.org/10.1002/art.41630.
Shkurnikov, Maxim, Stepan Nersisyan, Tatjana Jankevic, Alexei Galatenko, Ivan Gordeev, Valery Vechorko, and Alexander Tonevitsky. 2021. “Association of HLA Class I Genotypes With Severity of Coronavirus Disease-19.” Frontiers in Immunology 12 (February): 641900. https://doi.org/10.3389/fimmu.2021.641900.
Varade, Jezabel, Ning Wang, Che Kang Lim, Tao Zhang, Yuanwei Zhang, Xiaomin Liu, Fredrik Piehl, et al. 2018. “Novel Genetic Loci Associated HLA-B*08:01 Positive Myasthenia Gravis.” Journal of Autoimmunity 88 (March): 43-49. https://doi.org/10.1016/j.jaut.2017.10.002.
Wadelius, Mia, Niclas Eriksson, Reinhold Kreutz, Emmanuelle Bondon-Guitton, Luisa Ibanez, Alfonso Carvajal, M. Isabel Lucena, et al. 2018. “Sulfasalazine-Induced Agranulocytosis Is Associated With the Human Leukocyte Antigen Locus.” Clinical Pharmacology & Therapeutics 103 (5): 843-53. https://doi.org/10.1002/cpt.805.

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### EXAMPLES

### Example 1: Detailed Protocols

Recombinant TAPBPR protein expression and purification. The luminal domain of chicken, mouse and human TAPBPR protein was stably expressed in the Drosophila melanogaster S2 cell line. The cultures were induced with 1 mM CuSO4 and after 4 days the supernatant was collected. The secreted TAPBPR molecules tagged with 6-His were purified using high-density metal affinity agarose resin (ABT, Madrid). The eluted protein was further purified by size-exclusion chromatography using a Superdex 200 16/600 increase column at a flow rate of 1 mL/min in 150 mM NaCl and 20 mM sodium phosphate pH 7.4 (Jiang et al. 2017).

Protein expression, refolding, purification and biotinylation of recombinant pMHC-I molecules. Plasmid DNA encoding the BirA tagged luminal domain of MHC-I heavy chains and human β2-microglobulin (hβ₂m) were provided by the NIH tetramer facility (Emory University) and transformed into Escherichia coli BL21 (DE3) cells (Novagen). BirA-tagged MHC-I proteins were expressed in Luria-Broth media, and inclusion bodies were collected and purified using a standard protocol. In vitro refolding of BirA-tagged pMHC-I molecules was performed by slowly diluting a 200 mg mixture of BirA-tagged MHC-I and hβ₂m at 1:3 molar ratio over 24 hours in refolding buffer (0.4 μM L-Arginine, 100 mM Tris pH 8, 2 mM EDTA, 4.9 mM reduced glutathione, 0.57 mM oxidized glutathione) containing 10 mg of placeholder peptide. BirA-tagged pMHC-I refolding was proceeded for 96 hours and followed by size-exclusion chromatography for protein purification. BirA-tagged pMHC-I molecules were then biotinylated using the BirA biotin-protein ligase bulk reaction kit (Avidity), according to the manufacturer's instructions. Biotinylated pMHC-I was buffer exchanged into PBS pH 7.4 using Amicon Ultra centrifugal filter units with a 10 kDa membrane cut-off, and the level of biotinylation was evaluated by SDS-PAGE gel shift assay in the presence of excess streptavidin. A biotinylated pMHC-I solution at a final concentration of 5 mg/mL was obtained.

### Example 2: Peptides

All peptide sequences are given as standard single letter code. High-affinity epitopic peptides for different HLA allotypes were selected by netMHCpan4.1 and purchased from Genscript at a purity of >90%. The placeholder peptide was purchased from Genscript at a purity of 98%. Peptides were solubilized in distilled water and centrifuged at 14000 rpm for 15 minutes. The concentration of each peptide solution was measured and calculated using absorbance and extinction coefficient at 205 nm wavelength.

### Example 3: Native Gel Shift Assay

For native gel shift assays, purified MHC-I molecules refolded with photo-labeled peptides (pMHC-I) and TAPBPR proteins were loaded into polyacrylamide gels alone at 7 μM concentration and as mixtures. For the mixtures, 1:1 molar ratio of pMHC-I and TAPBPR samples at 7 μM each were prepared and incubated at room temperature (RT) for 1 hour. The samples were then UV-irradiated for another hour at 365 nm and were run at 90 V for 5.5 hours on a 12% polyacrylamide gel. Staining was performed using InstantBlue (Novus Biologicals).

### Example 4: TAPBPR Tetramerization

TAPBPR tetramerization was performed as previously described herein. Briefly, TAPBPR molecules were biotinylated as previously described for pMHC-I molecules and a 4:1 molar ratio of biotinylated TAPBPR was added to streptavidin-PE (Agilent Technologies, Inc.) in ten additions every 10 min at RT.

### Example 5: Single Antigen Bead Screen

For the Single Antigen Bead Screen, 7 μM of TAPBPR in tetramers were mixed with 4 μL of the LABScreen single antigen HLA-I bead suspension (OneLambda, Inc., CA, USA) in a 96-well plate. The samples were incubated for 1 hour, 550 rpm at RT, washed four times in Wash Buffer (OneLambda, Inc., CA, USA) to remove excess of tetramers and re-suspended in 1×PBS buffer (pH 7.2). For the negative controls, beads were incubated with the Anti-HLA Class I antibody W6/32 (Abcam, ab22432) for 30 min, 550 rpm at RT and washed three times prior tetramer addition. To test the levels of peptide loaded MHC-I molecules on the beads, Applicants used the same W6/32 antibody and the secondary anti-mouse PE-conjugated antibody (Abcam, ab97024) for detection. The levels of TAPBPR bound to the beads were measured using the Luminex 100 Liquid Array Analyzer System and the results were analyzed in GraphPad Prism v7.

### Example 6: Surface Plasmon Resonance Experiments and Data Analysis

SPR experiments were conducted using a BiaCore T200 instrument (Cytiva) in SPR buffer (50 mM NaCl, 20 mM sodium phosphate pH 7.2, 0.1% Tween-20). Approximately 1000 resonance units (RU) of biotinylated Chicken TAPBPR were immobilized at 30 μL/min on a streptavidin-coated chip (GE Healthcare). Various concentrations (0, 1, 2.5, 5, and 10 μM) of HLA-A*01:01/STAPGJLEY (SEQ ID NO: 9) and HLA-A*24:02/VYGJVRACL (SEQ IDNO: 3) were injected over the chip at 25° C. at a flow rate of 10 μL/min for 60 sec followed by a buffer wash with 180 seconds dissociation time. Equilibrium data were collected. The SPR sensorgrams and equilibrium dissociation constants KD values were analyzed using the surface-bound analysis settings in BiaCore T200 evaluation software (Cytiva). SPR sensorgrams were prepared in GraphPad Prism v7.

### Example 7: Differential Scanning Fluorimetry and Data Analysis

Differential scanning fluorimetry was used to measure the thermal stability of the TAPBPR proteins and the pMHC-I molecules. 7 μM of TAPBPR protein and for the latter 7 μM of pMHC-I, 70 uM peptide, and 1 uM human or Chicken TAPBPR were mixed with 10×SYPRO Orange dye in a buffer of 50 mM NaCl, 20 mM sodium phosphate pH 7.2 to a final volume of 20 μL. Samples were loaded into MicroAmp Optical 384 well plate and ran in triplicates. The experiment was performed on an QuantStudio™ 5 Real-Time PCR machine with excitation and emission wavelength set to 470 nm and 569 nm. The thermal stabilities of peptide MHC complexes were compared by plotting the first derivative of each melting curve and extracting the peak as the melting temperature (Tm). The thermal stabilities were measured by gradually increasing temperature at a rate of 0.017° C. per second between 25° C. to 95° C.

### Example 8: chTAPBPR-Mediated Ligand Exchange

pMHC-I molecules were mixed with TAPBPR protein (1:7 TAPBPR/pMHC-I molar ratio) and a desired peptide (1:10 pMHC-I/peptide molar ratio). Samples were incubated for 2 hours at room temperature and the exchanged pMHC-I molecules were used for differential scanning fluorimetry analysis and tetramerization. Streptavidin-PE or APC (4:1 pMHC-I/streptavidin molar ratio) or Klickmer-APC (30:1 pMHC-I/Klickmer molar ratio) were added to pMHC-I molecules over 10-time intervals every 10 minutes at room temperature in the dark. pMHC-I multimers were then washed with 1000 volumes of PBS using an Amicon Ultracel centrifuge filter with a 100 kDa membrane cut-off to remove Chicken TAPBPR and excess peptide. Purified pMHC-I multimers can be stored up to 3 weeks at 4° C. for fluorescence-activated cell sorting.

### Example 9: chTAPBPR-Mediated Ligand Exchange

Applicants outline detailed protocols for the design, synthesis, and application of MHC-I peptides with unnatural amino acids as conditional ligands for chaperone-mediated peptide exchange. Applicants provide an exemplar application using a β-Phenylalanine containing synthetic peptide as a placeholder, which forms a stable complex with HLA-A*02:01 upon in vitro refolding. Furthermore, Applicants elaborate conditional peptides for multiple common HLA allotypes from all three genes (HLA-A. -B, -C). Using the molecular chaperone TAPBPR, Applicants then perform an exchange reaction, completely replacing the conditional ligand with high-affinity peptides of interest which correspond to well-characterized viral and tumor T cell epitopes. β-Phenylalanine-containing peptides can be implemented for a range of common human HLA allotypes, to enable epitope discovery, T cell repertoire characterization and therapeutics development in several clinically relevant settings.

Class I MHC (MHC-I) proteins are expressed in all nucleated cells, and they are implicated in aspects of most, if not all, adaptive immune responses. They function by detecting aberrantly expressed proteins and alerting the immune system to the presence of intracellular threats by interacting with specialized receptors on T cells and Natural Killer (NK) cells (Rossjohn et al. 2015). There are thousands of different allotypes of class I HLA proteins (Human Leucocyte Antigen, the human MHC), encoded by the HLA locus found at the short arm of chromosome 6, and clear disease associations with specific HLA alleles (Vita et al. 2019). Classical HLA genes are further classified in 3 sub-classes HLA-A, HLA-B and HLA-C. An individual's genotype therefore comprises 6 class-I HLA genes, which, given the highly polymorphic nature of the MHC-I peptide binding groove, can create an unlimited number of combinations at the population level, ensuring species adaptability to emerging pathogenic strains. HLA allotypes can be further grouped into supertypes, according to their peptide binding specificities (Sidney et al. 2008). The repertoire of peptides which can bind to a given HLA allotype is often represented in compact form as a sequence “logo”, where for each position P1. P9 the relative frequency of different amino acid types is shown (Schneider and Stephens 1990). An in silico functional clustering and classification of 121 common HLA-A, -B and -C allotypes based on their corresponding peptide sequence logos was performed by Rassmusen et al. This analysis has shown a wide distribution of peptides which can be displayed by different HLA molecules including charged, polar or hydrophobic amino acids and their combinations at defined anchor positions, indicating that the MHC-I structure can accommodate a diverse set of peptide sequences.

The intrinsic instability of empty (peptide-receptive) major histocompatibility complex class I (MHC-I) molecules limits the high-throughput production of peptide: MHC complexes presenting diverse peptide epitopes as either monomers, tetramers, or nanoparticle conjugates to enable a wide range of immunological assays of interest for diagnostic and therapeutic applications (Ljunggren et al. 1990) (Schumacher et al. 1990). Therefore, peptide exchange technologies are essential for rapidly generating peptide-MHC-I (pMHC-I) antigens used to probe polyclonal T cell receptors (TCRs) in various settings. Peptide exchange technologies are essential for the generation of peptide:HLA tetramers which are commonly used as a screening tool to probe diverse, polyclonal T cell repertoires in various disease settings, as well as for the development of pHLA-targeted therapeutics such as single-chain antibody fragments (scFVs) with minimum off-target cross-reactivity (Altman et al. 1996). Alternatively, exchange of peptide ligands on the cell surface can be used to bypass the endogenous MHC-I antigen processing pathway, and methods to promote exchange has important ramifications for developing systems of antigen-presenting cells (APCs) for T cell expansion in vitro, an essential component of autologous T cell therapy. Previous approaches employ a range of methods to perform peptide exchange on recombinantly produced HLA molecules in vitro. Recently, a peptide exchange methodology using the human molecular chaperone TAPBPR (Boyle et al. 2013) (Teng et al. 2002) has provided a valuable tool for generating p:HLA libraries (Overall et al. 2020).

In order to perform a net peptide exchange reaction, one must first identify a suitable conditional ligand, which serves as a “placeholder” peptide, but rapidly dissociates from the MHC-I groove upon interactions with the chaperone TAPBPR. Previous approaches for generating such ligands use “goldilocks” peptides, which are derived from high-affinity peptide epitopes by truncation of the N-terminal amino acid (McShan et al. 2018). Here Applicants describe an alternative approach for generating conditional peptide ligands for a range of disease-related MHC-I allotypes, which form stable complexes with MHC-I molecules but can be exchanged for desired peptides through a chaperone-assisted process. Rather than using truncated high affinity peptides, the new approach involves introducing a modified unnatural amino acid near the peptide C-terminus. This results in a MHC-I protein complex with higher thermal stability and improved exchanged properties, relative to the conventional goldilocks peptides. Applicants outline an exemplar application for the HLA-A*02:01 allotype and detailed protocols for: i) peptide exchange on conditional peptide ligand-loaded MHC-I molecules using human TAPBPR, ii) the analysis of peptide binding by complementary differential scanning fluorimetry (DSF) and fluorescence polarization (FP) assays, and iii) the parallel production of pMHC-I tetramers for T cell detection.

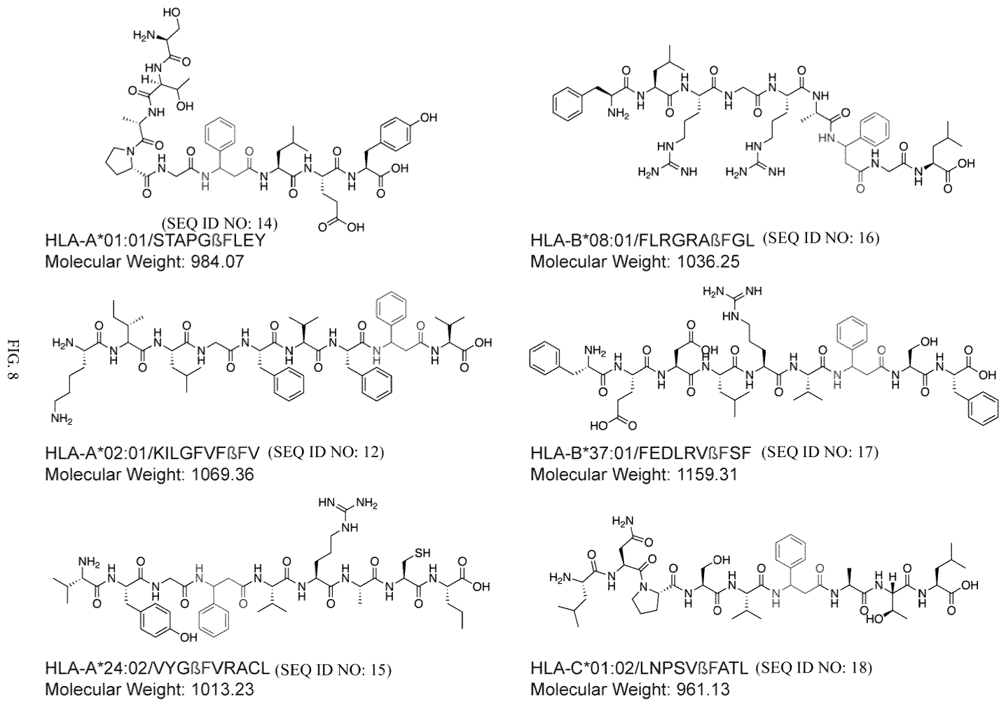

Conditional ligand beta-peptide synthesis (FIG. 8). All peptides were synthesized on a CEM Liberty Blue automated microwave peptide synthesizer using Fmoc protected amino acids (including Fmoc-β-Phe-OH) N, N′-Diisopropylcarbodiimide (DIC)/Ethyl cyanohydroxyiminoacetate (Oxyma) coupling on 2-chlorotrityl resin employing iterative cycles of the following methods:

Resin Loading: 2-chlorotrityl resin (0.1 mmol) was swollen in dichloromethane for 10 minutes before being treated with a solution of Fmoc-protected amino acid (4 eq.) and diisopropylethylamine (4 eq.) for 30 minutes with mixing. The resin was washed with DMF (3×10 ml) and treated with methanol (8 eq.) and diisopropylethylamine (4 eq.) for 45 minutes.

Fmoc Deprotection: The resin was treated with 4% piperidine in DMF and heated to 90° C. by microwave irradiation for 1.05 minutes before being washed with DMF (2×2 ml, 1×3 ml).

Coupling: The resin was treated with Fmoc-protected amino acid (5 eq.), N,N′-Diisopropylcarbodiimide (5 eq.) and Ethyl cyanohydroxyiminoacetate (5 eq.) heated to 90° C. by microwave irradiation for 1.05 minutes before being washed with DMF (1×2 ml).

Global Deprotection and Cleavage: The resin was treated with trifluoroacetic acid/water/triisoproylsilane/phenol (88:5:5:2) for 1 hour. The eluent was concentrated under a flow of nitrogen and then treated with ice-cold ether. The resulting precipitate was collected by centrifugation and dried in vacuo.

Peptide Purification. The peptides were purified by reverse phase chromatography eluting with 5-95% acetonitrile in water containing 0.05% trifluoroacetic anhydride over a C18 column. Peaks containing peptides were identified by LC-MS, pooled, and concentrated in vacuo to yield a colorless solid.

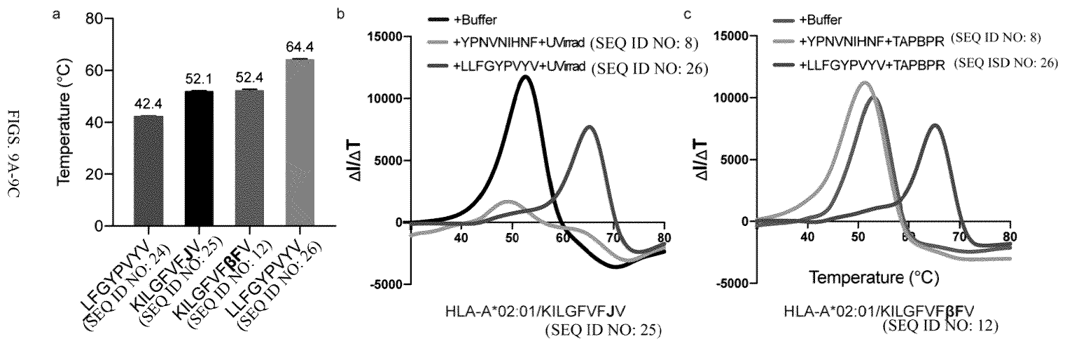

Recombinant TAPBPR expression and purification (FIG. 9). The luminal domain of the different TAPBPR proteins was stably expressed in the Drosophila melanogaster S2 cell line. The cultures were induced with 1 mM CuSO4 and after 4 days the supernatant was collected. The secreted TAPBPR molecules tagged with 6-His were purified using high-density metal affinity agarose resin (ABT, Madrid). The eluted protein was further purified by size-exclusion chromatography using a Superdex 200 16/600 increase column at a flow rate of 1 mL/min in 150 mM NaCl and 20 mM sodium phosphate pH 7.4 (Jiang et al. 2017).

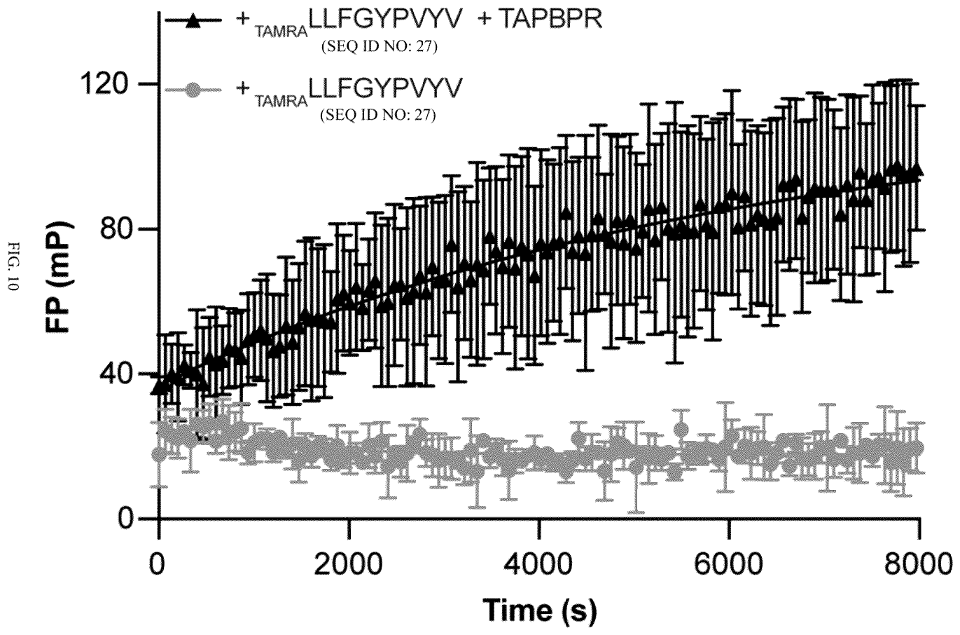

### Recombinant MHC-I Expression, Refolding, Purification, and Biotinylation (FIG. 10).

Expression of MHC-I heavy chain and light chain. Plasmid DNA encoding the BirA tagged luminal domain of MHC-I heavy chains and human 02-microglobulin (hβ₂m) were provided by the NIH tetramer facility (Emory University) and transformed into Escherichia coli BL21 (DE3) cells (Novagen). BirA-tagged MHC-I proteins were expressed in Luria-Broth media, and inclusion bodies were collected and purified using a standard protocol.

Refolding of human MHC-I with the conditional ligand beta-peptide. In vitro refolding of BirA-tagged HLA-A*02:01/KILGFVFfßFV was performed by slowly diluting a 200 mg mixture of BirA-tagged MHC-I heavy chain and hβ₂m at 1:3 molar ratio over 24 hours in refolding buffer (0.4 μM L-Arginine, 100 mM Tris pH 8, 2 mM EDTA, 4.9 mM reduced glutathione, 0.57 mM oxidized glutathione) containing 10 mg of the beta-peptide KILGFVFßFV. BirA-tagged HLA-A*02:01/KILGFVFßFV refolding was then proceeded for 96 hours and followed by size-exclusion chromatography for protein purification.

Biotinylation. BirA (LHHILDAQKMVWNHR (SEQ ID NO: 113))-tagged HLA-A*02:01/KILGFVFßFV (SEQ ID NO: 12) molecules were biotinylated using the BirA biotin-protein ligase bulk reaction kit (Avidity), according to the manufacturer's instructions. Note that high salt concentration can decrease the protein biotinylation efficiency. Biotinylated pMHC-I was washed, and buffer exchanged into 1×PBS pH 7.4 using Amicon Ultra centrifugal filter units with a 10 kDa membrane cut-off. The level of biotinylation was evaluated by the gel shift assay in the presence of excess streptavidin. A biotinylated pMHC-I solution at a final concentration of 5 mg/mL was obtained.

Use the biotinylated pMHC-I solution immediately or store at 4° C. for up to one week. For long-term storage, flash freeze the biotinylated monomer and store the samples with an end concentration of approximately 5 mL/mL at −80° C.

Biotinylated HLA-A*02:01/KILGFVFβFV (SEQ ID NO: 12) were mixed with TAPBPR (1:7 TAPBPR/pMHC-I molar ratio) and the desired peptide (1:10 pMHC-I/peptide molar ratio). Samples were incubated for 2 hours at room temperature, and the exchanged pMHC-I molecules were used for differential scanning fluorimetry (DSF), fluorescence polarization (FP), and tetramerization.

Thaw frozen biotinylated HLA-A*02:01/KILGFVFβFV (SEQ ID NO: 12) samples on ice before setting up the peptide exchange reaction.

Confirm and adjust the biotinylated monomer concentration to 100 μM, the peptide solution concentration to 1 mM, and the TAPBPR concentration to 10 μM.

Mix 5 μL (500 picomol) HLA-A*02:01/KILGFVFβFV (SEQ ID NO: 12) and 5 μL (5000 picomol) peptide solution with 5 μL (50 picomol) of human TAPBPR in a 50 μL peptide exchange reaction (the final concentration of pMHC-I, peptide, and TAPBPR is 10 μM, 1 μM, and 100 μM). Such reaction can be scale up for multiple uses of pMHC-I monomers.

Incubate in a drawer at room temperature for two hours.

The completed peptide exchange reaction can be used for a quality control by differential scanning fluorimetry or preferably by performing a fluorescence polarization assay. pMHC-I monomer with confirmed peptide exchange can be directly used for tetramerization.

Differential Scanning Fluorimetry and Data analysis (FIG. 11^{Box1}) Differential scanning fluorimetry was used to measure the thermal stability of the TAPBPR proteins and the pMHC-I molecules. 50 μL peptide exchange reaction obtained from step 5 were mixed with 12.5 μL 50×SYPRO Orange dye in a buffer of 150 mM NaCl, 20 mM sodium phosphate pH 7.2. Samples were loaded into MicroAmp Optical 384 well plate (20 μL per well) and ran in triplicates. The experiment was performed on an QuantStudio™ 5 Real-Time PCR machine with excitation and emission wavelength set to 470 nm and 569 nm. The thermal stabilities of peptide MHC complexes were compared by plotting the first derivative of each melting curve and extracting the peak as the melting temperature (Tm). The thermal stabilities were measured by gradually increasing temperature at a rate of 0.017° C. per second between 25° C. to 95° C.

Fluorescence Polarization and Data analysis (FIG. 11⁵). Fluorescence polarization (FP) of HLA-A*02:01/KILGFVFßFV was performed using a TAX9 peptide labeled with TAMRA dye (_{TAMRA}KLFGYPVYV, herein called TAMRA-TAX9) (Biopeptek Inc.). A fixed amount of HLA-A*02:01/KILGFVFßFV (200 nM) and TAPBPR (20 nM) were mixed. 40 nM TAMRA-TAX9 was added and incubated for 2 hours at RT. All FP experiments were performed in the dark to protect the TAMRA-TAX9 peptides from photolysis. Each experiment was performed at room temperature in a volume of 100 μL and loaded onto a black 96-well polystyrene assay plate (Corning®). FA data was recorded via a SpectraMax iD5 Multi-Mode Microplate Readers with an excitation filter of λₑₓ=531 nm and an emission filter of λₑₘ=595 nm. Kinetic association was monitored for 10 hours, and polarization measurements were recorded every 60 seconds. All reactions as well as kinetic monitoring via FP were performed at room temperature (20° C.). Each experiment was performed in triplicate. All samples were prepared in matching buffer (150 mM NaCl, 20 mM sodium phosphate pH 7.2, 0.05% (v/v) tween-20).

Tetramerization (FIG. 11⁶). Streptavidin-PE or APC (4:1 pMHC-I/streptavidin molar ratio) or Klickmer-APC (30:1 pMHC-I/Klickmer molar ratio) were added to pMHC-I molecules over 10-time intervals every 10 minutes at room temperature in the dark. pMHC-I tetramers/multimers were then washed with 1000 volumes of PBS using an Amicon Ultracel centrifuge filter with a 100 kDa membrane cut-off to remove TAPBPR and excess peptide. Purified pMHC-I multimers can be stored up to 1 month at 4° C. for antigen-specific T cell detection and other relevant applications.

To 50 μL peptide exchange reaction, add 1.8 μl streptavidin-PE (2 mg/ml) or 1 μl-APC (2 mg/ml) every 10 minutes for a total of 10 times. The volume of fluorophores can be scale up according to the volume of the peptide exchange reaction. The addition of streptavidin to the biotinylated monomers is performed at room temperature, with the samples kept in the dark between additions (usually in a drawer). The protocol will probably also work if the samples are kept on ice.

To remove excess peptide and TAPBPR from each individual tetramer, wash each tetramer using Amicon Ultracel centrifuge filter 100 kDa with 1000×1×PBS. The final tetramer for a particular peptide has a standard pMHC-I concentration of 0.5 mg/mL.

Flow cytometry and Data analysis (FIG. 117). Tetramer analysis was carried out by staining 2×10⁵ cells with 1 μg/mL of HLA-A02:01/SLLMWITQV (SEQ ID NO: 28), HLA-A02:01/ALLDRLRGVYA (SEQ ID NO: 29), HLA-A02:01/ALLMWITQV (SEQ ID NO: 30), HLA-A02:01/SLLAWITQV (SEQ ID NO: 31), and HLA-A02:01/SLLMAITQV (SEQ ID NO: 32) tetramers for 30 minutes at room temperature, followed by two washes with 30 volumes of FACS buffer (PBS, 1% BSA, 2 mM EDTA). Live/dead gating determined by staining with SYTOX™ Blue, a high-affinity nucleic acid stain that easily penetrates cells with compromised plasma membranes. All flow cytometric analysis was performed using a BD LSR II instrument. Live cells were gated based on forward and side scatter profiles and the data were analyzed using FlowJo software (FlowJo, LLC).

### Example 10: Xeno-Interactions Between MHC-I Proteins and Molecular Chaperones Enable Peptide Exchange on Disease-Relevant HLA Allotypes

Immunological chaperones Tapasin and TAPBPR play key roles in antigenic peptide repertoire optimization and quality control of nascent MHC-I molecules. The polymorphic nature of MHC-I proteins leads to a range of allelic dependencies on chaperones for assembly and cell-surface expression, limiting chaperone-mediated peptide exchange to a restricted set of human HLA allotypes. Here, Applicants demonstrate and characterize xeno-interactions between a chicken TAPBPR (chTAPBPR) ortholog and a set of HLA alleles. Applicants find that chTAPBPR recognizes empty MHC-I with broader allele specificity relative to its human counterpart and facilitates peptide exchange by maintaining a reservoir of receptive MHC-I molecules. Deep mutational scanning of hTAPBPR further identifies gain-of-function mutants, resembling the chicken sequence, which recognize the HLA-A*01 allele in situ and promote peptide exchange in vitro. These results highlight that polymorphic sites on MHC-I and chaperone surfaces can be engineered to manipulate their interactions, enabling chaperone-mediated peptide exchange on disease-relevant HLA alleles.

The proteins of class I major histocompatibility complex (MHC-I) display epitopic peptides on the cell surface, providing the foundation for immune surveillance of intracellular threats¹. CD8+ T cell receptors (TCRs) recognize aberrant antigens presented by MHC-I, triggering cytotoxic immune responses that can eliminate infected or malignant cells². MHC-I folding and peptide loading are subject to intricate cellular quality control. The peptide loading complex (PLC), comprising the TAP peptide transporter, the molecular chaperone tapasin, ERp57, and calreticulin, assemble peptide-MHC-I (pMHC-I) molecules with high-affinity peptides in the endoplasmic reticulum (ER), in a process termed peptide editing^{3,4}. In addition, TAPBPR, a homolog of tapasin that functions outside the PLC^{5,6}, plays a complementary role in MHC-I peptide cargo optimization and quality control⁷. Although both tapasin and TAPBPR function as peptide editors⁸⁻¹¹, TAPBPR also participates in the quality control and reglucosylation cycle of MHC-I molecules by promoting interactions with UDP-glucose:glycoprotein glucosyltransferase 1 (UGT1)¹². These unique functions of tapasin and TAPBPR ultimately lead to an optimized repertoire of stable pMHC-I molecules that traffic to the cell surface and are properly conformed for interactions with T cell receptors.

The classical HLA loci (Human Leucocyte Antigen, the human MHC) encode the most polymorphic proteins in the human genome with more than 35,000 known allotypes¹³. Polymorphic residues in the HLA peptide-binding groove result in a unique peptide repertoire displayed by each allotype¹⁴, enabling species adaptability to emerging infections. HLA-A, -B, and -C allotypes show divergent dependencies on molecular chaperones for proper assembly and cell-surface expression, which has important biological ramifications. Tapasin independence of MHC-I alleles correlates with an increased breadth of the peptide repertoire¹⁵ and can lead to enhanced control of HIV viral loads¹⁶. Likewise, TAPBPR-knockout cell lines express MHC-I molecules presenting a broader spectrum of peptides, relative to wild type (WT) cells¹⁷. Amino acid polymorphisms in the α₂ and α₃ domains can affect interactions with tapasin and cell-surface expression levels^{18,19}. While polymorphic residues located on the floor of the MHC-I groove lead to a gradient of tapasin dependencies for HLA-B alleles²⁰, TAPBPR preferentially interacts with HLA-A over HLA-B and -C alleles, and residues analogous to H114 and Y116 can confer gain-of-function binding to TAPBPR when introduced to non-interacting HLA allotypes²¹. Some of these effects can be explained by the interacting surfaces observed in the X-ray structures of chaperoned, peptide-deficient MHC-I complexes^{22,23}, including a conserved allosteric site underneath the α₂₋₁ helix revealed by solution NMR¹¹. Furthermore, highly polymorphic MHC-I residues distant to the chaperone binding sites can influence interactions with TAPBPR by modulating the dynamic sampling of an “open” conformation^{24,25}. Notwithstanding, chaperones can recognize a much broader allelic repertoire of partially folded MHC-I molecules, as shown by deep mutagenesis experiments^{24,26}. This adaptability of underlying interactions suggests that the corresponding conformational epitopes on MHC-I molecules are suboptimal for binding to chaperones. Notably, while in humans the antigen-processing genes are removed from the HLA-I loci, close gene association in the chicken MHC has led to co-evolution of antigen-processing and class-I genes, causing mirrored polymorphisms in TAP and tapasin that segregate with specific MHC-I alleles²⁷⁻²⁹. The orthologous TAPBPR gene was initially identified in mice and humans and later in fish and chickens, strongly suggesting a conserved function³⁰. Although modeling studies suggest a similar overall protein fold³¹, structural distinctions are evident and functional differences among TAPBPR orthologs have not yet been characterized, with most studies concentrating on the human protein.

Here, Applicants characterize and contrast the allelic specificity, and molecular and functional features of HLA interactions with TAPBPR orthologs from Homo sapiens (human), Gallus (chicken), and Mus musculus (mouse). Applicants discover novel xeno-interactions between chicken TAPBPR (chTAPBPR) and multiple HLA allotypes that are not competent for binding with human TAPBPR (hTAPBPR), such as HLA-A*01:01 and HLA-B*08:01, and demonstrate direct interactions with peptide-loaded or -deficient molecules covering all six classified HLA-A supertypes, B08, and B44 supertypes³², as well as HLA-E, HLA-G, and the major histocompatibility complex, class I-related (MR1). Deep mutational scanning of an hTAPBPR construct expressed at the plasma membrane further identified gain-of-function mutations that in part mimic substitutions found in chTAPBPR and which greatly enhance peptide exchange function on HLA-A*02:01, while also enabling exchange on HLA-A*01:01. Overall, Applicants' results underscore a strong correlation between the capacity of TAPBPR variants to bind empty molecules and maintain them in a peptide-receptive state for peptide exchange function in vitro. Applicants' findings highlight the plasticity of recognition surfaces on MHC-I and TAPBPR, which can be used to manipulate interactions for understanding the molecular basis of peptide exchange function. Insights from Applicants' work can be leveraged to enable peptide exchange technologies on a wider range of disease-associated HLA allotypes, including nonclassical MHC-I molecules.

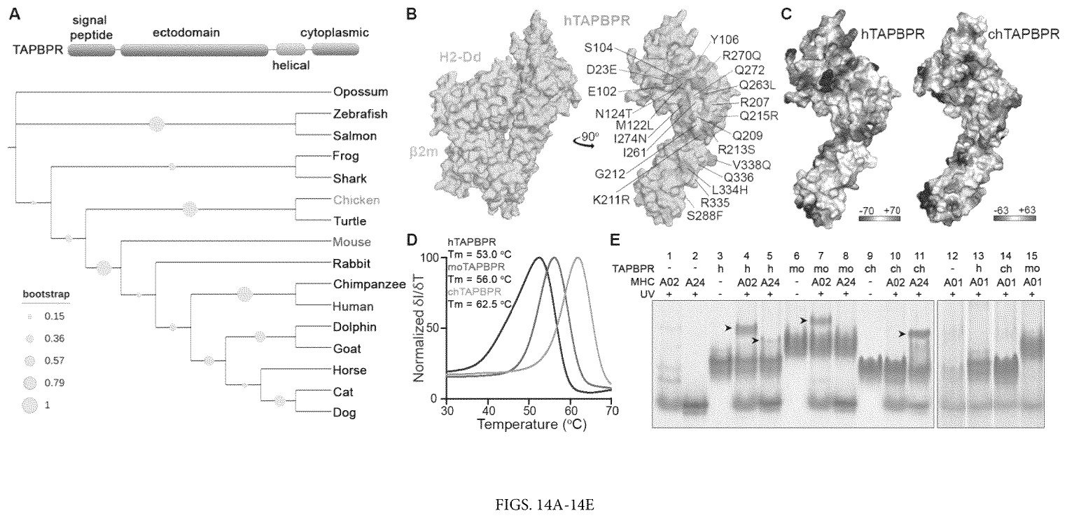

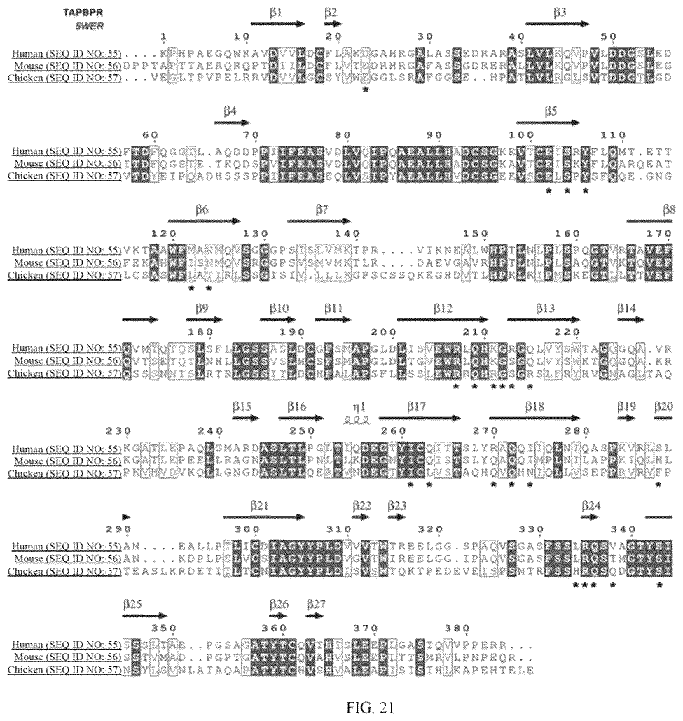

Divergent TAPBPR orthologs show increased stability and can interact with human MHC-I. To identify evolutionarily divergent TAPBPR orthologs with interesting features relevant to interactions with MHC-I molecules, Applicants first performed a phylogenetic analysis using the ectodomains of 16 TAPBPR sequences from different species (FIG. 14a). Human and chicken TAPBPR share 46% sequence identity with regions of relatively higher conservation. Mouse TAPBPR (moTAPBPR) is an intermediate between the two (77%/50% sequence identity relative to human/chicken, FIG. 21). The structure of hTAPBPR interacting with mouse H2-D^{d}/human β₂m (hβ₂m, PDB ID: 5WER) illustrates that 12 out of 23 residues in contact with the MHC-I heavy chain²³ are polymorphic between human and chicken TAPBPR (FIG. 14b). Due to the highly polymorphic nature of HLA-I, all interacting residues with TAPBPR can vary among allotypes¹⁴, in agreement with the established range of HLA dependencies on molecular chaperones for cell surface expression^{16,20}. Structure modeling of the chTAPBPR sequence based on the human ortholog structure followed by an electrostatic surface potential analysis reveals divergent features of MHC-I interaction footprints on TAPBPR (FIG. 14c). More specifically, contiguous surfaces formed by residues 16-20, 35-40, 139-145 and R270 define a positively charged epitope in human TAPBPR, but neutral in chicken. Together, the sequence and structure-based analyses highlight the potential for xeno-interactions between TAPBPR orthologs and complementary sets of HLA-I.

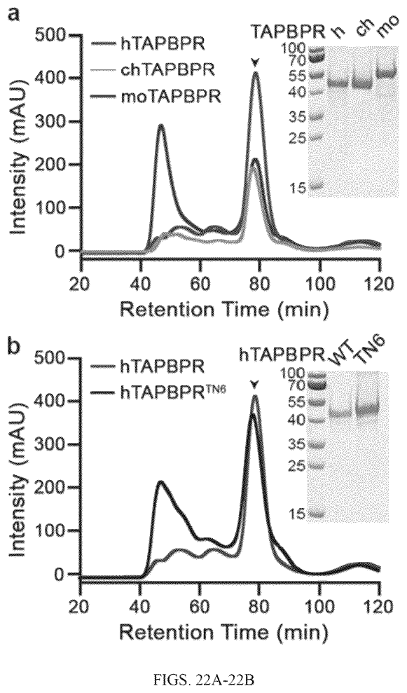

Applicants next sought to compare the biochemical properties of different TAPBPR orthologs expressed as recombinant proteins and purified by size exclusion chromatography (FIG. 22). Differential scanning fluorimetry (DSF) analysis showed a significant increase in the thermal stabilities of mouse and chicken TAPBPR, with melting temperatures (Tₘ) of 56° C. and 62.5° C., compared to hTAPBPR (Tₘ=53° C.) (FIG. 14d). Applicants further evaluated interactions of different TAPBPR orthologs with common HLA allotypes HLA-A*01:01, A*02:01, and A*24:02 using an electrophoretic mobility shift assay (EMSA), performed under native conditions. Here, Applicants characterized interactions with peptide-deficient MHC-I molecules, as previous studies showed the formation of high-affinity protein complexes^{10,22,23}. While producing empty MHC-I molecules is challenging due to their instability³³, Applicants refolded HLA molecules with photosensitive peptides, which could dissociate upon UV irradiation generating empty molecules^{34,35}. Different HLAs (lanes 1, 2, and 12) and TAPBPR orthologs (lanes 3, 6, and 9) presented characteristic mobilities (FIG. 14e), and the appearance of new bands revealed evidence of TAPBPR/MHC-I complex formation. Applicants observe complex formation between hTAPBPR and HLA-A*02:01 (lane 4) and to a lesser extent with HLA-A*24:02 (lane 5), but not with HLA-A*01:01 (lane 13). However, moTAPBPR formed a new band only with HLA-A*02:01 (lane 7) and chTAPBPR solely interacts with to HLA-A*24:02 (lane 11), showing no binding to HLA-A*01:01 (lanes 14 and 15). These results suggest that recombinant TAPBPR orthologs have distinct binding profiles to MHC-I allotypes.

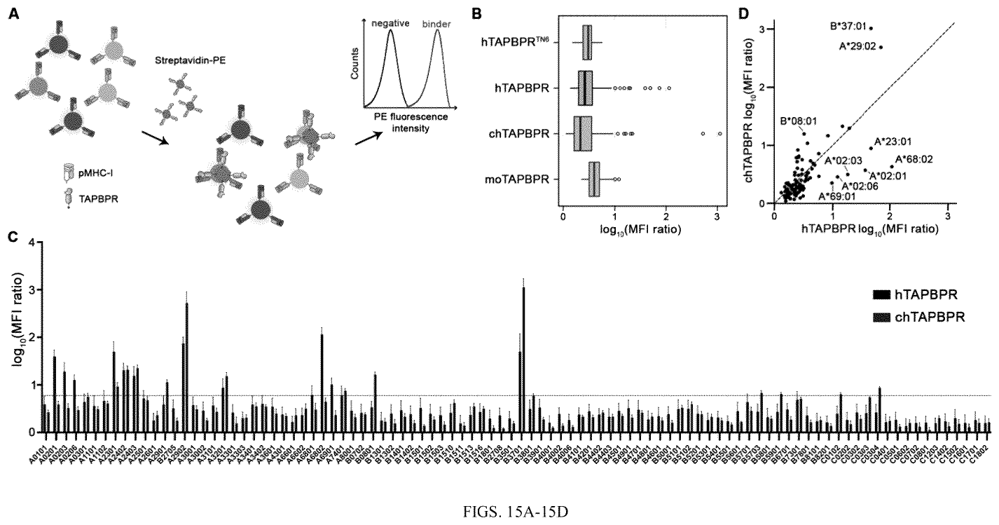

TAPBPR orthologs exhibit distinct binding profiles with HLAs on single-antigen beads. To evaluate interactions between recombinantly expressed TAPBPR from different organisms and a broad range of human MHC-I allotypes, Applicants first used single antigen HLA class I beads (SABs). The beads are fluorescently color-coded and coated with 96 different HLA allotypes^{36,37}, derived from expression in Epstein Barr virus (EBV)-transformed cell lines and thus loaded with a “garden variety” of cell-derived peptides. A SAB-based assay has been previously developed to study interactions between hTAPBPR and HLA-I²¹. Here, Applicants adapted the method to capture interactions of the different TAPBPR orthologs by creating multivalent tetramers (FIG. 15a), as previously described for pMHC-I tetramers³⁸. The advantages of using tetramers are to improve avidity for identifying low affinity interactions with MHC-I and allow direct comparisons of MHC-I binding across TAPBPR orthologs, bypassing the need to develop primary anti-TAPBPR antibodies for each ortholog.

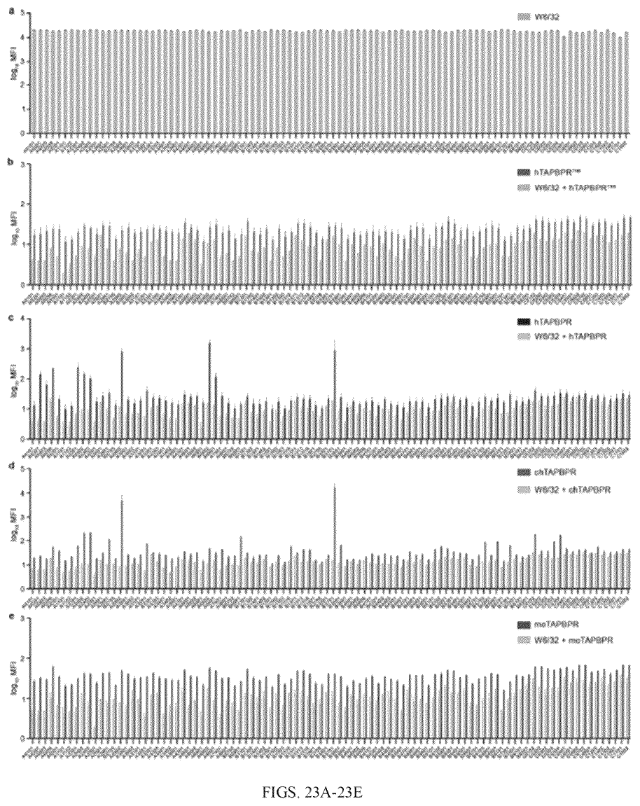

To confirm the conformational integrity of HLA-I molecules, Applicants compared the staining levels using the pan-HLA class I reactive monoclonal antibody W6/32, which recognized a conformational epitope of properly conformed heavy chain/2m complexes³⁹ with contributions from both polypeptide chains⁴⁰. Mean fluorescence intensity (MFI) levels were similar for all beads, revealing homogeneous protein levels across all HLA-I allotypes (FIG. 23a). Since the TAPBPR binding epitope on MHC-I was masked upon W6/32 binding, Applicants pre-incubated the beads with W6/32 followed by tetramer addition to measure and control for the non-specific background staining. The MFI levels of bound TAPBPR tetramers were lower upon W6/32 incubation (FIGS. 23b-e) and Applicants calculated the MFI ratio relative to the control experiment to depict the interactions with different HLA allotypes. Additionally, Applicants used the TN6 mutant of TAPBPR (hTAPBPR^{TN6}: E205K, R207E, Q209S, Q272S) (FIG. 22b, 16b) that does not interact with HLA-A*02:01^{9,10} to further exclude non-specific binding by setting a threshold of 5.97 (the mean MFI ratio of hTAPBPR^{TN6}+3G) to define significant interactions, relative to the control (FIGS. 15b, c).

Analysis of MFI ratios revealed significant differences in interaction profiles exhibited by human and chicken TAPBPR with different HLA supertype representatives. Particularly, hTAPBPR showed high levels of binding to HLA-A*32:01 (A01 supertype); HLA-A*29:02 (A01/A24 supertype); HLA-A*02:01, A*02:03, A*02:06, A*68:02 and A*69:01 (A02 supertype); HLA-A*68:01 and A*74:01 (A03 supertype); HLA-A*23:01, A*24:02, and A*24:03 (A24 supertype); and HLA-B*37:01 (B44 supertype) (FIG. 15c, Table 4). Chicken TAPBPR showed high levels of binding to HLA-A*32:01 (A01 supertype); HLA-A*29:01 and A*29:02 (A01/A24 supertype); HLA-A*74:01 (A03 supertype); HLA-A*23:01, A*24:02, and A*24:03 (A24 supertype); HLA-B*08:01 (B08 supertype); HLA-B*37:01 (B44 supertype); HLA-B*57:03 (B58 supertype); HLA-B*59:01 (unclassified); and HLA-C*01:02 and C*03:04 (C01 supertype), thereby demonstrating differential binding to HLA representatives and covering complementary sets of HLA-B and C supertypes (FIGS. 15c, d, Table 4). Conversely, chTAPBPR showed high levels of binding to A*32:01 (A01 supertype); A*29:01 and A*29:02 (A01/A24 supertype); HLA-A*24:02 and A*24:03 (A24 supertype); B*08:01 (B08 supertype); and B*37:01 (B44 supertype), thereby covering representatives from the A01 and B08 supertypes that are not amenable to interactions with hTAPBPR (FIGS. 15c, d). Experiments using the mouse ortholog showed MFI ratios less than 5.8, suggesting weak interactions across HLA allotypes (FIG. 23e). The high-throughput SABs assay demonstrated that different TAPBPR orthologs exhibited distinct interaction profiles with HLA-I allotypes, in agreement with Applicants' preliminary native gel analysis (FIG. 14e). These results further supports Applicants' hypothesis that the interplay of polymorphisms on both TAPBPR and HLA-I surfaces can fine-tune their interactions.

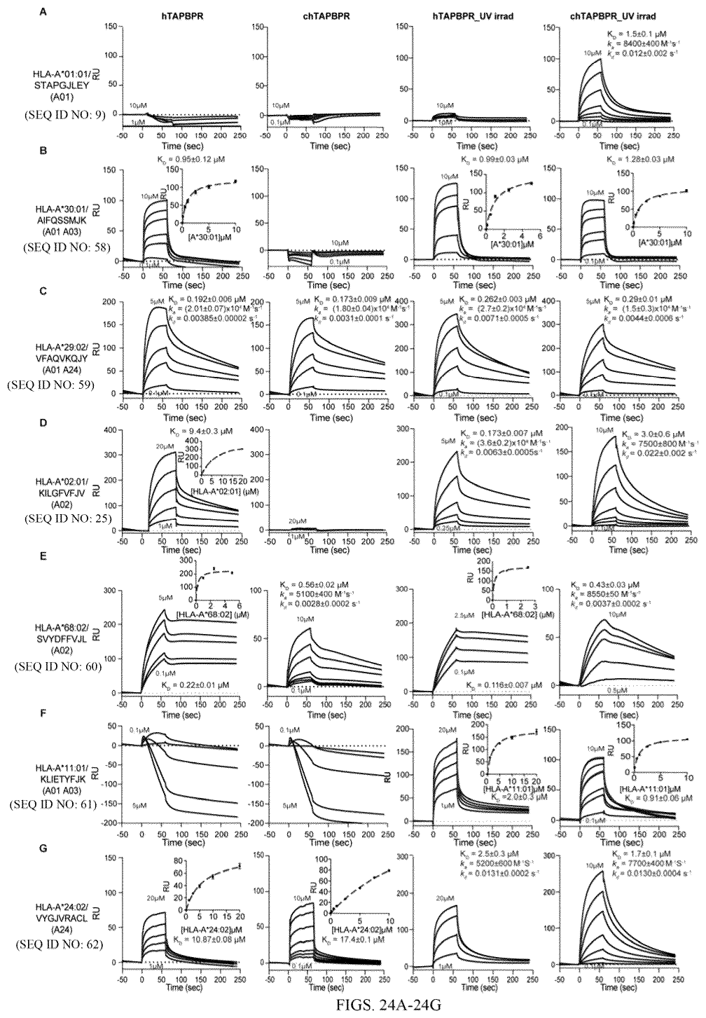

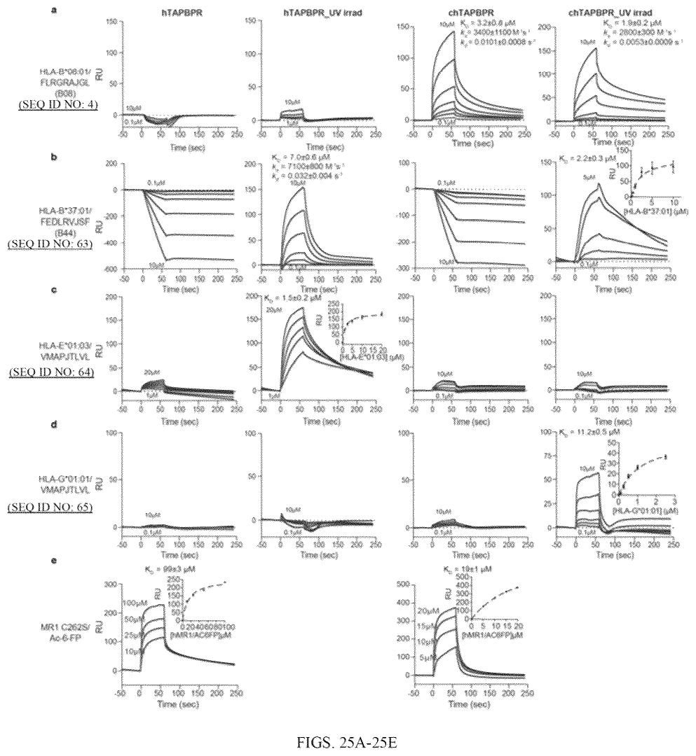

Differential recognition of peptide-deficient relative to loaded MHC-I molecules by TAPBPR. To further explore interactions between TAPBPR orthologs and different HLA allotypes in a peptide-dependent manner, Applicants used surface plasmon resonance (SPR), which allows for a quantitative assessment of protein-protein interactions (FIG. 16a). A set of recombinant HLA allotypes covering all six HLA-A supertypes and two HLA-B supertypes³² were refolded with hβ₂m and photolabile peptides⁴¹. Recombinant HLA proteins were refolded together with β₂m and photolabile peptides⁴¹. TAPBPR interactions were evaluated with empty HLA molecules by performing a 20-minute UV irradiation prior to SPR^{10,42} (FIG. 16a). Applicants confirmed that peptide-loaded HLA (pHLA)-B*08:01 interacted with chicken but not human TAPBPR with a low M-range K_{D}, in agreement with the SABs assay (FIGS. 16b, c, FIG. 25a). pHLA A*24:01 and pHLA-A*68:02 exhibited higher binding affinities towards human relative to chicken TAPBPR (FIG. 16c, FIGS. 24e, g). pHLA-A*29:02 and pHLA-A*68:02 bound to both TAPBPR orthologs with nanomolar-range dissociation equilibrium constants (K_{D}) and extremely slow dissociation rate constants (k_{d}) (FIG. 16c, FIGS. 24c, e). pHLA-A*02:01 and pHLA-A*30:01 interacted with human but not chicken TAPBPR (FIG. 16c, FIGS. 24b, d). As established previously^{10,21,26}, pHLA-A*01:01 showed no interaction with either TAPBPR ortholog (FIG. 16c, FIG. 24a). Notably, pHLA-B*37:01 showed no direct interactions with both chaperones (FIG. 16c, FIG. 25b), which seemed to contradict Applicants' SABs results. Notwithstanding, TAPBPR binding to MHC-I molecules is highly dependent on groove peptide occupancy^{10,22,24,42} and demonstrates a narrow specificity towards peptide-loaded MHC-I with moderate affinities (FIG. 16d).

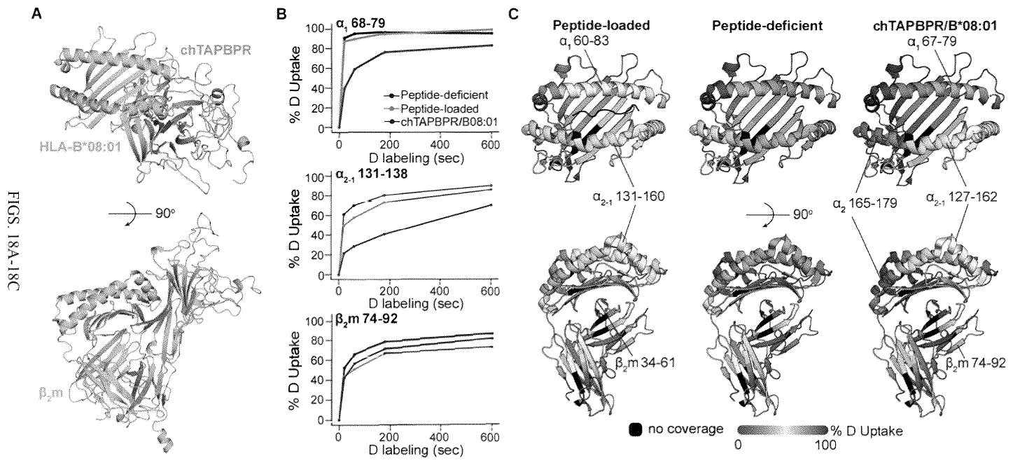

Applicants further investigate interactions between TAPBPR orthologs and empty HLA proteins by performing a 20-minute UV irradiation on these photolabile-peptide-loaded molecules prior to SPR^{10,42} (FIG. 16a). While pHLA-A*01:01 showed no direct interactions with both orthologs, empty HLA-A*01:01 demonstrated low micromolar range binding to chTAPBPR (FIG. 16e, FIG. 24a). Compared to pHLA-B*08:01, empty pHLA-B*08:01 showed tighter binding to chTAPBPR due to a decreased k_{d} by 2 folds (FIGS. 16b, f, FIG. 25a). Peptide-deficient HLA-A*68:02 and HLA-A*24:02 interact with both human and chicken TAPBPR with tighter affinities relative to their peptide-loaded states (FIGS. 16c, g, FIGS. 24e, g). Furthermore, both TAPBPR orthologs were bound to empty HLA-A*11:01 and HLA-B*37:01, but not to the respective peptide-loaded molecule (FIG. 16g, FIGS. 24f, 18b). Like HLA-A*02:01, HLA-A*30:01 showed direct binding to chTAPBPR upon UV irradiation (FIG. 16g, FIGS. 24b, d). HLA-A*29:02 in both peptide-loaded and -deficient states demonstrated low nanomolar range interaction with both orthologs (FIG. 16g, FIG. 24c). While empty HLA-A*30:01, A*29:02, A*02:01, and A*68:02 exhibit tighter binding to hTAPBPR, empty HLA-A*11:01, A*24:02, and B*37:01 show higher affinities towards chTAPBPR (FIG. 16g). In summary, peptide-deficient HLA allotypes showed direct binding to at least one TAPBPR ortholog with higher affinities compared to peptide-loaded molecules (FIG. 16h). While consistent with the previous native EMSA that both orthologs demonstrated binding to HLA-A*24:02, chTAPBPR showing interactions with HLA-A*02:01 upon UV irradiation only by SPR can be due to the instability of the complex with an almost doubled k_{d} compared to chTAPBPR/HLA-A*24:02. The in vitro SPR and SABs assay were also reconciled, since SABs contained MHC-I molecules in complex with a mixture of high, intermediate, and low affinity peptides in addition to meta-stable, peptide-deficient molecules.

Applicants then extended the study of TAPBPR interactions to cover the oligomorphic class Ib and non-classical MHC-I molecules, namely HLA-E*01:03, HLA-G*01:01, and MR1 C262S⁴². Applicants showed that acetyl-6-formylpterin (Ac-6-FP)-loaded MR1 C262S bound to hTAPBPR with moderate affinity, while the interaction with chTAPBPR was 5-fold tighter (99 vs. 19 μM, FIG. 25e). The class Ib HLA allotypes tested showed no interaction with either human or chicken TAPBPR when prepared as peptide-loaded molecules. However, following UV irradiation, peptide-deficient HLA-E*01:03 interacted with hTAPBPR while HLA-G*01:01 interacted with chTAPBPR with moderate M-range K_{D} (FIGS. 16c, g, FIGS. 25c, d). Taken together, Applicants' SPR data provide additional evidence that different TAPBPR orthologs can interact with complementary sets of HLA allotypes. Peptide-deficient MHC-I molecules generally exhibit tighter interactions with chaperones while, in some cases, both TAPBPR orthologs recognize the empty MHC-I state exclusively, in agreement with previous studies^{10,26}.

Design of conditional ligands to monitor peptide exchange across different HLA allotypes. To investigate effects on peptide exchange kinetics for HLA allotypes that showed direct interactions with TAPBPR orthologs, Applicants followed the binding of fluorescently labeled high-affinity peptides by real-time fluorescence polarization (FP)^{43,44}. The high-affinity fluorescent peptide probe exchanges for the placeholder peptide that is pre-loaded on the HLA molecule of interest. Applicants first identified suitable conditional ligands that served as “placeholder” peptides for various HLA allotypes. Previous approaches for generating such ligands use either photosensitive peptides treated with UV irradiation^{11,34,35,45} or “goldilocks” peptides, which are derived from high-affinity peptide epitopes by truncation of the N-terminal amino acid^{46,47}. However, both approaches can lead to significant levels of background exchange in the absence of chaperones, an effect that can mask any additional exchange promoted by chaperones.

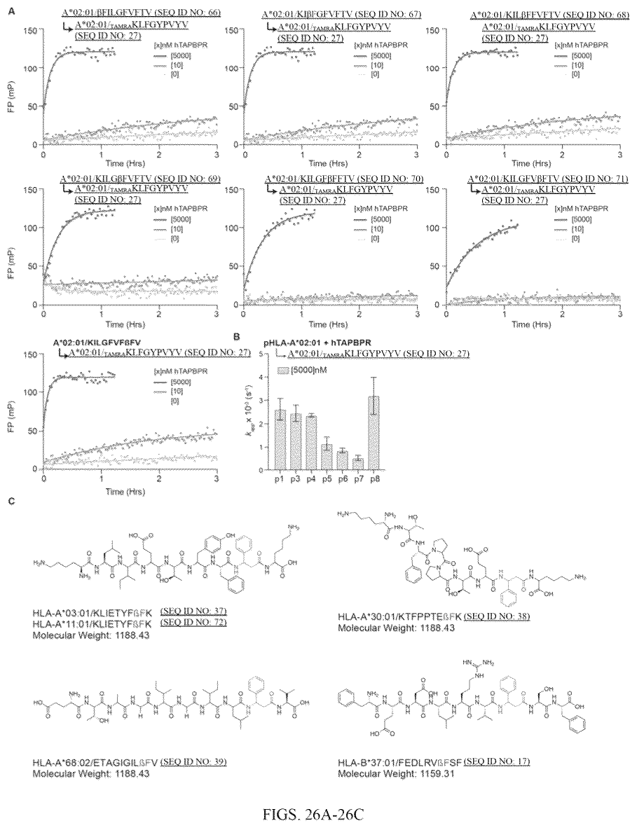

Here, Applicants used an alternative method to generate conditional ligands by introducing an unnatural amino acid L-β-Phenylalanine (βF), structurally akin to the photolabile amino acid 3-amino-3-(2-nitrophenyl)-propionic acid (J-amino acid)³⁵ but without peptide cleavage under the UV exploration (FIG. 17A). We explored all single residue substitutions of the epitope KILGFVFTV (SEQ ID NO: 33) with βF on the HLA-A*02:01 system (FIG. 17B). Applicants determined that βF modifications at either position P5, P6 and P7 had a minimal effect on peptide stability, yielding refolded A*02:01 proteins with Tₘ values of 62° C., 61° C. and 63° C., respectively, that were indicative of high-affinity pMHC-I complexes (FIG. 17C). However, introduction of βF at either P1, P4 or P8 had a significant destabilization effect, yielding complexes with thermal stabilities in the range from 53° C. (βF-P8) to 58° C. (βF-P3) (FIG. 17C). This effect could be attributed to both the short, bulky phenyl ring side chain in sterically confined regions of the pMHC-I groove and the extra carbon-carbon bond in the peptide backbone that may introduce conformational ‘strain’ to further destabilize the pMHC-I interaction compared to the conventional αF^{76,77}. Applicants then monitored the exchange of each OF-containing conditional ligand on the HLA-A*02:01 groove by a fluorophore-conjugated, high-affinity peptide with or without 10 nM hTAPBPR⁴⁶. Applicants found that the effectiveness of hTAPBPR to catalyze the exchange of different placeholder peptides inversely correlated with the thermal stability of the OF peptide/HLA-A*02:01 complexes, where βF-P3 showed the smaller and βF-P8 the larger effect upon incubation with the chaperone (FIGS. 17C, D). Moreover, at 10 nM hTAPBPR concentration, hTAPBPR only accelerated the rate of reaction for βF peptides that already demonstrated a baseline level of exchange, whereas βF-P6 and βF-P7 both showed a Tₘ value greater than 60° C. and no peptide exchange either in the absence or presence of hTAPBPR (FIG. 17D, FIG. 26A). Since hTAPBPR recognizes empty HLA-A*02:01 with low nanomolar-range K_{D} as opposed to micromolar-range for peptide-loaded molecules, hTAPBPR binds empty HLA-A*02:01 molecules and stabilizes them in a peptide-receptive conformational state for exchange, in agreement with previous studies^{24,46}.

Furthermore, Applicant monitored the exchange of the βF positional scanning peptides for HLA-A*02:01 with 5000 nM hTAPBPR. While hTAPBPR at a high concentration consistently promoted complete fluorescent peptide exchange for βF-P1, βF-P3, βF-P4, and βF-P8, it allowed peptide loading for βF-P5, βF-P6, and βF-P7 where no exchange was observed previously in the absence or presence of 10 nM hTAPBPR (FIG. 26A). Expectedly, peptide exchange efficiency (kₐₚₚ) was substantially enhanced in a molar excess of hTAPBPR (FIG. 17D, FIG. 26C). Therefore, hTAPBPR, at a molar excess, could recognize and chaperone the peptide-loaded molecules to unload their cargos and make empty molecules more accessible for the new incoming peptide. Based on the above results, Applicants selected the βF-P8 peptide as the most suitable conditional ligand to investigate the effects of different TAPBPR orthologs on peptide exchange for HLA-A*02:01. The peptide exchange reaction of βF-P8 loaded HLA-A*02:01 was most effectively promoted under both a catalytic and molar excess amount of hTAPBPR (FIG. 17D, FIG. 26C). Applicants used a similar strategy to design OF substituted peptides for HLA-A*03:01/11:01, HLA-A*30:01, HLA-A*68:02, and HLA-B*37:01 (KLIETYFβFK, KTFPPTEβFK, ETAGIGILβFV, and FEDLRVβFSF), starting from the sequences of well-described immunodominant epitopes (FIG. 26C). Using a combination of βF designs, destabilized peptides with J-amino acid⁷⁰, or other epitopic peptides (Table 2), Applicants obtained conditional pMHC-I protein complexes with sufficient thermal stabilities (>50° C.) to study the exchange properties for representative HLA allotypes covering five out of six HLA-A supertypes and two HLA-B supertypes in Applicants' study (Table 2).

Applicants introduced unnatural amino acid L-β-Phenylalanine (βF) near the peptide C-terminus upon screening the effect of βF at different peptide positions (FIG. 17A, FIGS. 26A, C). This resulted in MHC-I protein complexes with higher thermal stability relative to the conventional goldilocks peptides, and improved exchange properties triggered only by the addition of TAPBPR (FIGS. 17B, C, FIG. 26C). Peptide-receptive HLA molecules were chaperoned and stabilized by 5 nM to 5 μM of human or chicken TAPBPR and the observed rate constant (kₐₚₚ) for the association of fluorescent peptide at a fixed concentration to MHC-I was determined. The slope extrapolated from kₐₚₚ versus TAPBPR (catalyst) concentration is defined as k₂ₒᵥₑᵣₐₗₗ, the overall rate of the forward reaction forming fluorescent peptide-loaded MHC-I molecules from peptide association to either empty MHC-I or MHC-I/TAPBPR complexes.

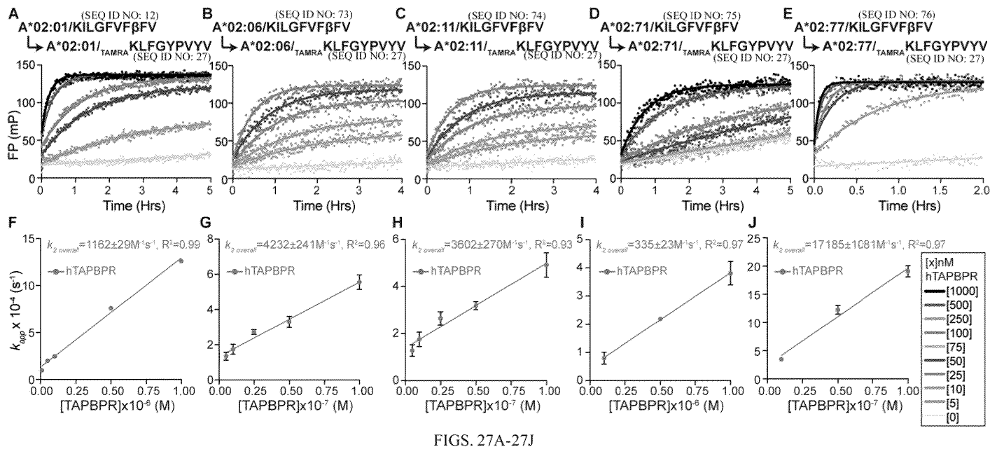

TAPBPR orthologs catalyze peptide exchange on a broad repertoire of HLA supertypes. To quantitatively compare TAPBPR effects on peptide exchange across different HLA allotypes, Applicants performed a series of fluorescence polarization (FP) experiments in which pHLA molecules and fluorescent peptide probes at a fixed concentration were incubated with graded concentrations of TAPBPR. The observed rate constants (kₐₚₚ) were determined by fitting a one-phase association equation to describe the fluorescent peptide ligand and MHC-I association. The slope extrapolated from the linear fit between kₐₚₚ and TAPBPR concentration was defined as an overall rate (k₂ₒᵥₑᵣₐₗₗ), which was analogous to a catalytic k_{cat} constant used to describe enzymatic reactions under steady-state conditions^{44,71}. A previous study has demonstrated that a small number of sequence variations between HLA subtypes, depending on their location, can influence TCR recognition⁴⁸. Here, Applicants hypothesized that such differences among micropolymorphic HLA-A*02 allotypes might also affect TAPBPR recognition and peptide exchange kinetics. Applicants determined that hTAPBPR accelerated peptide exchange on the HLA-A*02:06, A*02:11, A*02:71, and A*02:77 subtypes at similar levels relative to A*02:01. However, for HLA-A*02:71 containing the Q141R polymorphism in the α₂₋₁ helix, Applicants observed a reduced k₂ₒᵥₑᵣₐₗₗ (335M⁻¹ s⁻¹) by 3.5 folds compared to A*02:01 (FIGS. 17E, F, FIG. 27). Notably, for HLA-A*02:77, which contained an amino acid substitution on the opposite side of the α₂ helix (E166V), Applicants observed a significantly increased rate of peptide exchange relative to A*02:01 (17185M⁻¹ s⁻¹ vs. 1162 M⁻¹ s⁻¹) (FIG. 27). Given that both protein complexes with the same placeholder peptide exhibited a similar overall stability (Table 3), the presence of allosteric communication along the α₂ helix might also influence the MHC-I interactions with TAPBPR. Taken together, Applicants' results provide evidence that the interplay of polymorphic residues, both at the HLA-I/TAPBPR interface and at a distance, can fine-tune interactions and peptide exchange function.

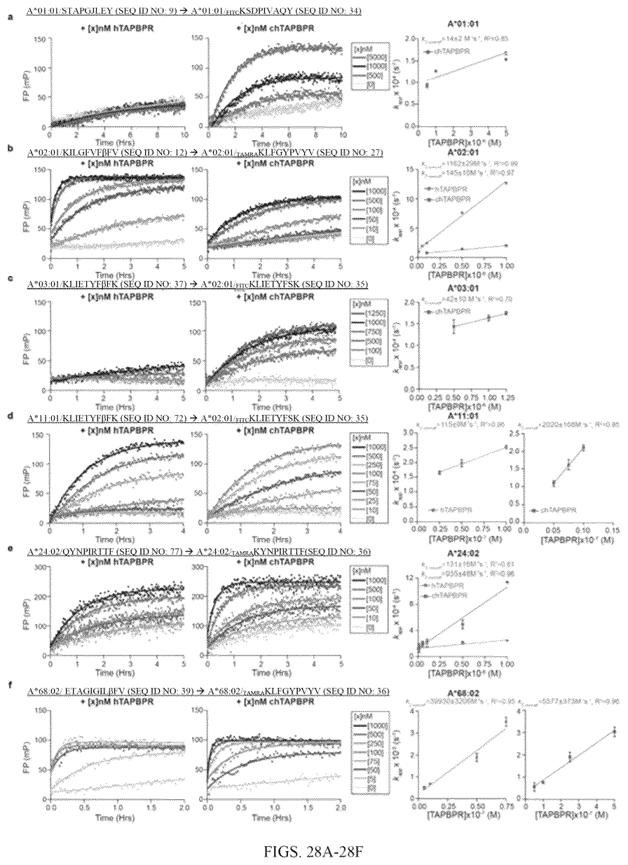

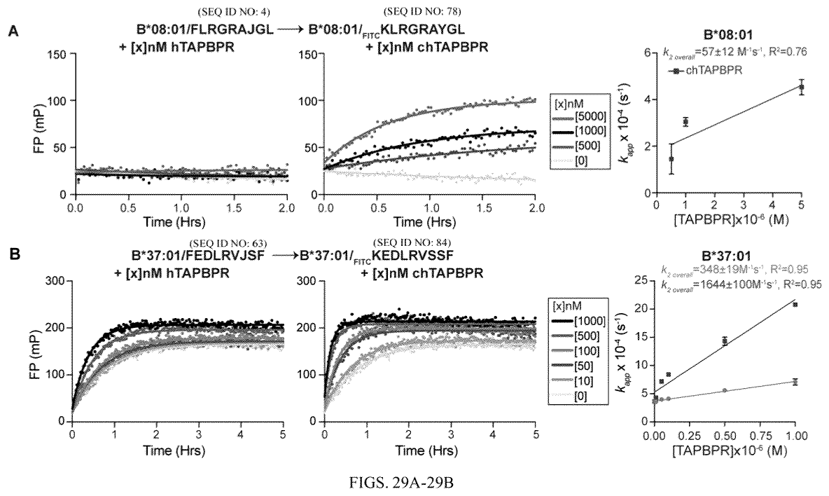

The binding of the fluorescent peptides to HLA-A*30:01, A*02:01, A*11:01, A*24:02, A*68:02, and B*37:01 was faster in the presence of both TAPBPR proteins, consistent with the direct binding to peptide-deficient MHC-I shown in previous SPR (FIG. 16g). Applicants next examined the effects of both TAPBPR orthologs on peptide exchange across Applicants' panel of HLA supertype representatives. Analysis of Applicants' FP data revealed that, while the k₂ₒᵥₑᵣₐₗₗ of HLA-A*02:01 was 8-fold higher for human than chicken TAPBPR, the k₂ₒᵥₑᵣₐₗₗ of B*37:01 in the presence of hTAPBPR was approximately 5-fold lower than chTAPBPR (FIGS. 17G, H, FIG. 28c, FIG. 29b). Likewise, the overall exchange rate of A*11:01 and A*24:02 with the addition of chTAPBPR is more than 17 and 7-fold higher than hTAPBPR (FIG. 17H, FIG. 28e, f). Placed within the context of Applicants' SPR data (FIGS. 16c, g), these results suggest that both human and chicken TAPBPR promote peptide exchange by recognizing empty HLA allotypes with different affinities. In agreement, HLA-A*02:01, HLA-A*30:01 and HLA-A*68:02 with a higher k₂ₒᵥₑᵣₐₗₗ for hTAPBPR had a higher affinity to human over chicken TAPBPR (0.17 vs. 2.96 μM for A*02:01, 0.99 vs. 1.28 μM for A*30:01, and 0.12 vs. 0.43 μM for A*68:02), and vice versa for A*11:01 (2.04 vs. 0.91 μM), A*24:02 (2.54 vs. 1.68 μM), and B*37:01 (7.02 vs. 2.19 μM) (FIG. 16g, FIG. 17H, FIG. 28). hTAPBPR showed no effect on peptide exchange for HLA-A*01:01, HLA-A*03:01 and HLA-B*08:01, whereas chTAPBPR effectively catalyzed their peptide loading for the same systems (FIG. 17H, FIGS. 28a, d, and FIG. 29a). Applicants' FP results on different HLA allotypes again support that TAPBPR catalysis proceeds through the recognition and stabilization of an empty MHC-I at a peptide-receptive conformation.

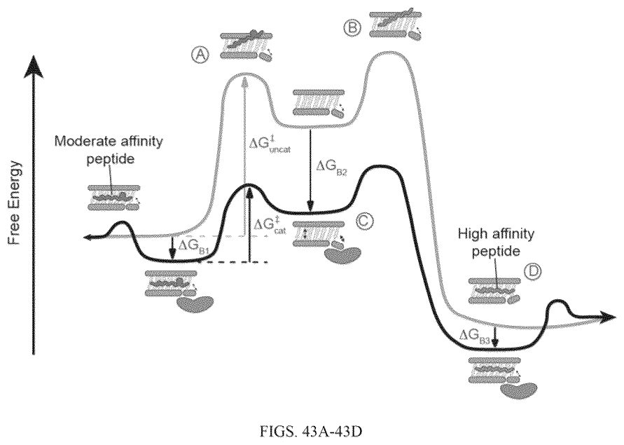

To further test the hypothesis that peptide exchange by TAPBPR orthologs is dependent upon recognition of a peptide-deficient MHC-I state, Applicants used DSF⁴⁹ and focused on HLA-A*01:01 and HLA-B*08:01 which are not subject to hTAPBPR binding and peptide exchange in vitro^{21,26}. Applicants established high-affinity peptides with increased thermal stabilities by at least 5 degrees relative to complexes refolded with suitable placeholder peptides for both systems (FIGS. 30A-D). HLA-A*01:01 refolded with a moderate affinity RAS self-epitope did not exchange in the presence of 10-fold molar excess of high-affinity peptides, even upon incubation with hTAPBPR (FIGS. 30A-D), consistent with the observed lack of binding between hTAPBPR and HLA-A*01:01 in either peptide-loaded or -deficient states by SPR (FIGS. 16c, g). Notably, incubating pHLA-A*1:01 with chTAPBPR in the presence of each high-affinity peptide led to significant (>10 degrees) thermal shifts for all three peptides, indicating net exchange of the placeholder for high affinity peptide epitopes (FIGS. 30A-D). The minor destabilization effect observed for chTAPBPR upon addition of a non-specific peptide (FIG. 30B) likely results from the formation of transient pMHC-I/chTAPBPR complexes with destabilized peptide-binding grooves²⁵. HLA-B*08:01 showed a partial thermal shift upon incubation with excess high affinity peptide, revealing a spontaneous, partial peptide loading mediated by empty molecules in the sample which is not affect by the addition of hTAPBPR (FIGS. 30A-D). However, addition of catalytic amounts of chTAPBPR promotes the complete exchange of placeholder for different high-affinity peptides, as indicated by a single, left-shifted Tₘ peak, while the non-specific peptide failed to bind the presence of either human or chicken TAPBPR (FIGS. 30A-D). Placed within the context of Applicants' SPR data, the DSF peptide exchange results demonstrate a trend where interactions with empty HLA-A*01:01 or HLA-B*08:01 molecules, as seen for chicken but not for human TAPBPR, promote the complete exchange of placeholder for higher affinity peptides (Table 1). HLA-A*02:01 and HLA-B*37:01, which demonstrated high-affinity binding to both orthologs (FIG. 16g), undergo complete peptide exchange as opposed to negative control in the presence of both orthologs (FIGS. 30E-H). Taken together, Applicants' data further supports the mechanism that TAPBPR orthologs accelerate peptide exchange by capturing and maintaining empty MHC-Is at their peptide-receptive, “open” conformation. To load high-affinity peptides, empty MHC-I molecules need to be at a peptide-receptive transition state, which is high in activation energy. Thus, TAPBPR orthologs function as a catalyst to lower the energy barrier by stabilizing the peptide-receptive form and permitting rapid peptide loading (FIG. 43). These findings suggest that both peptide unloading and chaperoning functions of TAPBPR are required for efficient catalysis of peptide exchange in vitro.

Moreover, a previous study has demonstrated that a small number of sequence variations between the subtypes of specific HLA alleles, depending on their location, might influence T cell recognition⁴⁸. Applicants then hypothesized that such differences among polymorphic HLA-A*02 allotypes might affect TAPBPR recognition and the peptide exchange kinetics. Applicants found that hTAPBPR accelerated peptide exchange on the HLA-A*02:06, A*02:11, A*02:71, and A*02:77 subtypes at similar levels relative to A*02:01. However, for HLA-A*02:71 containing the Q141R polymorphism in the α₂₋₁ helix, Applicants observed a reduced k₂ₒᵥₑᵣₐₗₗ (335 M⁻¹ s⁻¹) by 3.5 fold compared to A*02:01 (FIGS. 17E, F, FIG. 27). Notably, for HLA-A*02:77 which contained an amino acid substitution on the opposite side of the α₂ helix (E166V), we observed a significantly increased rate of peptide exchange relative to A*02:01 (17185 M⁻¹ s⁻¹ vs. 1162 M⁻¹ s⁻¹) (FIG. 27). Given that both protein complexes with the same placeholder peptide exhibited a similar overall stability (Table 3), the presence of allosteric communication along the α₂ helix might also influence the MHC-I interactions with TAPBPR. Taken together, Applicant's results provide evidence that the interplay of polymorphic residues, both at the HLA-I/TAPBPR interface and at a distance, can fine-tune interactions and peptide exchange function.

TABLE 1
Summary of binding affinities to TAPBPR orthologs and
peptide exchange results for 13 MHC-I representatives.

| | | Supertype | | | | | | |
|---|---|---|---|---|---|---|---|---|
| | | | A01 | A01 | | | | |
| | | A01 | A03 | A24 | A02 | | A03 | |
| | | Allele | | | | | | |
| | | A*01:01 | A*30:01 | A*29:02 | A*02:01 | A*68:02 | A*0301 | A*11:01 |
| | | Frequency among global populations (%) | | | | | | |
| | | 13.2 | 2.0 | 2.6 | 24.1 | 1.3 | 12.3 | 6.3 |
| K_{D} ¹ of | Loaded | NB+ | 0.95 | 0.19 | 9.44 | 0.22 | n.d.+³ | NB+ |
| hTAPB | | | | | | | | |
| PR | Empty | NB+ | 0.99 | 0.26 | 0.17 | 0.12 | n.d.+ | 2.04 |
| (μM) | | | | | | | | |
| K_{D} ¹ of | Loaded | NB+ | NB+ | 0.17 | NB | 0.56 | n.d.+ | NB+ |
| chTAP | | | | | | | | |
| BPR | Empty | 1.46 | 1.28 | 0.29 | 2.96 | 0.43 | n.d.+ | 0.91 |
| (μM) | | | | | | | | |
| Peptide | hTAPBPR | No | Yes | | Yes | | Yes¹¹ | Yes |
| exchange⁴ | chTAPBPR | Yes | Yes | | Yes | | Yes | Yes |

| | Supertype | | | | | |
|---|---|---|---|---|---|---|
| | A24 | B08 | B44 | | | |
| | Allele | | | | | |
| | A*24:02 | B*08:01 | B*37:01 | E*01:03 | G*0101 | MR1 |
| | Frequency among global populations (%) | | | | | |
| | 9.6 | 8.4 | 1.3 | 36.1 | 74.0 | |
| K_{D} ¹ of | 10.9 | NB+ | NB+ | NB+ | NB+ | 98.7 |
| hTAPB | | | | | | |
| PR | 2.54 | NB+ | 7.02 | 1.51 | NB+ | |
| (μM) | | | | | | |
| K_{D} ¹ of | 17.4 | 3.19 | NB+ | NB+ | NB+ | 19.2 |
| chTAP | | | | | | |
| BPR | 1.68 | 1.89 | 2.19 | NB+ | 11.22 | |
| (μM) | | | | | | |
| Peptide | Yes | No | Yes | | | Yes⁴² |
| exchange⁴ | Yes | Yes | Yes | | | |

¹K_{D} measured by SPR.
²NB, no or very weak binding.
³ND, K_{D} not determined by SPR.
⁴Based on DSF or FP assays.

Furthermore, Applicants found that both HLA-A*02:01 and HLA-B*37:01 can undergo complete peptide exchange in the presence of both TAPBPR orthologs. HLA-A*02:01 molecules refolded with a photosensitive placeholder peptide exhibit a lower Tₘ than those refolded with the high-affinity HTLV-1 TAX9 peptide (52° C. vs. 65° C., FIG. 30E). No exchange of the placeholder peptide was observed using 10-fold molar excess of TAX peptide in the absence of TAPBPR, suggesting that despite its reduced affinity, the placeholder peptide remains stably captured in the MHC-I groove on the timescale of the experiment (FIG. 30F). However, the addition of either human or chicken TAPBPR promoted complete peptide exchange upon 2-hour room temperature incubation, as indicated by the shift of Tₘ values from 53° C. to 66° C. (FIG. 30F). Meanwhile, no exchange was observed with the irrelevant p29 peptide in the presence of either TAPBPR ortholog (FIG. 30F). HLA-B*37:01 demonstrated a partial upshift of Tₘ in a 10-fold excess of a high-affinity influenza epitope NP338 peptide shown by two peaks (FIGS. 30G, H). The spontaneous loading of the high-affinity peptide may be attributed to the existence of empty HLA-B*37:01 molecules from the refolding because of the instability of the pMHC complex (Tₘ=47° C., FIG. 30G). Notably, the addition of sub-stoichiometric amounts of both TAPBPR orthologs promoted a complete exchange of the placeholder for high-affinity peptide on HLA-B*37:01 (FIG. 30H). These results also agree with Applicants' SPR data, where direct interactions with both HLA-A*02:01 and HLA-B*37:01 were observed when prepared as empty MHC-I molecules. Taken together, Applicant's data further supports a mechanism where TAPBPR orthologs, unless in large molar excess, primarily accelerate peptide exchange by capturing and maintaining empty MHC-I molecules in their peptide-receptive, “open” conformation.

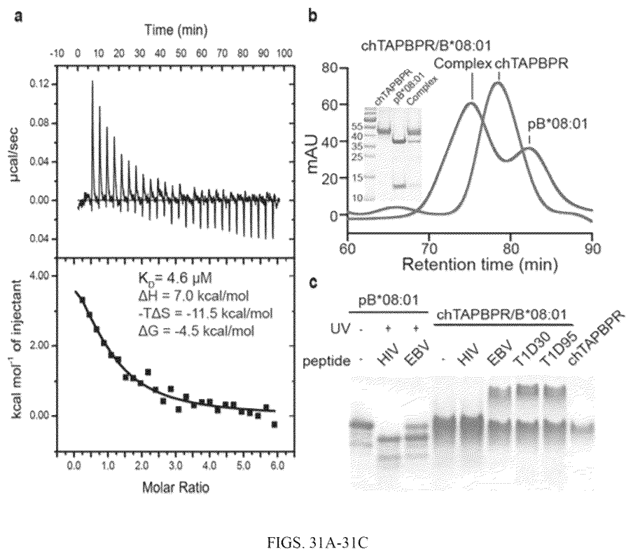

TAPBPR orthologs recognize conserved surfaces on emply MHC-I molecules. To establish thermodynamic parameters describing the binding process between peptide-loaded HLA-B*08:01 and chTAPBPR, Applicants performed isothermal calorimetry titration (ITC) experiments⁵⁰. ITC titrations of chTAPBPR on pMHC-I were performed in the presence of 3-fold molar excess (450 μM) of the high-affinity peptide to ensure saturation of the peptide-binding groove. Applicants' ITC data revealed that peptide-bound HLA-B*08:01 exhibited a strong endothermic binding interaction with chTAPBPR (FIG. 31a), similar to previous ITC results reporting on interactions between P18-I10/H2-D^{d} and p29/H2-L^{d} with hTAPBPR²⁶. Fitting of the sigmoidal isotherms using a 1-site binding model yielded an apparent K_{D} of 4.6 μM, comparable to 3.2 μM from Applicants' SPR measurements (FIG. 16b). Applicants also isolated empty HLA-B*08:01/chTAPBPR complexes by incubating UV-irradiated HLA-B*08:01 loaded with FLRGRAJGL (SEQ ID NO: 4) with chTAPBPR in a stoichiometric ratio, followed by size-exclusion chromatography (FIG. 31b). Purified chTAPBPR/HLA-B08:01 complexes were receptive to high-affinity peptides, as evidenced by the appearance of discrete pMHC-I bands in an EMSA upon incubation with high-affinity peptides EBV, T1D30, and T1D95, but not with an irrelevant HIV peptide (FIG. 31c). Taken together, Applicants' results demonstrate that chTAPBPR can recognize both peptide-loaded and -deficient molecules, albeit with different affinities, and maintain empty HLA-B*08:01 in a peptide receptive state, in a similar manner to hTAPBPR on HLA-A*02:01 molecules^{10,51}.

To further probe chTAPBPR interactions and map the binding epitopes on HLA-B*08:01 in a solution environment, Applicants used hydrogen deuterium exchange mass spectrometry (HDX-MS)⁵². A structural model of HLA-B*08:01/chTAPBPR was generated using RosettaCM, showing a conserved binding mode (FIG. 18a). HLA-B*08:01 prepared as either empty, peptide-bound, or chTAPBPR-bound was then incubated in a deuterated buffer for several time points, allowing for the incorporation of deuterium into the protein amide groups. The exchange reaction was quenched by a shift to acidic pH and digested by an acid-functional protease. Tandem analysis of the reaction products by mass-spectrometry identified three putative interaction regions with lower deuterium uptake when bound to chTAPBPR: residues 68-79 and 131-138 on the HLA-1B*08:01 α₁ and α₂₋₁ regions, and residues 74-92 on β₂m (FIG. 18b). Observed percent deuterium uptake was mapped onto a crystal structure of HLA-B*08:01 (PDB ID: 4QRU), revealing binding sites on the α₁ and α₂₋₁ helix of the heavy chain and β₇ of β₂m (FIG. 18c) that are conserved with interaction sites in the crystal structure of murine H2-D^{d} bound to hTAPBPR (PDB ID: 5WER). When comparing the peptide-loaded to the peptide-deficient state, deuterium uptake decreased in the peptide-binding domain, the membrane-proximal α₃ domain, and the non-covalently associated β₂m (FIG. 18c), highlighting a significantly reduced plasticity upon peptide loading that is consistent with the previous study⁵³. Deuterium uptake of chTAPBPR-bound HLA-B*08:01 relative to empty molecules reaches a maximum at the 60 s, and then gradually converges towards more similar levels (FIG. 18b), indicating reversible TAPBPR interactions in further agreement with Applicants' SPR data showing a low micromolar-range dissociation constant (FIG. 16g). Increased deuterium uptake at residues 165-179 on the α₂ helix upon chTAPBPR binding provides evidence of long-range effects, namely allosteric coupling between the F- and B-pockets in agreement with previous studies^{51,54,56}. In summary, Applicants' HDX-MS mapping reveals that chTAPBPR recognizes common epitopes on MHC-I, as seen in both the co-crystal structures of murine MHC-I/hTAPBPR complexes^{22,23}, and solution NMR-based mapping of HLA-A*02:01/hTAPBPR complexes^{26,51}.

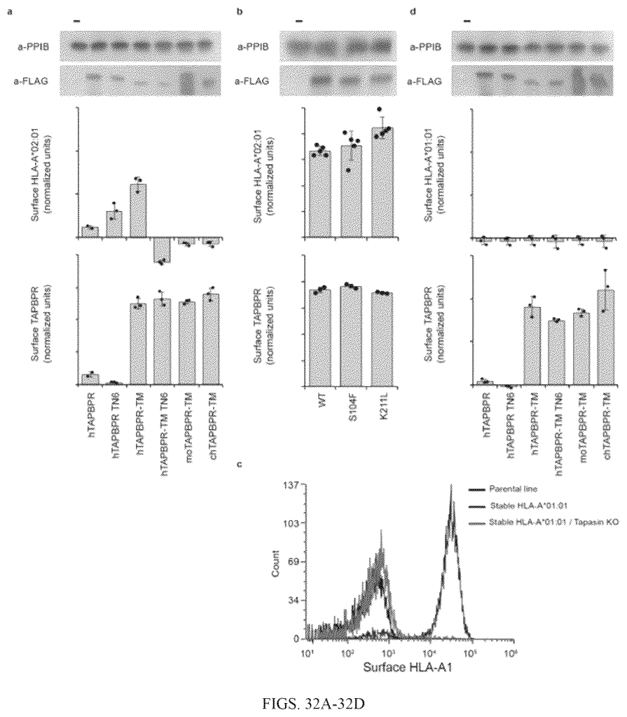

TAPBPR sequence variants enable gain of function interactions with targeted HLA allotypes. To further understand how TAPBPR sequence variation impacts interactions with HLAs, Applicants used cell-based assays to explore thousands of substitutions in hTAPBPR-TM, a chimeric construct containing the luminal domain of hTAPBPR fused to the canonical transmembrane domain of HLA-G. Overexpression of hTAPBPR could partially rescue the surface expression of HLA-A*02:01 that was reduced by tapasin knocked out (KO) in human Expi293F cells²⁶ (FIG. 32a). However, overexpressing the conventional negative control hTAPBPR^{TN6}, which disrupts binding to mature, folded MHC-I¹⁰, could also effectively rescue surface HLA-A*02:01 in a tapasin-KO background (FIG. 32a). Since hTAPBPR and hTAPBPR^{TN6} were trapped in ER and Golgi during this assay³, they both participated in intracellular chaperoning of nascent MHC-I. Alternatively, we used hTAPBPR-TM, which could be expressed within the ER and on the cell surface²⁶. An analogous construct to hTAPBPR-TM was previously found to associate with MHC-I on the cell surface to promote peptide editing⁵⁷. Therefore, not only did hTAPBPR-TM rescue HLA-A*02:01 surface expression in tapasin-KO Expi293F cells, but this effect was now lost in the hTAPBPR-TM^{TN6} mutant. The hTAPBPR-TM-mediated rescue of surface HLA-A*02:01 is an indirect readout of the physical association between hTAPBPR and folded MHC-I rather than nascent molecules, an essential feature of peptide editing. Based on these observations, Applicants used deep mutational scanning to identify hTAPBPR mutants with enhanced peptide editing function for HLA-A*02:01.

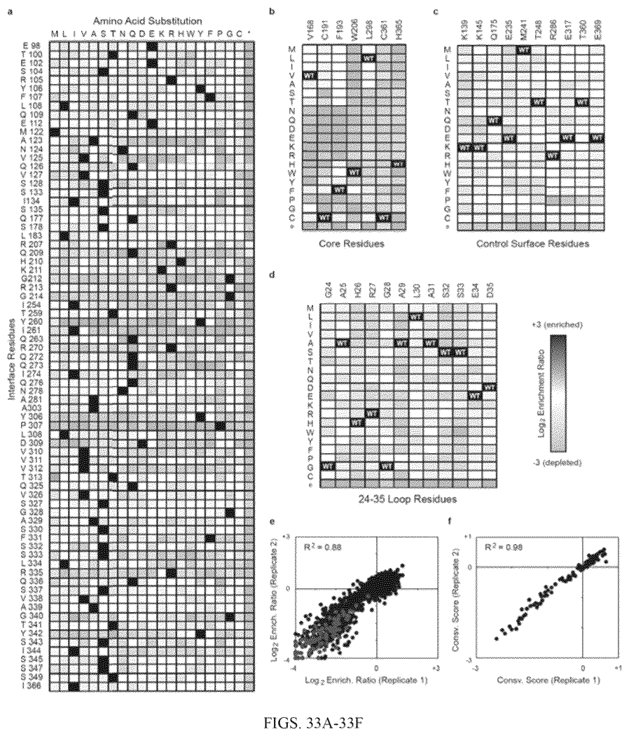

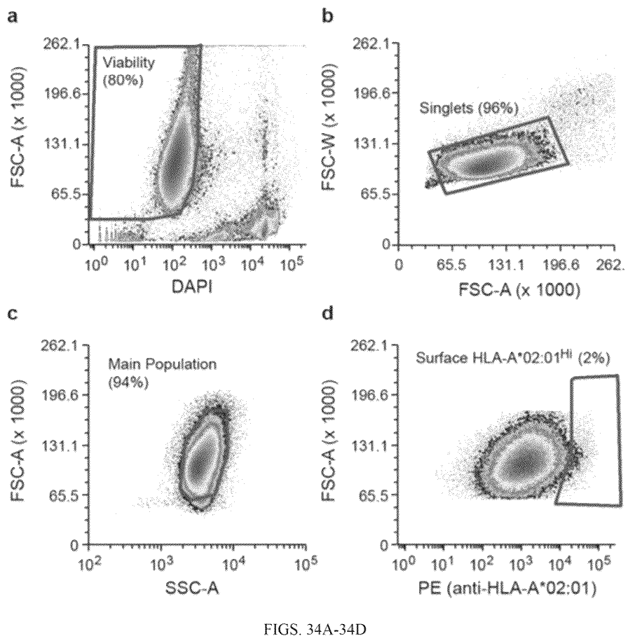

A single site-saturation mutagenesis (SSM) library of hTAPBPR-TM was created, focused on 75 residues in the hTAPBPR luminal domain that interface with MHC-I based on the crystal structures (FIG. 33a). Applicants also mutated an additional 17 residues at control sites: (i) on the opposite side of hTAPBPR to the interface where mutations were expected to have a minimal effect (FIG. 33c) and (ii) in the protein core where most mutations are expected to be highly deleterious (FIG. 33b). The library was transfected into the tapasin-KO Expi293F line and cells with high levels of surface HLA-A*02:01 were collected by fluorescence-activated cell sorting (FACS) (FIG. 34). The naive and sorted libraries were deep sequenced, and the enrichment of functional mutations or depletion of deleterious mutations was calculated to determine a mutational landscape. The data were closely correlated between two independent selection experiments and mutations at control sites had the anticipated effects (FIGS. 33e, f).

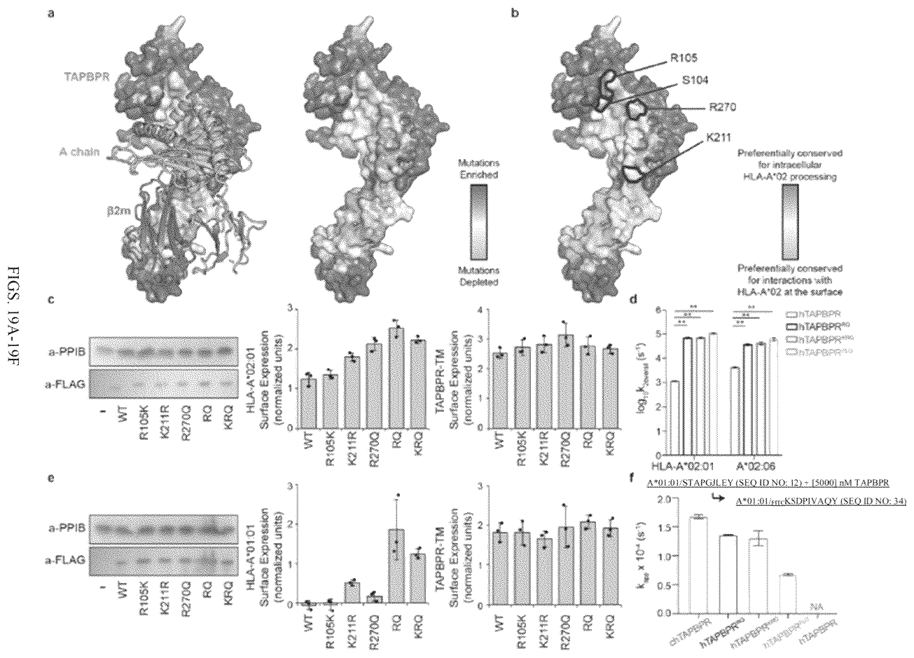

In the deep mutational scan, most hTAPBPR residues that contact MHC-I, especially β₂m, tend to be conserved and intolerant of mutations, consistent with the hTAPBPR-TM chimera engaging folded MHC-I at the cell surface with β₂m interactions contributing to recognition (FIG. 19a). However, there are two prominent regions at the interface where mutational tolerance is high. The first region comprises the 24-35 loop that hovers above the F pocket of the peptide groove and acts as a ‘trap’ to prevent high affinity peptides from dissociating when TAPBPR forms transient associations with peptide-loaded MHC-I⁵¹ (FIG. 33d). The second interface region of mutational tolerance is formed by hTAPBPR residues that contact the MHC-I α₂ helix (FIG. 19a); the same TAPBPR residues are mildly conserved for chaperoning function⁵¹ (FIG. 19b), suggesting this region may have different roles in chaperoning versus peptide editing.

Applicants hypothesized that Applicants could engineer an hTAPBPR-TM mutant with enhanced HLA-A*02:01 interactions by focusing substitutions to the two regions in proximity to MHC-I yet of high mutational tolerance in the deep mutational scan. Applicants identified five highly enriched mutations, S104F, R105K, K211R, K211L, and R270Q, in the scan of hTAPBPR-TM for the rescue of HLA-A*02:01 surface expression, yet not as enriched in the previously published mutational scan of intracellular hTAPBPR chaperoning HLA-A*02:01 (FIG. 19b), suggesting the mutations may enhance interactions with folded molecules. Therefore, single and combination mutants at the selected position of hTAPBPR-TM were tested for their ability to rescue HLA-A*02:01 surface expression in tapasin-KO cells. TAPBPR-TM mutants S104F and R105K mediated minor increases in HLA-A*02:01 surface expression that was not statistically significant, while TAPBPR-TM mutants containing individual or combined mutations at position R105, K211, and R270 had enhanced rescuing activity for surface HLA-A*02:01 compared to WT TAPBPR-TM (FIG. 19c, FIG. 32b). Other than R105K, which had no significant change from wild type (WT) hTAPBPR-TM, all the mutants containing the two substitutions which were coincidentally shared with chTAPBPR displayed enhanced activity for the rescue of surface HLA-A*02:01 (FIG. 19c). Applicants note, however, that in this cell-based assay, chTAPBPR-TM and moTAPBPR-TM were unable to rescue surface HLA-A*02:01 levels (FIG. 32a), possibly because both orthologous proteins were expressed at much higher levels intracellularly where they might bind and sequester MHC-I substrates.

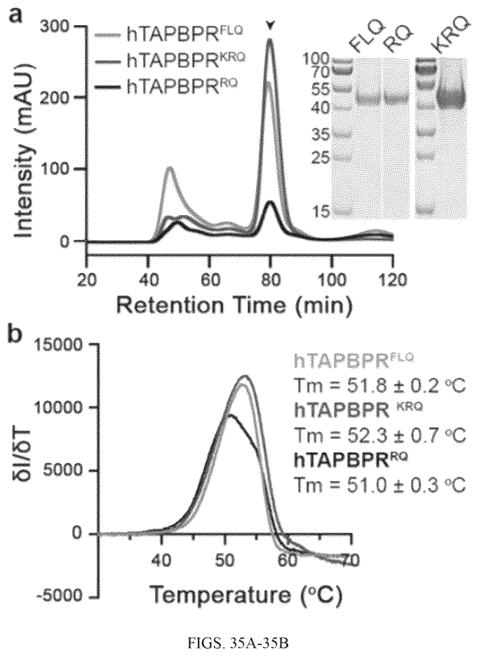

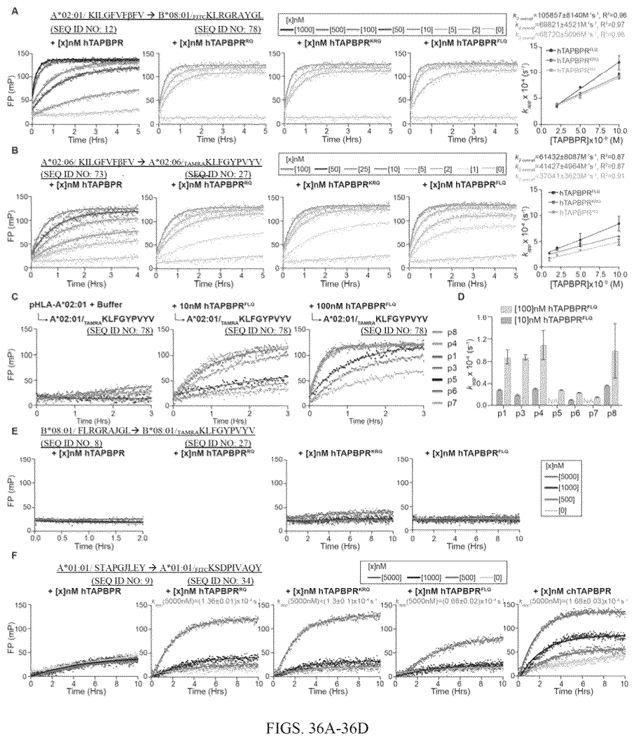

Applicants then purified recombinantly expressed proteins encompassing the soluble ectodomain of three hTAPBPR mutants carrying different combinations of all five mutations: hTAPBPR^{RQ} (K211R, R270Q) and hTAPBPR^{KRQ} (R105K, K211R, R270Q) carried substitutions to polar amino acids, while hTAPBPR^{FLQ} (S104F, K211L, R270Q) carried two substitutions for hydrophobic amino acids (FIG. 35). Using a FP peptide exchange assay, Applicants found that the mutants were extraordinarily efficient at catalyzing the exchange of KILGFVFβFV (SEQ ID NO: 12) loaded HLA-A*02:01 with the high-affinity fluorescent peptide _{TAMRA}KLFGYPVYV (SEQ ID NO: 27). The k₂ₒᵥₑᵣₐₗₗ of hTAPBPR^{RQ} and hTAPBPR^{KRQ} for the HLA-A*02:01 peptide editing reaction was approximately 60-fold higher than for hTAPBPR (FIG. 19d, FIG. 36A). Surprisingly, the k₂ₒᵥₑᵣₐₗₗ of pHLA-A*02:01 loading fluorescent peptide in the presence of hTAPBPR^{FLQ} increased by 90 folds compared to hTAPBPR (FIG. 19d, FIG. 36A). Consistent with the previous FP results that hTAPBPR effectively promotes peptide exchange on micropolymorphic HLA-A*02 subtypes (FIG. 17F), Applicants demonstrated that all three mutants enhanced the peptide exchange kinetics of HLA-A*02:06 (FIG. 19d, FIG. 36B). Both hTAPBPR^{RQ} and hTAPBPR^{KRQ} improved the k₂ₒᵥₑᵣₐₗₗ of pHLA-A*02:06 by approximately 9 folds compared to hTAPBPR, while hTAPBPR^{FLQ} showed the most pronounced effect of 14 fold among the mutants (FIG. 36B). Additionally, hTAPBPR^{FLQ} at 10 nM concentration promoted exchange on HLA-A*02:01 loaded with βF peptides at P1, P3, P4, and P8 more efficiently than hTAPBPR at the same concentration, as indicated by the enhanced kₐₚₚ (FIG. 19d, FIG. 36E, F). HLA-A*02:01/βF-P6, previously showing no peptide exchange in the presence or absence of 10 nM hTAPBPR, was exchanged for the fluorescent peptide with a fitted kₐₚₚ by 10 nM hTAPBPR^{FLQ} (FIG. 36E). HLA-A*02:01 proteins loaded with each of the seven βF peptides, including βF-P5 and βF-P7, were all observed for peptide exchange under 100 nM hTAPBPR^{FLQ} condition (FIG. 36E, F). Therefore, selected mutations not only can enhance the surface expression of HLA-A*02:01 in the cell-based assay and are functionally active in vitro with improved catalysis of peptide exchange.

Additionally, recombinant hTAPBPR does not interact with or catalyze peptide exchange on HLA-A*01:01 in vitro, and likewise over-expression of hTAPBPR-TM had no ability to increase surface HLA-A*01:01 levels in a tapasin-KO Expi293F cell line stably transfected with HLA-A*01:01 (FIGS. 32c, d). However, Applicants hypothesized that hTAPBPR-TM mutants with enhanced activity for the rescue of surface HLA-A*02:01 in tapasin-KO cells might have gained activity towards HLA-A*01:01. Indeed, hTAPBPR-TM carrying the substitutions K211R and/or R270Q, which were coincidentally aligned with the chTAPBPR sequence, stabilized HLA-A*01:01 at the cell surface (FIG. 19e). Furthermore, whereas purified soluble hTAPBPR had no effect on the exchange of a placeholder peptide on HLA-A*01:01 for a fluorescent peptide, the apparent rate constants kₐₚₚ of hTAPBPR^{RQ} (1.4×10⁻⁴ s⁻¹) and hTAPBPR^{KRQ} (1.3×10⁻⁴ s⁻¹) at 5 μM were close to the kₐₚₚ of chTAPBPR (1.7×10⁻⁴ s⁻¹) on HLA-A*01:01 (FIG. 19f, FIG. 36F). Recombinant hTAPBPR^{FLQ} also promoted peptide exchange on HLA-A*01:01 but was most inefficient among mutants with a kₐₚₚ of 6.8×10⁻⁴ s⁻¹ at 5 μM (FIG. 19f, FIG. 36F). Unlike chTAPBPR, both triple mutants hTAPBPR^{KRQ} and hTAPBPR^{FLQ} failed to catalyze peptide exchange on HLA-B*08:01 (FIG. 36E), demonstrating that the mutations do not universally enhance TAPBPR activity but are specific for a subset of MHC-I allotypes. Overall, Applicants' engineering efforts based on deep mutagenesis in a cell-based assay demonstrate how even a small number of mutations can manipulate the HLA allele specificity of TAPBPR and show congruence with natural mutations that have occurred during evolution.

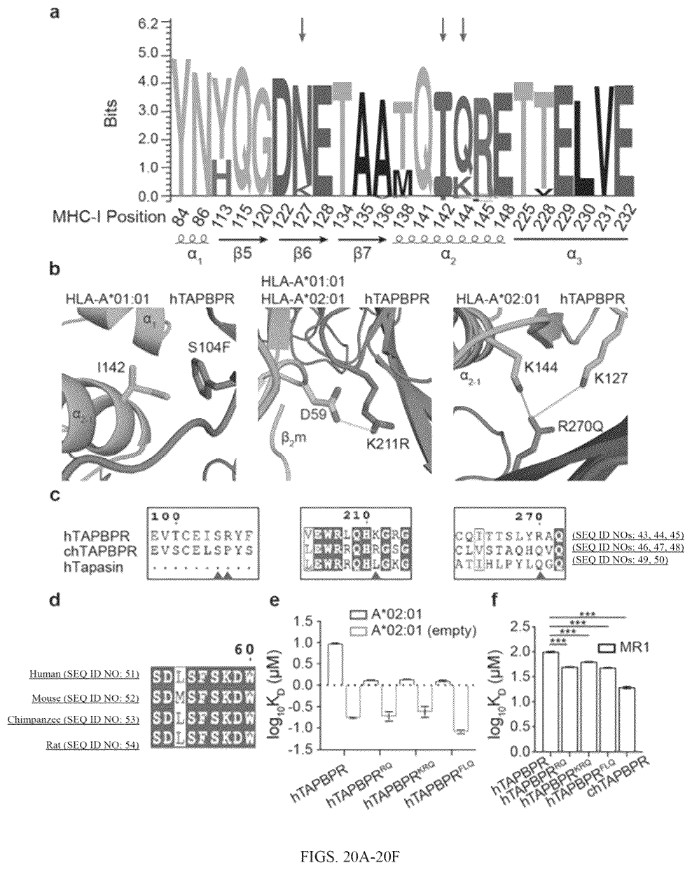

Polymorphisms between MHC-I and TAPBPR determine the allele-specific interactions. To further understand the sequence and structural basis of how specific TAPBPR amino acid substitutions can promote interactions with HLA-A*02:01, HLA-A*01:01 and other relevant allotypes, Applicants analyzed MHC-I polymorphic residues located on the TAPBPR interaction surfaces from 75 common (>1% population prevalence) HLA allotypes. There is a strong sequence conservation pattern of TAPBPR interacting residues on the MHC-I with some variations at specific sites (FIG. 20a). The selection of HLA supertype representatives covering the polymorphisms within the peptide binding groove also sufficiently depicts the level of sequence variation within the TAPBPR contacting residues of different HLA allotypes (Table 5). Applicants then performed structure-based modeling of the identified TAPBPR mutations using the X-ray structure of a mouse MHC-I/hTAPBPR complex²³ as a template, together with either HLA-A*01:01 or HLA-A*02:01. Inspection of the resulting models revealed that in hTAPBPR^{FLQ} S104F is located on the N-terminal IgV domain where it interacts with the outer rim of the MHC-I α₂₋₁ helix, specifically the hydrophobic residue 1142 of HLA-A*01:01 (FIG. 20b). Within HLA-A and HLA-B alleles, position 142 is either Ile or Thr. In models generated using hTAPBPR^{RQ} and hTAPBPR^{KRQ}, K211R is positioned at a surface that cradles the underside of the MHC-I peptide groove (FIG. 20b). The side chain of R211 interacted with the conserved residue D59 within the 58-60 loop of β₂m hypothesized to be involved with peptide sensing²³ (FIGS. 20b, d). Finally, models generated using R270Q remove the positively charged residue and reveal possible polar interactions with the side chains from residues K144 and K127 in HLA-A*02:01 (FIG. 20b); HLA-A alleles have either a Lys or Gln at position 127, suggesting a basis for the divergence of different alleles in their interactions with chaperones which can be leveraged to design allele-specific catalysts, as exemplified in Applicants' study (FIG. 20a). The mutational tolerance of these hTAPBPR residues in the deep mutational scan may therefore in part reflect a lack of close structural complementarity to allow for recognition of polymorphic sites among HLA alleles. Applicants note that the sequences of TAPBPR orthologs were not considered in selecting mutations, as an objective was to explore different routes to enhance the peptide editing function of TAPBPR. Nonetheless, a sequence alignment revealed that hTAPBPR amino acid substitutions K211R/L and R270Q are shared with chTAPBPR and hTapasin (FIG. 20c), which has preferential binding to HLA-B allotypes. Thus, structure modeling underscores different mechanisms for how TAPBPR interactions with both conserved and polymorphic residues of HLA molecules, including the β₂m subunit, can be exploited to design gain-of-function variants, showing congruence with natural mutations that have occurred during evolution.

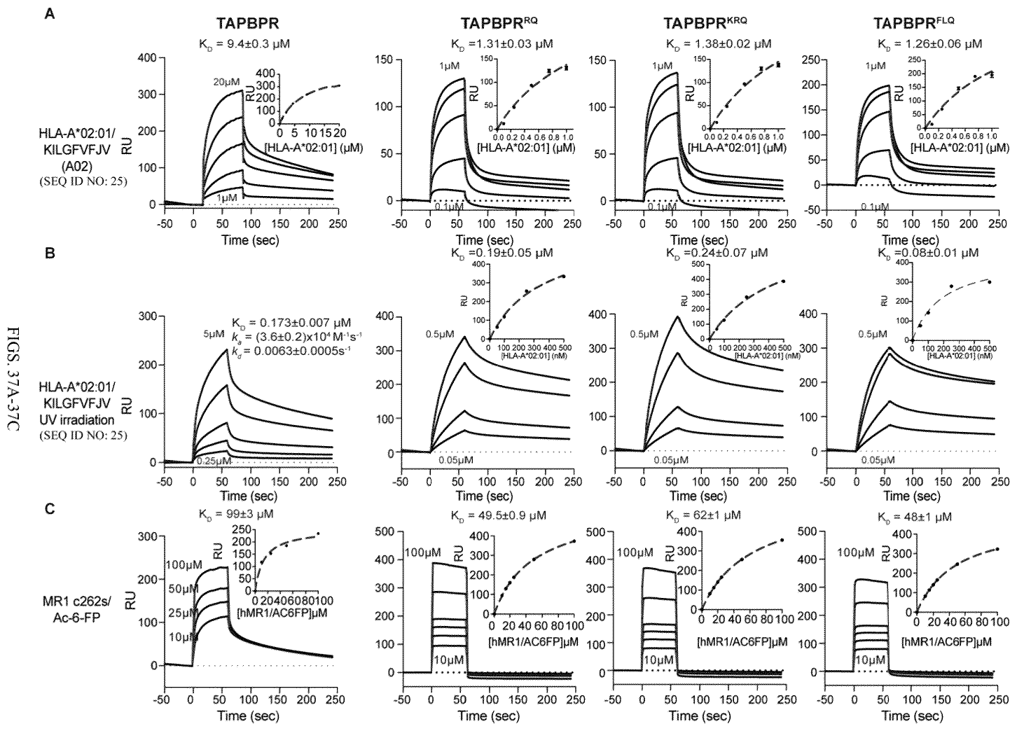

Applicants have demonstrated that recombinantly expressed TAPBPR mutants can increase the k₂ₒᵥₑᵣₐₗₗ of HLA-A*02:01 peptide exchange by nearly two orders of magnitude relative to hTAPBPR, suggesting an increase in binding affinity in agreement with Applicants' results from measuring TAPBPR-TM mediated surface trafficking of HLA-A*02:01 in tapasin-KO cells suggesting an increased binding affinity (FIGS. 19c, d). To quantitively characterize this effect, Applicants measured binding affinities of peptide-loaded or empty HLA-A*02:01 molecules to the top three hTAPBPR mutants using SPR. Peptide-loaded HLA-A*02:01 has a similar dissociation equilibrium constant, K_{D}, towards all three TAPBPR mutants (FIG. 20e), corresponding to approximately 7 fold lower than hTAPBPR (FIG. 37a). All three mutants have similarly low nanomolar-range K_{D}s for empty HLA-A*02:01 (FIG. 20e). hTAPBPR^{FLQ} consistent with its remarkable performance on peptide exchange and tight binding towards peptide-loaded HLA-A*02:01, has the lowest K_{D} for peptide-deficient molecules (81 nM), compared to hTAPBPR^{RQ} (188 nM) and hTAPBPR^{KRQ} (243 nM) (FIG. 37b). Finally, Applicants evaluated TAPBPR interactions with the non-classical MHC molecule MR1 C262S, which have been shown to catalyze the exchange of noncovalent metabolite ligands⁴². While chTAPBPR binds MR1 C262S 5-fold tighter than hTAPBPR (19 μM versus 99 μM, respectively), the K_{D}s of the TAPBPR mutants were reduced by approximately 2 fold (FIG. 20f, FIG. 37c). Akin to classical HLA, hTAPBPR^{FLQ} also demonstrated the tightest binding to MR1 C262S among all three mutants. In summary, Applicants' results demonstrate that TAPBPR residues contacting different MHC-I surfaces, including the α₂₋₁ helix, are tolerant of optimization, by either introducing a minimal set of hTAPBPR substitutions or by selecting desired TAPBPR orthologs, Applicants can enhance binding affinities through the optimization of specific interactions. Notably, TAPBPR provides a stable structural scaffold for obtaining gain-of-function interactions with allotypes that were previously not susceptible to interactions with the chaperone, such as HLA-A*01:01 and HLA-B*08:01.

TAPBPR, a homolog of tapasin, is known for its dual roles as a chaperone and a peptide exchange catalyst with exquisite HLA allelic specificity. A comprehensive study demonstrated how polymorphic residues removed from the MHC-I/chaperone binding surfaces can shape allele-dependent interactions toward a strong preference for HLA-A over B and C allotypes, while NMR data have shown that allele-dependent conformational dynamics of MHC-I molecules contribute to the recognition of a sparse protein state by TAPBPR²⁶. Although predicted structures of TAPBPR from different species are remarkably similar, whether different orthologs can mediate peptide exchange on human MHC-I proteins has not been addressed. Here, Applicants demonstrate novel xeno-interactions between a set of HLA allotypes and chicken TAPBPR that are distinct from the interaction profile with the human ortholog. Like hTAPBPR, chTAPBPR acts on properly conformed, peptide-loaded MHC-I molecules with a restricted allele-selectivity to unload suboptimal peptides (FIGS. 15c, 16c, 32a). Applicants further investigated interactions between TAPBPR orthologs and MHC-I in its peptide-deficient form, and demonstrated a broad allelic specificity and relatively high affinities towards empty molecules covering all six A supertypes, B08, and B44 supertypes as well as HLA-E and HLA-G (FIG. 16g). Applicants showed chTAPBPR binding epitopes on empty, peptide-receptive HLA-B*08:01 located on the α₁ and α₂-1 helix of the heavy chain with a contribution from β₂m (FIG. 18c), consistent with the footprint of hTAPBPR on murine MHC-I^{22,23}, as well as the conserved allosteric site underneath the α₂₋₁ helix of MHC-I that is crucial for tapasin interactions¹¹. The interactions with peptide-deficient MHC-I molecules have been shown to provide an essential protein homeostasis mechanism by stabilizing misfolded, nascent MHC species inside the cells^{12,56}. Applicants' results also underline a strong correlation between the capability of TAPBPR orthologs to stabilize empty MHC-I molecules and maintain them in a peptide-receptive state for peptide exchange. Both stoichiometric and sub-stoichiometric amounts of TAPBPR orthologs can accelerate peptide loading kinetics to allow complete exchange (FIG. 17E, FIGS. 28-29, 30). Together, Applicants' data support two important hallmarks of TAPBPR interactions, namely that they i) are both peptide- and MHC allele-dependent, however they demonstrate a broader allelic specificity with respect to recognition of empty MHC-I molecules; and ii) can stabilize empty molecules and maintain them in a peptide-receptive state to facilitate peptide exchange.

Applicants' results further underline a strong correlation between the recognition of empty MHC-I molecules by different TAPBPR variants, the stabilization of a peptide-receptive conformation, and the rate of peptide exchange reactions. Applicants have determined that sub-stoichiometric amounts of TAPBPR can significantly accelerate peptide exchange kinetics through tight interactions with empty MHC-I molecules (FIGS. 16g, 17f, h). According to this model, MHC-I molecules loaded with a moderate-affinity, placeholder peptide can undergo a slow, spontaneous process of peptide dissociation to create empty molecules (FIG. 43A). Subsequent binding of incoming peptides requires empty MHC-I to transit from a “closed” to an “open” groove state^{24,72}, which involves a new free energy barrier (FIG. 43B). TAPBPR enhances the transition rate between conformational states and stabilizes the empty MHC-I groove in an “open” conformation, thereby lowering the activation free energy⁷²⁻⁷⁴ (FIG. 43C). Loading of high-affinity peptides induces a “closed” MHC-I groove conformation leading to TAPBPR dissociation due to its substantially increased K_{D} towards peptide-loaded molecules^{24,46} (FIG. 43D). TAPBPR variants may lower the activation free energy barrier to varying levels for different allotypes, resulting in different peptide exchange kinetics (FIG. 43).

Nevertheless, in contrast to classical, naturally occurring enzymes, TAPBPR exhibits a relatively minor catalytic effect (10-10² vs. 10⁶-10¹² fold faster than the rate of uncatalyzed peptide exchange reaction⁷⁵). A possible reason for the reduced catalytic efficiency of TAPBPR is its additional requirement to function as a molecular chaperone of nascent MHC-I molecules inside the cell. According to its chaperone function, TAPBPR must bind empty MHC-I molecules with high affinity (the substrate for peptide loading). This requirement may compromise its ability to stabilize and extend the “active” state of MHC-I for the loading reaction, which is one hallmark of catalysis by naturally occurring enzymes. Although previous studies have shown that TAPBPR can recognize peptide-loaded molecules to promote dissociation of sub-optimal peptides^{17,22,55} this unloading activity typically requires micromolar-range concentrations of TAPBPR (FIGS. 26A, C), and is likely not the primary cellular function of TAPBPR. TAPBPR orthologs in a molar excess substantially enhance the overall exchange rate (FIG. 17H, FIGS. 27-29) and complete bulk peptide exchange in a short period of incubation (FIG. 30). This mechanism of recognizing peptide-loaded MHC-I and forming a peptide/MHC-I/TAPBPR intermediate for peptide unloading may also explain why engineered hTAPBPR variants show improved peptide exchange function (FIG. 19d, FIGS. 36A-F). Human TAPBPR mutants at a low concentration rather than a conventional molar excess can now promote peptide unloading, due to their reduced K_{D} (increased affinity) to the peptide-loaded MHC-I (FIG. 20e, f, FIG. 37). TAPBPR orthologs and engineered variants have differential respective binding free energy (ΔG_{B1}, ΔG_{B1}, and ΔG_{B3}) to distinct HLA allotypes, resulting in various levels of activation free energy barrier and, therefore, different peptide exchange kinetics. Taken together, Applicant's results demonstrate the catalytic effect of TAPBPR to promote the unloading of the peptide-loaded MHC-I and to stabilize a reservoir of empty, peptide-receptive molecules to capture new incoming peptides (FIG. 43).

To understand the molecular basis of HLA recognition by chTAPBPR, Applicants further isolate and characterize a functionally competent HLA-B*08:01/chTAPBPR complex (FIGS. 31b, c). Hydrogen deuterium exchange identifies chTAPBPR recognition surfaces located on the α₂₋₁ helix of the heavy chain with a contribution from α₁ and β₇ on β₂m (FIG. 18c). These identified epitopes on MHC-I are consistent with the footprint of hTAPBPR in the two co-crystal structures with murine MHC-I molecules^{22,23}, as well as a conserved allosteric site underneath the α₂₋₁ helix of MHC-I that is crucial for tapasin interactions¹¹.

The molecular basis for MHC-I allele dependence of chaperones is highly complex and can arise from interactions with nascent, partially folded, or peptide-loaded molecules. Previous studies have shown that TAPBPR can recognize peptide-loaded MHC-I molecules to promote unloading of the bound peptides by widening the MHC-I peptide binding groove^{10,55}. Consistently, Applicants find that chTAPBPR can bind to multiple peptide-loaded HLA-A allotypes and HLA-B*08:01 molecules with a low micromolar affinity by SPR and ITC (FIG. 16c, FIG. 31a). Furthermore, both TAPBPR orthologs can maintain empty MHC-I molecules in a receptive state and promote peptide loading (FIG. 16g). Incoming peptides may be captured by empty MHC-I molecules released by TAPBPR or might bind directly to chaperoned MHC-I protein complexes to trigger TAPBPR release through a negative allosteric coupling mechanism²⁴. While empty MHC-I/TAPBPR complexes can be purified in a stable form, Applicants' measured hydrogen/deuterium exchange rates suggest that interactions with TAPBPR are reversible (low micromolar range K_{D}). However, these transient interactions can still result in efficient catalysis of peptide exchange under sub-stoichiometric TAPBPR concentrations (FIGS. 17C, E, G, 19d). In conclusion, depending on the cellular conditions, TAPBPR orthologs might enact multiple pathways for peptide exchange, including i) on-groove unloading of suboptimal peptides from pMHC molecules, ii) on-groove loading of high-affinity peptides, and iii) passive editing, by maintaining a reservoir of peptide-receptive MHC-I molecules which are loaded with peptides upon their release from TAPBPR. Notably, all three mechanisms can contribute to peptide exchange function in vitro.

Since the discovery of molecular chaperones tapasin^{5,59} and TAPBPR^{7,8}, efforts to identify HLA alleles that are susceptible to chaperone-assisted peptide exchange in vitro or on the cell surface have been carried out using a range of biochemical and in vitro approaches^{10,11,17,21,60}. Due to its ability to function independently of the peptide-loading complex, TAPBPR is best suited for peptide exchange applications. Previous applications include the loading of immunogenic peptides onto surface-expressed MHC-I molecules⁵⁷ and the generation of pMHC-I tetramer libraries in a high-throughput manner for T cell epitope selection and antigen-specific T cell monitoring⁴⁷. However, TAPBPR-mediated peptide exchange has only been demonstrated for a limited set of common HLA allotypes, mainly from the A02 and A24 supertypes, limiting wide adoption of these technologies in biomedical applications^{10,17,21,47}. Here, Applicants demonstrate novel, optimized peptide exchange functions on HLA-A*01:01, HLA-A*03:01, HLA-A*11:01, HLA-A*24:02, HLA-B*08:01, and HLA-B*37:01 using chTAPBPR (FIG. 1711), as well as identified hTAPBPR mutants identified from Applicants' deep mutagenesis studies that dramatically enhance peptide exchange efficiency on HLA-A*02:01 and enabled peptide exchange on HLA-A*01:01 (FIGS. 19d, f). Deep scanning mutagenesis, based on this indirect cell assay for interactions between hTAPBPR and folded HLA-A*02:01 localized to the cell surface, demonstrated that hTAPBPR residues in contact with the α₂₋₁ helix of MHC-I had a higher tolerance for mutations compared to other regions at the interaction surface (FIGS. 19a, b), possibly reflecting imperfect complementarity at the interface to support MHC-I polymorphisms. Although the highly polymorphic nature of MHC-I molecules challenges the engineering of “universal” chaperones, Applicants' results demonstrate a relatively conserved TAPBPR binding epitope on MHC-I (FIG. 20a) and the possibility of designing and engineering off-the-shelf TAPBPR variants with minimal adjustments to enable peptide exchange on a broad range of HLA allotypes of choice. Applicants' study further opens the use of TAPBPR orthologs to perform ligand exchange on oligomorphic MHC-Ib and MR1 molecules. TAPBPR orthologs can also be utilized in various cancer immunotherapeutic settings to narrow the peptide repertoire, thereby increasing neoepitope immunogenicity. In conclusion, the knowledge gained by Applicants' studies can guide the design of engineered TAPBPR variants with tailored HLA specificity and catalytic efficiency for peptide exchange applications both in vitro and in vivo.

Phylogenetic analysis. TAPBPR sequence references used for the phylogenetic analysis are as follows: Cat (Felis catus) XP_023112473.2; Chicken (Gallus gallus) NP_001382952.1; Chimpanzee (Pan troglodytes) XP_009422944.1; Dog (Canis lupus familiaris) XP_005637308.1; Dolphin (Tursiops truncatus) XP_033723028.1; Frog (Xenopus laevis) XP_018080550.1; Goat (Capra hircus) XP_017904083.1; Horse (Equus caballus) XP_023498789.1; Human (Homo sapiens) Q9BX59; Mouse (Mus musculus) XP_030111162.1; Opossum (Monodelphis domestica) XP_007485846.1; Rabbit (Oryctolagus cuniculus) XP_017198892.1; Salmon (Salmo salar) NP_001133983.1; Shark (Carcharodon carcharias) XP_041036048.1; Turtle (Chelydra serpentina) KAG6922351.1; Zebrafish (Danio rerio) XP_001919985.2. All amino acid sequence alignments were performed using ClustalOmega⁶¹ and processed using ESPript⁶², the tree was inferred using best-fit models as calculated by MEGA7⁶³ and bootstrapped using 100 replicates.

For comparing HLA-I alleles, sequences were collated from IPD-IMGT/HLA¹³ and a multiple sequence alignment was made using ClustalOmega⁶¹ and processed using ESPript⁶². The predicted complex structures were generated using RosettaCM^{64,65}.

Peptides and ligands. All peptide sequences are given as standard single-letter codes. High affinity epitopic peptides for different HLA allotypes were selected by NetMHCpan4.1 and purchased from Genscript, Piscataway, USA, at >90% purity. L-β-Phenylalanine (βF) containing placeholder peptides were synthesized in-house on 2-chlorotrityl resin using a CEM Liberty Blue automated microwave peptide synthesizer from Fmoc protected amino acids (including Fmoc-β-Phe-OH) employing iterative cycles of N, N′-Diisopropylcarbodiimide (DIC)/Ethyl cyanohydroxyiminoacetate (Oxyma) mediated coupling and piperidine mediated deprotection, both under microwave irradiation. Peptides were deprotected and cleaved from resin by treatment with trifluoroacetic acid/water/triisoproylsilane/phenol (88:5:5:2) for 1-3 hours. Solvent was removed under a flow of nitrogen and peptides were precipitated with ice cold ether. Peptides were subsequently purified by reverse phase chromatography eluting with 5-95% acetonitrile in water containing 0.05% trifluoroacetic acid over a C18 column. Peaks containing peptides were identified by LC-MS, pooled, and concentrated in vacuo to yield a colorless solid. Photolabile peptides were purchased from Biopeptek Inc, Malvern, USA, or synthesized in-house as above and using J as Fmoc-3-amino-3-(2-nitrophenyl)-propionic acid. Peptides were solubilized in distilled water and centrifuged at 14000 rpm for 15 minutes. The concentration of each peptide solution was measured and calculated using the respective absorbance and extinction coefficient at 205 nm wavelength. The MR1 ligand acetyl-6-formylpterin (AC-6-FP) was purchased from Cayman Chemical no. 23303.

Recombinant protein expression, refolding, and purification. Plasmid DNA encoding the BirA Substrate Peptide (BSP, LHHILDAQKMVWNHR)-tagged luminal domain of MIC-I heavy chains and human 02-microglobulin light chain (hβ₂m) were provided by the NIH tetramer facility (Emory University) and transformed into Escherichia coli BL21 (DE3) cells (New England Biolabs). Proteins were expressed in the Luria-Broth medium, and inclusion bodies were collected and purified as previously described⁶⁶. For the generation of pMHC-I molecules, in vitro refolding was performed by slowly diluting a 200 mg mixture of BirA-tagged MHC-I and hβ₂m at a 1:3 molar ratio over 24 hours in refolding buffer (0.4 μM L-Arginine-HCl, 100 mM Tris pH 8, 2 mM EDTA, 4.9 mM reduced L-glutathione, 0.57 mM oxidized L-glutathione) containing 10 mg of the placeholder peptide. The mixture was protected from light when refolded with photolabile peptides. Refolding proceeded for 4 days, and proteins were purified by size-exclusion chromatography (SEC) using a HiLoad 16/600 Superdex 75 pg column at 1 mL/min with 150 mM NaCl, 20 mM Tris buffer, pH 8.0.

The luminal domain of human WT and mutants, chicken, and mouse TAPBPR protein tagged with BSP and 6-His was stably expressed in the Drosophila melanogaster S2 cell line¹⁰. The cultures were induced with 1 mM CuSO4 and after 4 days the supernatant was collected. The secreted TAPBPR molecules were purified using a high-density metal affinity agarose resin (ABT, Madrid). The eluted proteins were further purified by SEC using a HiLoad 16/600 Superdex 200 pg column at a flow rate of 1 mL/min in 150 mM NaCl, 20 mM sodium phosphate buffer pH 7.4.

Purification of peptide-receptive MHC-I TAPBPR. chTAPBPR was mixed with HLA-B*08:01/FLRGRAJGL (SEQ ID NO: 4) at a 1:1.5 molar ratio. The mixture was incubated at room temperature (RT) for 1 hour, followed by 40 min UV irradiation at 365 nm and 30 minutes additional RT incubation. The complex was purified by SEC using a Superdex 200 pg Increase 10/300 GL and the eluted peaks were further analyzed by SDS/PAGE electrophoresis to identify all components.

Native gel shift assay. Purified MHC-I molecules refolded with photo-labeled peptides (pMHC-I) and TAPBPR proteins were loaded into polyacrylamide gels alone at 7 μM concentration and as mixtures. For the mixtures, a 1:1 molar ratio of pMHC-I and TAPBPR samples at 7 μM each was prepared and incubated at RT for 1 hour. The samples were then UV-irradiated for another hour at 365 nm and were run at 90 V for 5.5 hours on a 12% polyacrylamide gel. Staining was performed using InstantBlue (Novus Biologicals).

Biotinylation and Tetramer Formation. BSP-tagged TAPBPR proteins were biotinylated using the BirA biotin-protein ligase bulk reaction kit (Avidity), according to the manufacturer's instructions. Biotinylated molecules were washed using Amicon Ultra centrifugal filter units with a 10 kDa membrane cut-off, and the level of biotinylation was evaluated by SDS-PAGE gel shift assay in the presence of excess streptavidin. Biotinylated TAPBPR were prepared at a final concentration of 2 mg/mL. Streptavidin-PE (Agilent Technologies, Inc.) at 4:1 monomer/streptavidin molar ratio were added to pMHC-I over 10-time intervals every 10 min at room temperature (RT) in the dark. The resulting TAPBPR tetramers can be stored at 4° C. for up to 4 weeks.

SABs screen. 7 μM of TAPBPR in tetramers were mixed with 4 μL of the LABScreen single antigen HLA-I bead suspension (OneLambda, Inc., CA, USA) in a 96-well plate. The samples were incubated for 1 hour, 550 rpm at RT, washed four times in Wash Buffer (OneLambda, Inc., CA, USA) to remove excess tetramers and resuspended in phosphate-buffered saline (PBS), pH 7.2. For the negative controls, beads were incubated with the Anti-HLA Class I antibody W6/32 (Abcam, ab22432) for 30 min, 550 rpm at RT and washed three times prior to tetramer addition. To test the levels of peptide loaded MHC-I molecules on the beads, Applicants used the same W6/32 antibody and the secondary anti-mouse PE-conjugated antibody (Abcam, ab97024) for detection. The levels of TAPBPR bound to the beads were measured using the Luminex 100 Liquid Array Analyzer System and the results were analyzed in GraphPad Prism v9.

DSF. DSF was used to measure the thermal stability of the TAPBPR proteins and the pMHC-I molecules. 7 μM of TAPBPR protein or pMHC-I molecules with TAPBPR and the desired peptide at 1:7:70 TAPBPR/pMHC-I/peptide molar ratio was incubated for 2 hours at RT in the dark and then mixed with 10×SYPRO Orange dye in a buffer of 150 mM NaCl, 20 mM sodium phosphate pH 7.2 to a final volume of 20 μL. Samples were loaded into MicroAmp Optical 384 well plate and ran in triplicates. The experiment was performed on a QuantStudio™ 5 Real-Time PCR machine with excitation and emission wavelength set to 470 nm and 569 nm. The thermal stabilities of pMIHC complexes were compared by plotting the first derivative of each melting curve and extracting the peak as the melting temperature (Tₘ). The thermal stability was measured by gradually increasing temperature at a rate of 1° C. per minute between 25° C. and 95° C. Data analysis and fitting were performed in GraphPad Prism v9.

SPR. SPR experiments were conducted in duplicate or triplicate using a BiaCore T200 instrument (Cytiva) in SPR buffer (150 mM NaCl, 20 mM sodium phosphate pH 7.4, 0.1% Tween-20). Approximately 1000 resonance units (RU) of biotinylated human or chicken TAPBPR or hTAPBPR mutants were immobilized at 10 μL/min on a streptavidin-coated chip (GE Healthcare). Various concentrations (0, 0.05, 0.1, 0.25, 0.5, 0.75, 1, 2.5, 5, 7.5, 10, and 20 μM) of HLA molecules were selected and injected over the chip at 25° C. at a flow rate of 30 μL/min for 60 s followed by a buffer wash with 180 s dissociation time. For MR1 experiments, various concentrations (0, 5, 10, 15, 20, 25, 50, 100 μM) were selected and used on hTAPBPR, chTAPBPR, or hTAPBPR mutants immobilized streptavidin chip. Before SPR experiments of UV-irradiated molecules, HLA molecules were diluted to the desired concentrations then UV irradiated for 20 minutes at 365 nm. SPR data were then immediately acquired following 20-minute UV irradiation. The SPR sensorgrams, association/dissociation rate constants (kₐ, k_{d}) and equilibrium dissociation constants (K_{D}) were analyzed using the surface-bound analysis settings in BiaCore T200 evaluation software (Cytiva) or fitted using one site specific binding by GraphPad Prism v9. SPR sensorgrams and saturation curves were prepared in GraphPad Prism v9.

FP. Kinetic association of fluorescently labeled peptides and various pHLA-I was monitored by FP. pHLA-I was combined with graded concentrations of TAPBPR in FP buffer (150 mM NaCl, 20 mM sodium phosphate, 0.05% Tween-20, pH 7.4) and the optimized concentration of fluorescently labeled peptide to achieve a polarization baseline between 0 and 50 mP, determined via serial dilution. _{TAMRA}KLFGYPVYV (SEQ ID NO: 27) (40 nM), _{TAMRA}KYNPIRTTF (SEQ ID NO: 36) (6 nM), _{FITC}KSDPIVAQY (SEQ ID NO: 34) (20 nM), _{FITC}KLRGRAYGL (SEQ ID NO: 78) (20 nM), _{FITC}KEDLRVSSF (SEQ ID NO: 84) (20 nM), and _{FITC}KLIETYFSK (SEQ ID NO: 35) (20 nM) were added to HLA-A*02:01/KILGFVFβFV (SEQ ID NO: 12), HLA-A*24:02/VYGJVRACL (SEQ ID NO: 3), HLA-A*01:01/STAPGJLEY (SEQ ID NO: 9), HLA-B*08:01/KLRGRAJGL (SEQ ID NO: 85), HLA-B*37:01/FEDLRVβFSF (SEQ ID NO: 17), and both HLA-A*03:01/KLIETYFβFK (SEQ ID NO: 37) and HLA-A*11:01/KLIETYFβFK (SEQ ID NO: 72) in the presence of TAPBPR orthologs and mutant at various concentrations as indicated. The pMHC-I concentration remained constant across experiments at 200 nM. Fluorescently labeled peptides were directly added into the well plate to avoid extended incubation and loss of data. Wells were loaded with 100 μL of reaction and triplicates for each condition were performed. The kinetic association was monitored for 2-12 hours dependent on the kinetics, and polarization measurements were recorded every 60-90 seconds. Reaction setup as well as kinetic monitoring via FP were performed at room temperature. Excitation and emission values used to monitor the fluorescence of TAMRA- and FITC-labeled peptides were 531 & 595 nm and 475 & 525 nm.

Raw parallel and perpendicular emission intensities (I_{II} and I_{⊥}, respectively) were collected and converted to polarization (mP) values using the equation 1000*[(I_{II}−(G*I_{⊥}))/(I_{II}+(G*I_{⊥}))]. G-factors of 0.33 and 0.4 were optimized for TAMRA- and FITC-labeled peptides in calculating the overall fluorescence polarization. Data analysis method⁴³ was adapted and data fitting were performed in GraphPad Prism v9.

ITC. ITC experiments between pHLA-B*08:01 and chTAPBPR constructs were obtained using a MicroCal VP-ITC system (Malvern analytical). All proteins were exhaustively dialyzed into the buffer (150 mM NaCl, 20 mM sodium phosphate pH 7.2) and filtered through a 0.22 m PES membrane. A syringe containing pHLA-B*08:01 at 150 μM with 0.45 mM peptide (FLRGRAJGL (SEQ ID NO: 4)) was titrated into the calorimetry cell containing 5 μM TAPBPR and the same peptide (0.45 mM). Injection volumes were 10 μL performed for a duration of 10 s and spaced 220 s apart to allow a complete return to baseline. Data were subtracted from a control performed where a syringe containing pMHC-I at 150 μM with 0.45 mM FLRGRAJGL (SEQ ID NO: 4) was titrated into the calorimetry cell containing buffer and the peptide (0.45 mM). Data were processed and analyzed with Origin software. Isotherms were fit using a one-site ITC binding model. The first data point was excluded from the analysis. Reported K_{D}, −T*ΔS, and ΔH values are determined using the 1-site binding model.

HDX-MS. HLA-B*08:01/FLRGRAJGL (SEQ ID NO: 4) and purified HLA-B*08:01/chTAPBPR were dialyzed into equilibration buffer (150 mM NaCl, 20 mM sodium phosphate pH 6.5 in H₂O) and dilute to a stock concentration of 30 μM. HLA-B*08:01/FLRGRAJGL (SEQ ID NO: 4) were then either 1) kept on ice without exposure to UV light or 2) UV-exposed for 40 min at 4° C.

Samples were prepared and injected manually for several deuterium-exchange incubation periods. HLA-B*08:01/FLRGRAJGL (SEQ ID NO: 4) with or without UV irradiation and purified HLA-B*08:01/chTAPBPR (5 μL) were diluted with 20 μL equilibration buffer (all H experiments) or deuterium buffer (150 mM NaCl, 20 mM sodium phosphate pD 6.5 in D₂O) to 6 μM. The proteins were incubated with deuterium buffer for 20 s, 1 min, 3 min, and 10 min at RT, and 15 min at 46° C. for HLA-B*08:01/FLRGRAJGL (SEQ ID NO: 4) or at 32° C. for UV-irradiated HLA-B*08:01 and purified HLA-B*08:01/chTAPBPR as the all D samples to calculate ΔMass_{100%}. The samples were then quenched with an equal volume of acidic buffer (150 mM NaCl, 1 μM TCEP, 20 mM sodium phosphate pH 2.35 in H₂O, 25 μL). Quenched proteins (3 μM) were immediately injected for LC-MS/MS in which integrated pepsin digestion was performed using a C8 5 μM column and a Q Exactive Orbitrap Mass Spectrometer. Peptide fragments corresponding to HLA-B*08:01 (97% coverage) and hβ₂m (94% coverage) were identified using Thermo Proteome Discoverer v2.4.

The percent deuterium uptake is back-exchange corrected for each time point using the following equation:

$$Δ HDX = \frac{Δ {Mass}_{T = X} - Δ {Mass}_{0 \%}}{Δ {Mass}_{100 \%} - Δ {Mass}_{0 \%}} .$$

ExMs2 program was used to identify and analyze deuterated peptides. The kinetic plots and the scaled B factor for the final structure plot were generated by python3 and PyMOL.

Cell lines and expression plasmids for cell assays. Expi293F (ThermoFisher) cells were cultured in Expi293 Expression media (ThermoFisher) at 37° C., 125 rpm, 8% CO₂. The generation of tapasin-KO cells are previously described⁵¹. HLA-A*01:01-knockin/tapasin-KO cells were generated by first transfecting Expi293F cells with EcoRV linearized pCEP4-HLA-A*01:01 (untagged). The cells were selected with 100 μg/mL hygromycin B and FACS sorted for HLA-A*01:01 positive cells after staining with anti-HLA-A*01:01 clone 8.L.101 (USBio H6098-05). Following sorting, the HLA-A*01:01 positive cells were transfected with plasmids encoding human codon-optimized Cas9 and tapasin gRNA as previously described⁵¹ and then FACS sorted for cells that no longer processed HLA-A*01:01 to the cell surface.

TAPBPR-TM was cloned into pCEP4 (Invitrogen) with a canonical N-terminal MHC-I signal peptide, FLAG tag, linker, luminal domain of human TAPBPR, linker, and transmembrane domain of HLA-G (Addgene No. 135500). Mouse and chicken TAPBPR were cloned using the same design and included C94A and C100A, respectively. HLA-A*01:01 was subcloned by removing the myc tag from pCEP4-myc-HLA-A*01:01 as previously described²⁶. Site directed mutagenesis of TAPBPR-TM mutants was achieved using overlap extension PCR. The inserts of all plasmids were confirmed by Sanger sequencing.

Deep mutational scan. A single site-saturation mutagenesis (SSM) library was constructed for TAPBPR-TM, focused on specific residues in the luminal domain that interface with MHC-I or are at control sites. The SSM library was created by subcloning the previously published TAPBPR-tapasinCT libraries⁵¹; a DNA fragment corresponding to the TAPBPR luminal domain was PCR amplified and a canonical TM tail from HLA-G added in a second round of PCR-based assembly. The resulting TAPBPR-TM library products were pooled, restriction enzyme digested and ligated into pCEP4 before electroporation into NEB10beta cells (New England Biolabs). The transformation efficiency was at least 10-fold higher than the theoretical sequence diversity. Purified library plasmid DNA (1 ng per mL culture) was diluted with pCEP4-ΔCMV⁶⁷ (1500 ng per mL culture) and transfected using expifectamine (Thermo Fisher) into tapasin-KO Expi293F cells (2×106/ml). The medium was replaced after 2 hours. Under these conditions, cells typically receive no more than a single coding plasmid. Cells were collected 24 hours post-transfection (500×g, 3 minutes, 4° C.), washed with PBS containing 0.2% bovine serum albumin (PBS-BSA), incubated for 30 minutes at 4° C. with 1:200 antiT-HLA-A*02:01 PE (clone BB7.2, BioLegend), washed twice with PBS-BSA, and sorted on a BD FACSAria II. 4′,6-Diamidino-2-phenylindole (DAPI, 300 nM final concentration) was added and cells were gated for viability. The main singlet population was then gated by scattering and the top 2% of cells for PE fluorescence were collected (FIG. 34). RNA was extracted (GeneJet RNA purification kit, ThermoFisher), first strand cDNA was synthesized using Accuscript (Agilent) primed with EBV reverse primer, which anneals to the 3′ UTR of pCEP4-encoded transcripts. PCR products covering the mutated regions in the TAPBPR-TM library were amplified, followed by a second round of PCR to add Illumina adapters and sample-specific barcodes. The amplicons were Illumina sequenced on a NovaSeq 6000 and analyzed with Enrich⁶⁸. In brief, the frequency of each sequence variant in the sorted library is divided by its frequency in the pre-sorted naive library to calculate an enrichment ratio. The enrichment ratios are presented on a log base 2 scale. Conservation scores from the sorting experiment are calculated by averaging the log (2) enrichment ratios for all amino acid substitutions at a given residue (excluding nonsense mutations).

Flow cytometry. Expi293F cells were transfected at 2×10⁶ cells/ml using expifectamine (ThermoFisher) and collected 24 hours post transfection. For testing surface HLA-A*02:01, cells were washed with PBS supplemented with 0.2% bovine serum albumin (PBS-BSA), incubated with 1:200 antiT-HLA-A*02:01 PE (clone BB7.2, Biolegend 343306) and 1:200 anti-DYKDDDK APC (clone L5, Biolegend 637308) for staining surface TAPBPR, washed twice with PBS-BSA, and analyzed on a BD Accuri cytometer. For measuring surface HLA-A1, cells were washed with PBS-BSA, incubated with 1:200 mouse anti-HLA-A*01:01 (Clone 8.L.101, USBio H6098-05), washed twice with PBS-BSA, incubated with 1:200 goat anti-mouse IgG+IgM (H+L) (Jackson ImmunoResearch), washed twice with PBS-BSA, and analyzed on a BD Accuri. Results were processed using FCS Express 6.

Immunoblots. Equal cell quantities were lysed with sodium dodecyl sulfate (SDS) loading dye. Proteins were separated by gel electrophoresis and transferred to a polyvinylidene difluoride (PVDF) membrane. PVDF membranes for FLAG-tagged TAPBPR proteins were blocked with β% bovine serum albumin (BSA) in Tris buffered saline supplemented with 0.2% Tween20 (TBS-T) for 30 mins, incubated with 1:2,000 anti-FLAG AP (Sigma-Aldrich) in 1% BSA/TBS-T, washed 5 times with TBS-T, and then visualized with One step BCIP (ThermoFisher). PP1B was used as a loading control; blots were blocked with 5% non-fat milk/TBS-T, incubated with 1:2,000 rabbit anti-cyclophilin B (Invitrogen), washed five times with TBS-T, incubated with 1:10,000 goat anti-rabbit HRP (Jackson ImmunoResearch Laboratories), washed five times with TBS-T, and visualized using Clarity Western ECL Substrate (Biorad).

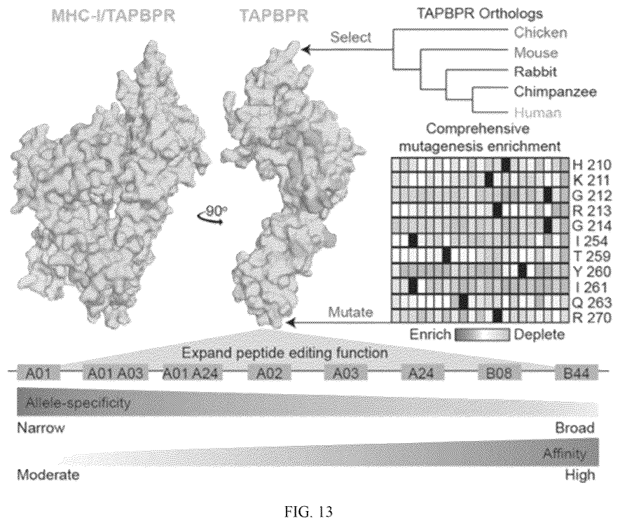

### REFERENCES OF EXAMPLE 10 AND FIGS. 13-20

1. Rossjohn, J. et al. T cell antigen receptor recognition of antigen-presenting molecules. Annu Rev Immunol 33, 169-200 (2015).
2. Rock, K. L., Reits, E. & Neefjes, J. Present Yourself! By MHC Class I and MHC Class II Molecules. Trends in Immunology 37, 724-737 (2016).
3. Germain, R. N. & Margulies, D. H. The biochemistry and cell biology of antigen processing and presentation. Annu Rev Immunol 11, 403-450 (1993).
4. Blum, J. S., Wearsch, P. A. & Cresswell, P. Pathways of Antigen Processing. Annu Rev Immunol 31, 443-473 (2013).
5. Sadasivan, B., Lehner, P. J., Ortmann, B., Spies, T. & Cresswell, P. Roles for Calreticulin and a Novel Glycoprotein, Tapasin, in the Interaction of MHC Class I Molecules with TAP. Immunity 5, 103-114 (1996).
6. Ortmann, B. et al. A Critical Role for Tapasin in the Assembly and Function of Multimeric MHC Class I-TAP Complexes. Science 277, 1306-1309 (1997).
7. Teng, M. S. et al. A human TAPBP (TAPASIN)-related gene, TAPBP-R. European Journal of Immunology 32, 1059-1068 (2002).
8. Boyle, L. H. et al. Tapasin-related protein TAPBPR is an additional component of the MHC class I presentation pathway. Proc Natl Acad Sci USA 110, 3465-3470 (2013).
9. Hermann, C., Strittmatter, L. M., Deane, J. E. & Boyle, L. H. TAPBPR and tapasin binding to MHC class I is mutually exclusive. J Immunol 191, 10.4049/jimmunol.1300929 (2013).
10. Morozov, G. I. et al. Interaction of TAPBPR, a tapasin homolog, with MHC-I molecules promotes peptide editing. Proc Natl Acad Sci USA 113, E1006-E1015 (2016).
11. Lan, H. et al. Exchange catalysis by tapasin exploits conserved and allele-specific features of MHC-I molecules. Nat Commun 12, 1-13 (2021).
12. Neerincx, A. et al. TAPBPR bridges UDP-glucose:glycoprotein glucosyltransferase 1 onto MHC class I to provide quality control in the antigen presentation pathway. Elife 6, e23049 (2017).
13. Robinson, J. et al. IPD-IMGT/HLA Database. Nucleic Acids Research 48, D948-D955 (2020).
14. Robinson, J. et al. Distinguishing functional polymorphism from random variation in the sequences of >10,000 HLA-A, -B and -C alleles. PLoS Genet 13, e1006862 (2017).
15. Chappell, P. E. et al. Expression levels of MHC class I molecules are inversely correlated with promiscuity of peptide binding. eLife 4, e05345 (2015).
16. Bashirova, A. A. et al. HLA tapasin independence: broader peptide repertoire and HIV control. Proceedings of the National Academy of Sciences 117, 28232-28238 (2020).
17. Hermann, C. et al. TAPBPR alters MHC class I peptide presentation by functioning as a peptide exchange catalyst. eLife 4, e09617 (2015).
18. Yu, Y. Y. L. et al. An Extensive Region of an MHC Class I α₂ Domain Loop Influences Interaction with the Assembly Complex. The Journal of Immunology 163, 4427-4433 (1999).
19. Peace-Brewer, A. L. et al. A Point Mutation in HLA-A*0201 Results in Failure to Bind the TAP Complex and to Present Virus-Derived Peptides to CTL. Immunity 4, 505-514 (1996).
20. Rizvi, S. M. et al. Distinct assembly profiles of HLA-B molecules. J Immunol 192, 4967-4976 (2014).
21. Ilca, F. T., Drexhage, L. Z., Brewin, G., Peacock, S. & Boyle, L. H. Distinct Polymorphisms in HLA Class I Molecules Govern Their Susceptibility to Peptide Editing by TAPBPR. Cell Rep 29, 1621-1632.e3 (2019).
22. Thomas, C. & Tampe, R. Structure of the TAPBPR-MHC I complex defines the mechanism of peptide loading and editing. Science 358, 1060-1064 (2017).
23. Jiang, J. et al. Crystal structure of a TAPBPR-MHC I complex reveals the mechanism of peptide editing in antigen presentation. Science 358, 1064-1068 (2017).
24. McShan, A. C. et al. Peptide exchange on MHC-I by TAPBPR is driven by a negative allostery release cycle. Nat Chem Biol 14, 811-820 (2018).
25. Zaitouna, A. J., Kaur, A. & Raghavan, M. Variations in MHC class I antigen presentation and immunopeptidome selection pathways. F1000Res 9, F1000 Faculty Rev-1177 (2020).
26. McShan, A. C. et al. Molecular determinants of chaperone interactions on MHC-I for folding and antigen repertoire selection. Proc. Natl. Acad. Sci. U.S.A. 116, 25602-25613 (2019).
27. Walker, B. A. et al. The dominantly expressed class I molecule of the chicken MHC is explained by coevolution with the polymorphic peptide transporter (TAP) genes. Proceedings of the National Academy of Sciences 108, 8396-8401 (2011).
28. Grimholt, U. Whole genome duplications have provided teleosts with many roads to peptide loaded MHC class I molecules. BMC Evol Biol 18, 1-14 (2018).
29. da Silva, A. P. & Gallardo, R. A. The Chicken MHC: Insights into Genetic Resistance, Immunity, and Inflammation Following Infectious Bronchitis Virus Infections. Vaccines (Basel) 8, 637 (2020).
30. Landis, E. D. et al. Identification and regulatory analysis of rainbow trout tapasin and tapasin-related genes. Immunogenetics 58, 56-69 (2006).
31. Margulies, D. H. et al. Chaperones and Catalysts: How Antigen Presentation Pathways Cope With Biological Necessity. Frontiers in Immunology 13, (2022).
32. Sidney, J., Peters, B., Frahm, N., Brander, C. & Sette, A. HLA class I supertypes: a revised and updated classification. BMC Immunology 9, 1 (2008).
33. Bouvier, M. & Wiley, D. C. Structural characterization of a soluble and partially folded class I major histocompatibility heavy chain/2m heterodimer. Nat Struct Mol Biol 5, 377-384 (1998).
34. Toebes, M. et al. Design and use of conditional MHC class I ligands. Nat Med 12, 246-251 (2006).
35. Rodenko, B. et al. Generation of peptide-MHC class I complexes through UV-mediated ligand exchange. Nat Protoc 1, 1120-1132 (2006).
36. Pei, R., Lee, J., Shih, N.-J., Chen, M. & Terasaki, P. I. Single human leukocyte antigen flow cytometry beads for accurate identification of human leukocyte antigen antibody specificities: Transplantation 75, 43-49 (2003).
37. Wittenbrink, N. et al. A novel approach reveals that HLA class 1 single antigen bead-signatures provide a means of high-accuracy pre-transplant risk assessment of acute cellular rejection in renal transplantation. BMC Immunol 20, 11 (2019).
38. Altman, J. D. et al. Phenotypic Analysis of Antigen-Specific T Lymphocytes. Science 274, 94-96 (1996).
39. Parham, P., Androlewicz, M. J., Brodsky, F. M., Holmes, N. J. & Ways, J. P. Monoclonal antibodies: purification, fragmentation and application to structural and functional studies of class I MHC antigens. J Immunol Methods 53, 133-173 (1982).
40. Ladasky, J., Shum, B., Canavez, F., Seuinez, H. & Parham, P. Residue 3 of 02-microglobulin affects binding of class I MHC molecules by the W6/32 antibody. Immunogenetics 49, 312-20 (1999).
41. Garboczi, D. N., Hung, D. T. & Wiley, D. C. HLA-A2-peptide complexes: refolding and crystallization of molecules expressed in Escherichia coli and complexed with single antigenic peptides. Proc Natl Acad Sci USA 89, 3429-3433 (1992).
42. McShan, A. C. et al. TAPBPR employs a ligand-independent docking mechanism to chaperone MR1 molecules. Nat Chem Biol 1-10 (2022) doi:10.1038/s41589-022-01049-9.
43. Buchli, R. et al. Real-Time Measurement of in Vitro Peptide Binding to Soluble HLA-A*0201 by Fluorescence Polarization. Biochemistry 43, 14852-14863 (2004).
44. Buchli, R. et al. Development and Validation of a Fluorescence Polarization-Based Competitive Peptide-Binding Assay for HLA-A*0201A New Tool for Epitope Discovery. Biochemistry 44, 12491-12507 (2005).
45. Bakker, A. H. et al. Conditional MHC class I ligands and peptide exchange technology for the human MHC gene products HLA-A1, -A3, -A11, and -B7. Proc Natl Acad Sci USA 105, 3825-3830 (2008).
46. McShan, A. C. et al. Peptide exchange on MHC-I by TAPBPR is driven by a negative allostery release cycle. Nat Chem Biol 14, 811-820 (2018).
47. Overall, S. A. et al. High throughput pMHC-I tetramer library production using chaperone-mediated peptide exchange. Nature Communications 11, 1909 (2020).
48. Buuren, M. M. van et al. HLA Micropolymorphisms Strongly Affect Peptide-MHC Multimer-Based Monitoring of Antigen-Specific CD8+ T Cell Responses. The Journal of Immunology 192, 641-648 (2014).
49. Hellman, L. M. et al. Differential scanning fluorimetry based assessments of the thermal and kinetic stability of peptide-MHC complexes. J Immunol Methods 432, 95-101 (2016).
50. Velazquez-Campoy, A., Leavitt, S. A. & Freire, E. Characterization of Protein-Protein Interactions by Isothermal Titration Calorimetry. in Protein-Protein Interactions: Methods and Applications (ed. Fu, H.) 35-54 (Humana Press, 2004). doi:10.1385/1-59259-762-9:035.
51. McShan, A. C. et al. TAPBPR promotes antigen loading on MHC-I molecules using a peptide trap. Nat Commun 12, 3174 (2021).
52. Masson, G. R. et al. Recommendations for performing, interpreting and reporting hydrogen deuterium exchange mass spectrometry (HDX-MS) experiments. Nat Methods 16, 595-602 (2019).
53. van Hateren, A. et al. Direct evidence for conformational dynamics in major histocompatibility complex class I molecules. J Biol Chem 292, 20255-20269 (2017).
54. Sagert, L., Hennig, F., Thomas, C. & Tampe, R. A loop structure allows TAPBPR to exert its dual function as MHC I chaperone and peptide editor. eLife 9, e55326 (2020).
55. Ilca, F. T. et al. TAPBPR mediates peptide dissociation from MHC class I using a leucine lever. eLife 7, e40126.
56. Xu, H., Song, K. & Da, L.-T. Dynamics of peptide loading into major histocompatibility complex class I molecules chaperoned by TAPBPR. Phys. Chem. Chem. Phys. (2022) doi:10.1039/D2CP00423B.
57. Ilca, F. T., Neerincx, A., Wills, M. R., de la Roche, M. & Boyle, L. H. Utilizing TAPBPR to promote exogenous peptide loading onto cell surface MHC I molecules. Proc Natl Acad Sci USA 115, E9353-E9361 (2018).
58. Ilca, F. T. & Boyle, L. H. The glycosylation status of MHC class I molecules impacts their interactions with TAPBPR. Mol Immunol 139, 168-176 (2021).
59. Grandea, A. G., Androlewicz, M. J., Athwal, R. S., Geraghty, D. E. & Spies, T. Dependence of peptide binding by MHC class I molecules on their interaction with TAP. Science 270, 105-108 (1995).
60. Chen, M. & Bouvier, M. Analysis of interactions in a tapasin/class I complex provides a mechanism for peptide selection. EMBO J 26, 1681-1690 (2007).
61. Sievers, F. et al. Fast, scalable generation of high-quality protein multiple sequence alignments using Clustal Omega. Mol Syst Biol 7, 539 (2011).
62. Robert, X. & Gouet, P. Deciphering key features in protein structures with the new ENDscript server. Nucleic Acids Res 42, W320-W324 (2014).
63. Kumar, S., Stecher, G. & Tamura, K. MEGA7: Molecular Evolutionary Genetics Analysis Version 7.0 for Bigger Datasets. Molecular Biology and Evolution 33, 1870-1874 (2016).
64. Raman, S. et al. Structure prediction for CASP8 with all-atom refinement using Rosetta. Proteins 77, 89-99 (2009).
65. Song, Y. et al. High resolution comparative modeling with RosettaCM. Structure 21, 10.1016/j.str.2013.08.005 (2013).
66. Li, H., Natarajan, K., Malchiodi, E. L., Margulies, D. H. & Mariuzza, R. A. Three-dimensional structure of H-2Dd complexed with an immunodominant peptide from human immunodeficiency virus envelope glycoprotein 120. Journal of Molecular Biology 283, 179-191 (1998).
67. Park, J. et al. Structural architecture of a dimeric class C GPCR based on co-trafficking of sweet taste receptor subunits. J Biol Chem 294, 4759-4774 (2019).
68. Fowler, D. M., Araya, C. L., Gerard, W. & Fields, S. Enrich: software for analysis of protein function by enrichment and depletion of variants. Bioinformatics 27, 3430-3431 (2011).
69. Thomsen, M. C. F. & Nielsen, M. Seq2Logo: a method for construction and visualization of amino acid binding motifs and sequence profiles including sequence weighting, pseudo counts and two-sided representation of amino acid enrichment and depletion. Nucleic Acids Research 40, W281-W287 (2012).
70. Rodenko, B. et al. Class I Major Histocompatibility Complexes Loaded by a Periodate Trigger. J. Am. Chem. Soc. 131, 12305-12313 (2009).
71. Johnson, K. A. & Goody, R. S. The Original Michaelis Constant: Translation of the 1913 Michaelis-Menten Paper. Biochemistry 50, 8264-8269 (2011).
72. Mage, M. G. et al. THE PEPTIDE-RECEPTIVE TRANSITION STATE OF MHC-I MOLECULES: INSIGHT FROM STRUCTURE AND MOLECULAR DYNAMICS. J Immunol 189, 1391-1399 (2012).
73. van Hateren, A. & Elliott, T. The role of MHC I protein dynamics in tapasin and TAPBPR-assisted immunopeptidome editing. Current Opinion in Immunology 70, 138-143 (2021).
74. Bailey, A. et al. Selector function of MHC I molecules is determined by protein plasticity. Sci Rep 5, 14928 (2015).
75. Hilvert, D. Critical Analysis of Antibody Catalysis. Annual Review of Biochemistry 69, 751-793 (2000).
76. R. P. Cheng, S. H. Gellman, W. F. DeGrado, β-Peptides: From Structure to Function. Chem. Rev. 101, 3219-3232 (2001).
77. J. R. Allison, M. Müller, W. F. van Gunsteren, A comparison of the different helices adopted by α- and β-peptides suggests different reasons for their stability. Protein Sci. 19, 2186-2195 (2010).

TABLE 2
Summary of peptides used in this Example.

| Peptide Name | Allotype | Sequence |
|---|---|---|
| PhotoA01 | A*01:01 | STAPGJLEY (SEQ ID NO: 9) |
| PhotoA02 | A*02:01 | KILGFVFJV (SEQ ID NO: 25) |
| PhotoA24 | A*24:02 | VYGJVRACL (SEQ ID NO: 3) |
| PhotoA30 | A*30:01 | AIFQSSMJK (SEQ ID NO: 58) |
| PhotoA29 | A*29:02 | VFAQVKQJY (SEQ ID NO: 59) |
| PhotoA68 | A*68:02 | SVYDFFVJL (SEQ ID NO: 101) |
| PhotoA11 | A*11:01 | KLIETYFJK (SEQ ID NO: 102) |
| PhotoB08 | B*08:01 | FLRGRAJGL (SEQ ID NO: 4) |
| PhotoB37 | B*37:01 | FEDLRVJSF (SEQ ID NO: 63) |
| PhotoE01 | E*01:03 | VMAPJTLVL (SEQ ID NO: 64) |
| PhotoG01 | G*01:01 | VMAPJTLVL (SEQ ID NO: 65) |
| BetaA01 | A*01:01 | STAPG(BF)LEY (SEQ ID NO: 14) |
| BetaA02 | A*02:01 | KILGFVF(BF)V (SEQ ID NO: 12) |
| BetaA03_A11 | A*03:01 A*11:01 | KLIETYF(BF)K (SEQ ID NO: 103) |
| BetaB37 | B*37:01 | FEDLRV(BF)SF (SEQ ID NO: 17) |
| FITCA01 | A*01:01 | FITC-KSDPIVAQY (SEQ ID NO: 34) |
| TAMRAA02 | A*02:01 | TAMRA-KLFGYPVYV (SEQ ID NO: 27) |
| TAMRAA24 | A*24:02 | TAMRA-KYNPIRTTF (SEQ ID NO: 36) |
| FITCA03_A11 | A*03:01 A*11:01 | FITC-KLIETYFSK (SEQ ID NO: 35) |
| FITCB08 | B*08:01 | FITC-KLRGRAYGL (SEQ ID NO: 78) |
| FITCB37 | B*37:01 | FITC-KEDLRVSSF (SEQ ID NO: 84) |
| Phox2b | A*24:02 | QYNPIRTTF (SEQ ID NO: 77) |
| TAX9 | A*02:01 | LLFGYPVYV (SEQ ID NO: 26) |
| p29 | A*02:01 & A*01:01 nonbinder | YPNVNIHNF (SEQ ID NO: 8) |
| RAS | A*01:01 | ILDTAGQEEY (SEQ ID NO: 83) |
| Titin | A*01:01 | ESDPIVAQY (SEQ ID NO: 6) |
| SARS P37 | A*01:01 | CTDDNALAYY (SEQ ID NO: 5) |
| SARS P39 | A*01:01 | PTDNYITTY (SEQ ID NO: 7) |
| HCMV | B*08:01 | RPHERNGFTVL (SEQ ID NO: 10) |
| EBV | B*08:01 | FLRGRAYGL (SEQ ID NO: 21) |
| T1D30 | B*08:01 | ALWMRLLPL (SEQ ID NO: 79) |
| T1D95 | B*08:01 | MLYQHLLPL (SEQ ID NO: 80) |
| HIV | B*08:01 nonbinder | AIFQSSMTK (SEQ ID NO: 81) |
| NP338 | B*37:01 | FEDLRVLSF (SEQ ID NO: 22) |
| CMV pp65 | B*37:01 nonbinder | NLVPMVATV (SEQ ID NO: 82) |
| PhotoA01 | A*01:01 | STAPGJLEY (SEQ ID NO: 9) |
| PhotoA02 | A*02:01 | KILGFVFJV (SEQ ID NO: 25) |
| PhotoA24 | A*24:02 | VYGJVRACL (SEQ ID NO: 3) |
| PhotoA30 | A*30:01 | AIFQSSMJK (SEQ ID NO: 58) |
| PhotoA29 | A*29:02 | VFAQVKQJY (SEQ ID NO: 59) |
| PhotoA68 | A*68:02 | SVYDFFVJL (SEQ ID NO: 101) |
| PhotoA11 | A*11:01 | KLIETYFJK (SEQ ID NO: 102) |
| PhotoB08 | B*08:01 | FLRGRAJGL (SEQ ID NO: 4) |
| PhotoB37 | B*37:01 | FEDLRVJSF (SEQ ID NO: 63) |
| PhotoE01 | E*01:03 | VMAPJTLVL (SEQ ID NO: 64) |
| PhotoG01 | G*01:01 | VMAPJTLVL (SEQ ID NO: 65) |
| BetaA01 | A*01:01 | STAPG(BF)LEY (SEQ ID NO: 14) |
| BetaA02 | A*02:01 | KILGFVF(BF)V (SEQ ID NO: 12) |
| BetaA03_A11 | A*03:01 A*11:01 | KLIETYF(BF)K (SEQ ID NO: 103) |
| BetaB37 | B*37:01 | FEDLRV(BF)SF (SEQ ID NO: 17) |
| FITCA01 | A*01:01 | FITC-KSDPIVAQY (SEQ ID NO: 34) |
| TAMRAA02 | A*02:01 | TAMRA-KLFGYPVYV (SEQ ID NO: 27) |
| TAMRAA24 | A*24:02 | TAMRA-KYNPIRTTF (SEQ ID NO: 36) |
| FITCA03_A11 | A*03:01 A*11:01 | FITC-KLIETYFSK (SEQ ID NO: 40) |
| FITCB08 | B*08:01 | FITC-KLRGRAYGL (SEQ ID NO: 78) |
| FITCB37 | B*37:01 | FITC-KEDLRVSSF (SEQ ID NO: 84) |
| Phox2b | A*24:02 | QYNPIRTTF (SEQ ID NO: 77) |
| TAX9 | A*02:01 | LLFGYPVYV (SEQ ID NO: 26) |
| p29 | A*02:01 & A*01:01 nonbinder | YPNVNIHNF (SEQ ID NO: 8) |
| RAS | A*01:01 | ILDTAGQEEY (SEQ ID NO: 83) |
| Titin | A*01:01 | ESDPIVAQY (SEQ ID NO: 6) |
| SARS P37 | A*01:01 | CTDDNALAYY (SEQ ID NO: 5) |
| SARS P39 | A*01:01 | PTDNYITTY (SEQ ID NO: 7) |
| HCMV | B*08:01 | RPHERNGFTVL (SEQ ID NO: 10) |
| EBV | B*08:01 | FLRGRAYGL (SEQ ID NO: 21) |
| T1D30 | B*08:01 | ALWMRLLPL (SEQ ID NO: 79) |
| T1D95 | B*08:01 | MLYQHLLPL (SEQ ID NO: 80) |
| HIV | B*08:01 nonbinder | AIFQSSMTK (SEQ ID NO: 81) |
| NP338 | B*37:01 | FEDLRVLSF (SEQ ID NO: 22) |
| CMV pp65 | B*37:01 nonbinder | NLVPMVATV (SEQ ID NO: 82) |

### Example 11: Recombinant Chicken Tapasin (a Homolog of TAPBPR)

TABLE 3
Summary of Tₘ of HLA-A*02 subtypes loaded with KILGFVFβFV
(SEQ ID NO: 12) and HLA-A*02:01 loaded with peptides from
the βF scanning peptide panel.

| Micropolymorphic A*02 | Tₘ (° C.) |
|---|---|
| A*02:01/BetaA02 | 52.6 |
| A*02:06/BetaA02 | 53.1 |
| A*02:11/BetaA02 | 53.8 |
| A*02:71/BetaA02 | 54.2 |
| A*02:77/BetaA02 | 54.2 |
| A*02:01/βFILGFVFTV (SEQ ID NO: 66) | 53.9 |
| A*02:01/KIβFGFVFTV (SEQ ID NO: 67) | 58.1 |
| A*02:01/KILβFFVFTV (SEQ ID NO: 68) | 54.3 |
| A*02:01/KILGβFVFTV (SEQ ID NO: 69) | 61.8 |
| A*02:01/KILGFβFFTV (SEQ ID NO: 70) | 60.7 |
| A*02:01/KILGFVβFTV (SEQ ID NO: 71) | 63.3 |
| A*02:01/KILGFVFβFV (SEQ ID NO: 12) | 53.2 |

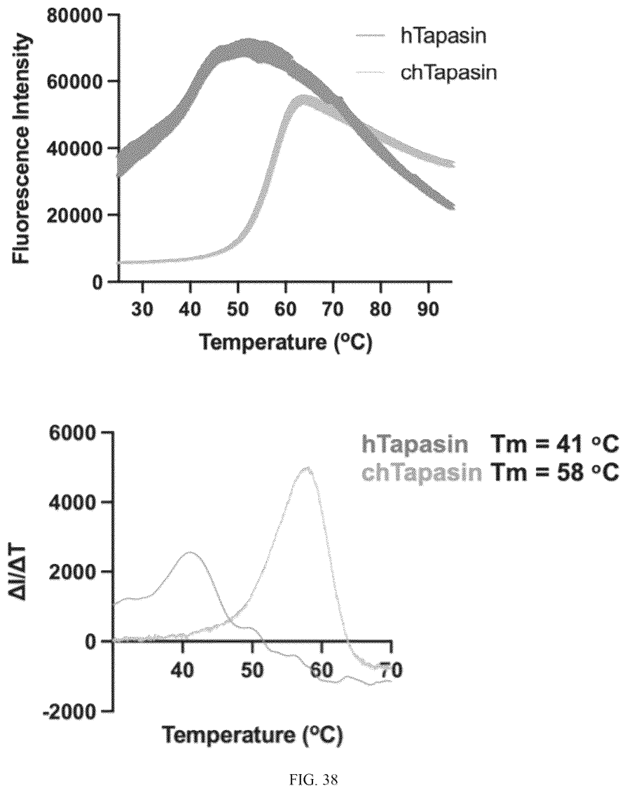

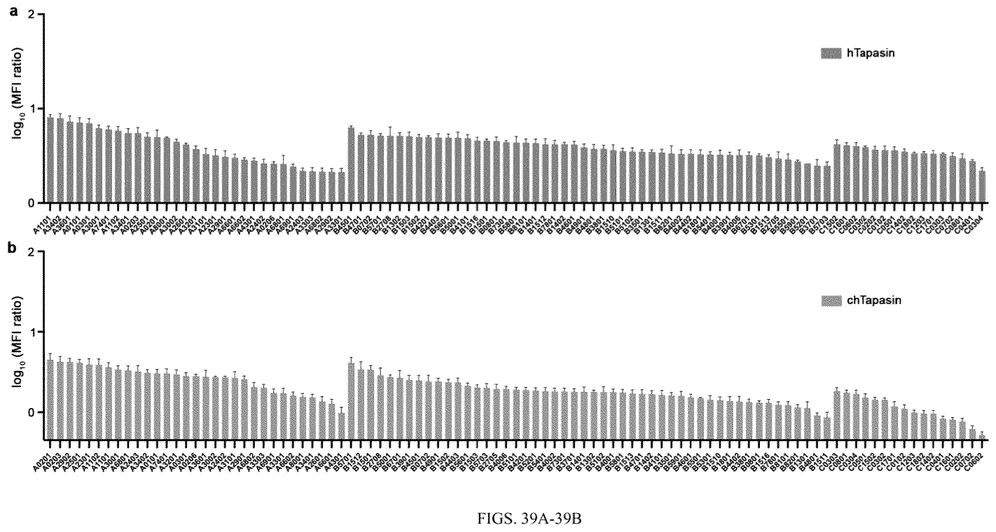

Applicants' results using recombinant chicken Tapasin (a homolog of TAPBPR), which has favorable properties over its human version are presented in FIGS. 38 and 39. While human Tapasin has been studied extensively and its uses as a peptide exchange catalyst has been considered (albeit with limited efficacy, due to its unstable nature), there are currently no published data exploring Xeno-interactions between chicken Tapasin and HLAs. chTapasin is a much more stable protein scaffold to use for peptide exchange and protein engineering applications according to its thermal stability measurements (FIG. 38). The WT chTapasin protein can interact with a range of disease-relevant HLAs.

### Example 12: Tapasins and Mafa

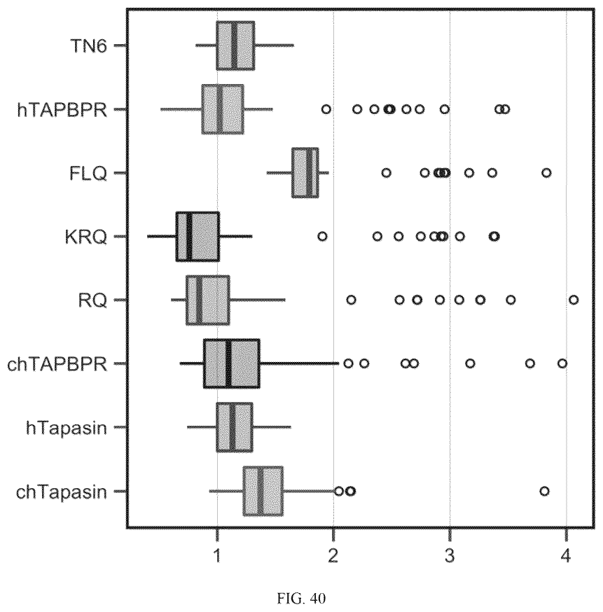

FIG. 40. chTapasin MFI ratios plot. Molecular chaperones and their engineered variants can interact with different sets of folded HLA-I allotypes. Box plot showing distributions of log₁₀ Mean Fluorescence Intensity (MFI) levels for binding of fluorescent tetramers conjugated with either human TAPBPR (hTAPBPR), three engineered mutants of human TAPBPR (FLQ, KRQ, RQ corresponding to the hTAPBPR^{FLQ} hTAPBPR^{KRQ} and hTAPBPR^{RQ} variants, respectively), chicken TAPBPR (chTAPBPR), human Tapasin (hTapasin) or chicken Tapasin (chTapasin) to single-antigen beads (SABs) conjugated with 97 common HLA-I allotypes. The left boundary of the box indicates the 25^{th} percentile, the line within the box marks the median and the right boundary of the box indicates the 75^{th} percentile. Whiskers above and below the box indicate the 10^{th} and 90^{th} percentiles. The points above the whiskers indicate outliers outside the 90^{th} percentile. A mutant form of the human chaperone TAPBPR, hTAPBPR^{TN6} (TN6) shown previously to abrogate binding to MHC-I molecules (Dong et al., Immunity; Hermann et al., J. Immunol) was used as a negative control for background staining levels, to determine significant interactions between TAPBPR orthologs and MHC-I molecules, and a cutoff value of 1.8 log₁₀(MFI) was used to determine significance levels. HLA allotypes detected to bind above this threshold and are therefore indicated to interact with each chaperone in either the peptide-loaded or peptide-deficient states are outlined in Table 4.

TABLE 4
HLA allotypes identified to interact with indicated molecular chaperones using
a single-antigen bead assay, according to the data shown in FIG. 40.

| chTapasin | hTAPBPR | chTAPBPR | hTAPBPR^{RQ} | hTAPBPR^{KRQ} | hTAPBPR^{FLQ} | | | |
|---|---|---|---|---|---|---|---|---|
| A2902 | A0201 | A2301 | A0201 | A0201 | A0201 | B1401 | B3901 | C0202 |
| B3701 | A0203 | A2402 | A0203 | A0203 | A0203 | B1501 | B4001 | C0302 |
| C0102 | A0206 | A2403 | A0206 | A0206 | A0206 | B1502 | B4002 | C0303 |
| C0202 | A2301 | A2901 | A2301 | A2301 | A2301 | B1503 | B4006 | C0304 |
| C0401 | A2402 | A2902 | A2402 | A2402 | A2402 | B1510 | B4101 | C0501 |
| C0602 | A2403 | A6802 | A2403 | A2403 | A2403 | B1512 | B4201 | C0602 |
| C0702 | A2901 | B0801 | A2902 | A2902 | A2902 | B1516 | B4901 | C0801 |
| C1402 | A2902 | B3701 | A6802 | A6802 | A6801 | B1801 | B4601 | C1203 |
| C1601 | A6802 | C0102 | A6901 | A6901 | A6802 | B2708 | B5401 | C1402 |
| C1701 | A6901 | C0303 | B3701 | B3701 | A6901 | B3501 | B5501 | C1502 |
| C1802 | B3701 | C0304 | | | B0702 | B3701 | B6701 | C1802 |
| | | | | | | | | C0102 |

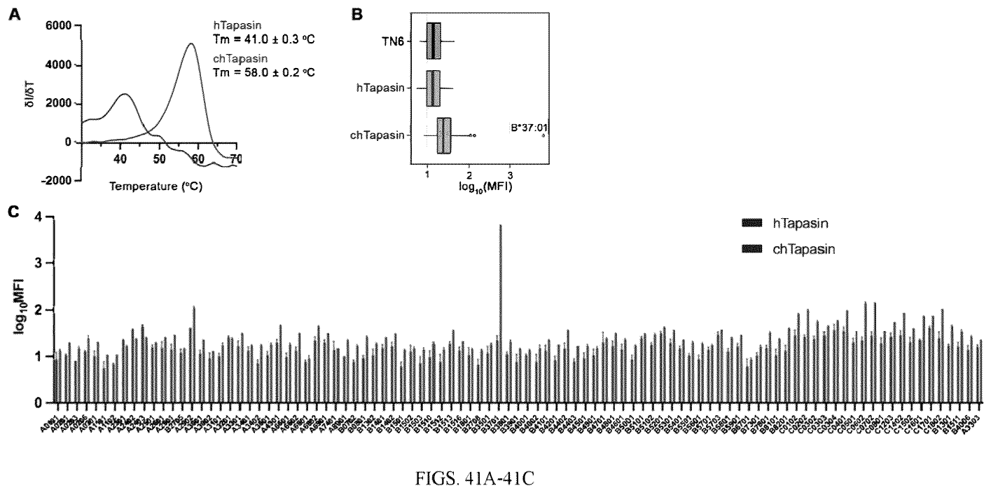

FIGS. 41A-41C. chTapasin MFI ratios plot. Molecular chaperones and their engineered variants can interact with different sets of folded HLA-I allotypes. Chicken Tapasin shows enhanced association with human HLA-I allotypes and forms a strong interaction with HLA-B*37:01. A. Differential Scanning Fluorimetry of recombinantly expressed chicken Tapasin (chTapasin) showing an increased thermal stability (Tₘ 58° C.), relative to its human ortholog (hTapasin, Tₘ 41.0° C.). B. Box plot showing distributions of Mean Fluorescence Intensity (MFI) levels for binding of fluorescent tetramers conjugated with either human or chicken Tapasin to single-antigen beads (SABs) conjugated with 97 common HLA-I allotypes. A mutant form of the human chaperone TAPBPR, hTAPBPR^{TN6}, shown previously to aburaage binding to MIC-I molecules (Dong et al., Immunity. 2009; 30:21-32; Hermann et al., J. Immunol 2013 Dec. 1; 191(11):5743-50) was used as a negative control for background staining levels, to determine significant interactions between TAPBPR orthologs and MIC-I molecules. The left boundary of the box indicates the 25^{th} percentile, the line within the box marks the median and the right boundary of the box indicates the 75^{th} percentile. Whiskers above and below the box indicate the 10^{th} and 90^{th} percentiles. The points above the whiskers indicate outliers outside the 90^{th} percentile. Overall, chicken Tapasin shows enhanced association with human MHC-I allotypes.HLA-B*37:01, which shows tight binding to chTapasin, is indicated. C. Bar graph showing log₁₀ Mean Fluorescence Intensity (MFI) levels for binding of fluorescent tetramers conjugated with either human or chicken Tapasin to single-antigen beads (SABs) conjugated with 97 common HLA-I allotypes. Overall, chicken Tapasin shows enhanced association with human MHC-I allotypes. HLA-B*37:01, which exhibits strong association with chicken Tapasin, is indicated. Plotted data are mean±SD from 2 independent experiments.

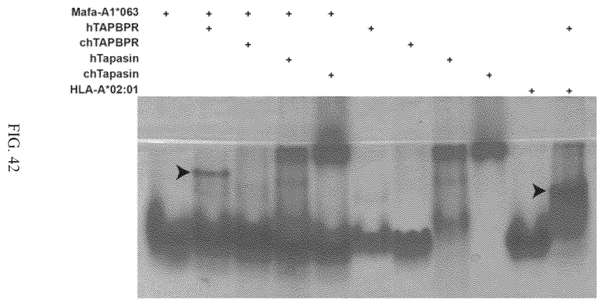

FIG. 42. The macaque MHC-I Mafa-A1*063 forms a tight complex with the human chaperone TAPBPR. Native polyacrylamide gel electrophoresis analysis of interactions between different TAPBPR or Tapasin orthologs and Mafa-A1*063. Each well in lanes 1-9 was loaded with Mafa-A1*063/hβ₂m/GPRKPIKCW (SEQ ID NO: 86) complex, hTAPBPR, chTAPBPR, hTapasin, and chTapasin, or 1:1 molar ratio mixture, as indicated. As a positive control (lanes 10 and 11), we incubated HLA-A*02:01/hβ₂m/KILGFVFJV (SEQ ID NO: 5), where J=3-amino-3-(2-nitrophenyl)-propionic acid with human TAPBPR for 1 hour under 365 nm UV light, leading to peptide dissociation and formation of an empty TAPBPR/HLA-A*02:01/hβ₂m protein complex. New protein bands corresponding to the formed Mafa-A1*063/hTAPBPR and HLA-A*02:01/hTAPBPR complexes are indicated with arrows.

TABLE 5
Mean fluorescence intensity (MFI) ratio of human and chicken
TAPBPR relative to the control W6/32 experiments. Alleles with
ratios above the threshold (MFI ratio = 5.97) are in bold.

MHC-I MFI ratio

| Allotype | hTAPBPR | chTAPBPR |
|---|---|---|
| A*01:01 | 3.90 | 2.67 |
| A*02:01 | 39.68 | 3.88 |
| A*02:03 | 19.10 | 3.28 |
| A*02:06 | 12.75 | 2.97 |
| A*03:01 | 4.38 | 5.63 |
| A*11:01 | 3.63 | 3.11 |
| A*11:02 | 4.63 | 4.09 |
| A*23:01 | 49.93 | 9.25 |
| A*24:02 | 20.32 | 20.80 |
| A*24:03 | 15.50 | 22.43 |
| A*25:01 | 5.20 | 4.77 |
| A*26:01 | 1.79 | 2.32 |
| A*29:01 | 3.88 | 11.42 |
| A*29:02 | 74.26 | 528.17 |
| A*30:01 | 3.75 | 3.08 |
| A*30:02 | 2.88 | 1.80 |
| A*31:01 | 3.69 | 2.73 |
| A*32:01 | 8.71 | 15.37 |
| A*33:01 | 2.63 | 1.56 |
| A*33:03 | 2.00 | 2.05 |
| A*34:01 | 3.87 | 3.52 |
| A*34:02 | 4.00 | 3.50 |
| A*36:01 | 3.50 | 2.50 |
| A*43:01 | 2.39 | 2.23 |
| A*66:01 | 1.66 | 2.30 |
| A*66:02 | 2.32 | 3.19 |
| A*68:01 | 6.13 | 3.04 |
| A*68:02 | 115.17 | 4.45 |
| A*69:01 | 10.17 | 2.33 |
| A*74:01 | 6.08 | 7.53 |
| A*80:01 | 2.88 | 2.10 |
| B*07:02 | 2.58 | 2.41 |
| B*08:01 | 3.38 | 16.59 |
| B*13:01 | 1.79 | 1.70 |
| B*13:02 | 2.50 | 1.56 |
| B*14:01 | 2.93 | 2.15 |
| B*14:02 | 2.43 | 1.58 |
| B*15:01 | 3.28 | 1.39 |
| B*15:02 | 2.48 | 1.87 |
| B*15:03 | 2.31 | 1.46 |
| B*15:10 | 2.59 | 4.20 |
| B*15:11 | 1.55 | 1.39 |
| B*15:13 | 2.34 | 3.54 |
| B*15:16 | 2.71 | 3.18 |
| B*18:01 | 1.95 | 1.24 |
| B*27:05 | 3.23 | 1.77 |
| B*27:08 | 2.29 | 1.21 |
| B*35:01 | 1.97 | 1.55 |
| B*37:01 | 50.02 | 1126.35 |
| B*38:01 | 3.13 | 5.91 |
| B*39:01 | 3.35 | 1.88 |
| B*40:01 | 1.62 | 1.27 |
| B*40:02 | 2.44 | 1.39 |
| B*40:06 | 1.82 | 1.31 |
| B*41:01 | 2.39 | 2.14 |
| B*42:01 | 2.79 | 2.09 |
| B*44:02 | 2.38 | 2.63 |
| B*44:03 | 2.14 | 2.16 |
| B*45:01 | 2.86 | 2.23 |
| B*46:01 | 2.21 | 1.80 |
| B*47:01 | 3.00 | 2.60 |
| B*48:01 | 1.88 | 2.16 |
| B*49:01 | 3.24 | 2.04 |
| B*50:01 | 2.43 | 1.62 |
| B*51:01 | 3.10 | 3.32 |
| B*51:02 | 3.14 | 3.95 |
| B*52:01 | 2.48 | 2.43 |
| B*53:01 | 1.83 | 2.12 |
| B*54:01 | 2.07 | 1.80 |
| B*55:01 | 1.74 | 1.47 |
| B*56:01 | 2.81 | 1.67 |
| B*57:01 | 4.33 | 2.86 |
| B*57:03 | 2.74 | 6.79 |
| B*58:01 | 2.09 | 1.95 |
| B*59:01 | 2.71 | 6.49 |
| B*67:01 | 3.06 | 1.87 |
| B*73:01 | 4.86 | 5.09 |
| B*78:01 | 2.28 | 1.64 |
| B*81:01 | 1.73 | 1.82 |
| B*82:01 | 1.63 | 1.38 |
| C*01:02 | 2.49 | 6.40 |
| C*02:02 | 1.84 | 1.49 |
| C*03:02 | 2.64 | 1.95 |
| C*03:03 | 2.56 | 5.46 |
| C*03:04 | 2.65 | 8.72 |
| C*04:01 | 1.65 | 1.76 |
| C*05:01 | 1.85 | 1.34 |
| C*06:02 | 1.36 | 1.59 |
| C*07:02 | 1.58 | 1.32 |
| C*08:01 | 1.62 | 1.12 |
| C*12:03 | 1.57 | 2.00 |
| C*14:02 | 1.71 | 1.36 |
| C*15:02 | 2.00 | 1.52 |
| C*16:01 | 1.60 | 1.29 |
| C*17:01 | 1.83 | 1.59 |
| C*18:02 | 1.58 | 1.64 |

TABLE 6
Summary of MHC-I residues contacting TAPBPR for selected HLA alleles
used in this study, including HLA-A*01:01, A*30:01, A*29:02, A*02:01, A*68:02,
A*03:01, A*11:01, A*24:02, B*08:01, B*37:01, E*01:03, and G*01:01.

| | Amino Acid Position [84, 86, 113, 115, 120, 122, 127, 128, 134, 135, |
|---|---|
| HLA Allele | 136, 138, 141, 142, 144, 145, 148, 225, 228, 229, 230, 231, 232, 244] |
| A*01:01 | YNYQGDNETAAMQIKRETTELVEW (SEQ ID NO: 104) |
| A*30:01 | YNYQGDNETAAMQIQRETTELVEW (SEQ ID NO: 105) |
| A*29:02 | YNYQGDNETAAMQIQRETTELVEW (SEQ ID NO: 105) |
| A*02:01 | YNYQGDKETAAMQTKHETTELVEW (SEQ ID NO: 106) |
| A*68:02 | YNYQGDKETAAMQTKHETTELVEW (SEQ ID NO: 106) |
| A*03:01 | YNYQGDNETAAMQIKRETTELVEW (SEQ ID NO: 104) |
| A*11:01 | YNYQGDNETAAMQIKRETTELVEW (SEQ ID NO: 104) |
| A*24:02 | YNYQGDKETAAMQIKRETTELVEW (SEQ ID NO: 107) |
| B*08:01 | YNHQGDNETAATQIQRETTELVEW (SEQ ID NO: 108) |
| B*37:01 | YNYQGDNETAATQIQRETTELVEW (SEQ ID NO: 109) |
| E*01:03 | YNYQGDNETAVTQIEQNTTELVEW (SEQ ID NO: 110) |
| G*01:01 | YNYQGDNETAATQIKRETVELVEW (SEQ ID NO: 111) |

### Example 13

FIGS. 43A-43D. A graphic depiction of the free energy landscape for TAPBPR-mediated peptide exchange. A. MHC-I loaded with a moderate affinity placeholder peptide can spontaneously but slowly generate empty molecules. B. These empty molecules overcome a high activation energy barrier (ΔG^{‡}_{uncat}) to be in the transient peptide-receptive state for peptide loading. C. A catalytic amount of TAPBPR functions to lower the activation energy barrier (ΔG^{‡}_{cat}) by recognizing the empty molecules with high affinity (binding free energy ΔG_{B2}) and stabilizing an “open” conformation for peptide loading. D. Loading of high affinity peptides induces a “closed” MHC-I groove conformation leading to TAPBPR dissociation, due to the substantially reduced affinity of TAPBPR towards peptide-loaded molecules (indicated by binding free energies ΔG_{B1} and ΔG_{B3}). The reaction coordinate diagram from left-to-right shows the exchange of a moderate affinity peptide to a high affinity peptide, which can happen in the opposite direction with a much higher activation energy barrier and thus slower kinetics.

### Example 14

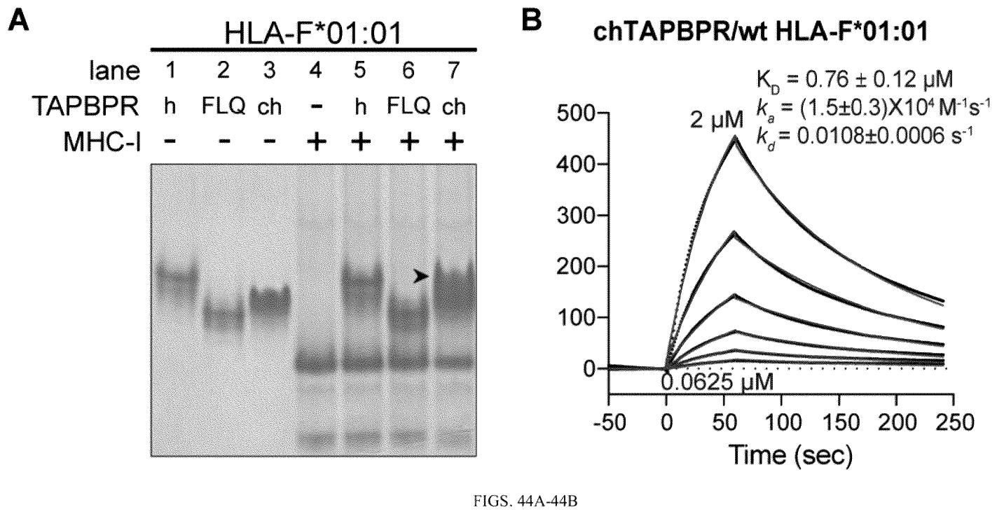

FIGS. 44A-44B. The chicken chaperone TAPBPR forms a tight complex with the HLA-F*01:01 allotype. A. Native polyacrylamide gel electrophoresis analysis of interactions between human TAPBPR wild-type (hTAPBPR), hTAPBPR carrying the mutations S104F, K21 IL and K270Q (FLQ), and chicken TAPBPR with HLA-F*01:01. Each well in lanes 1-7 was loaded with HLA-F*01:01/hβ₂m/LILRWE(bF)D (SEQ ID NO: 112) complex, hTAPBPR, hTAPBPR^{FLQ} and chTAPBPR, or 1:1 molar ratio mixture, as indicated. A new protein band corresponding to the formed HLA-F*01:01/chTAPBPR complex is indicated by the arrow (lane 7). B. Representative sensorgram of HLA-F*01:01/hβ₂m/LILRWE(bF)D (SEQ ID NO: 112) flowed over a streptavidin chip coupled with chTAPBPR-biotin. K_{D}, equilibrium constant; kₐ, association rate constant; k_{d}, dissociation rate constant; RU, resonance units. The concentrations of analyte for the top and the bottom sensorgrams are noted. Fits from the kinetic analysis are shown with red lines.

### REFERENCES

Altman, John D., Paul A. H. Moss, Philip J. R. Goulder, Dan H. Barouch, Michael G. McHeyzer-Williams, John I. Bell, Andrew J. McMichael, and Mark M. Davis. 1996. “Phenotypic Analysis of Antigen-Specific T Lymphocytes.” Science 274 (5284): 94-96. https://doi.org/10.1126/science.274.5284.94.
Bakker, A. H., R. Hoppes, C. Linnemann, M. Toebes, B. Rodenko, C. R. Berkers, S. R. Hadrup, et al. 2008. “Conditional MHC Class I Ligands and Ligand exchange Technology for the Human MHC Gene Products HLA-A1, -A3, -A11, and -B7.” Proceedings of the National Academy of Sciences 105 (10): 3825-30. https://doi.org/10.1073/pnas.0709717105.
Boyle, Louise H., Clemens Hermann, Jessica M. Boname, Keith M. Porter, Peysh A. Patel, Marian L. Burr, Lidia M. Duncan, et al. 2013. “Tapasin-Related Protein TAPBPR Is an Additional Component of the MHC Class I Presentation Pathway.” Proceedings of the National Academy of Sciences 110 (9): 3465-70. https://doi.org/10.1073/pnas.1222342110.
Ilca, F. Tudor, Andreas Neerincx, Mark R. Wills, Maike de la Roche, and Louise H. Boyle. 2018. “Utilizing TAPBPR to Promote Exogenous Peptide Loading onto Cell Surface MHC I Molecules.” Proceedings of the National Academy of Sciences 115 (40): E9353-61. https://doi.org/10.1073/pnas.1809465115.
Jiang, Jiansheng, Kannan Natarajan, Lisa F. Boyd, Giora I. Morozov, Michael G. Mage, and David H. Margulies. 2017. “Crystal Structure of a TAPBPR-MHC I Complex Reveals the Mechanism of Peptide Editing in Antigen Presentation.” Science 358 (6366): 1064-68. https://doi.org/10.1126/science.aao5154.
O'Rourke, Sara M, Giora I Morozov, Jacob T Roberts, Adam W Barb, and Nikolaos G Sgourakis. 2019. “Production of Soluble PMHC-I Molecules in Mammalian Cells Using the Molecular Chaperone TAPBPR.” Protein Engineering, Design and Selection 32 (12): 525-32. https://doi.org/10.1093/protein/gzaa015.
Overall, Sarah A., Jugmohit S. Toor, Stephanie Hao, Mark Yarmarkovich, Sara M. O'Rourke, Giora I. Morozov, Son Nguyen, et al. 2020. “High Throughput PMHC-I Tetramer Library Production Using Chaperone-Mediated Ligand exchange.” Nature Communications 11 (1): 1909. https://doi.org/10.1038/s41467-020-15710-1.
Rossjohn, Jamie, Stephanie Gras, John J. Miles, Stephen J. Turner, Dale I. Godfrey, and James McCluskey. 2015. “T Cell Antigen Receptor Recognition of Antigen-Presenting Molecules.” Annual Review of Immunology 33 (1): 169-200. https://doi.org/10.1146/annurev-immunol-032414-112334.
Sidney, John, Bjoern Peters, Nicole Frahm, Christian Brander, and Alessandro Sette. 2008. “HLA Class I Supertypes: A Revised and Updated Classification.” BMC Immunology 9 (1): 1. https://doi.org/10.1186/1471-2172-9-1.
Teng, Michelle S., Richard Stephens, Louis Du Pasquier, Tom Freeman, Jonathan A. Lindquist, and John Trowsdale. 2002. “A HumanTAPBP (TAPASIN)-Related Gene, TAPBP-R.” European Journal of Immunology 32 (4): 1059-68. https://doi.org/10.1002/1521-4141(200204)32:4<1059::AID-IMMU1059>3.0.CO;2-G.
Vita, Randi, Swapnil Mahajan, James A Overton, Sandeep Kumar Dhanda, Sheridan Martini, Jason R Cantrell, Daniel K Wheeler, Alessandro Sette, and Bjoern Peters. 2019. “The Immune Epitope Database (IEDB): 2018 Update.” Nucleic Acids Research 47 (D1): D339-43.
https://doi.org/10.1093/nar/gky1006.
Corbett, A. J., Awad, W., Wang, H. & Chen, Z. Antigen Recognition by MR1-Reactive T Cells; MAIT Cells, Metabolites, and Remaining Mysteries. Front. Immunol. 11, 1961 (2020).
Crowther, M. D. et al. Genome-wide CRISPR-Cas9 screening reveals ubiquitous T cell cancer targeting via the monomorphic MHC class I-related protein MR1. Nat. Immunol. 21, 178-185 (2020).
Chen, Z. et al. Mucosal-associated invariant T-cell activation and accumulation after in vivo infection depends on microbial riboflavin synthesis and co-stimulatory signals. Mucosal Immunol. 10, 58-68 (2017).
Rouxel, O. et al. Cytotoxic and regulatory roles of mucosal-associated invariant T cells in type 1 diabetes. Nat. Immunol. 18, 1321-1331 (2017).
Kjer-Nielsen, L. et al. MR1 presents microbial vitamin B metabolites to MAIT cells. Nature 491, 717-723 (2012).
Salio, M. et al. Ligand-dependent downregulation of MR1 cell surface expression. Proc. Natl. Acad. Sci. U.S.A 117, 10465-10475 (2020).
Keller, A. N. et al. Drugs and drug-like molecules can modulate the function of mucosal-associated invariant T cells. Nat. Immunol. 18, 402-411 (2017).
Corbett, A. J. et al. T-cell activation by transitory neo-antigens derived from distinct microbial pathways. Nature 509, 361-365 (2014).
Lepore, M. et al. Functionally diverse human T cells recognize non-microbial antigens presented by MR1. eLife 6, e24476 (2017).
Altman, J. D., P. A. Moss, P. J. Goulder, D. H. Barouch, M. G. McHeyzer-Williams, J. I. Bell, A. J. McMichael, and M. M. Davis. 1996. “Phenotypic Analysis of Antigen-Specific T Lymphocytes.” Science (New York, N.Y.) 274 (5284): 94-96. https://doi.org/10.1126/science.274.5284.94.
Boyle, Louise H., Clemens Hermann, Jessica M. Boname, Keith M. Porter, Peysh A. Patel, Marian L. Burr, Lidia M. Duncan, et al. 2013. “Tapasin-Related Protein TAPBPR Is an Additional Component of the MHC Class I Presentation Pathway.” Proceedings of the National Academy of Sciences 110 (9): 3465-70. https://doi.org/10.1073/pnas.1222342110.
Jiang, Jiansheng, Kannan Natarajan, Lisa F. Boyd, Giora I Morozov, Michael G. Mage, and David H. Margulies. 2017. “Crystal Structure of a TAPBPR-MHC I Complex Reveals the Mechanism of Peptide Editing in Antigen Presentation.” Science (New York, N.Y.) 358 (6366): 1064-68. https://doi.org/10.1126/science.aao5154.
Ljunggren, Hans-Gustaf, Nico J. Stam, Claes Ohlen, Jacques J. Neefjes, Petter Höglund, Marie-Thérèse Heemels, Judy Bastin, et al. 1990. “Empty MHC Class I Molecules Come out in the Cold.” Nature 346 (6283): 476-80. https://doi.org/10.1038/346476a0.
McShan, Andrew C., Kannan Natarajan, Vlad K. Kumirov, David Flores-Solis, Jiansheng Jiang, Mareike Badstubner, Jugmohit S. Toor, et al. 2018. “Peptide Exchange on MHC-I by TAPBPR Is Driven by a Negative Allostery Release Cycle.” Nature Chemical Biology 14 (8): 811-20. https://doi.org/10.1038/s41589-018-0096-2.
Overall, Sarah A., Jugmohit S. Toor, Stephanie Hao, Mark Yarmarkovich, Sara M. O'Rourke, Giora I. Morozov, Son Nguyen, et al. 2020. “High Throughput PMHC-I Tetramer Library Production Using Chaperone-Mediated Peptide Exchange.” Nature Communications 11 (1):1909. https://doi.org/10.1038/s41467-020-15710-1.
Rossjohn, Jamie, Stephanie Gras, John J. Miles, Stephen J. Turner, Dale I. Godfrey, and James McCluskey. 2015. “T Cell Antigen Receptor Recognition of Antigen-Presenting Molecules.” Annual Review of Immunology 33: 169-200. https://doi.org/10.1146/annurev-immunol-032414-112334.
Schneider, T D, and R M Stephens. 1990. “Sequence Logos: A New Way to Display Consensus Sequences.” Nucleic Acids Research 18 (20): 6097-6100.
Schumacher, Ton N. M., Marie-Therese Heemels, Jacques J. Neefjes, W. Martin Kast, Cees J. M. Melief, and Hidde L. Ploegh. 1990. “Direct Binding of Peptide to Empty MHC Class I Molecules on Intact Cells and in Vitro.” Cell 62 (3): 563-67. https://doi.org/10.1016/0092-8674(90)90020-F.
Sidney, John, Bjoern Peters, Nicole Frahm, Christian Brander, and Alessandro Sette. 2008. “HLA Class I Supertypes: A Revised and Updated Classification.” BMC Immunology 9 (1): 1. https://doi.org/10.1186/1471-2172-9-1.
Teng, Michelle S., Richard Stephens, Louis Du Pasquier, Tom Freeman, Jonathan A. Lindquist, and John Trowsdale. 2002. “A Human TAPBP (TAPASIN)-Related Gene, TAPBP-R.” European Journal of Immunology 32 (4): 1059-68. https://doi.org/10.1002/1521-4141(200204)32:4<1059::AID-IMMU1059>3.0.CO;2-G.
Vita, Randi, Swapnil Mahajan, James A Overton, Sandeep Kumar Dhanda, Sheridan Martini, Jason R Cantrell, Daniel K Wheeler, Alessandro Sette, and Bjoern Peters. 2019. “The Immune Epitope Database (IEDB): 2018 Update.” Nucleic Acids Research 47 (D1): D339-43. https://doi.org/10.1093/nar/gky1006.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

1. An isolated ligand-exchange protein or a mutant thereof comprising a fragment of TAP-binding protein-related (TAPBPR), said fragment consisting of the TAPBPR luminal domain.

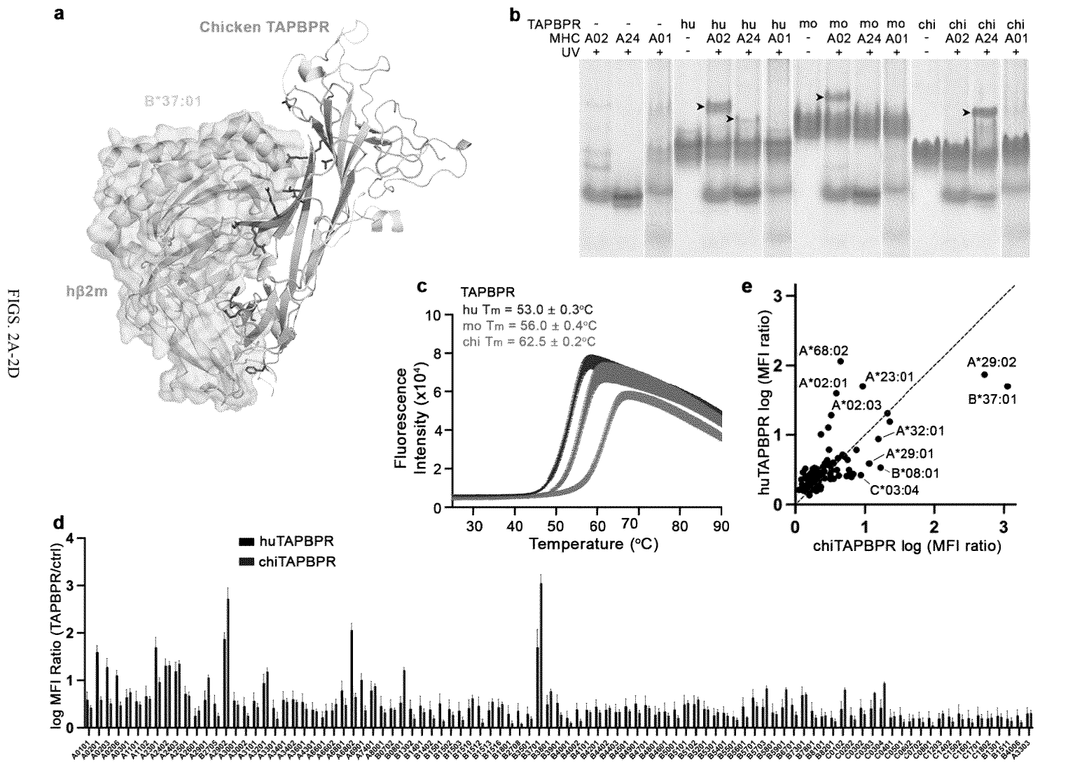

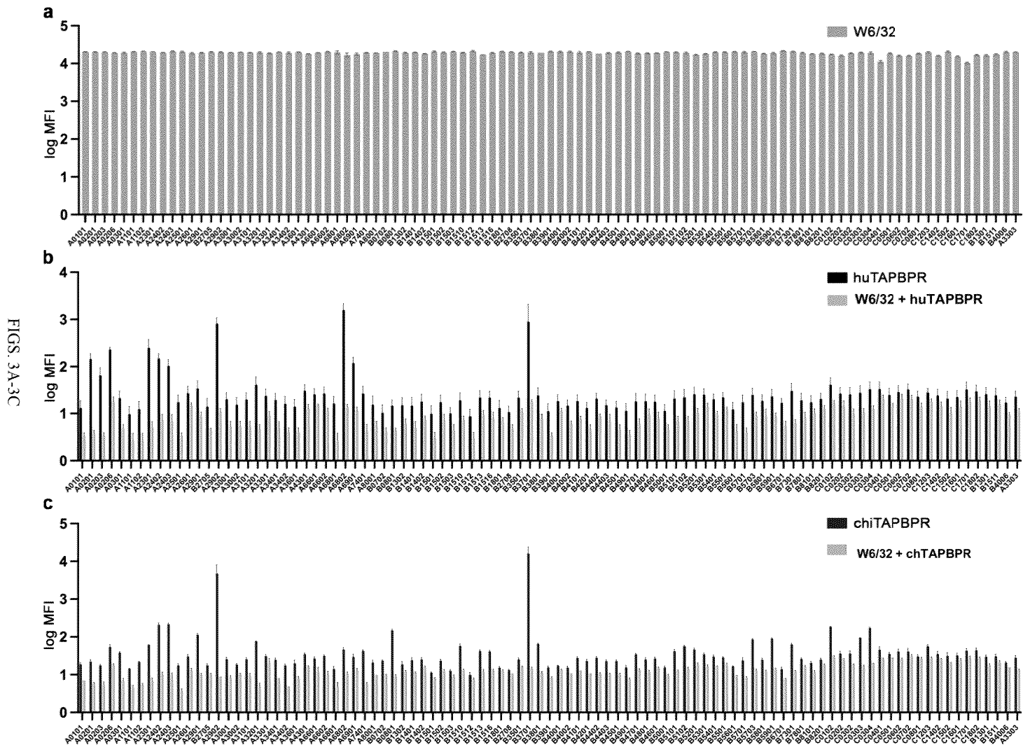

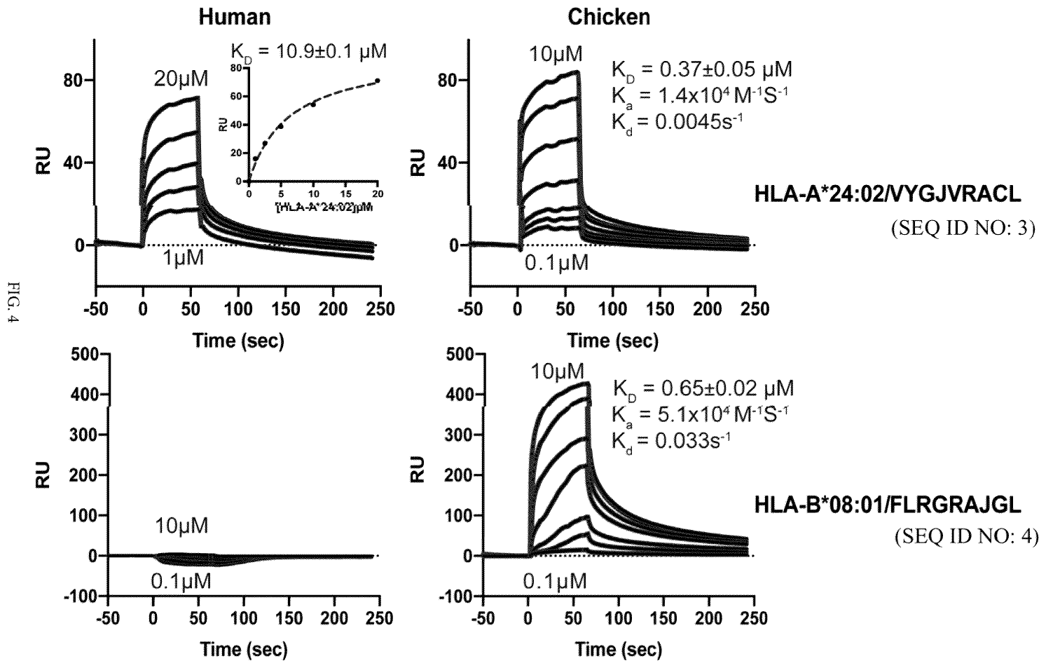

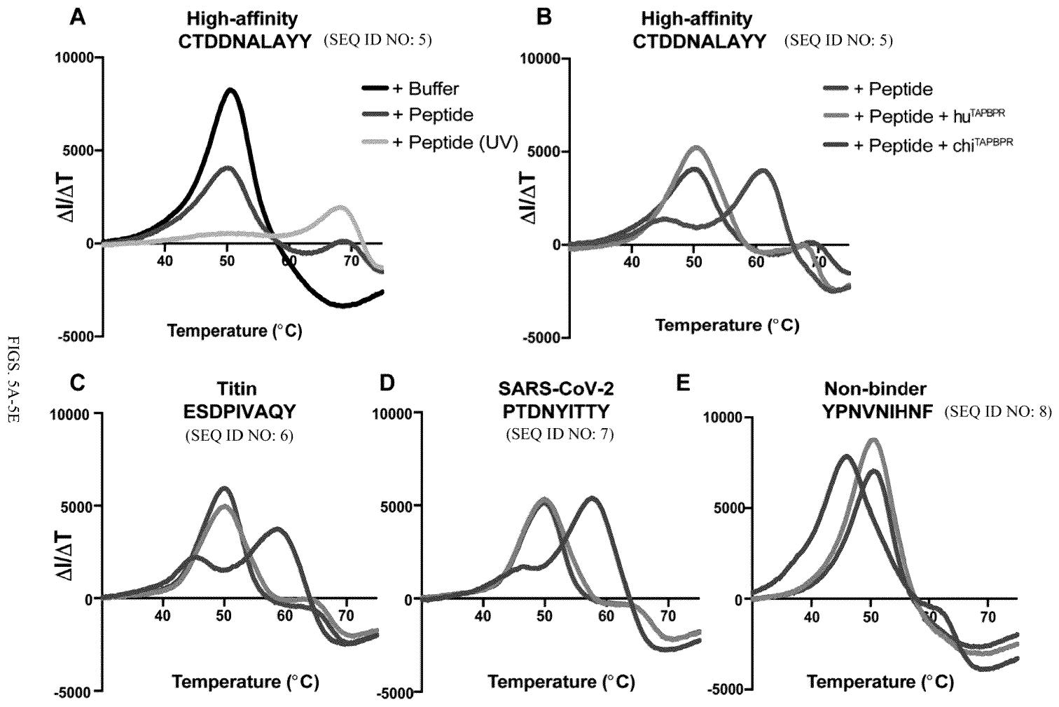

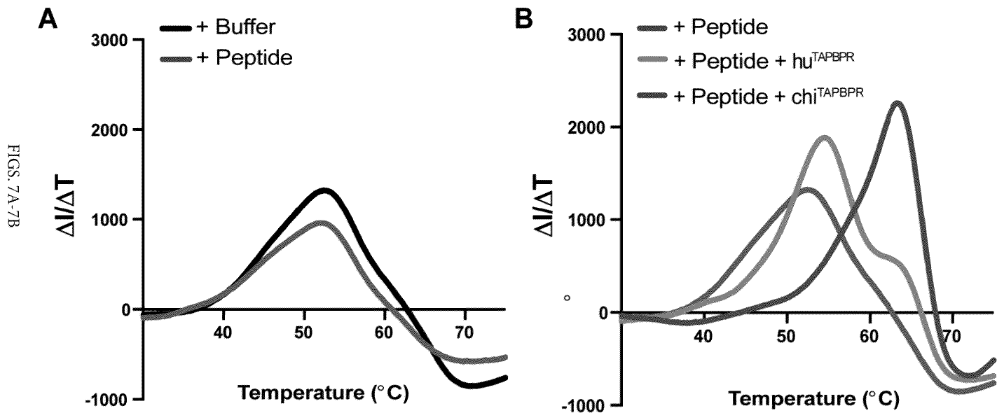

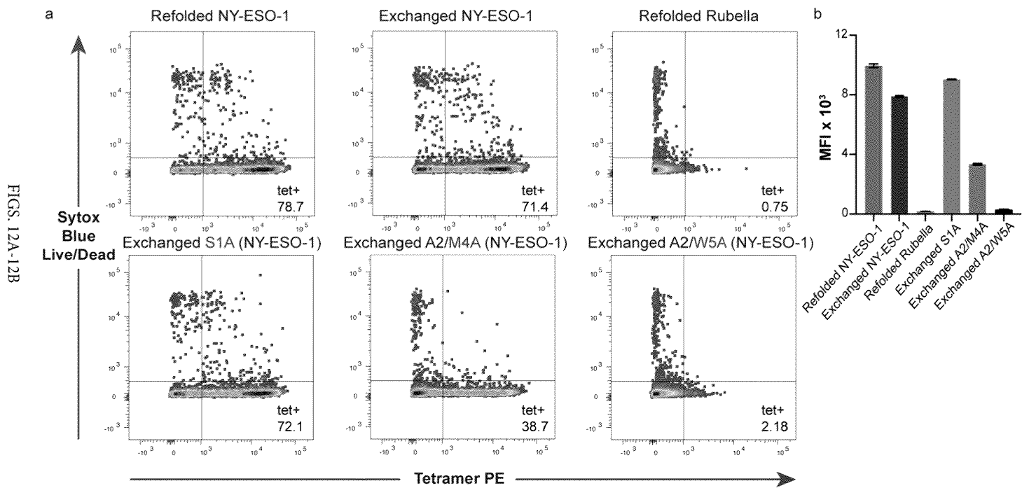

2. An isolated ligand exchange protein according to claim 1, wherein the TAPBPR fragment consists of an amino acid sequence having at least 50% or at least 60% or at least 70% or at least 80% or at least 85% or at least 86% or at least 87% or at least 88% or at least 89% or at least 90% or at least 91% or at least 92% or at least 93% or at least 94% or at least 95% or at least 96% or at least 97% at least or 98% or at least 99% sequence identity to the chicken or human TAPBR sequence of any of FIG. 1 through FIG. 43D.

3. An isolated ligand exchange protein according to claim 1 further comprising a targeting domain.

4. An isolated ligand exchange protein according to claim 3 wherein the targeting domain is linked to the TAPBPR fragment via a linker.

5. An isolated ligand exchange protein according to claim 3 wherein the targeting domain specifically binds to target cells.

6. An isolated ligand exchange protein according to claim 5 wherein the target cells are cancer cells.

7. An isolated ligand exchange protein according to claim 6 wherein the targeting domain specifically binds to a target molecule on the surface of the cancer cells.

8. An isolated ligand exchange protein according to claim 7 wherein the target molecule is ErbB2, PDL1 or CD20.

9. An isolated ligand exchange protein according to claim 5 wherein the target cells are pathogen infected cells.

10. An isolated ligand exchange protein according to claim 9 wherein the pathogen infected cells are cells infected with HIV, CMV, EBV, HPV, Influenza or hepatitis.

11. An isolated ligand exchange protein according to claim 9 wherein the targeting domain specifically binds to a pathogen antigen or a molecule upregulated by pathogen infection on the surface of the infected cells.

12. An isolated ligand exchange protein according to claim 10 wherein the pathogen antigen is an HIV antigen selected from gp120 and gp41; a CMV antigen selected from UL11, UL142, UL9, UL1, UL5, UL16, UL55, UL74, UL75 and UL155 (gL) or an influenza antigen selected from hemagglutinin and neuramidase.

13. An isolated ligand exchange protein according to claim 3 wherein the target cells are antigen presenting cells.

14. An isolated ligand exchange protein according to claim 13 wherein the antigen presenting cells are dendritic cells.

15. An isolated ligand exchange protein according to claim 3 wherein the targeting domain is an antibody molecule, optionally an scFv or nanobody.

16. An isolated ligand exchange protein according to claim 3 wherein the targeting domain is a ligand for a surface receptor on the target cells.

17. An isolated ligand exchange protein according to claim 16 wherein the targeting domain is CD4, CD20, PD1 or an antibody Fc domain.

18. An isolated ligand exchange protein according to claim 3 comprising an amino acid sequence having at least 50% or at least 60% or at least 70% or at least 80% or at least 85% or at least 86% or at least 87% or at least 88% or at least 89% or at least 90% or at least 91% or at least 92% or at least 93% or at least 94% or at least 95% or at least 96% or at least 97% at least or 98% or at least 99% sequence identity to the chicken or human TAPBPR sequence of FIG. 1.

19. A ligand exchange protein comprising;
(i) a fragment of TAP-binding protein-related (TAPBPR), said fragment comprising a TAPBPR luminal domain and a TAPBPR transmembrane domain; or
(ii) a fragment of TAP-binding protein-related (TAPBPR) comprising a TAPBPR luminal domain and a heterologous transmembrane domain.

20. A ligand exchange protein according to claim 19 wherein said fragment lacks the TAPBPR cytoplasmic domain.

21. A ligand exchange protein according to claim 19 further comprising a heterologous cell surface targeting sequence.

22. An isolated ligand exchange protein according to claim 21 wherein the heterologous cell surface targeting sequence comprises the cytoplasmic domain of CD8.

23. An isolated ligand exchange protein according to claim 22 comprising an amino acid sequence having at least 50% or at least 60% or at least 70% or at least 80% or at least 85% or at least 86% or at least 87% or at least 88% or at least 89% or at least 90% or at least 91% or at least 92% or at least 93% or at least 94% or at least 95% or at least 96% or at least 97% at least or 98% or at least 99% sequence identity to the chicken or human TAPBR sequence of FIG. 1 or FIG. 21.

24. A nucleic acid encoding an isolated ligand exchange protein according to any one of claims 1 to 23.

25. A vector comprising a nucleic acid according to claim 24.

26. A mammalian cell comprising a nucleic acid according to claim 24 or a vector according to claim 25.

27. A mammalian cell comprising a ligand exchange protein according to any one of claims 19 to 26 at its surface.

28. A method of increasing the immunogenicity of mammalian cells comprising contacting a population of mammalian cells having surface MHC class I molecules (including classical HLA-A, HLA-B, HLA-C and non-classical HLA-E, HLA-G, HLA-F, MR1, CD1, molecules) with an immunogenic peptide/metabolite/lipid/phospholipid/fatty acid ligand and a ligand exchange protein according to any one of claims 1 to 23 or a mammalian cell according to claim 27, such that the ligand exchange protein loads the immunogenic ligand onto the surface MHC class I molecules of the cells in the population, thereby increasing the immunogenicity of the mammalian cells.

29. The method of claim 28, wherein the ligand-exchange protein is a chicken tapasin.

30. The method of claim 28, wherein the mammalian cell is a human cell.

31. The method of claim 30, wherein the MHC class I molecule is a human MHC class I molecule.

32. The method of claim 28, wherein the tapasin is a human tapasin.

33. The method of claim 28, wherein the mammalian cell is a macaque cell.

34. The method of claim 33, wherein the MHC class I molecule is a macaque MHC class I molecule

35. A method of producing antigen presenting cells to stimulate an immune response in an individual comprising contacting antigen presenting cells in vitro with a ligand exchange protein according to any one of claims 1 to 23 or a mammalian cell according to claim 27 and an immunogenic peptide, such that the ligand exchange protein loads the immunogenic peptide onto surface MHC class I molecules of the antigen presenting cells, wherein the population of antigen presenting cells are previously obtained from the individual.

36. A method according to claim 35 wherein the antigen presenting cells are dendritic cells.

37. A method according to any one of claim 35 further comprising activating a population of T cells with the antigen presenting cells.

38. A method according to claim 35 that is performed in vitro or ex vivo.

39. A method according to any one of claims 28 to 38 further comprising administering the mammalian cells, antigen presenting cells or activated T cells to an individual.

40. A method of increasing the immunogenicity of target cells in an individual comprising administering a ligand exchange protein according to claim 3 to the individual, wherein the ligand exchange protein comprises a targeting domain that binds to target cells in the individual, and administering an immunogenic peptide to the individual, such that the ligand exchange protein loads the immunogenic peptide onto surface MHC class I molecules of the target cells, thereby increasing the immunogenicity of said target cells.

41. A ligand exchange protein according to any one of claims 3 to 18 for use in a method of increasing the immunogenicity of target cells in an individual according to claim 34.

42. A method or protein according to claim 40 wherein the target cells are disease cells.

43. A method or protein for use according to claim 42 wherein the disease cells are cancer cells or pathogen infected cells.

44. A method of stimulating an immune response in an individual comprising administering a ligand exchange protein according to claim 3 to the individual, wherein the targeting domain of the ligand exchange protein binds to antigen presenting cells in the individual, and administering an immunogenic peptide to the individual, such that the ligand exchange protein loads the immunogenic ligand onto surface MHC class I molecules of the antigen presenting cells, such that said antigen presenting cells stimulate an immune response in the individual.

45. A method according to claim 44 wherein the antigen presenting cells are dendritic cells.

46. A method or use or protein for use according to any one of claims 26-45 wherein the immunogenic peptide is a vaccine.

47. A method of producing a MHC class I or MHC class I-like molecule displaying a target ligand comprising contacting an MHC class I or MHC class I-like molecule with a ligand exchange protein of claim 1 and a target ligand, such that the ligand exchange protein loads the target ligand onto the MHC class I molecule, thereby producing an MHC class I or MHC class I-like molecule displaying the target ligand.

48. A method according to claim 47 wherein the MHC class I or MHC class I-like molecule displays an initial ligand that is replaced by the target peptide following contact with the ligand exchange protein.

49. A method according to claim 47 wherein the MHC class I or MHC class I-like molecule is immobilised on a solid support.

50. A method according to claim 47 wherein the MHC class I or MHC class I-like molecule is a sub-unit of a multimer comprising multiple MHC class I molecules.

51. A method according to claim 50 wherein the multimer is a tetramer comprising biotinylated MHC class I or MHC class I-like molecules linked by streptavidin.

52. A method according to claim 47 further comprising contacting the MHC class I or MHC class I-like molecule displaying the target ligand with a population of T cells and determining the binding of the MHC class I or MHC class I-like molecule to T cells in the population.

53. A method for generating a conditional peptide ligand for a HLA allotype which forms a stable complex with a MHC-I molecule comprising:
choosing a high affinity peptide for an HLA allele of interest, wherein the high affinity peptide has a melting temperature greater than 60 degrees centigrade,
introducing a modified unnatural amino acid near the C-terminus of the high affinity peptide, thereby generating a placeholder peptide,
performing a preliminary refolding reaction with the placeholder peptide, wherein the placeholder peptide has a melting temperature of about 50 degrees centigrade,
dislodging the conditional peptide ligand when interacting with a chaperone with a high affinity peptide of interest such that the high affinity peptide of interest binds to an empty MHC groove, and
selecting a top placeholder peptide to perform a chaperone-mediated exchange with the high affinity peptide of interest,
wherein the top placeholder peptide is the conditional peptide ligand.

54. The method of claim 53, wherein the HLA allele of interest is selected from the Immune Epitope Database.

55. The method of claim 53, wherein the modified unnatural amino acid is introduced at positions 8, 7 or 6 of the high affinity peptide.

56. The method of claim 53, wherein the modified unnatural amino acid is a β-amino acid such as 0-phenylalanine.

57. The method of claim 53, wherein the chaperone is tapasin or TAPBPR.

58. The method of claim 53, wherein peptide binding is analyzed by differential scanning fluorimetry (DSF) or fluorescence polarization (FP).

59. A method of generating a tetramer library comprising repeating the method of claim 53, wherein the library comprises a plurality of high affinity peptides of interest.

60. The high affinity peptide of interest generated by the method of claim 53.

61. The isolated ligand-exchange protein of any one of the preceding claims wherein the protein is a chaperone.

62. Use of the isolated ligand-exchange chaperone of claim 61, wherein the stabilization or recognition of empty MHC-I.

63. A method for use according to claim 62, wherein the stabilization or recognition of empty MHC-I optionally promotes ligand exchange.


SEQUENCE LISTING (113 sequences)

SEQ ID NO: 1 - protein, 384 aa, Homo sapiens
KPHPAEGQWRAVDVVLDCFLVKDGAHRGALASSEDRARASLVLKQVPVLDDGSLEDFTDFQGGTLAQDDPPIIFEASVDLVQIPQAEALLHADCSGKEVTCEISRYFLQMTETTVKTAAWFMANVQVSGGGPSISLVMKTPRVAKNEVLWHPTLNLPLSPQGTVRTAVEFQVMTQTQSLSFLLGSSASLDCGFSMAPGLDLISVEWRLQHKGRGQLVYSWTAGQGQAVRKGATLEPAQLGMARDASLTLPGLTIQDEGTYICQITTSLYRAQQIIQLNIQASPKVRLSLANEALLPTLICDIAGYYPLDVVVTWTREELGGSPAQVSGASFSSLRQSVAGTYSISSSLTAEPGSAGATYTCQVTHISLEEPLGASTQVVPPERR

SEQ ID NO: 2 - protein, 398 aa, Gallus gallus
VEGLTPVPELRRVDVVLGCSYVWEGGLSRAFGGSEHPATLVLRGLSVTDDGTLGDVTDYEIPQADHSSSPPIIFEASEQLVSIPYAEALLHVDASGEEVSCELSPYSFQQEGNGLCSASWFLATIRLSSGISIVLLLRGPSCSSQKEGHDVTLHPKLRIPMSKEGTLLTTVEFQSSSNNTSLRTRLGSSITLDCHFALAPSFLLSSLEWRRQHRGSGRSLFRYRVGNAGLTAQPKVHVDVEQLLGNGDASLTLQEATVNDEGTYICLVSTAQHQVQHNIQLLVSEPPRVRVFPTEASLKRDETITLTCNIAGYYPLDISVSWTQKTPEDEVEISPSNTRFSSHRQSQDGTYSINSYLSVNLATAQAPATYTCHVSHVALEAPISISTHLKAPEHTELE

SEQ ID NO: 3 - protein, 9 aa, synthetic construct
VYGJVRACL

SEQ ID NO: 4 - protein, 9 aa, synthetic construct
FLRGRAJGL

SEQ ID NO: 5 - protein, 10 aa, Betacoronavirus 1
CTDDNALAYY

SEQ ID NO: 6 - protein, 9 aa, synthetic construct
ESDPIVAQY

SEQ ID NO: 7 - protein, 9 aa, Betacoronavirus 1
PTDNYITTY

SEQ ID NO: 8 - protein, 9 aa, synthetic construct
YPNVNIHNF

SEQ ID NO: 9 - protein, 9 aa, synthetic construct
  VARIANT 6: note = J=3-amino-3-(2-nitrophenyl)-propionic acid)
STAPGJLEY

SEQ ID NO: 10 - protein, 11 aa, synthetic construct
RPHERNGFTVL

SEQ ID NO: 11 - protein, 9 aa, synthetic construct
FMKQMNDAL

SEQ ID NO: 12 - protein, 9 aa, synthetic construct
  MOD_RES 8: note = DL-beta-Phenylalanine
KILGFVFFV

SEQ ID NO: 13 - protein, 9 aa, synthetic construct
GILGFVFTL

SEQ ID NO: 14 - protein, 9 aa, synthetic construct
  MOD_RES 6: note = DL-beta-Phenylalanine
STAPGFLEY

SEQ ID NO: 15 - protein, 9 aa, synthetic construct
  MOD_RES 4: note = DL-beta-Phenylalanine
VYGFVRACL

SEQ ID NO: 16 - protein, 9 aa, synthetic construct
  MOD_RES 7: note = DL-beta-Phenylalanine
FLRGRAFGL

SEQ ID NO: 17 - protein, 9 aa, synthetic construct
  MOD_RES 7: note = DL-beta-Phenylalanine
FEDLRVFSF

SEQ ID NO: 18 - protein, 9 aa, synthetic construct
  MOD_RES 6: note = DL-beta-Phenylalanine
LNPSVFATL

SEQ ID NO: 19 - protein, 9 aa, Homo sapiens
STAPGVLEY

SEQ ID NO: 20 - protein, 9 aa, synthetic construct
VYGFVRACL

SEQ ID NO: 21 - protein, 9 aa, synthetic construct
FLRGRAYGL

SEQ ID NO: 22 - protein, 9 aa, synthetic construct
FEDLRVLSF

SEQ ID NO: 23 - protein, 9 aa, synthetic construct
LNPSVAATL

SEQ ID NO: 24 - protein, 8 aa, Homo sapiens
LFGYPVYV

SEQ ID NO: 25 - protein, 9 aa, synthetic construct
  VARIANT 8: note = J=3-amino-3-(2-nitrophenyl)-propionic acid
KILGFVFJV

SEQ ID NO: 26 - protein, 9 aa, Homo sapiens
LLFGYPVYV

SEQ ID NO: 27 - protein, 9 aa, synthetic construct
  CROSSLNK 1: note = 5-Carboxytetramethylrhodamine
KLFGYPVYV

SEQ ID NO: 28 - protein, 9 aa, Homo sapiens
SLLMWITQV

SEQ ID NO: 29 - protein, 11 aa, Rubivirus rubellae
ALLDRLRGVYA

SEQ ID NO: 30 - protein, 9 aa, synthetic construct
ALLMWITQV

SEQ ID NO: 31 - protein, 9 aa, synthetic construct
SLLAWITQV

SEQ ID NO: 32 - protein, 9 aa, synthetic construct
SLLMAITQV

SEQ ID NO: 33 - protein, 9 aa, synthetic construct
KILGFVFTV

SEQ ID NO: 34 - protein, 9 aa, synthetic construct
  CROSSLNK 1: note = 5-Fluorescein Isothiocyanate
KSDPIVAQY

SEQ ID NO: 35 - protein, 13 aa, synthetic construct
  CROSSLNK 1: note = 5-Fluorescein Isothiocyanate
FITCKLIETYFSK

SEQ ID NO: 36 - protein, 9 aa, synthetic construct
  CROSSLNK 1: note = 5-Carboxytetramethylrhodamine
KYNPIRTTF

SEQ ID NO: 37 - protein, 9 aa, synthetic construct
  VARIANT 8: note = DL-beta-Phenylalanine
KLIETYFFK

SEQ ID NO: 38 - protein, 9 aa, synthetic construct
  VARIANT 8: note = DL-beta-Phenylalanine
KTFPPTEFK

SEQ ID NO: 39 - protein, 10 aa, synthetic construct
  VARIANT 9: note = DL-beta-Phenylalanine
ETAGIGILFV

SEQ ID NO: 40 - protein, 9 aa, synthetic construct
KLIETYFSK

SEQ ID NO: 41 - protein, 9 aa, SARS-CoV-2
KTFPPTEPK

SEQ ID NO: 42 - protein, 10 aa, Homo sapiens
ETAGIGILTV

SEQ ID NO: 43 - protein, 10 aa, Homo sapiens
EVTCEISRYF

SEQ ID NO: 44 - protein, 11 aa, Homo sapiens
VEWRLQHKGRG

SEQ ID NO: 45 - protein, 11 aa, Homo sapiens
CQITTSLYRAQ

SEQ ID NO: 46 - protein, 10 aa, Pan troglodytes
EVSCELSPYS

SEQ ID NO: 47 - protein, 11 aa, Pan troglodytes
LEWRRQHRGSG

SEQ ID NO: 48 - protein, 11 aa, Pan troglodytes
CLVSTAQHQVQ

SEQ ID NO: 49 - protein, 11 aa, Homo sapiens
LEWRRQHLGKG

SEQ ID NO: 50 - protein, 11 aa, Homo sapiens
ATIHLPYLQGQ

SEQ ID NO: 51 - protein, 9 aa, Homo sapiens
SDLSFSKDW

SEQ ID NO: 52 - protein, 9 aa, Mus musculus
SDMSFSKDW

SEQ ID NO: 53 - protein, 9 aa, Pan troglodytes
SDLSFSKDW

SEQ ID NO: 54 - protein, 9 aa, Rattus norvegicus
SDLSFSKDW

SEQ ID NO: 55 - protein, 327 aa, Homo sapiens
FTDFQGGTLAQDDPPIIFEASVDLVQIPQAEALLHADCSGKEVTCEISRYFLQMTETTVKTAAWFMANMQVSGGGPSISLVMKTPRVTKNEALWHPTLNLPLSPQGTVRTAVEFQVMTQTQSLSFLLGSSASLDCGFSMAPGLDLISVEWRLQHKGRGQLVYSWTAGQGQVRKGATLEPAQLGMARDASLTLPGLTIQDEGTYICQITTSLYRAQQIIQLNIQASPKVRLSLANEALLPTLICDIAGYYPLDVVVTWTREELGGSPAQVSGASFSSLRQSVAGTYSISSSLTAEPGSAGATYTCQVTHISLEEPLGASTQVVPPERR

SEQ ID NO: 56 - protein, 390 aa, Mus musculus
DPPTAPTTAERQRQPTDIILDCFLVTEDRHRGAFASSGDRERALLVLKQVPVLDDGSLEGITDFQGSTETKQDSPVIFEASVDLVQIPQAEALLHADCSGKAVTCEISKYFLQARQEATFEKAHWFISNMQVSRGGPSVSMVMKTLRDAEVGAVRHPTLNLPLSAQGTVKTQVEFQVTSETQTLNHLLGSSVSLHCSFSMAPGLDLTGVEWRLQHKGSGQLVYSWKTGQGQAKRKGATLEPEELLRAGNASLTLPNLTLKDEGNYICQISTSLYQAQQIMPLNILAPPKIQLHLANKDPLPSLVCSIAGYYPLDVGVTWIREELGGIPAQVSGASFSSLRQSTMGTYSISSTVMADPGPTGATYTCQVAHVSLEEPLTTSMRVLPNPEQR

SEQ ID NO: 57 - protein, 398 aa, Gallus gallus
VEGLTPVPELRRVDVVLGCSYVWEGGLSRAFGGSEHPATLVLRGLSVTDDGTLGDVTDYEIPQADHSSSPPIIFEASEQLVSIPYAEALLHVDCSGEEVSCELSPYSFQQEGNGLCSASWFLATIRLSSGISIVLLLRGPSCSSQKEGHDVTLHPKLRIPMSKEGTLLTTVEFQSSSNNTSLRTRLGSSITLDCHFALAPSFLLSSLEWRRQHRGSGRSLFRYRVGNAGLTAQPKVHVDVKQLLGNGDASLTLQEATVNDEGTYICLVSTAQHQVQHNIQLLVSEPPRVRVFPTEASLKRDETITLTCNIAGYYPLDISVSWTQKTPEDEVEISPSNTRFSSHRQSQDGTYSINSYLSVNLATAQAPATYTCHVSHVALEAPISISTHLKAPEHTELE

SEQ ID NO: 58 - protein, 9 aa, synthetic construct
  MOD_RES 8: note = J=3-amino-3-(2-nitrophenyl)-propionic acid
AIFQSSMJK

SEQ ID NO: 59 - protein, 9 aa, synthetic construct
  MOD_RES 8: note = J=3-amino-3-(2-nitrophenyl)-propionic acid
VFAQVKQJY

SEQ ID NO: 60 - protein, 9 aa, synthetic construct
  MOD_RES 8: note = J=3-amino-3-(2-nitrophenyl)-propionic acid
SVYDFFVJL

SEQ ID NO: 61 - protein, 9 aa, synthetic construct
  MOD_RES 8: note = J=3-amino-3-(2-nitrophenyl)-propionic acid
KLIETYFJK

SEQ ID NO: 62 - protein, 9 aa, synthetic construct
  MOD_RES 4: note = J=3-amino-3-(2-nitrophenyl)-propionic acid
VYGJVRACL

SEQ ID NO: 63 - protein, 9 aa, synthetic construct
  MOD_RES 7: note = J=3-amino-3-(2-nitrophenyl)-propionic acid
FEDLRVJSF

SEQ ID NO: 64 - protein, 9 aa, synthetic construct
  MOD_RES 5: note = J=3-amino-3-(2-nitrophenyl)-propionic acid
VMAPJTLVL

SEQ ID NO: 65 - protein, 9 aa, synthetic construct
  MOD_RES 5: note = J=3-amino-3-(2-nitrophenyl)-propionic acid
VMAPJTLVL

SEQ ID NO: 66 - protein, 9 aa, synthetic construct
  VARIANT 1: note = DL-beta-Phenylalanine
FILGFVFTV

SEQ ID NO: 67 - protein, 9 aa, synthetic construct
  VARIANT 3: note = DL-beta-Phenylalanine
KIFGFVFTV

SEQ ID NO: 68 - protein, 9 aa, synthetic construct
  VARIANT 4: note = DL-beta-Phenylalanine
KILFFVFTV

SEQ ID NO: 69 - protein, 9 aa, synthetic construct
  VARIANT 5: note = DL-beta-Phenylalanine
KILGFVFTV

SEQ ID NO: 70 - protein, 9 aa, synthetic construct
  VARIANT 6: note = DL-beta-Phenylalanine
KILGFFFTV

SEQ ID NO: 71 - protein, 9 aa, synthetic construct
  MOD_RES 7: note = DL-beta-Phenylalanine
KILGFVFTV

SEQ ID NO: 72 - protein, 9 aa, synthetic construct
  MOD_RES 8: note = DL-beta-Phenylalanine
KLIETYFFK

SEQ ID NO: 73 - protein, 9 aa, synthetic construct
  MOD_RES 8: note = DL-beta-Phenylalanine
KILGFVFFV

SEQ ID NO: 74 - protein, 9 aa, synthetic construct
  MOD_RES 8: note = DL-beta-Phenylalanine
KILGFVFFV

SEQ ID NO: 75 - protein, 9 aa, synthetic construct
  MOD_RES 8: note = DL-beta-Phenylalanine
KILGFVFFV

SEQ ID NO: 76 - protein, 9 aa, synthetic construct
  MOD_RES 8: note = DL-beta-Phenylalanine
KILGFVFFV

SEQ ID NO: 77 - protein, 9 aa, synthetic construct
QYNPIRTTF

SEQ ID NO: 78 - protein, 9 aa, synthetic construct
  CROSSLNK 1: note = 5-Fluorescein Isothiocyanate
KLRGRAYGL

SEQ ID NO: 79 - protein, 9 aa, synthetic construct
ALWMRLLPL

SEQ ID NO: 80 - protein, 9 aa, synthetic construct
MLYQHLLPL

SEQ ID NO: 81 - protein, 9 aa, synthetic construct
AIFQSSMTK

SEQ ID NO: 82 - protein, 9 aa, human cytomegalovirus
NLVPMVATV

SEQ ID NO: 83 - protein, 10 aa, synthetic construct
ILDTAGQEEY

SEQ ID NO: 84 - protein, 9 aa, synthetic construct
  CROSSLNK 1: note = 5-Fluorescein Isothiocyanate
KEDLRVSSF

SEQ ID NO: 85 - protein, 9 aa, synthetic construct
  MOD_RES 7: note = J=3-amino-3-(2-nitrophenyl)-propionic acid
  CROSSLNK 1: note = 5-Fluorescein Isothiocyanate
KLRGRAJGL

SEQ ID NO: 86 - protein, 9 aa, synthetic construct
GPRKPIKCW

SEQ ID NO: 87 - protein, 9 aa, Influenza virus
GILGFVFTL

SEQ ID NO: 88 - protein, 9 aa, Influenza virus
AIMDKNIIL

SEQ ID NO: 89 - protein, 9 aa, Human immunodeficiency virus 1 and 2
ILKEPVHGV

SEQ ID NO: 90 - protein, 9 aa, Human immunodeficiency virus 1 and 2
SLYNTVATL

SEQ ID NO: 91 - protein, 9 aa, Human immunodeficiency virus 1 and 2
KLTPLCVTL

SEQ ID NO: 92 - protein, 10 aa, Hepatitis B virus
FLPSDFFPSV

SEQ ID NO: 93 - protein, 9 aa, Hepatitis B virus
WLSLLVPFV

SEQ ID NO: 94 - protein, 9 aa, human cytomegalovirus
VLEETSVML

SEQ ID NO: 95 - protein, 9 aa, Epstein Barr virus
YLLEMLWRL

SEQ ID NO: 96 - protein, 9 aa, Epstein Barr virus
CLGGLLTMV

SEQ ID NO: 97 - protein, 9 aa, Varicella-zoster virus
ILIEGIFFV

SEQ ID NO: 98 - protein, 10 aa, Measles morbillivirus
ILPGQDLQYV

SEQ ID NO: 99 - protein, 10 aa, Zika virus
FLVEDHGFGV

SEQ ID NO: 100 - protein, 9 aa, Zika virus
KSYFVRAAK

SEQ ID NO: 101 - protein, 9 aa, synthetic construct
  MOD_RES 8: note = J=3-amino-3-(2-nitrophenyl)-propionic acid
SVYDFFVJL

SEQ ID NO: 102 - protein, 9 aa, synthetic construct
  MOD_RES 8: note = J=3-amino-3-(2-nitrophenyl)-propionic acid
KLIETYFJK

SEQ ID NO: 103 - protein, 9 aa, synthetic construct
  VARIANT 7: note = DL-beta-Phenylalanine
KLIETYFFK

SEQ ID NO: 104 - protein, 24 aa, Homo sapiens
YNYQGDNETAAMQIKRETTELVEW

SEQ ID NO: 105 - protein, 24 aa, Homo sapiens
YNYQGDNETAAMQIQRETTELVEW

SEQ ID NO: 106 - protein, 24 aa, Homo sapiens
YNYQGDKETAAMQTKHETTELVEW

SEQ ID NO: 107 - protein, 24 aa, Homo sapiens
YNYQGDKETAAMQIKRETTELVEW

SEQ ID NO: 108 - protein, 24 aa, Homo sapiens
YNHQGDNETAATQIQRETTELVEW

SEQ ID NO: 109 - protein, 24 aa, Homo sapiens
YNYQGDNETAATQIQRETTELVEW

SEQ ID NO: 110 - protein, 24 aa, Homo sapiens
YNYQGDNETAVTQIEQNTTELVEW

SEQ ID NO: 111 - protein, 24 aa, Homo sapiens
YNYQGDNETAATQIKRETVELVEW

SEQ ID NO: 112 - protein, 8 aa, synthetic construct
  VARIANT 7: note = DL-beta-Phenylalanine
LILRWEFD

SEQ ID NO: 113 - protein, 15 aa, Homo sapiens
LHHILDAQKMVWNHR